(19) United States
(12) Patent Application Publication
(10) Pub. No.: US 2024/0196639 A1
(43) Pub. Date: Jun. 13, 2024

(54) LIGHT EMITTING ELEMENT AND AMINE COMPOUND FOR THE SAME

(71) Applicant: Samsung Display Co., Ltd., Yongin-si (KR)

(72) Inventors: MINJI KIM, Yongin-si (KR); HANKYU PAK, Yongin-si (KR); HYUNBIN PARK, Yongin-si (KR); BYEONGWOOK YOO, Yongin-si (KR); Jeong Min LEE, Yongin-si (KR); SOHEE JO, Yongin-si (KR)

(21) Appl. No.: 18/229,115

(22) Filed: Aug. 1, 2023

(30) Foreign Application Priority Data
Oct. 25, 2022 (KR) 10-2022-0138723

(51) Int. Cl. H10K 50/15; H10K 85/60

(52) U.S. Cl. CPC H10K 50/15; H10K 85/633

(57) ABSTRACT

A light emitting element includes a first electrode, a second electrode on the first electrode, an emission layer between the first electrode and the second electrode, and a hole transport region between the first electrode and the emission layer and including an amine compound represented by Formula 1, where at least one selected from R₁₂, R₂₃, and R₃₄ is a substituted or unsubstituted methyl group.

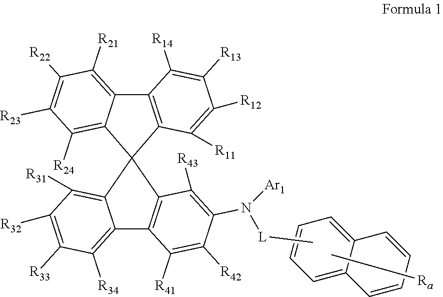

### CROSS-REFERENCE TO RELATED APPLICATIONS

This application claims priority to and the benefit of Korean Patent Application No. 10-2022-0138723, filed on Oct. 25, 2022, in the Korean Intellectual Property Office, the entire content of which is hereby incorporated by reference.

### BACKGROUND

### 1. Field

One or more embodiments of the present disclosure relate to a light emitting element and an amine compound utilized in the light emitting element.

### 2. Description of the Related Art

Recently, the development of an organic electroluminescence display device as an image display device is being actively conducted. The organic electroluminescence display device is a display device with a self-luminescent-type or kind light emitting element in which holes and electrons separately injected from a first electrode and a second electrode recombine in an emission layer so that a light emitting material in the emission layer emits light to achieve display (e.g., of an image).

In the application of a light emitting element to a display device, the decrease of a driving voltage and the increase of luminance, efficiency and lifetime are required and/or desired, and development on materials for a light emitting element, stably achieving the requirement(s) is being consistently required and/or pursued.

### SUMMARY

One or more aspects of embodiments of the present disclosure are directed toward a light emitting element showing a reduced driving voltage, improved luminance and efficiency, and increased lifetime.

One or more aspects of embodiments of the present disclosure are directed towards an amine compound which is a material for a light emitting element having a reduced driving voltage, improved luminance and efficiency, and increased lifetime.

Additional aspects will be set forth in part in the description which follows and, in part, will be apparent from the description, or may be learned by practice of the presented embodiments.

One or more embodiments of the present disclosure provide a light emitting element including a first electrode, a second electrode on the first electrode, an emission layer between the first electrode and the second electrode, and a hole transport region between the first electrode and the emission layer and including an amine compound represented by Formula 1.

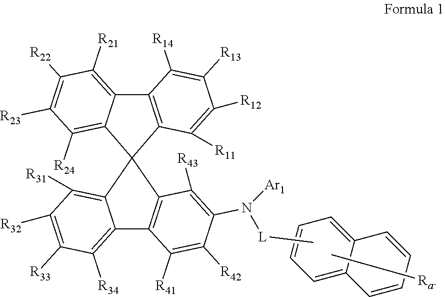

In Formula 1, at least one selected from among R₁₁ to R₁₄, R₂₁ to R₂₄, R₃₁ to R₃₄, and R₄₁ to R₄₃ may be a substituted or unsubstituted methyl group, and the remainder may each independently be hydrogen, deuterium, a halogen, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, Ar₁ may be a substituted or unsubstituted aryl group of 6 to 30 ring-forming carbon atoms, or a substituted or unsubstituted heteroaryl group of 2 to 30 ring-forming carbon atoms, L may be a direct linkage, a substituted or unsubstituted arylene group of 6 to 30 ring-forming carbon atoms, or a substituted or unsubstituted heteroarylene group of 2 to 30 ring-forming carbon atoms, and Rₐ may hydrogen, deuterium, a halogen, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In one or more embodiments, in Formula 1, at least one selected from among R₁₂, R₂₃, and R₃₄ may be an unsubstituted methyl group.

In one or more embodiments, Formula 1 may be represented by any one selected from among Formula 1-1 to Formula 1-4.

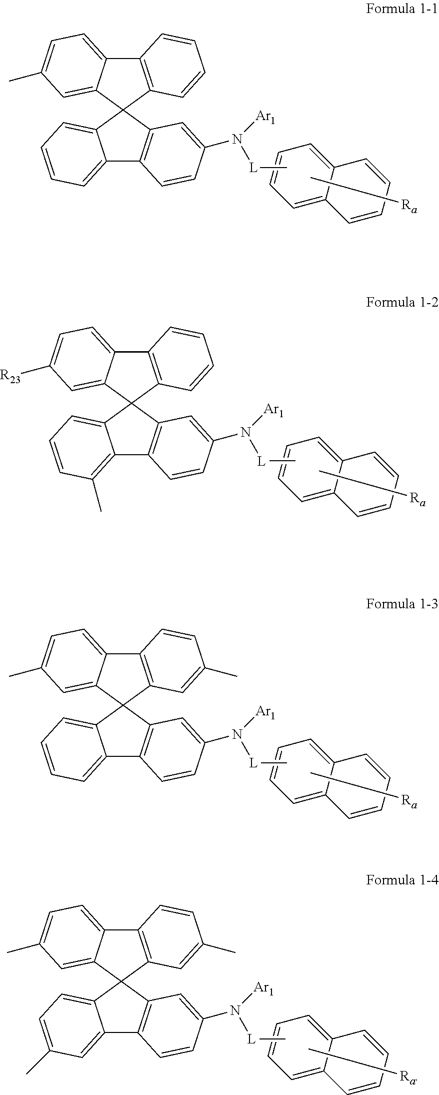

In Formula 1-1 to Formula 1-4, R₂₃, Ar₁, L, and Rₐ may each independently be the same as defined in Formula 1.

In one or more embodiments, in Formula 1-2, R₂₃ may be hydrogen, an unsubstituted methyl group, an unsubstituted phenyl group, or an unsubstituted t-butyl group.

In one or more embodiments, Formula 1 may be represented by Formula 2.

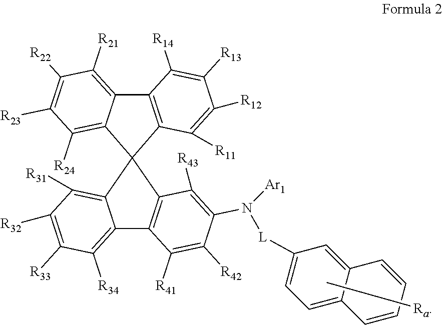

In Formula 2, R₁₁ to R₁₄, R₂₁ to R₂₄, R₃₁ to R₃₄, R₄₁ to R₄₃, Ar₁, L, and Rₐ may each independently be the same as defined in Formula 1.

In one or more embodiments, Formula 2 may be represented by Formula 3-1 or Formula 3-2.

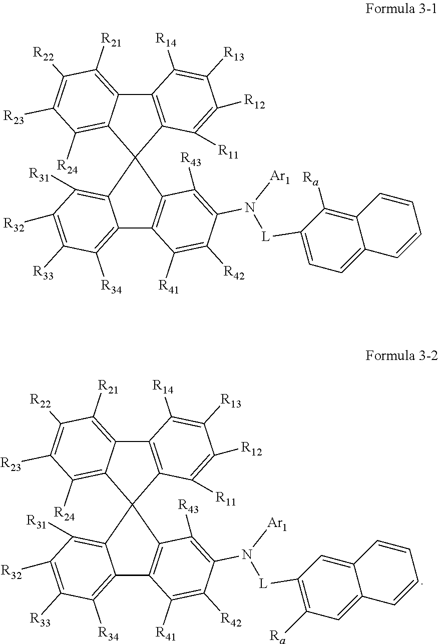

In Formula 3-1 and Formula 3-2, R₁₁ to R₁₄, R₂₁ to R₂₄, R₃₁ to R₃₄, R₄₁ to R₄₃, Ar₁, L, and Rₐ may each independently be the same as defined in Formula 1.

In one or more embodiments, in Formula 1, Rₐ may be a substituted or unsubstituted phenyl group, or a substituted or unsubstituted methyl group.

In one or more embodiments, Formula 1 may be represented by Formula 4-1 or Formula 4-2.

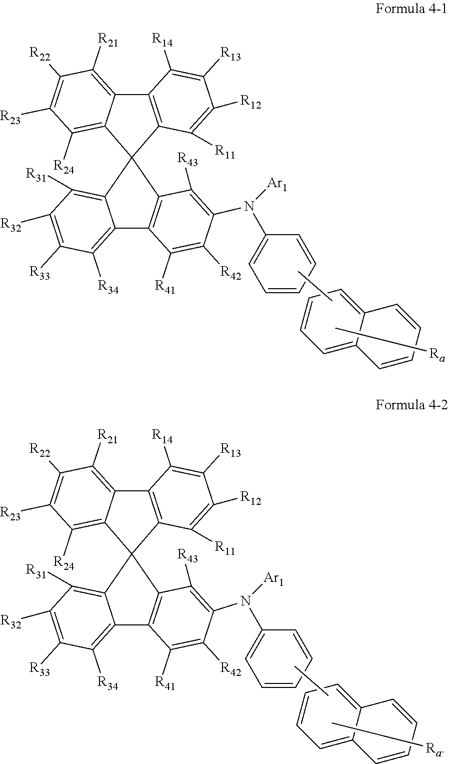

In Formula 4-1 and Formula 4-2, R₁₁ to R₁₄, R₂₁ to R₂₄, R₃₁ to R₃₄, R₄₁ to R₄₃, Ar₁, and Rₐ may each independently be the same as defined in Formula 1.

In one or more embodiments, Formula 4-1 may be represented by Formula 5.

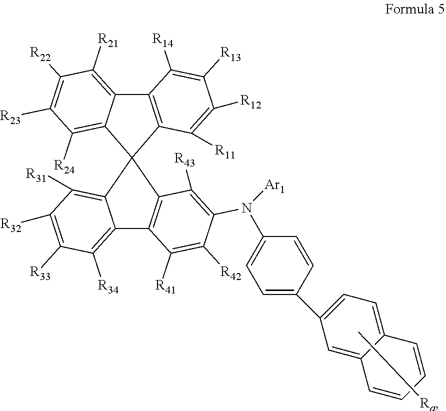

In Formula 5, R₁₁ to R₁₄, R₂₁ to R₂₄, R₃₁ to R₃₄, R₄₁ to R₄₃, Ar₁, and Rₐ may each independently be the same as defined in Formula 1.

In one or more embodiments, the amine compound represented by Formula 1 may be a monoamine compound.

In one or more embodiments, the hole transport region may include a hole injection layer on the first electrode, and a hole transport layer between the hole injection layer and the emission layer, and the hole transport layer may include the amine compound represented by Formula 1.

In one or more embodiments, the emission layer may be to emit blue light.

In one or more embodiments, the amine compound may include at least one selected from among compounds in Compound Group 1.

According to one or more embodiments of the present disclosure, there is provided an amine compound represented by Formula 1.

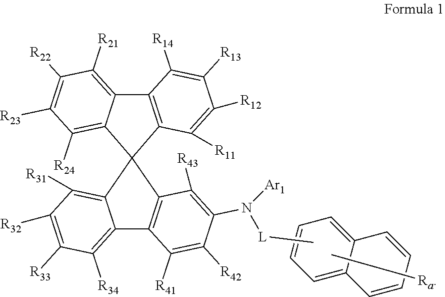

In Formula 1, at least one selected from among R₁₁ to R₁₄, R₂₁ to R₂₄, R₃₁ to R₃₄, and R₄₁ to R₄₃ may be a substituted or unsubstituted methyl group, and the remainder may each independently be hydrogen, deuterium, a halogen, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, Ar₁ may be a substituted or unsubstituted aryl group of 6 to 30 ring-forming carbon atoms, or a substituted or unsubstituted heteroaryl group of 2 to 30 ring-forming carbon atoms, L may be a direct linkage, a substituted or unsubstituted arylene group of 6 to 30 ring-forming carbon atoms, or a substituted or unsubstituted heteroarylene group of 2 to 30 ring-forming carbon atoms, and Rₐ may be hydrogen, deuterium, a halogen, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In one or more embodiments, in Formula 1, at least one selected from among R₁₂, R₂₃, and R₃₄ may be an unsubstituted methyl group.

In one or more embodiments, Formula 1 may be represented by any one selected from among Formula 1-1 to Formula 1-4.

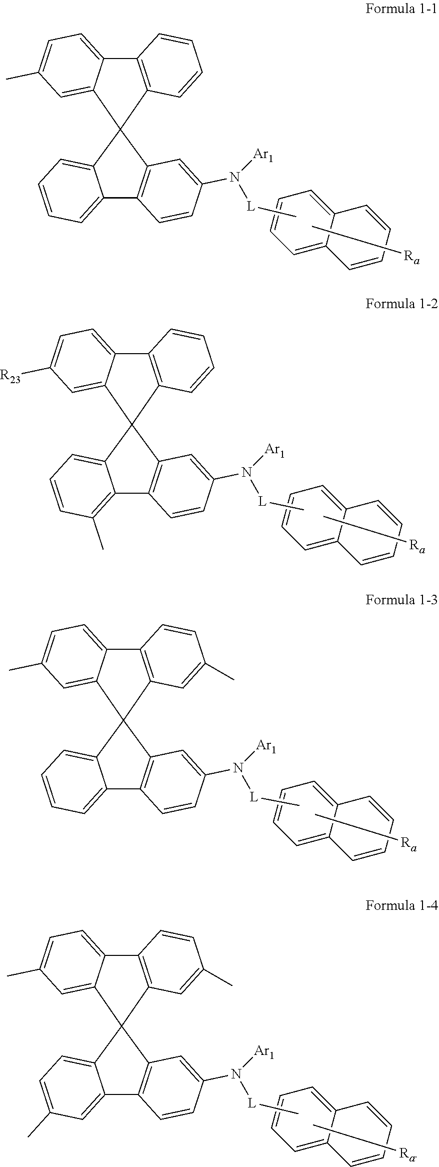

In Formula 1-1 to Formula 1-4, R₂₃, Ar₁, L, and Rₐ may each independently be the same as defined in Formula 1.

In one or more embodiments, in Formula 1-2, R₂₃ may be any one selected from among hydrogen, an unsubstituted methyl group, an unsubstituted phenyl group, and an unsubstituted t-butyl group.

In one or more embodiments, Formula 1 may be represented by Formula 2.

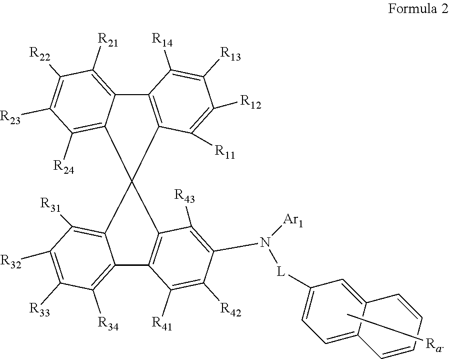

In Formula 2, R₁₁ to R₁₄, R₂₁ to R₂₄, R₃₁ to R₃₄, R₄₁ to R₄₃, Ar₁, L, and Rₐ may each independently be the same as defined in Formula 1.

In one or more embodiments, Formula 2 may be represented by Formula 3-1 or Formula 3-2.

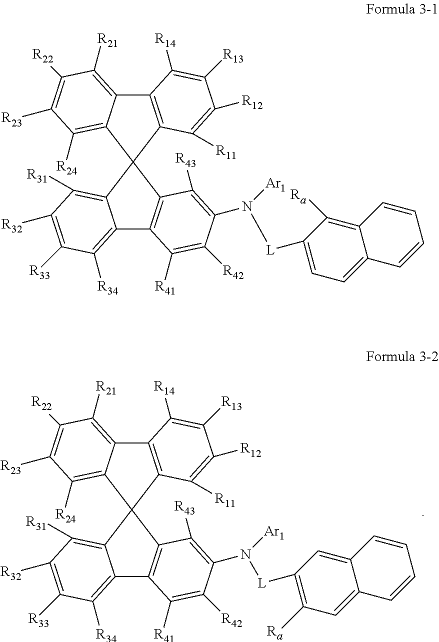

In Formula 3-1 and Formula 3-2, R₁₁ to R₁₄, R₂₁ to R₂₄, R₃₁ to R₃₄, R₄₁ to R₄₃, Ar₁, L, and Rₐ may each independently be the same as defined in Formula 1.

In one or more embodiments, in Formula 1, Rₐ may be a substituted or unsubstituted phenyl group, or a substituted or unsubstituted methyl group.

In one or more embodiments, Formula 1 may be represented by Formula 4-1 or Formula 4-2.

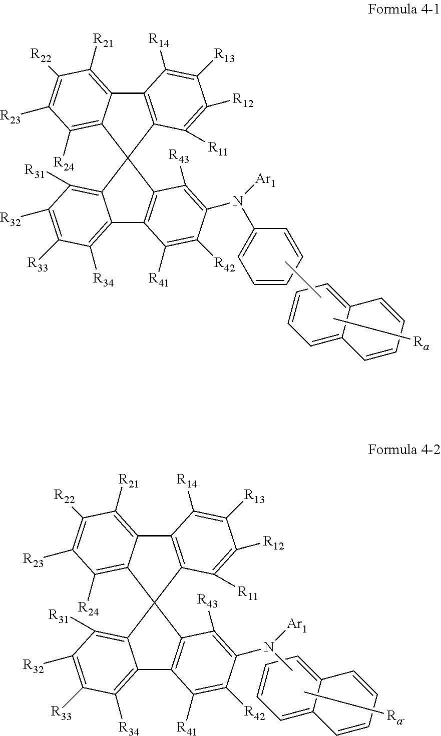

In Formula 4-1 and Formula 4-2, R₁₁ to R₁₄, R₂₁ to R₂₄, R₃₁ to R₃₄, R₄₁ to R₄₃, Ar₁, and Rₐ may each independently be the same as defined in Formula 1.

In one or more embodiments, Formula 4-1 may be represented by Formula 5.

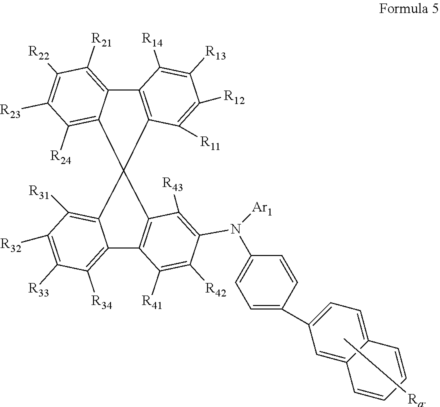

In Formula 5, R₁₁ to R₁₄, R₂₁ to R₂₄, R₃₁ to R₃₄, R₄₁ to R₄₃, Ar₁, and Rₐ may each independently be the same as defined in Formula 1.

In one or more embodiments, the amine compound represented by Formula 1 may be any one selected from among compounds of Compound Group 1.

### BRIEF DESCRIPTION OF THE DRAWINGS

The accompanying drawings are included to provide a further understanding of the present disclosure and are incorporated in and constitute a part of this specification. The drawings illustrate embodiments of the present disclosure and, together with the description, serve to explain principles of the present disclosure. Above and/or other aspects of the present disclosure should become apparent and appreciated from the following description of embodiments taken in conjunction with the accompanying drawings. In the drawings:

FIG. 1 is a plan view showing a display device according to one or more embodiments of the present disclosure;

FIG. 2 is a cross-sectional view showing a part corresponding to line I-I′ in FIG. 1;

FIG. 3 is a cross-sectional view schematically showing a light emitting element according to one or more embodiments of the present disclosure;

FIG. 4 is a cross-sectional view schematically showing a light emitting element according to one or more embodiments of the present disclosure;

FIG. 5 is a cross-sectional view schematically showing a light emitting element according to one or more embodiments of the present disclosure;

FIG. 6 is a cross-sectional view schematically showing a light emitting element according to one or more embodiments of the present disclosure;

FIG. 7 is a cross-sectional view of a display device according to one or more embodiments of the present disclosure;

FIG. 8 is a cross-sectional view of a display device according to one or more embodiments of the present disclosure;

FIG. 9 is a cross-sectional view showing a display device according to one or more embodiments of the present disclosure; and

FIG. 10 is a cross-sectional view showing a display device according to one or more embodiments of the present disclosure.

### DETAILED DESCRIPTION

The present disclosure may have one or more suitable modifications and may be embodied in different forms, and example embodiments will be explained in more detail with reference to the accompany drawings. The present disclosure may, however, be embodied in different forms and should not be construed as limited to the embodiments set forth herein. Rather, all modifications, equivalents, and substituents which are included in the spirit and technical scope of the present disclosure should be included in the present disclosure.

Like reference numerals refer to like elements throughout the present disclosure, and duplicative descriptions thereof may not be provided for conciseness. In the drawings, the dimensions of structures may be exaggerated for clarity of illustration. It will be understood that, although the terms “first,” “second,” etc. may be utilized herein to describe one or more suitable elements, these elements should not be limited by these terms. These terms are only utilized to distinguish one element from another element. Thus, a first element could be termed a second element without departing from the teachings of the present disclosure. Similarly, a second element could be termed a first element. As utilized herein, the singular forms are intended to include the plural forms as well, unless the context clearly indicates otherwise.

In the present disclosure, it will be further understood that the terms “comprises/includes,” “have(has)/having,” and/or “comprising/including,” when utilized in the present disclosure, specify the presence of stated features, numerals, steps, operations, elements, parts, or the combination thereof, but do not preclude the presence or addition of one or more other features, numerals, steps, operations, elements, parts, or the combination thereof.

In the present disclosure, when a layer, a film, a region, a plate, etc. is referred to as being “on” or “above” another part, it can be “directly on” the other part, or intervening layers may also be present. When a layer, a film, a region, a plate, etc. is referred to as being “under” or “below” another part, it can be “directly under” the other part, or intervening layers may also be present. Also, when an element is referred to as being disposed “on” another element, it can be disposed under the other element. As used herein, the terms “and”, “or”, and “and/or” may include any and all combinations of one or more of the associated listed items. Expressions such as “at least one of,” “one of,” and “selected from,” when preceding a list of elements, modify the entire list of elements and do not modify the individual elements of the list. For example, “at least one of a, b, or c”, “at least one selected from a, b, and c”, “at least one selected from among a to c”, etc., may indicate only a, only b, only c, both (e.g., simultaneously) a and b, both (e.g., simultaneously) a and c, both (e.g., simultaneously) b and c, all of a, b, and c, or variations thereof. The “/” utilized herein may be interpreted as “and” or as “or” depending on the situation.

In the present disclosure, the term “substituted or unsubstituted” corresponds to substituted or unsubstituted with at least one substituent selected from the group consisting of deuterium, a halogen, a cyano group, a nitro group, an amino group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In some embodiments, each of the exemplified substituents may be substituted or unsubstituted. For example, a biphenyl group may be interpreted as an aryl group or a phenyl group substituted with a phenyl group.

In the present disclosure, the term “forming a ring via the combination with an adjacent group” may refer to forming a substituted or unsubstituted hydrocarbon ring, or a substituted or unsubstituted heterocycle via the combination with an adjacent group. The hydrocarbon ring may include an aliphatic hydrocarbon ring and/or an aromatic hydrocarbon ring. The heterocycle may include an aliphatic heterocycle and/or an aromatic heterocycle. The hydrocarbon ring and the heterocycle may be monocycles or polycycles. In some embodiments, the ring formed via the combination with an adjacent group may be combined with another ring to form a spiro structure.

In the present disclosure, the term “adjacent group” may refer to a substituent substituted for an atom which is directly combined with an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups thereof may be interpreted as “adjacent groups” to each other, and in 1,1-diethylcyclopentene, two ethyl groups thereof may be interpreted as “adjacent groups” to each other. In some embodiments, in 4,5-dimethylphenanthrene, two methyl groups thereof may be interpreted as “adjacent groups” to each other.

In the present disclosure, a halogen may be fluorine, chlorine, bromine, or iodine.

In the present disclosure, an alkyl group may be a linear, branched, or cyclic type or kind. The carbon number of the alkyl group may be 1 to 50, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-heneicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the present disclosure, an alkyl group may be a linear or branched type or kind. The carbon number of the alkyl group may be 1 to 50, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, 1-methylpentyl, 3-methylpentyl, 2-ethylpentyl, 4-methyl-2-pentyl, n-hexyl, 1-methylhexyl, 2-ethylhexyl, 2-butylhexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the present disclosure, a cycloalkyl group may refer to a ring-type or kind alkyl group. The carbon number of the cycloalkyl group may be 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, etc., without limitation.

In the present disclosure, an alkenyl group may refer to a hydrocarbon group including one or more carbon-carbon double bonds in the middle or at the terminal of an alkyl group having a carbon number of 2 or more. The alkenyl group may be a linear chain or a branched chain. The carbon number is not specifically limited, for example, may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styrylvinyl group, etc., without limitation.

In the present disclosure, an alkynyl group may refer to a hydrocarbon group including one or more carbon-carbon triple bonds in the middle or at the terminal of an alkyl group having a carbon number of 2 or more. The alkynyl group may be a linear chain or a branched chain. The carbon number is not specifically limited, for example, may be 2 to 30, 2 to 20, or 2 to 10. Examples of the alkynyl group include an ethynyl group, a propionyl group, etc., without limitation.

In the present disclosure, a hydrocarbon ring group may refer to an optional functional group or substituent derived from an aliphatic hydrocarbon ring. A hydrocarbon ring group may be a saturated hydrocarbon ring group of 5 to 20 ring-forming carbon atoms.

In the present disclosure, an aryl group may refer to an optional functional group or substituent derived from an aromatic hydrocarbon ring. The aryl group may be a monocyclic aryl group or a polycyclic aryl group. The number of carbon atoms forming rings in the aryl group may be 6 to 30, 6 to 20, or 6 to 15. Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, etc., without limitation.

In the present disclosure, a fluorenyl group may be substituted, and two substituents may be combined with each other to form a spiro structure. Examples of a substituted fluorenyl group are as follows, but embodiments of the present disclosure are not limited thereto.

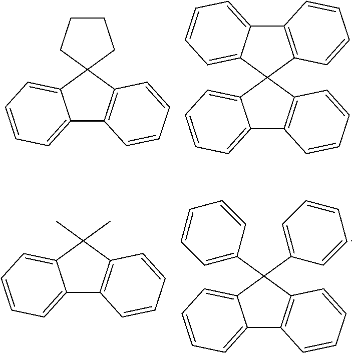

In the present disclosure, a heterocyclic group may refer to an optional functional group or substituent derived from a ring including one or more selected from among B, O, N, P, Si, and S as heteroatoms. The heterocyclic group may include an aliphatic heterocyclic group and an aromatic heterocyclic group. The aromatic heterocyclic group may be a heteroaryl group. The aliphatic heterocyclic group and the aromatic heterocyclic group may be a monocycle or a polycycle.

In the present disclosure, a heterocyclic group may include one or more selected from among B, O, N, P, Si and S as heteroatoms. When the heterocyclic group includes two or more heteroatoms, the two or more heteroatoms may be the same or different. The heterocyclic group may be a monocyclic heterocyclic group or polycyclic heterocyclic group and has the concept including a heteroaryl group. The number of carbon atoms forming rings of the heterocyclic group may be 2 to 30, 2 to 20, or 2 to 10.

In the present disclosure, an aliphatic heterocyclic group may include one or more selected from among B, O, N, P, Si, and S as heteroatoms. The number of ring-forming carbon atoms of the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., without limitation.

In the present disclosure, a heteroaryl group may include one or more selected from among B, O, N, P, Si, and S as heteroatoms. When the heteroaryl group includes two or more heteroatoms, the two or more heteroatoms may be the same or different. The heteroaryl group may be a monocyclic heterocyclic group or polycyclic heterocyclic group. The number of carbon atoms forming rings of the heteroaryl group may be 2 to 30, 2 to 20, or 2 to 10. Examples of the heteroaryl group may include thiophene, furan, pyrrole, imidazole, pyridine, bipyridine, pyrimidine, triazine, triazole, acridyl, pyridazine, pyrazinyl, quinoline, quinazoline, quinoxaline, phenoxazine, phthalazine, pyrido pyrimidine, pyrido pyrazine, pyrazino pyrazine, isoquinoline, indole, carbazole, N-arylcarbazole, N-heteroarylcarbazole, N-alkylcarbazole, benzoxazole, benzimidazole, benzothiazole, benzocarbazole, benzothiophene, dibenzothiophene, thienothiophene, benzofuran, phenanthroline, thiazole, isooxazole, oxazole, oxadiazole, thiadiazole, phenothiazine, dibenzosilole, dibenzofuran, etc., without limitation.

In the present disclosure, the same explanation on the above-described aryl group may be applied to an arylene group except that the arylene group is a divalent group. The same explanation on the above-described heteroaryl group may be applied to a heteroarylene group except that the heteroarylene group is a divalent group.

In the present disclosure, a silyl group may include an alkyl silyl group and/or an aryl silyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., without limitation.

In the present disclosure, the carbon number of an amino group is not specifically limited, for example, may be 1 to 30. The amino group may include an alkyl amino group, an aryl amino group, or a heteroaryl amino group. Examples of the amine group may include a methylamino group, a dimethylamino group, a phenylamino group, a diphenylamino group, a naphthylamino group, a 9-methyl-anthracenylamino group, a triphenylamino group, etc., without limitation.

In the present disclosure, the carbon number of a carbonyl group is not specifically limited, for example, may be 1 to 40, 1 to 30, or 1 to 20. For example, the carbonyl group may have the following structures, but embodiments of the present disclosure are not limited thereto.

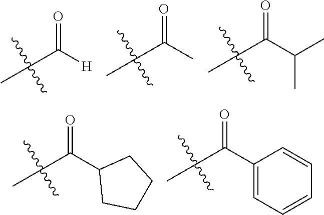

In the present disclosure, the carbon number of a sulfinyl group or sulfonyl group is not specifically limited, for example, may be 1 to 30. The sulfinyl group may include an alkyl sulfinyl group and/or an aryl sulfinyl group. The sulfonyl group may include an alkyl sulfonyl group and/or an aryl sulfonyl group.

In the present disclosure, a thio group may include an alkyl thio group and/or an aryl thio group. The thio group may refer to the above-defined alkyl group or aryl group combined with a sulfur atom. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., without limitation.

In the present disclosure, an oxy group may refer to the above-defined alkyl group or aryl group which is combined with an oxygen atom. The oxy group may include an alkoxy group and/or an aryl oxy group. The alkoxy group may be a linear, branched, or cyclic chain. The carbon number of the alkoxy group is not specifically limited but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, etc. However, embodiments of the present disclosure are not limited thereto.

In the present disclosure, a boron group may refer to the above-defined alkyl group or aryl group combined with a boron atom. The boron group may include an alkyl boron group and/or an aryl boron group. Examples of the boron group may include a dimethylboron group, a diethylboron group, a t-butylboron group, a diphenylboron group, a phenylboron group, and/or the like, without limitation.

In the present disclosure, an alkenyl group may be a linear chain or a branched chain. The carbon number is not specifically limited, but may be 2 to 30, 2 to 20, or 2 to 10. Examples of the alkenyl group may include a vinyl group, a 1-butenyl group, a 1-pentenyl group, a 1,3-butadienyl aryl group, a styrenyl group, a styrylvinyl group, etc., without limitation.

In the present disclosure, the carbon number of an amine group is not specifically limited, for example, may be 1 to 30. The amine group may include an alkyl amine group and/or an aryl amine group. Examples of the amine group may include a methylamine group, a dimethylamine group, a phenylamine group, a diphenylamine group, a naphthylamine group, a 9-methyl-anthracenylamine group, a triphenylamine group, etc., without limitation.

In the present disclosure, alkyl groups in an alkylthio group, alkylsulfoxy group, alkylaryl group, alkylamino group, alkylboron group, alkyl silyl group, and alkyl amine group may be the same as the examples of the above-described alkyl group.

In the present disclosure, aryl groups in an aryloxy group, arylthio group, arylsulfoxy group, aryl amino group, arylboron group, and aryl silyl group may be the same as the examples of the above-described aryl group.

In the present disclosure, a direct linkage may refer to a single bond.

In the present disclosure,

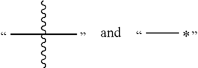

may refer to positions to be connected.

Hereinafter, light emitting elements of one or more embodiments of the present disclosure will be explained in more detail referring to the drawings.

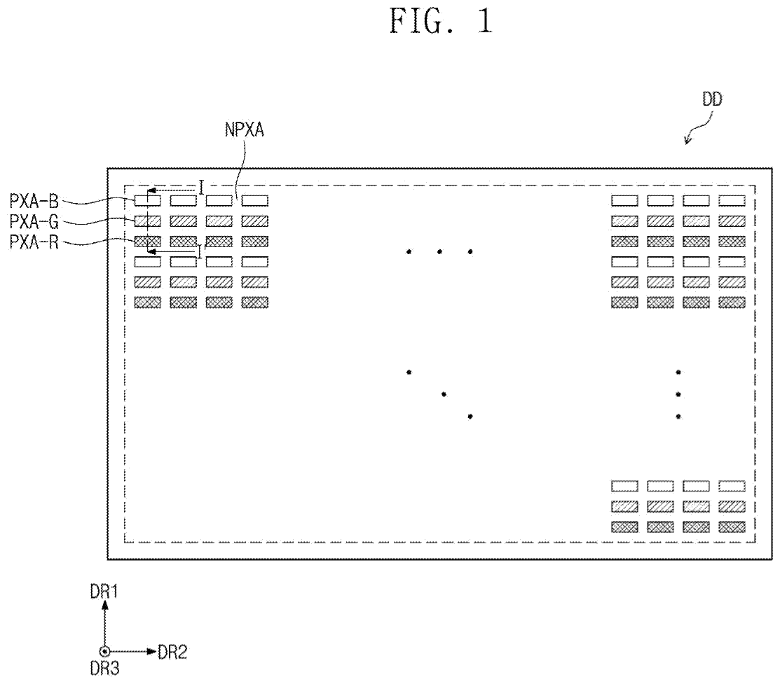

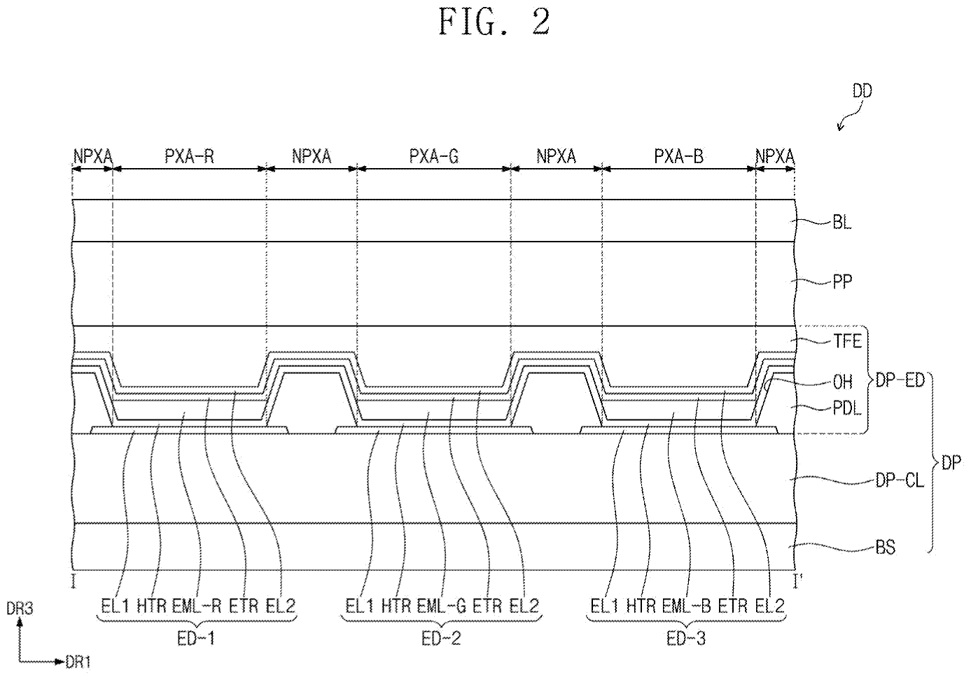

FIG. 1 is a plan view showing a display device DD according to one or more embodiments. FIG. 2 is a cross-sectional view of a display device DD according to one or more embodiments. FIG. 2 is a cross-sectional view showing a part corresponding to line I-I′ of FIG. 1.

The display device DD may include a display panel DP and an optical layer PP disposed on the display panel DP. The display panel DP may include light emitting elements ED-1, ED-2, and ED-3. The display device DD may include multiple light emitting elements ED-1, ED-2, and ED-3. The optical layer PP may be disposed on the display panel DP to control reflected light by external light at the display panel DP. The optical layer PP may include, for example, a polarization layer or a color filter layer. In some embodiments, different from the drawings, the optical layer PP may not be provided in the display device DD.

On the optical layer PP, a base substrate BL may be disposed or provided. The base substrate BL may be a member providing a base surface where the optical layer PP is disposed. The base substrate BL may be a glass substrate, a metal substrate, a plastic substrate, etc. However, embodiments of the present disclosure are not limited thereto, and the base substrate BL may be an inorganic layer, an organic layer, or a composite material layer. In some embodiments, different from the drawings, the base substrate BL may not be provided.

The display device DD according to one or more embodiments may further include a plugging layer. The plugging layer may be disposed between a display element layer DP-ED and a base substrate BL. The plugging layer may be an organic layer. The plugging layer may include at least one selected from among an acrylic resin, a silicon-based resin, and an epoxy-based resin.

The display panel DP may include a base layer BS, a circuit layer DP-CL provided on the base layer BS, and a display element layer DP-ED. The display element layer DP-ED may include a pixel definition layer PDL, light emitting elements ED-1, ED-2, and ED-3 disposed in the pixel definition layer PDL, and an encapsulating layer TFE disposed on the light emitting elements ED-1, ED-2, and ED-3.

The base layer BS may be a member providing a base surface on which the display element layer DP-ED is disposed. The base layer BS may be a glass substrate, a metal substrate, a plastic substrate, etc. However, embodiments of the present disclosure are not limited thereto, and the base layer BS may be an inorganic layer, an organic layer, or a composite material layer.

In one or more embodiments, the circuit layer DP-CL may be disposed on the base layer BS, and the circuit layer DP-CL may include multiple transistors. Each of the transistors may include a control electrode, an input electrode, and an output electrode. For example, in some embodiments, the circuit layer DP-CL may include switching transistors and driving transistors for driving the light emitting elements ED-1, ED-2, and ED-3 of the display element layer DP-ED.

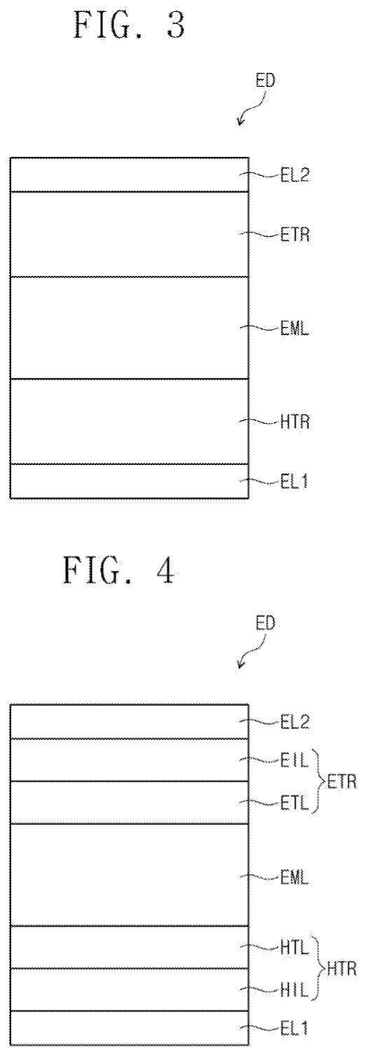

The light emitting elements ED-1, ED-2, and ED-3 may have the structures of the light emitting elements ED of embodiments according to FIG. 3 to FIG. 6, which will be explained later. The light emitting elements ED-1, ED-2, and ED-3 may include a first electrode EL1, a hole transport region HTR, respective emission layers EML-R, EML-G, and EML-B, an electron transport region ETR, and a second electrode EL2.

In FIG. 2, shown is an embodiment where the respective emission layers EML-R, EML-G, and EML-B of light emitting elements ED-1, ED-2, and ED-3 are disposed in opening portions OH defined in a pixel definition layer PDL, and a hole transport region HTR, an electron transport region ETR, and a second electrode EL2 are provided as common layers in all light emitting elements ED-1, ED-2, and ED-3. However, embodiments of the present disclosure are not limited thereto. In one or more embodiments, the hole transport region HTR and the electron transport region ETR may be patterned and provided in the opening portions OH defined in the pixel definition layer PDL. For example, in some embodiments, the hole transport region HTR, the respective emission layers EML-R, EML-G, and EML-B, and the electron transport region ETR of the light emitting elements ED-1, ED-2 and ED-3 may be provided and patterned by an ink jet printing method.

The encapsulating layer TFE may cover the light emitting elements ED-1, ED-2, and ED-3. The encapsulating layer TFE may encapsulate the display element layer DP-ED. The encapsulating layer TFE may be a thin film encapsulating layer. The encapsulating layer TFE may be one layer or a stacked layer of multiple layers. The encapsulating layer TFE may include at least one insulating layer. In some embodiments, the encapsulating layer TFE may include at least one inorganic layer (hereinafter, encapsulating inorganic layer). In some embodiments, the encapsulating layer TFE may include at least one organic layer (hereinafter, encapsulating organic layer) and at least one encapsulating inorganic layer.

The encapsulating inorganic layer protects the display element layer DP-ED from moisture/oxygen, and the encapsulating organic layer protects the display element layer DP-ED from foreign materials such as dust particles. The encapsulating inorganic layer may include silicon nitride, silicon oxy nitride, silicon oxide, titanium oxide, and/or aluminum oxide, without specific limitation. The encapsulating organic layer may include an acrylic compound, an epoxy-based compound, etc. In one or more embodiments, the encapsulating organic layer may include a photopolymerizable organic material, without specific limitation.

The encapsulating layer TFE may be disposed on the second electrode EL2 and may be disposed while filling the opening portion OH.

Referring to FIG. 1 and FIG. 2, the display device DD may include a non-luminous area NPXA and luminous areas PXA-R, PXA-G, and PXA-B. The luminous areas PXA-R, PXA-G, and PXA-B may be areas emitting light produced from the light emitting elements ED-1, ED-2, and ED-3, respectively. The luminous areas PXA-R, PXA-G, and PXA-B may be separated or apart from each other on a plane.

The luminous areas PXA-R, PXA-G, and PXA-B may be areas separated by the pixel definition layer PDL. The non-luminous areas NPXA may be areas between neighboring luminous areas PXA-R, PXA-G, and PXA-B and may be areas corresponding to the pixel definition layer PDL. In some embodiments of the present disclosure, each of the luminous areas PXA-R, PXA-G, and PXA-B may correspond to each pixel. The pixel definition layer PDL may divide the light emitting elements ED-1, ED-2, and ED-3. The respective emission layers EML-R, EML-G, and EML-B of the light emitting elements ED-1, ED-2, and ED-3 may be disposed and divided in the opening portions OH defined in the pixel definition layer PDL.

The luminous areas PXA-R, PXA-G, and PXA-B may be divided into multiple groups according to the color of light produced from the light emitting elements ED-1, ED-2, and ED-3. In the display device DD of one or more embodiments, as shown in FIG. 1 and FIG. 2, three luminous areas PXA-R, PXA-G, and PXA-B emitting red light, green light, and blue light, respectively, are illustrated as an example. For example, in one or more embodiments, the display device DD may include a red luminous area PXA-R, a green luminous area PXA-G, and a blue luminous area PXA-B, which are separated or apart from each other.

In the display device DD according to one or more embodiments, multiple light emitting elements ED-1, ED-2 and ED-3 may be to emit light having different wavelength regions. For example, in some embodiments, the display device DD may include a first light emitting element ED-1 to emit red light, a second light emitting element ED-2 to emit green light, and a third light emitting element ED-3 to emit blue light. For example, in one or more embodiments, the red luminous area PXA-R, the green luminous area PXA-G, and the blue luminous area PXA-B of the display device DD may respectively correspond to the first light emitting element ED-1, the second light emitting element ED-2, and the third light emitting element ED-3.

However, embodiments of the present disclosure are not limited thereto, and the first to third light emitting elements ED-1, ED-2, and ED-3 may be to emit light in substantially the same wavelength region, or at least one thereof may be to emit light in a different wavelength region. For example, in some embodiments, all the first to third light emitting elements ED-1, ED-2, and ED-3 may be to emit blue light.

The luminous areas PXA-R, PXA-G, and PXA-B in the display device DD according to one or more embodiments may be arranged in a stripe shape. Referring to FIG. 1, multiple red luminous areas PXA-R may be arranged with each other along a second direction axis DR2, multiple green luminous areas PXA-G may be arranged with each other along the second direction axis DR2, and multiple blue luminous areas PXA-B may be arranged with each other along the second direction axis DR2. In some embodiments, the red luminous area PXA-R, the green luminous area PXA-G, and the blue luminous area PXA-B may be arranged by turns along a first direction axis DR1.

In FIG. 1 and FIG. 2, the areas of the luminous areas PXA-R, PXA-G, and PXA-B are shown similar, but embodiments of the present disclosure are not limited thereto. The areas of the luminous areas PXA-R, PXA-G, and PXA-B may be different from each other according to the wavelength region of light emitted. The areas of the luminous areas PXA-R, PXA-G, and PXA-B may refer to areas on a plane defined by the first direction axis DR1 and the second direction axis DR2 (e.g., the areas in a plan view).

In some embodiments, the arrangement type or kind of the luminous areas PXA-R, PXA-G, and PXA-B is not limited to the configuration shown in FIG. 1, and the arrangement order of the red luminous areas PXA-R, the green luminous areas PXA-G, and the blue luminous areas PXA-B may be provided in one or more suitable combinations according to the properties of display quality required for the display device DD. For example, in one or more embodiments, the arrangement type or kind of the luminous areas PXA-R, PXA-G, and PXA-B may be a pentile (PENTILE©) arrangement type or kind (for example, an RGBG matrix, an RGBG structure, or an RGBG matrix structure), or a diamond (Diamond Pixel™) arrangement type or kind (e.g., a display (e.g., an OLED display) containing red, blue, and green (RGB) light-emitting regions arranged in the shape of diamonds). PENTILE© is a duly registered trademark of Samsung Display Co., Ltd. Diamond Pixel™ is a trademark of Samsung Display Co., Ltd.

In some embodiments, the areas of the luminous areas PXA-R, PXA-G, and PXA-B may be different from each other. For example, in some embodiments, the area of the green luminous area PXA-G may be smaller than the area of the blue luminous area PXA-B, but embodiments of the present disclosure are not limited thereto.

Hereinafter, FIG. 3 to FIG. 6 are cross-sectional views schematically showing light emitting elements according to one or more embodiments of the present disclosure. The light emitting elements ED of one or more embodiments each may include a first electrode EL1, a hole transport region HTR, an emission layer EML, an electron transport region ETR, and a second electrode EL2, stacked in order (e.g., in the stated order).

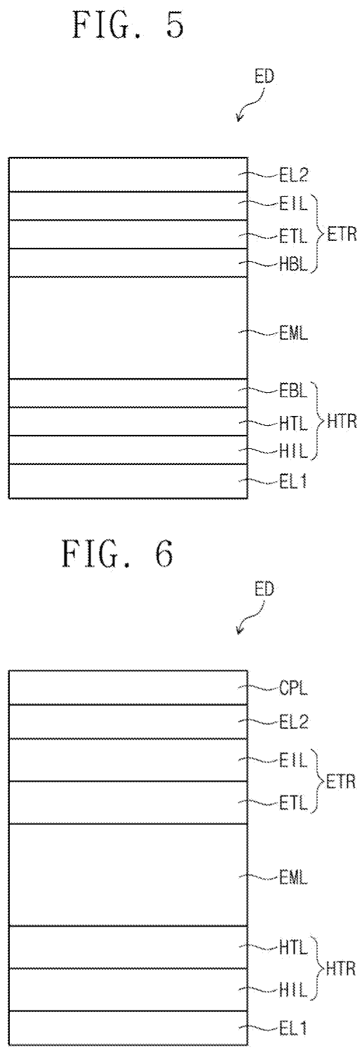

Compared with FIG. 3, FIG. 4 shows the cross-sectional view of a light emitting element ED according to one or more embodiments, wherein a hole transport region HTR may include a hole injection layer HIL and a hole transport layer HTL, and an electron transport region ETR may include an electron injection layer EIL and an electron transport layer ETL. In some embodiments, compared with FIG. 3, FIG. 5 shows the cross-sectional view of a light emitting element ED according to one or more embodiments, wherein a hole transport region HTR may include a hole injection layer HIL, a hole transport layer HTL, and an electron blocking layer EBL, and an electron transport region ETR may include an electron injection layer EIL, an electron transport layer ETL, and a hole blocking layer HBL. Compared with FIG. 4, FIG. 6 shows the cross-sectional view of a light emitting element ED according to one or more embodiments, including a capping layer CPL disposed on the second electrode EL2.

In one or more embodiments, the light emitting element ED may include the amine compound of one or more embodiments of the present disclosure in at least one functional layer disposed between the first electrode EL1 and the second electrode EL2. The at least one functional layer may include a hole transport region HTR, an emission layer EML, and/or an electron transport region ETR. For example, in some embodiments, the hole transport region HTR may include the amine compound of one or more embodiments of the present disclosure.

The light emitting element ED including the amine compound of one or more embodiments may show a reduced driving voltage, high efficiency, high luminance, and long-life characteristics. The amine compound of one or more embodiments may include a structure in which first to third substituents are connected with the nitrogen atom of the amine. In one or more embodiments, the first substituent may include a substituted or unsubstituted naphthyl group. The second substituent may include a spiro fluorene moiety substituted with at least one substituted or unsubstituted methyl group. The naphthyl group may be directly bonded to the nitrogen atom of the amine, or combined therewith via a linker. The spiro fluorene moiety may be directly bonded to the nitrogen atom of the amine. In some embodiments, the amine compound may be a monoamine compound.

In one or more embodiments, the light emitting element ED may include the amine compound of one or more embodiments of the present disclosure. The amine compound of one or more embodiments may be represented by Formula 1.

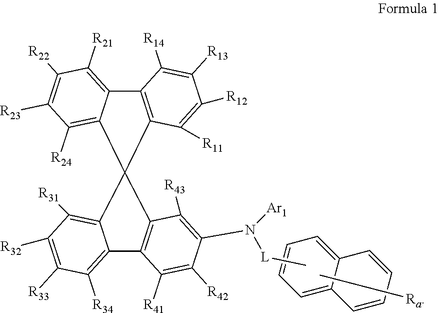

The amine compound of one or more embodiments may include a spiro fluorene moiety bonded to the nitrogen atom of the amine and substituted with at least one substituted or unsubstituted methyl group. In Formula 1, at least one selected from among R₁₁ to R₁₄, R₂₁ to R₂₄, R₃₁ to R₃₄, and R₄₁ to R₄₃ may be a substituted or unsubstituted methyl group, and the remainder may each independently be hydrogen, deuterium, a halogen, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring. For example, in one or more embodiments, in Formula 1, at least one selected from among R₁₂, R₂₃, and R₃₄ may be an unsubstituted methyl group. For example, in some embodiments, any one selected from among R₂₃ and R₃₄ may be an unsubstituted methyl group, R₁₂ and R₂₃ may be unsubstituted methyl groups, R₂₃ and R₃₄ may be unsubstituted methyl groups, or R₁₂, R₂₃, and R₃₄ may be unsubstituted methyl groups. The amine compound of one or more embodiments may include at least one substituted or unsubstituted methyl group, as such, the stacking of molecules is suppressed or reduced, and a low refractive index may be achieved. In some embodiments, the amine compound of one or more embodiments has high glass transition temperature to prevent or reduce crystallization, and may show excellent or suitable hole transport capacity. Accordingly, the light emitting element ED of one or more embodiments may include the amine compound of one or more embodiments in a hole transport region HTR, and may show a low driving voltage, high luminance, high efficiency, and long-life characteristics.

Ar₁ may be a substituted or unsubstituted aryl group of 6 to 30 ring-forming carbon atoms, or a substituted or unsubstituted heteroaryl group of 2 to 30 ring-forming carbon atoms. For example, in one or more embodiments, Ar₁ may be a substituted or unsubstituted phenyl group, a substituted or unsubstituted naphthyl group, or a substituted or unsubstituted tetrahydro naphthyl group.

L may be a direct linkage, a substituted or unsubstituted arylene group of 6 to 30 ring-forming carbon atoms, or a substituted or unsubstituted heteroarylene group of 2 to 30 ring-forming carbon atoms. When L is a direct linkage, the naphthyl group substituted with Rₐ may be directly bonded to the nitrogen atom of the amine. When L is a substituted or unsubstituted arylene group of 6 to 30 ring-forming carbon atoms, or a substituted or unsubstituted heteroarylene group of 2 to 30 ring-forming carbon atoms, the naphthyl group substituted with Rₐ may be combined with the nitrogen atom of the amine via L.

Rₐ may be a substituent that is substituted at the naphthyl group combined with the nitrogen atom of the amine. Rₐ may be hydrogen, deuterium, a halogen, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group from each other to form a ring. For example, in one or more embodiments, Rₐ may be a substituted or unsubstituted phenyl group, or a substituted or unsubstituted methyl group.

An amine compound having a spiro fluorene moiety and a naphthyl group together shows better hole transport properties when compared to an amine compound unsubstituted with a naphthyl group (e.g., without a naphthyl group). Accordingly, a light emitting element including a hole transport layer including an amine compound having a spiro fluorene moiety and a naphthyl group together may have a low voltage, high efficiency, and high luminance properties.

In one or more embodiments, Formula 1 may be represented by any one selected from among Formula 1-1 to Formula 1-4. Formula 1-1 is an embodiment of Formula 1 in which R₂₃ is an unsubstituted methyl group, and R₁₁ to R₁₄, R₂₁, R₂₂, R₂₄, R₃₁ to R₃₄, and R₄₁ to R₄₃ are hydrogen atoms. Formula 1-2 is an embodiment of Formula 1 where R₃₄ is an unsubstituted methyl group, R₁₁ to R₁₄, R₂₁, R₂₂, R₂₄, R₃₁ to R₃₃, and R₄₁ to R₄₃ are hydrogen atoms, wherein R₂₃ is the same as defined in Formula 1. Formula 1-3 is an embodiment of Formula 1 in which R₁₂ and R₂₃ are unsubstituted methyl groups, and R₁₁, R₁₃, R₁₄, R₂₁, R₂₂, R₂₄, R₃₁ to R₃₄, and R₄₁ to R₄₃ are hydrogen atoms. Formula 1-4 is an embodiment of Formula 1 in which R₁₂, R₂₃, and R₃₃ are unsubstituted methyl groups, and R₁₁, R₁₃, R₁₄, R₂₁, R₂₂, R₂₄, R₃₁, R₃₂, R₃₄, and R₄₁ to R₄₃ are hydrogen atoms.

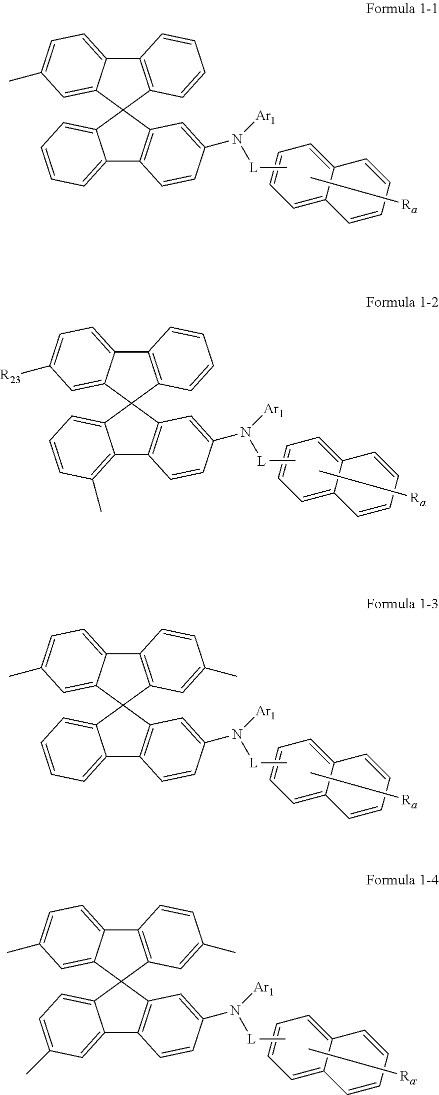

In Formula 1-1 to Formula 1-4, the same content defined in Formula 1 may be applied for R₂₃, Ar₁, L, and Rₐ. For example, in Formula 1-2, R₂₃ may be hydrogen, an unsubstituted methyl group, an unsubstituted phenyl group, or an unsubstituted t-butyl group.

In one or more embodiments, Formula 1 may be represented by Formula 2. Formula 2 corresponds to Formula 1 where the substitution position of L is specified in a naphthyl group substituted with Rₐ. In some embodiments, when L is a direct linkage, the substitution position of the nitrogen atom of the amine may be specified in the naphthyl group substituted with Rₐ.

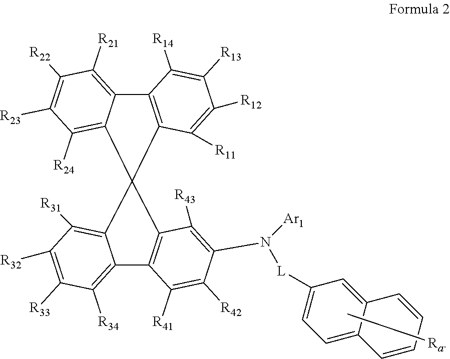

In Formula 2, the same content defined in Formula 1 may be applied for R₁₁ to R₁₄, R₂₁ to R₂₄, R₃₁ to R₃₄, R₄₁ to R₄₃, Ar₁, L, and Rₐ.

In one or more embodiments, Formula 2 may be represented by Formula 3-1 or Formula 3-2. Formula 3-1 and Formula 3-2 correspond Formula 2 where the substitution position of Rₐ in the naphthyl group which is bonded to the nitrogen atom of the amine via L is specified. L and Rₐ may be bonded to the same ring in the naphthyl group.

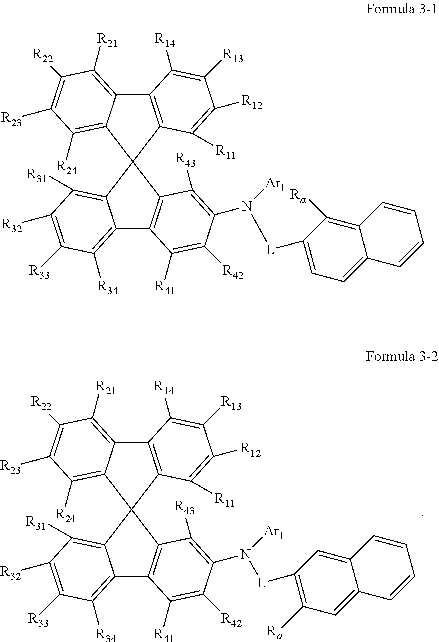

In Formula 3-1 and Formula 3-2, the same content defined in Formula 1 may be applied for R₁₁ to R₁₄, R₂₁ to R₂₄, R₃₁ to R₃₄, R₄₁ to R₄₃, Ar₁, L, and Rₐ.

In one or more embodiments, Formula 1 may be represented by Formula 4-1 or Formula 4-2. Formula 4-1 is an embodiment of Formula 1 where L is an unsubstituted phenylene group. Formula 4-2 is an embodiment of Formula 1 where L is a direct linkage.

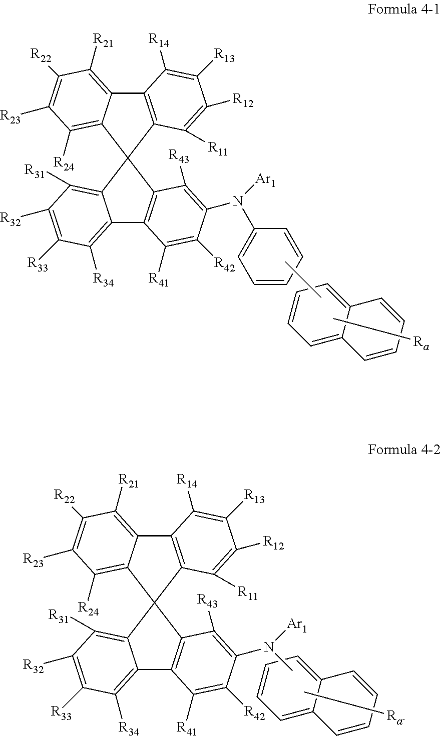

In Formula 4-1 and Formula 4-2, the same content defined in Formula 1 may be applied for R₁₁ to R₁₄, R₂₁ to R₂₄, R₃₁ to R₃₄, R₄₁ to R₄₃, Ar₁, and Rₐ.

In one or more embodiments, Formula 4-1 may be represented by Formula 5. Formula 5 corresponds to Formula 4-1 where the bonding position of the naphthyl group to an unsubstituted phenyl group which is bonded to the nitrogen atom of the amine, and the bonding position of an unsubstituted phenyl group which is bonded to the nitrogen atom of the amine to the naphthyl group are specified. Here, the unsubstituted phenyl group bonded to the nitrogen atom of the amine refers to L of Formula 1.

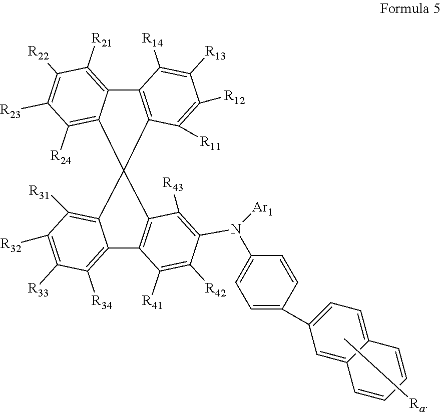

In Formula 5, the same content defined in Formula 1 may be applied for R₁₁ to R₁₄, R₂₁ to R₂₄, R₃₁ to R₃₄, R₄₁ to R₄₃, Ar₁, and Rₐ.

In one or more embodiments, the amine compound represented by Formula 1 may be represented by any one selected from among compounds in Compound Group 1. The light emitting element Ed of one or more embodiments may include at least one selected from among the compounds in Compound Group 1.

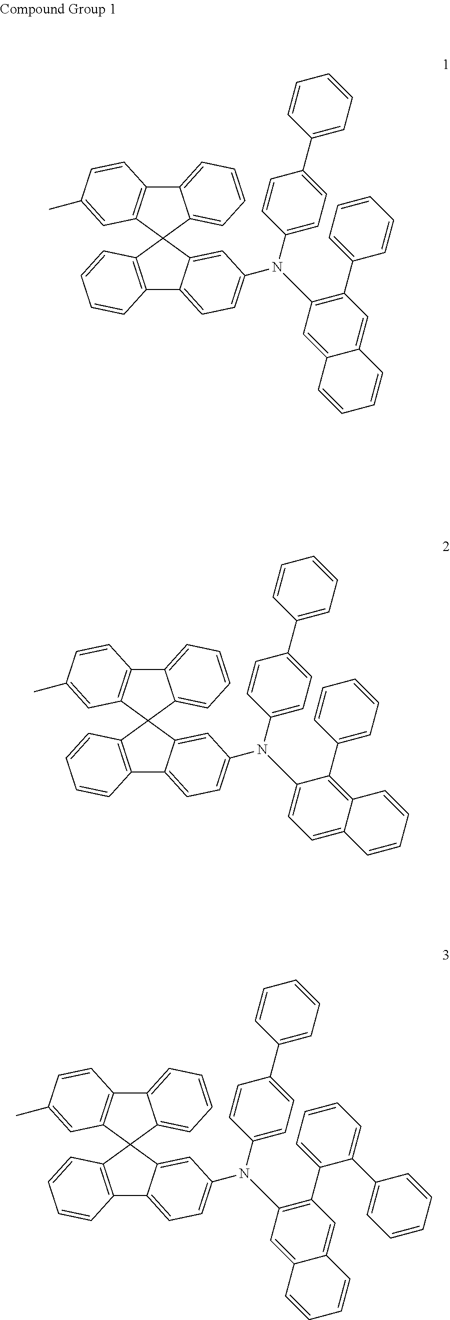 [SMILES: Cc1ccc2c(c1)C1(c3ccccc3-2)c2ccccc2-c2ccc(N(c3ccc(-c4ccccc4)cc3)c3cc4ccccc4cc3-c3ccccc3)cc21 ; Cc1ccc2c(c1)C1(c3ccccc3-2)c2ccccc2-c2ccc(N(c3ccc(-c4ccccc4)cc3)c3ccc4ccccc4c3-c3ccccc3)cc21 ; Cc1ccc2c(c1)C1(c3ccccc3-2)c2ccccc2-c2ccc(N(c3ccc(-c4ccccc4)cc3)c3cc4ccccc4cc3-c3ccccc3-c3ccccc3)cc21]

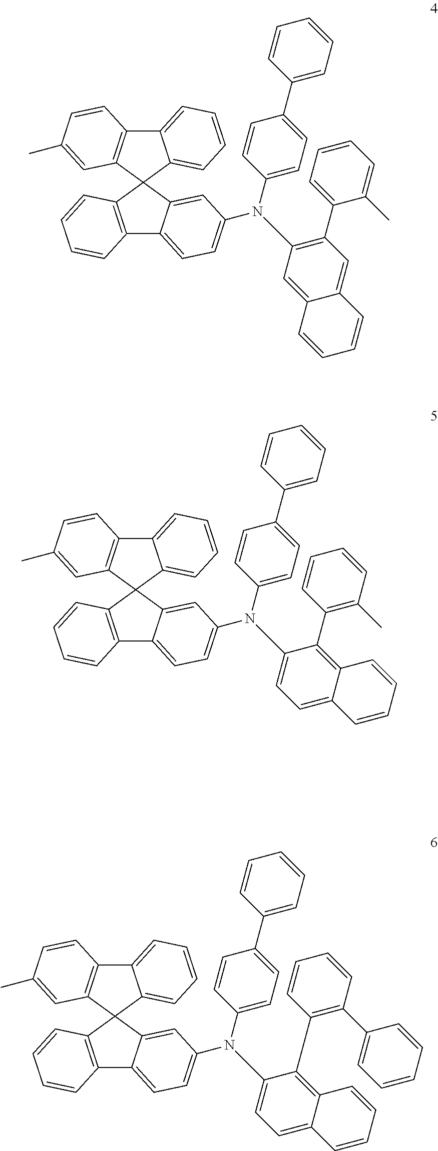

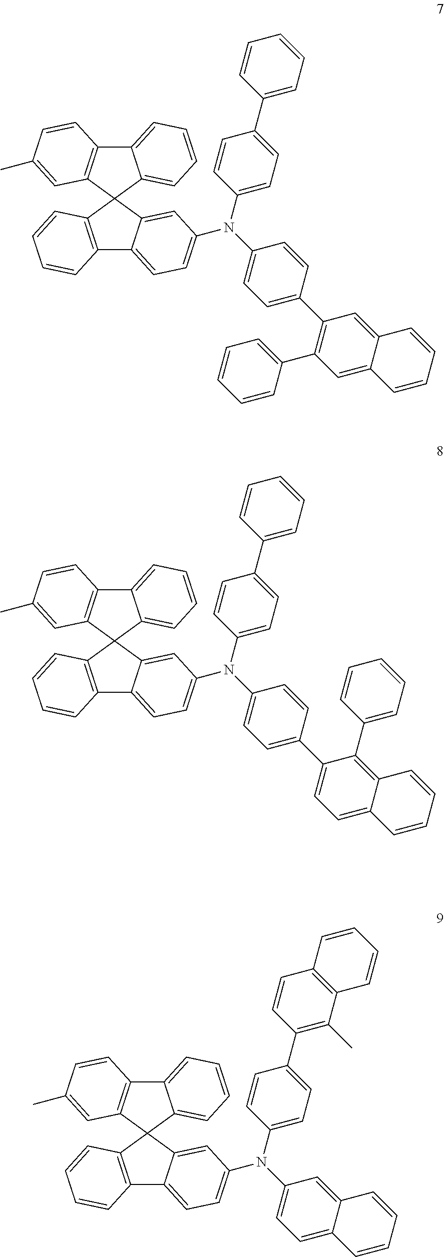 [SMILES: Cc1ccc2c(c1)C1(c3ccccc3-2)c2ccccc2-c2ccc(N(c3ccc(-c4ccccc4)cc3)c3ccc(-c4cc5ccccc5cc4-c4ccccc4)cc3)cc21 ; Cc1ccc2c(c1)C1(c3ccccc3-2)c2ccccc2-c2ccc(N(c3ccc(-c4ccccc4)cc3)c3ccc(-c4ccc5ccccc5c4-c4ccccc4)cc3)cc21 ; Cc1ccc2c(c1)C1(c3ccccc3-2)c2ccccc2-c2ccc(N(c3ccc(-c4ccc5ccccc5c4C)cc3)c3ccc4ccccc4c3)cc21]

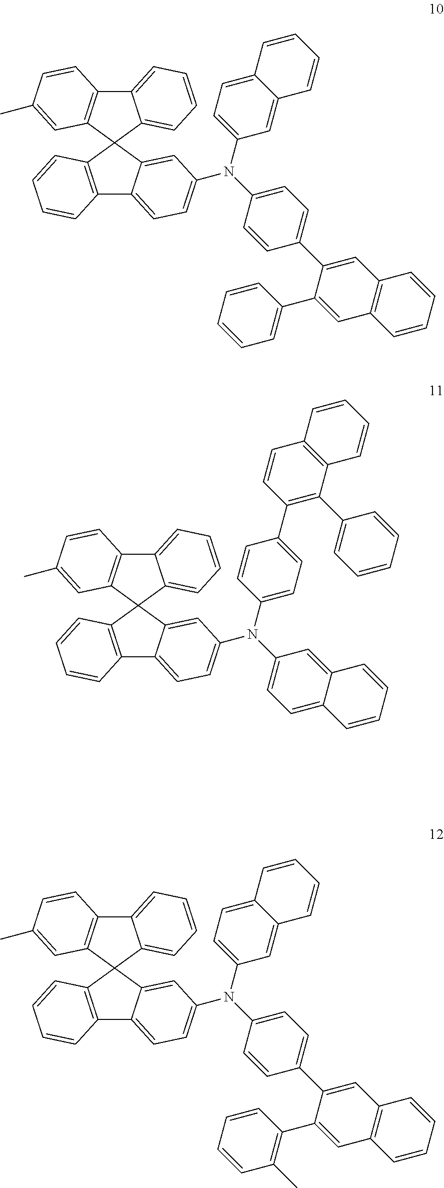 [SMILES: Cc1ccc2c(c1)C1(c3ccccc3-2)c2ccccc2-c2ccc(N(c3ccc(-c4cc5ccccc5cc4-c4ccccc4)cc3)c3ccc4ccccc4c3)cc21 ; Cc1ccc2c(c1)C1(c3ccccc3-2)c2ccccc2-c2ccc(N(c3ccc(-c4ccc5ccccc5c4-c4ccccc4)cc3)c3ccc4ccccc4c3)cc21 ; Cc1ccc2c(c1)C1(c3ccccc3-2)c2ccccc2-c2ccc(N(c3ccc(-c4cc5ccccc5cc4-c4ccccc4C)cc3)c3ccc4ccccc4c3)cc21]

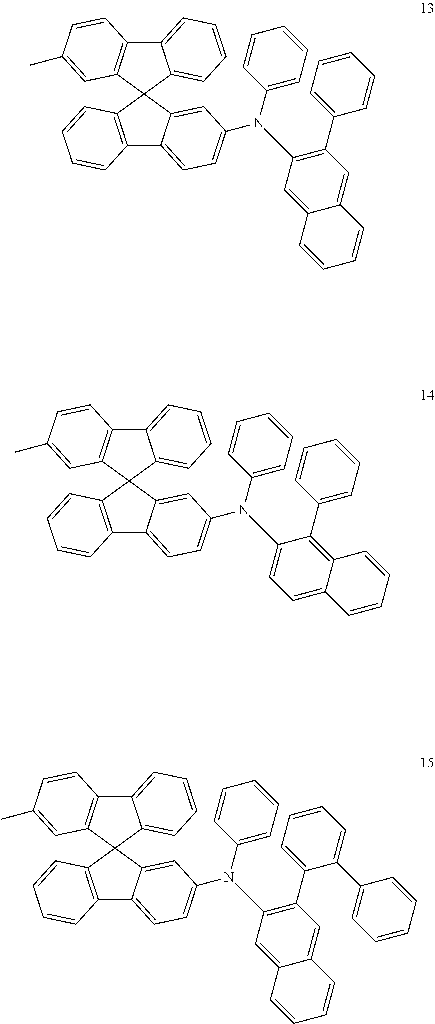 [SMILES: Cc1ccc2c(c1)C1(c3ccccc3-2)c2ccccc2-c2ccc(N(c3ccccc3)c3cc4ccccc4cc3-c3ccccc3)cc21 ; Cc1ccc2c(c1)C1(c3ccccc3-2)c2ccccc2-c2ccc(N(c3ccccc3)c3ccc4ccccc4c3-c3ccccc3)cc21 ; Cc1ccc2c(c1)C1(c3ccccc3-2)c2ccccc2-c2ccc(N(c3ccccc3)c3cc4ccccc4cc3-c3ccccc3-c3ccccc3)cc21]

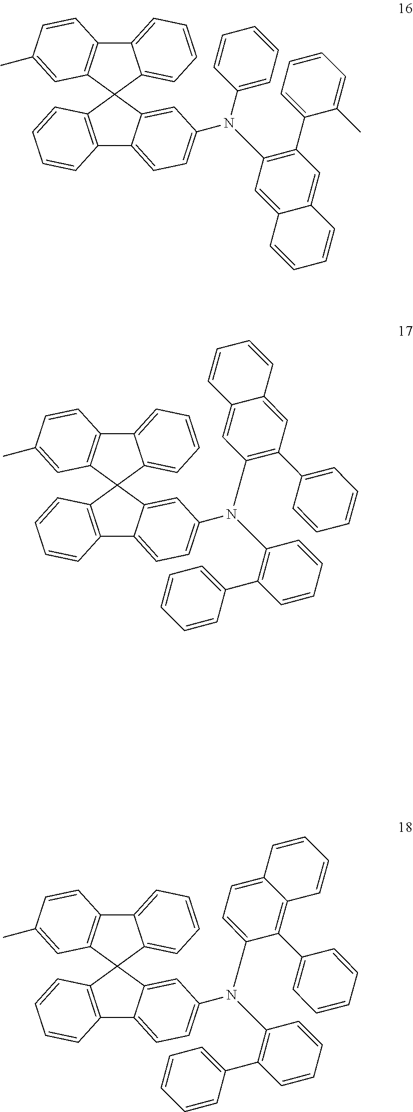

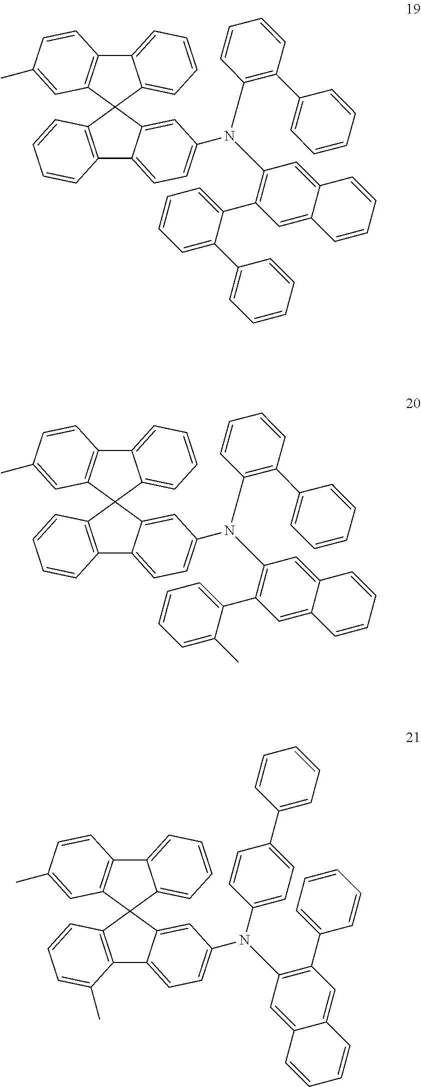 [SMILES: Cc1ccc2c(c1)C1(c3ccccc3-2)c2ccccc2-c2ccc(N(c3ccccc3-c3ccccc3)c3cc4ccccc4cc3-c3ccccc3-c3ccccc3)cc21 ; Cc1ccc2c(c1)C1(c3ccccc3-2)c2ccccc2-c2ccc(N(c3ccccc3-c3ccccc3)c3cc4ccccc4cc3-c3ccccc3C)cc21 ; Cc1ccc2c(c1)C1(c3ccccc3-2)c2cc(N(c3ccc(-c4ccccc4)cc3)c3cc4ccccc4cc3-c3ccccc3)ccc2-c2c(C)cccc21]

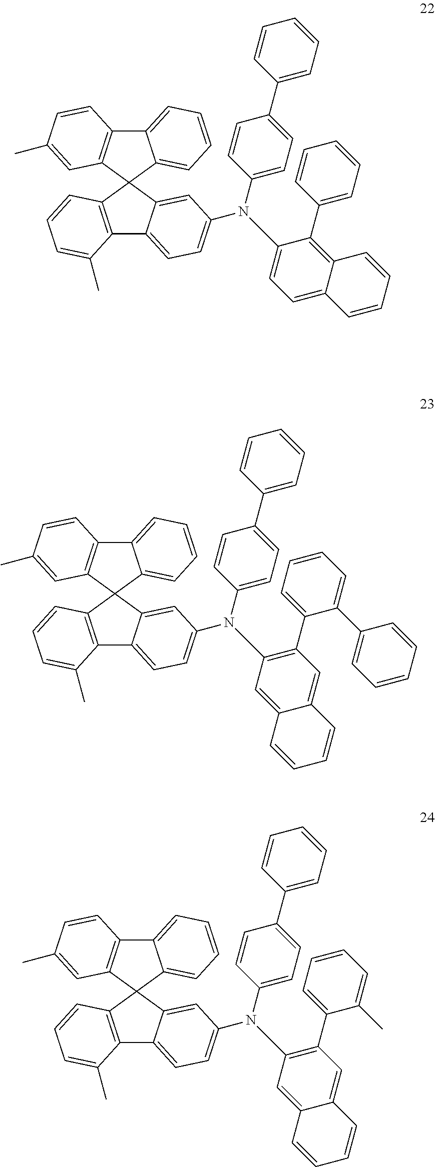

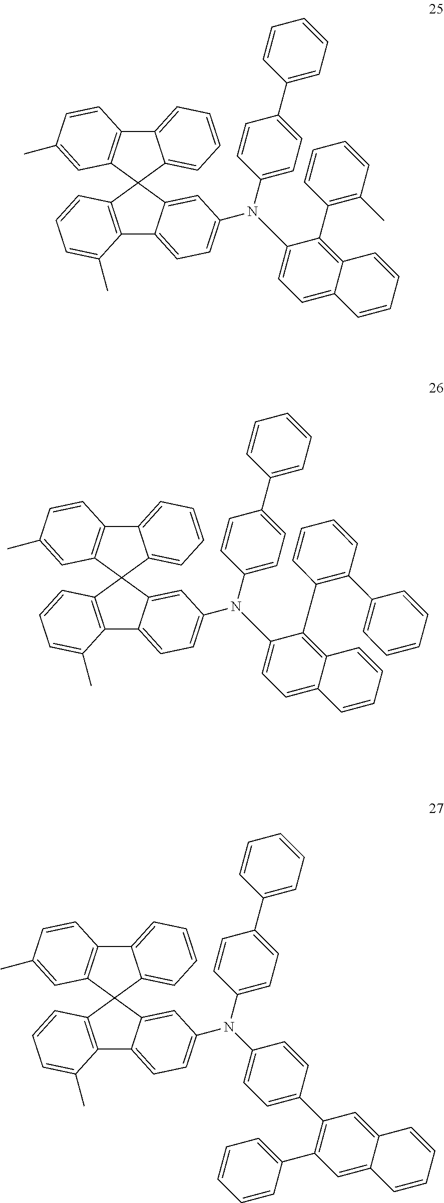 [SMILES: Cc1ccc2c(c1)C1(c3ccccc3-2)c2cc(N(c3ccc(-c4ccccc4)cc3)c3ccc4ccccc4c3-c3ccccc3C)ccc2-c2c(C)cccc21 ; Cc1ccc2c(c1)C1(c3ccccc3-2)c2cc(N(c3ccc(-c4ccccc4)cc3)c3ccc4ccccc4c3-c3ccccc3-c3ccccc3)ccc2-c2c(C)cccc21 ; Cc1ccc2c(c1)C1(c3ccccc3-2)c2cc(N(c3ccc(-c4ccccc4)cc3)c3ccc(-c4cc5ccccc5cc4-c4ccccc4)cc3)ccc2-c2c(C)cccc21]

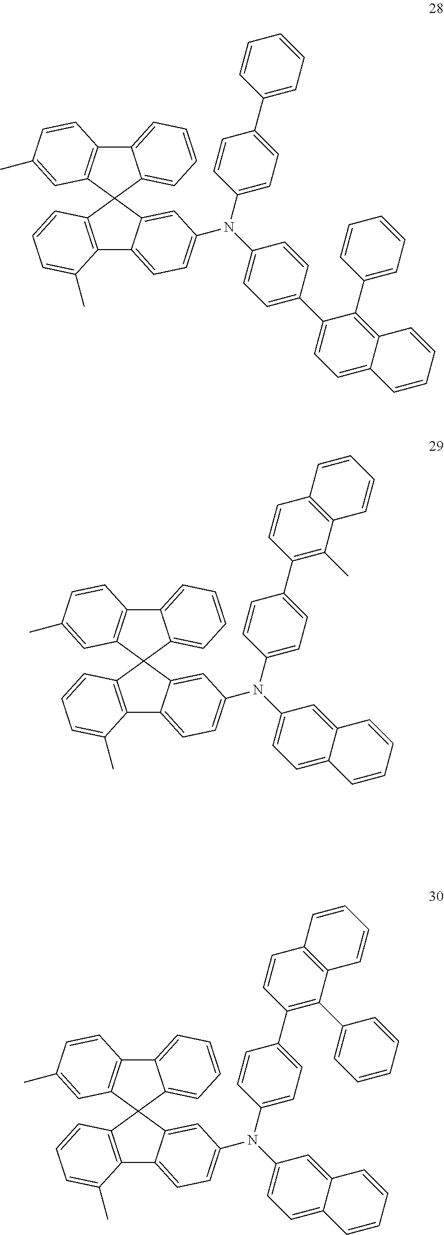 [SMILES: Cc1ccc2c(c1)C1(c3ccccc3-2)c2cc(N(c3ccc(-c4ccccc4)cc3)c3ccc(-c4ccc5ccccc5c4-c4ccccc4)cc3)ccc2-c2c(C)cccc21 ; Cc1ccc2c(c1)C1(c3ccccc3-2)c2cc(N(c3ccc(-c4ccc5ccccc5c4C)cc3)c3ccc4ccccc4c3)ccc2-c2c(C)cccc21 ; Cc1ccc2c(c1)C1(c3ccccc3-2)c2cc(N(c3ccc(-c4ccc5ccccc5c4-c4ccccc4)cc3)c3ccc4ccccc4c3)ccc2-c2c(C)cccc21]

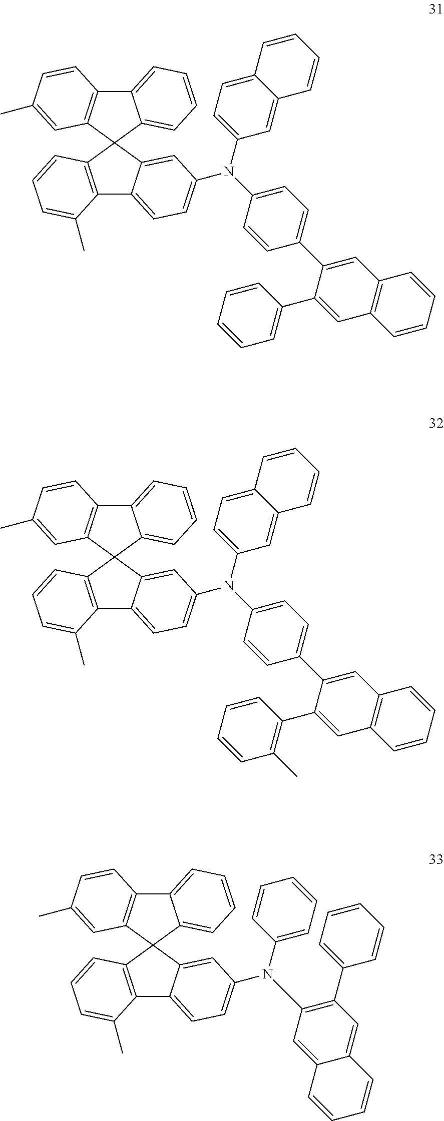 [SMILES: Cc1ccc2c(c1)C1(c3ccccc3-2)c2cc(N(c3ccc(-c4cc5ccccc5cc4-c4ccccc4)cc3)c3ccc4ccccc4c3)ccc2-c2c(C)cccc21 ; Cc1ccc2c(c1)C1(c3ccccc3-2)c2cc(N(c3ccc(-c4cc5ccccc5cc4-c4ccccc4C)cc3)c3ccc4ccccc4c3)ccc2-c2c(C)cccc21 ; Cc1ccc2c(c1)C1(c3ccccc3-2)c2cc(N(c3ccccc3)c3cc4ccccc4cc3-c3ccccc3)ccc2-c2c(C)cccc21]

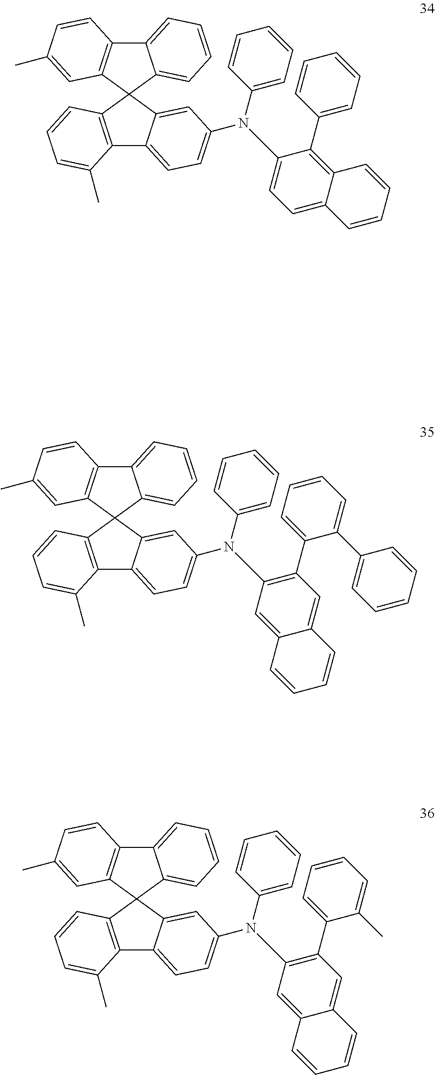

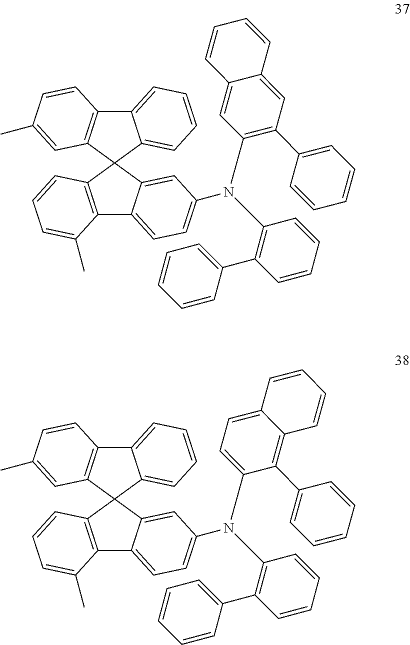 [SMILES: Cc1ccc2c(c1)C1(c3ccccc3-2)c2cc(N(c3ccccc3-c3ccccc3)c3cc4ccccc4cc3-c3ccccc3)ccc2-c2c(C)cccc21 ; Cc1ccc2c(c1)C1(c3ccccc3-2)c2cc(N(c3ccccc3-c3ccccc3)c3ccc4ccccc4c3-c3ccccc3)ccc2-c2c(C)cccc21]

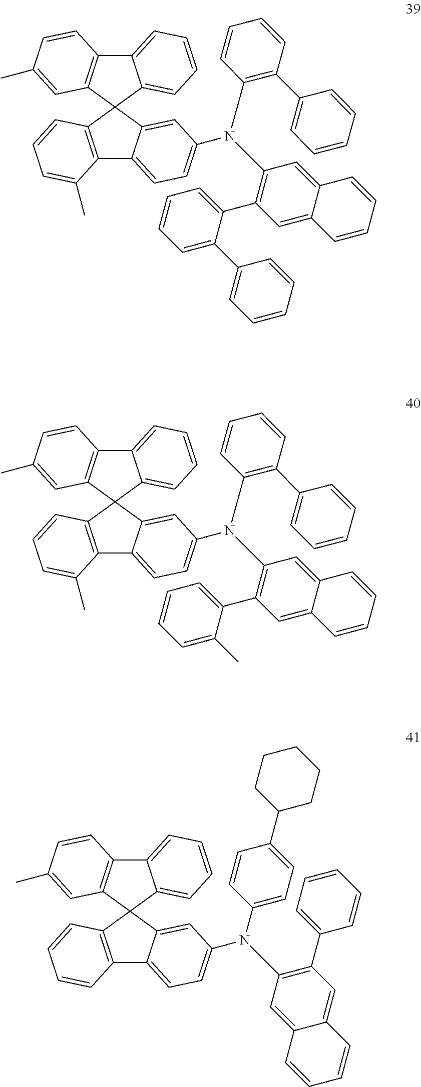 [SMILES: Cc1ccc2c(c1)C1(c3ccccc3-2)c2cc(N(c3ccccc3-c3ccccc3)c3cc4ccccc4cc3-c3ccccc3-c3ccccc3)ccc2-c2c(C)cccc21 ; Cc1ccc2c(c1)C1(c3ccccc3-2)c2cc(N(c3ccccc3-c3ccccc3)c3cc4ccccc4cc3-c3ccccc3C)ccc2-c2c(C)cccc21 ; Cc1ccc2c(c1)C1(c3ccccc3-2)c2ccccc2-c2ccc(N(c3ccc(C4CCCCC4)cc3)c3cc4ccccc4cc3-c3ccccc3)cc21]

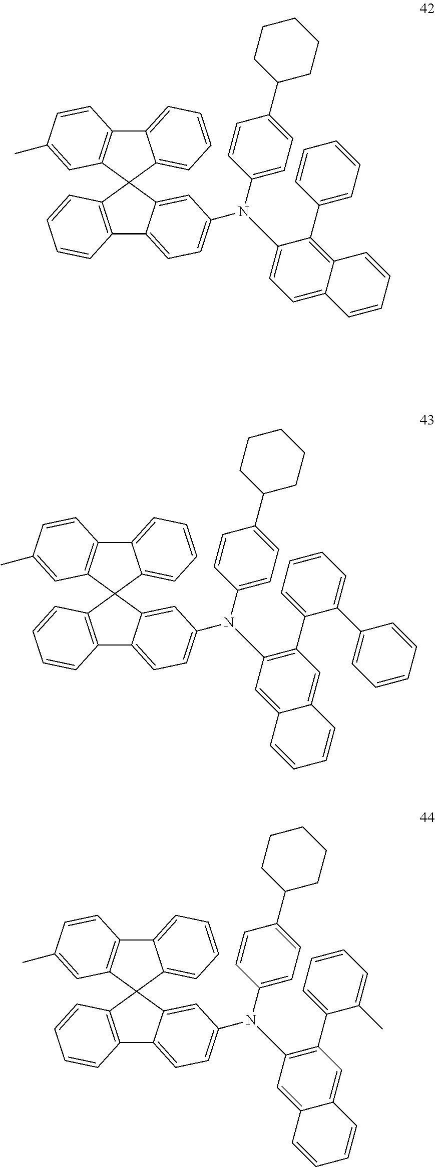

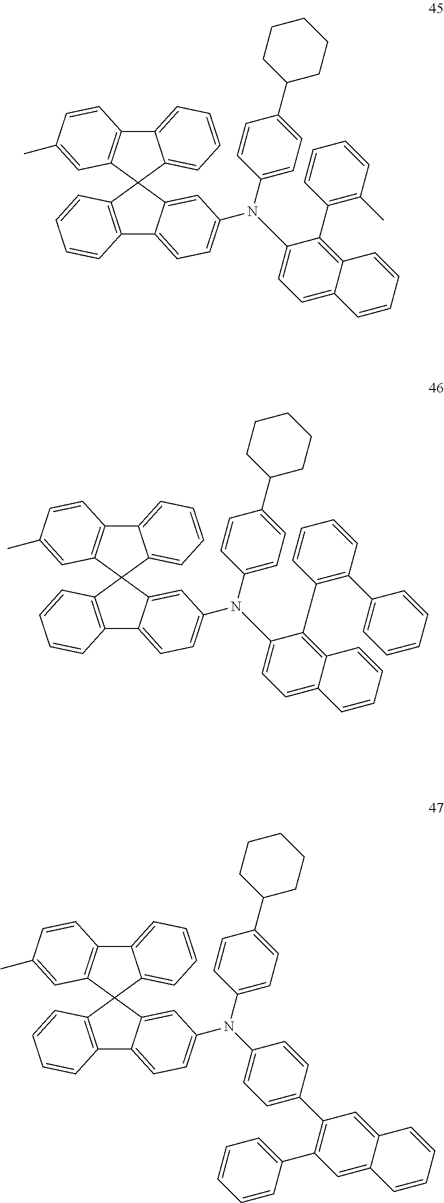 [SMILES: Cc1ccc2c(c1)C1(c3ccccc3-2)c2ccccc2-c2ccc(N(c3ccc(C4CCCCC4)cc3)c3ccc4ccccc4c3-c3ccccc3C)cc21 ; Cc1ccc2c(c1)C1(c3ccccc3-2)c2ccccc2-c2ccc(N(c3ccc(C4CCCCC4)cc3)c3ccc4ccccc4c3-c3ccccc3-c3ccccc3)cc21 ; Cc1ccc2c(c1)C1(c3ccccc3-2)c2ccccc2-c2ccc(N(c3ccc(-c4cc5ccccc5cc4-c4ccccc4)cc3)c3ccc(C4CCCCC4)cc3)cc21]

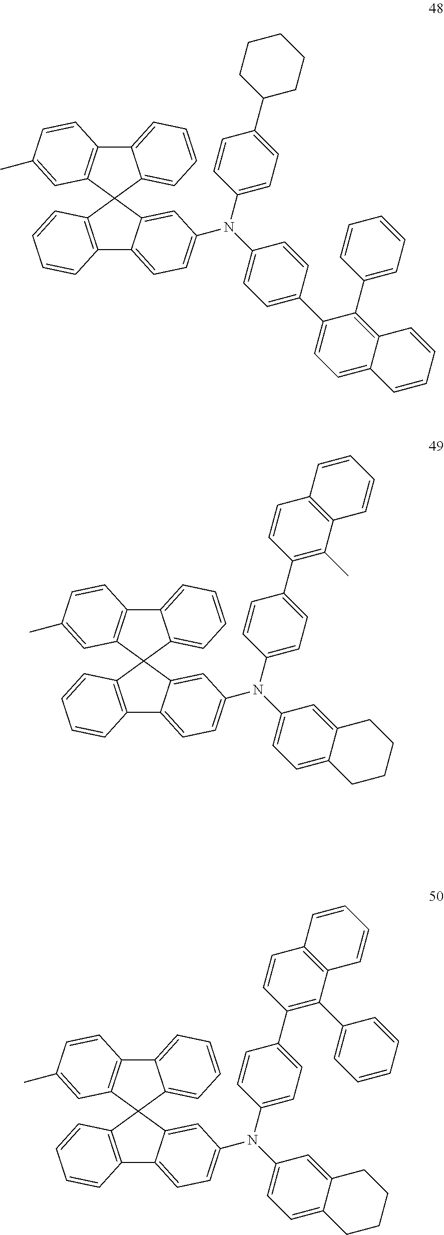 [SMILES: Cc1ccc2c(c1)C1(c3ccccc3-2)c2ccccc2-c2ccc(N(c3ccc(-c4ccc5ccccc5c4-c4ccccc4)cc3)c3ccc(C4CCCCC4)cc3)cc21 ; Cc1ccc2c(c1)C1(c3ccccc3-2)c2ccccc2-c2ccc(N(c3ccc(-c4ccc5ccccc5c4C)cc3)c3ccc4c(c3)CCCC4)cc21 ; Cc1ccc2c(c1)C1(c3ccccc3-2)c2ccccc2-c2ccc(N(c3ccc(-c4ccc5ccccc5c4-c4ccccc4)cc3)c3ccc4c(c3)CCCC4)cc21]

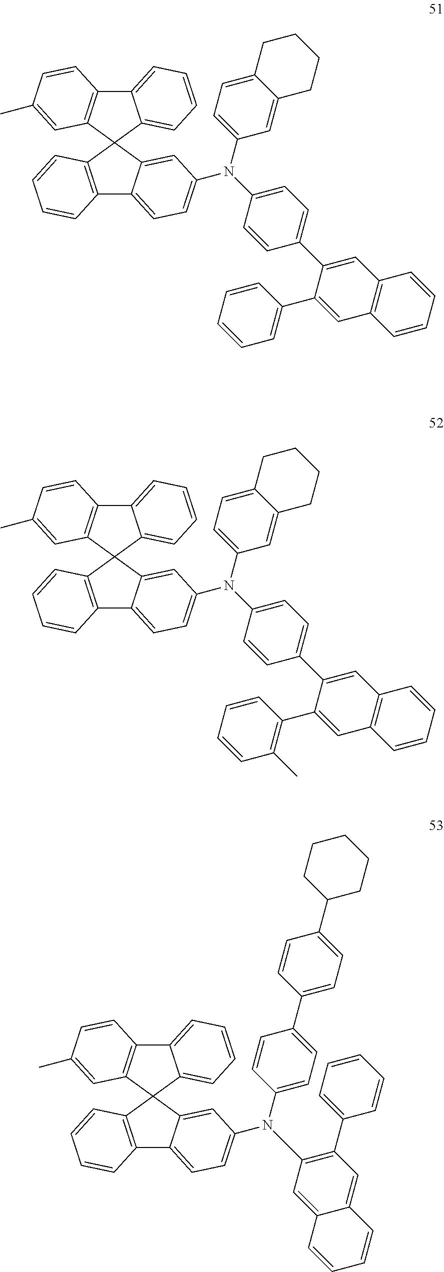 [SMILES: Cc1ccc2c(c1)C1(c3ccccc3-2)c2ccccc2-c2ccc(N(c3ccc(-c4cc5ccccc5cc4-c4ccccc4)cc3)c3ccc4c(c3)CCCC4)cc21 ; Cc1ccc2c(c1)C1(c3ccccc3-2)c2ccccc2-c2ccc(N(c3ccc(-c4cc5ccccc5cc4-c4ccccc4C)cc3)c3ccc4c(c3)CCCC4)cc21 ; Cc1ccc2c(c1)C1(c3ccccc3-2)c2ccccc2-c2ccc(N(c3ccc(-c4ccc(C5CCCCC5)cc4)cc3)c3cc4ccccc4cc3-c3ccccc3)cc21]

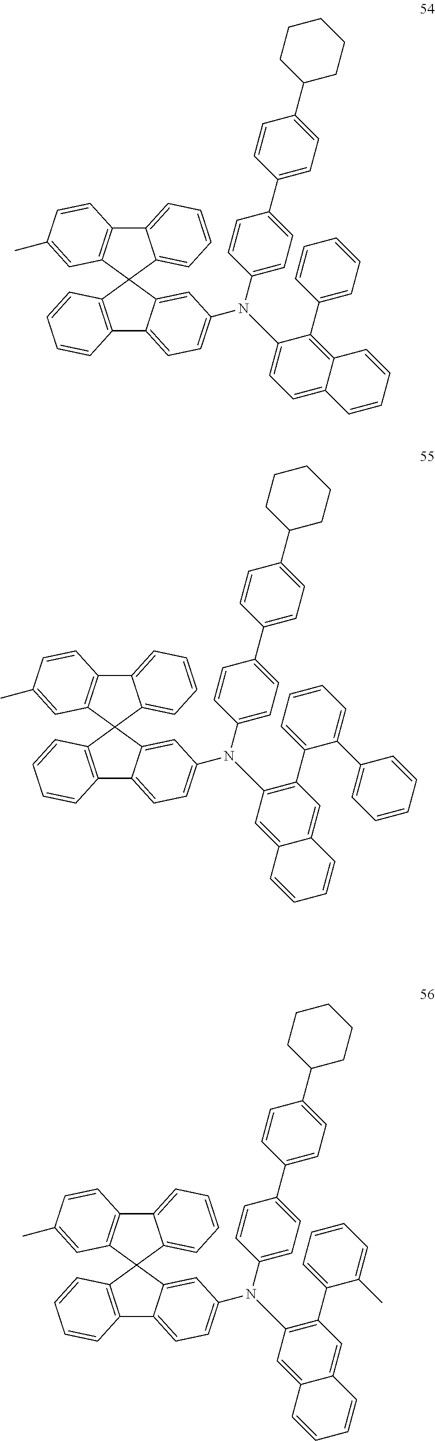

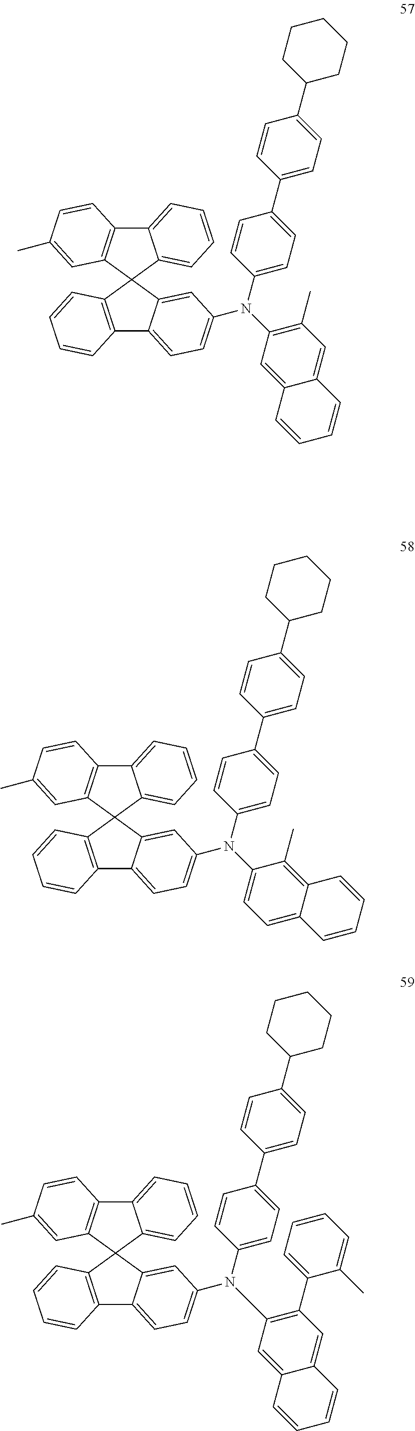

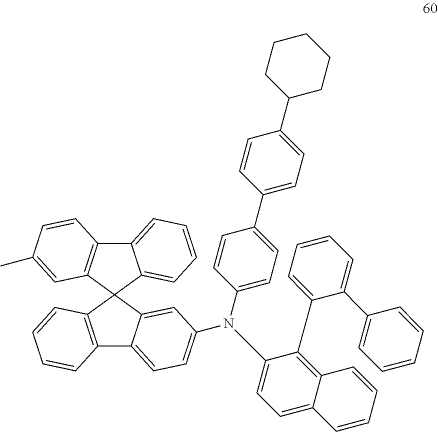 [SMILES: Cc1ccc2c(c1)C1(c3ccccc3-2)c2ccccc2-c2ccc(N(c3ccc(-c4ccc(C5CCCCC5)cc4)cc3)c3ccc4ccccc4c3-c3ccccc3-c3ccccc3)cc21]

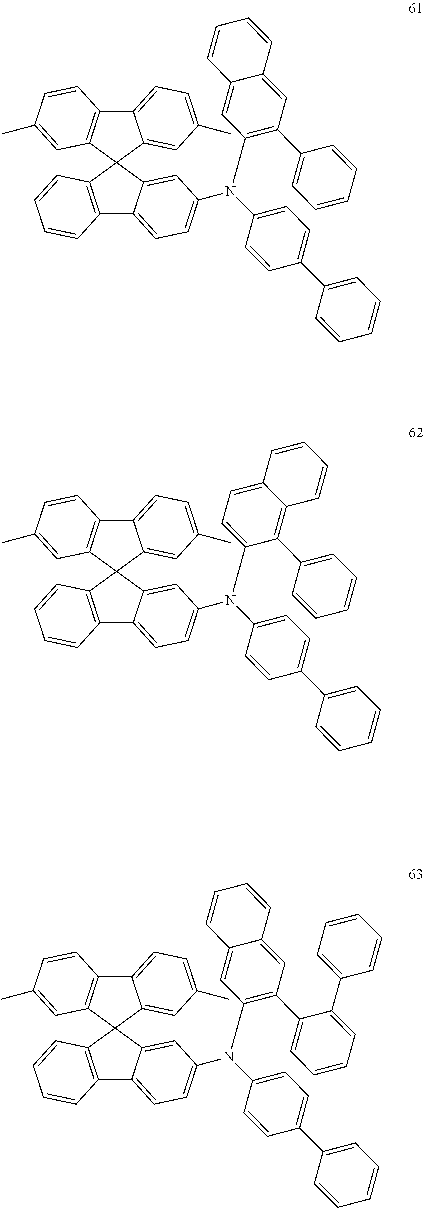 [SMILES: Cc1ccc2c(c1)C1(c3ccccc3-c3ccc(N(c4ccc(-c5ccccc5)cc4)c4cc5ccccc5cc4-c4ccccc4)cc31)c1cc(C)ccc1-2 ; Cc1ccc2c(c1)C1(c3ccccc3-c3ccc(N(c4ccc(-c5ccccc5)cc4)c4ccc5ccccc5c4-c4ccccc4)cc31)c1cc(C)ccc1-2 ; Cc1ccc2c(c1)C1(c3ccccc3-c3ccc(N(c4ccc(-c5ccccc5)cc4)c4cc5ccccc5cc4-c4ccccc4-c4ccccc4)cc31)c1cc(C)ccc1-2]

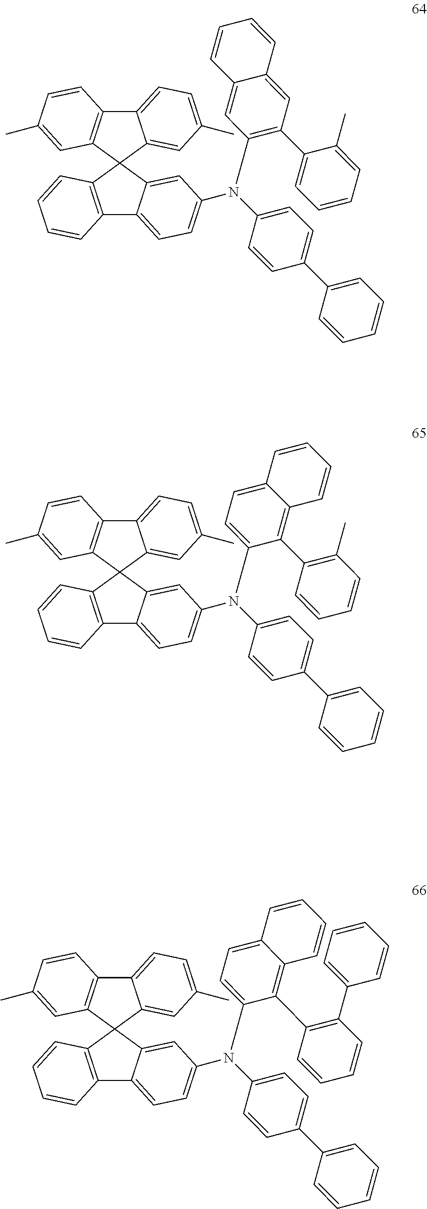 [SMILES: Cc1ccc2c(c1)C1(c3ccccc3-c3ccc(N(c4ccc(-c5ccccc5)cc4)c4cc5ccccc5cc4-c4ccccc4C)cc31)c1cc(C)ccc1-2 ; Cc1ccc2c(c1)C1(c3ccccc3-c3ccc(N(c4ccc(-c5ccccc5)cc4)c4ccc5ccccc5c4-c4ccccc4C)cc31)c1cc(C)ccc1-2 ; Cc1ccc2c(c1)C1(c3ccccc3-c3ccc(N(c4ccc(-c5ccccc5)cc4)c4ccc5ccccc5c4-c4ccccc4-c4ccccc4)cc31)c1cc(C)ccc1-2]

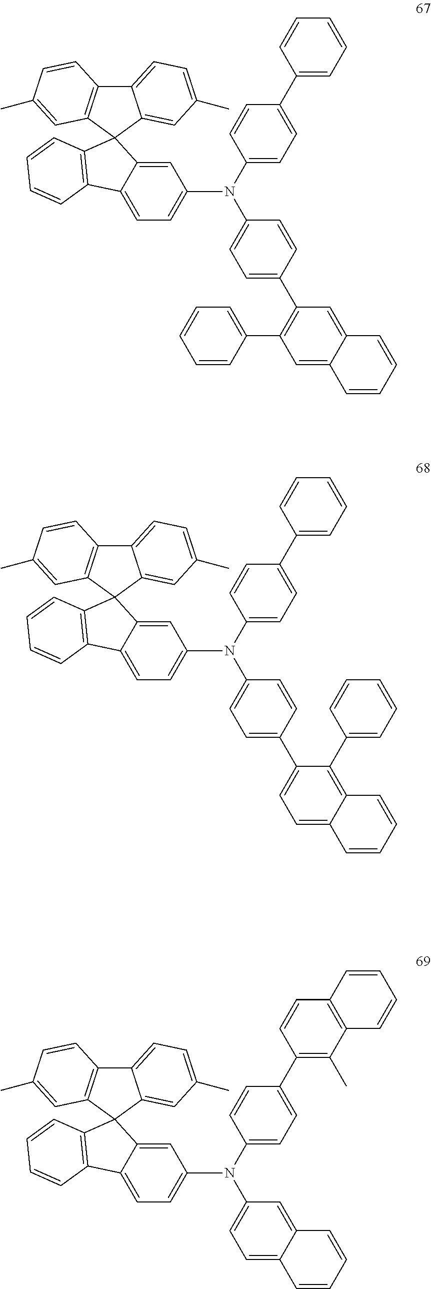 [SMILES: Cc1ccc2c(c1)C1(c3ccccc3-c3ccc(N(c4ccc(-c5ccccc5)cc4)c4ccc(-c5cc6ccccc6cc5-c5ccccc5)cc4)cc31)c1cc(C)ccc1-2 ; Cc1ccc2c(c1)C1(c3ccccc3-c3ccc(N(c4ccc(-c5ccccc5)cc4)c4ccc(-c5ccc6ccccc6c5-c5ccccc5)cc4)cc31)c1cc(C)ccc1-2 ; Cc1ccc2c(c1)C1(c3ccccc3-c3ccc(N(c4ccc(-c5ccc6ccccc6c5C)cc4)c4ccc5ccccc5c4)cc31)c1cc(C)ccc1-2]

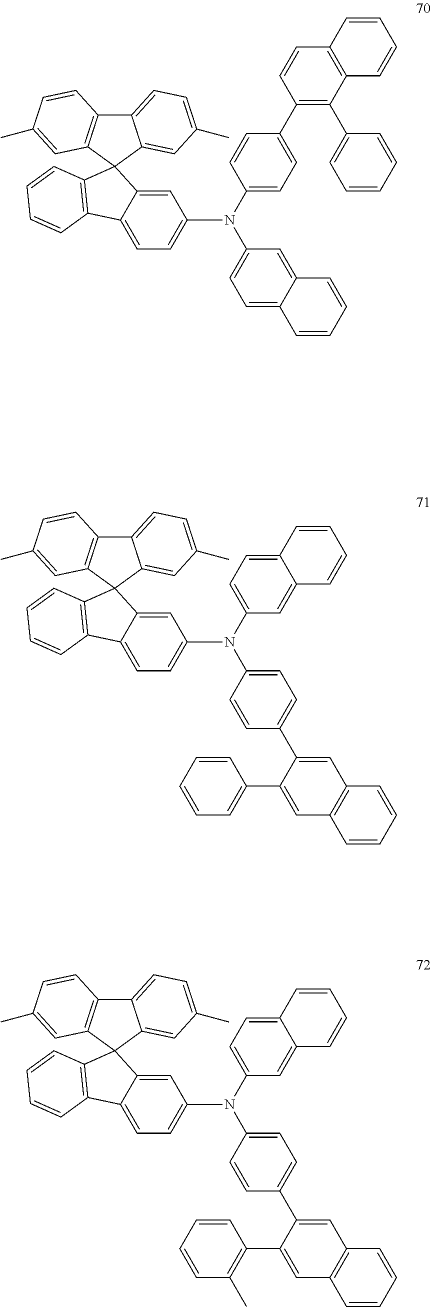 [SMILES: Cc1ccc2c(c1)C1(c3ccccc3-c3ccc(N(c4ccc(-c5ccc6ccccc6c5-c5ccccc5)cc4)c4ccc5ccccc5c4)cc31)c1cc(C)ccc1-2 ; Cc1ccc2c(c1)C1(c3ccccc3-c3ccc(N(c4ccc(-c5cc6ccccc6cc5-c5ccccc5)cc4)c4ccc5ccccc5c4)cc31)c1cc(C)ccc1-2 ; Cc1ccc2c(c1)C1(c3ccccc3-c3ccc(N(c4ccc(-c5cc6ccccc6cc5-c5ccccc5C)cc4)c4ccc5ccccc5c4)cc31)c1cc(C)ccc1-2]

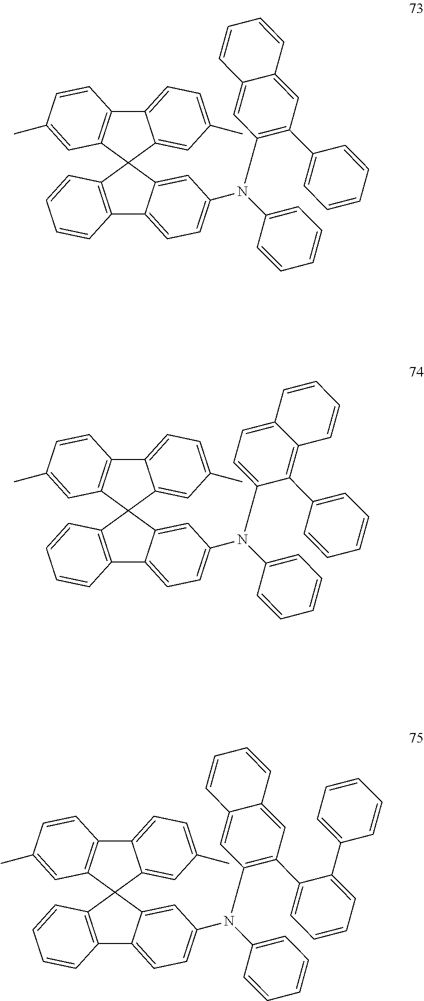 [SMILES: Cc1ccc2c(c1)C1(c3ccccc3-c3ccc(N(c4ccccc4)c4cc5ccccc5cc4-c4ccccc4)cc31)c1cc(C)ccc1-2 ; Cc1ccc2c(c1)C1(c3ccccc3-c3ccc(N(c4ccccc4)c4ccc5ccccc5c4-c4ccccc4)cc31)c1cc(C)ccc1-2 ; Cc1ccc2c(c1)C1(c3ccccc3-c3ccc(N(c4ccccc4)c4cc5ccccc5cc4-c4ccccc4-c4ccccc4)cc31)c1cc(C)ccc1-2]

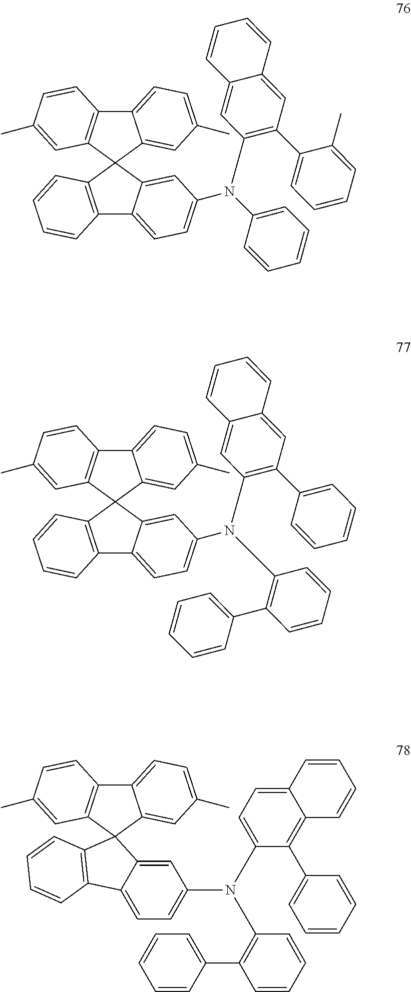 [SMILES: Cc1ccc2c(c1)C1(c3ccccc3-c3ccc(N(c4ccccc4)c4cc5ccccc5cc4-c4ccccc4C)cc31)c1cc(C)ccc1-2 ; Cc1ccc2c(c1)C1(c3ccccc3-c3ccc(N(c4ccccc4-c4ccccc4)c4cc5ccccc5cc4-c4ccccc4)cc31)c1cc(C)ccc1-2 ; Cc1ccc2c(c1)C1(c3ccccc3-c3ccc(N(c4ccccc4-c4ccccc4)c4ccc5ccccc5c4-c4ccccc4)cc31)c1cc(C)ccc1-2]

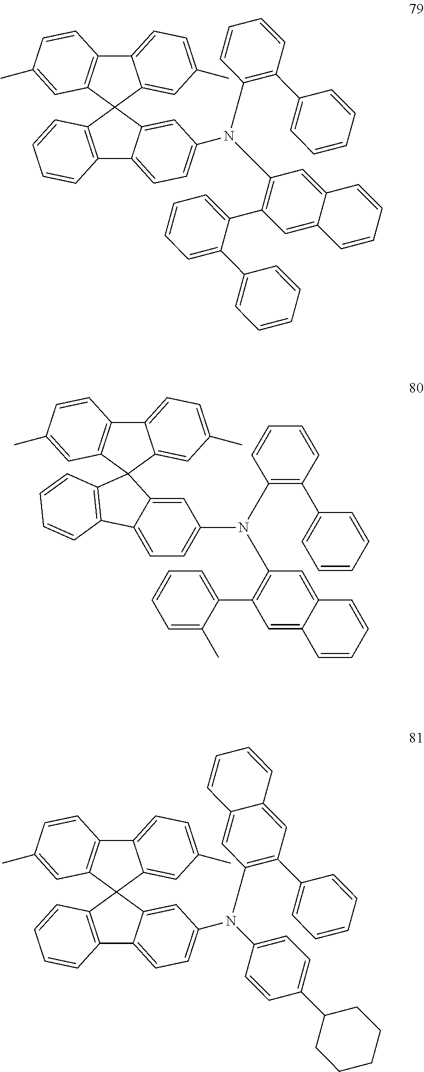 [SMILES: Cc1ccc2c(c1)C1(c3ccccc3-c3ccc(N(c4ccccc4-c4ccccc4)c4cc5ccccc5cc4-c4ccccc4-c4ccccc4)cc31)c1cc(C)ccc1-2 ; Cc1ccc2c(c1)C1(c3ccccc3-c3ccc(N(c4ccccc4-c4ccccc4)c4cc5ccccc5cc4-c4ccccc4C)cc31)c1cc(C)ccc1-2 ; Cc1ccc2c(c1)C1(c3ccccc3-c3ccc(N(c4ccc(C5CCCCC5)cc4)c4cc5ccccc5cc4-c4ccccc4)cc31)c1cc(C)ccc1-2]

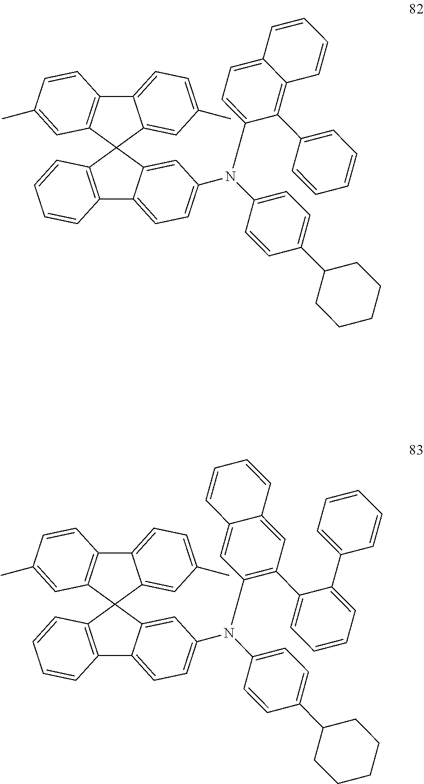 [SMILES: Cc1ccc2c(c1)C1(c3ccccc3-c3ccc(N(c4ccc(C5CCCCC5)cc4)c4ccc5ccccc5c4-c4ccccc4)cc31)c1cc(C)ccc1-2 ; Cc1ccc2c(c1)C1(c3ccccc3-c3ccc(N(c4ccc(C5CCCCC5)cc4)c4cc5ccccc5cc4-c4ccccc4-c4ccccc4)cc31)c1cc(C)ccc1-2]

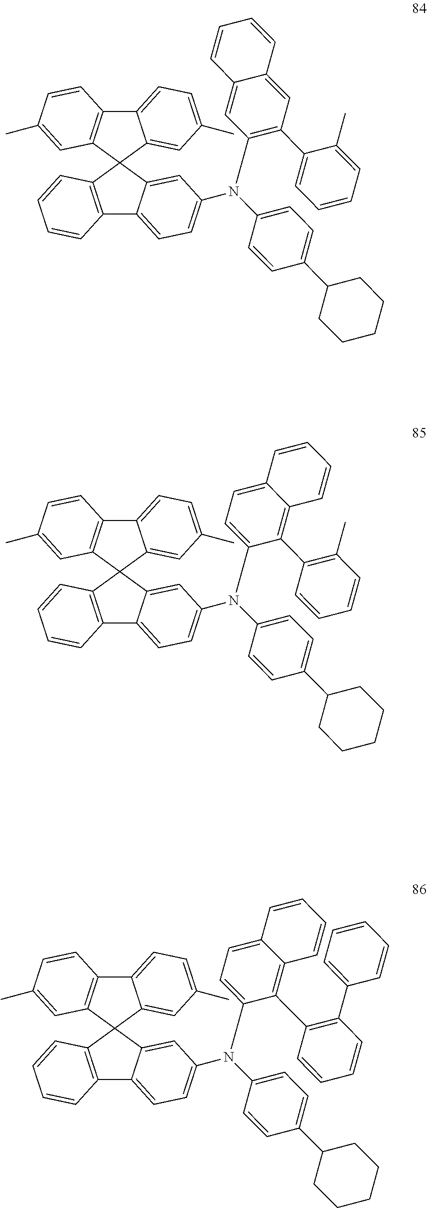 [SMILES: Cc1ccc2c(c1)C1(c3ccccc3-c3ccc(N(c4ccc(C5CCCCC5)cc4)c4cc5ccccc5cc4-c4ccccc4C)cc31)c1cc(C)ccc1-2 ; Cc1ccc2c(c1)C1(c3ccccc3-c3ccc(N(c4ccc(C5CCCCC5)cc4)c4ccc5ccccc5c4-c4ccccc4C)cc31)c1cc(C)ccc1-2 ; Cc1ccc2c(c1)C1(c3ccccc3-c3ccc(N(c4ccc(C5CCCCC5)cc4)c4ccc5ccccc5c4-c4ccccc4-c4ccccc4)cc31)c1cc(C)ccc1-2]

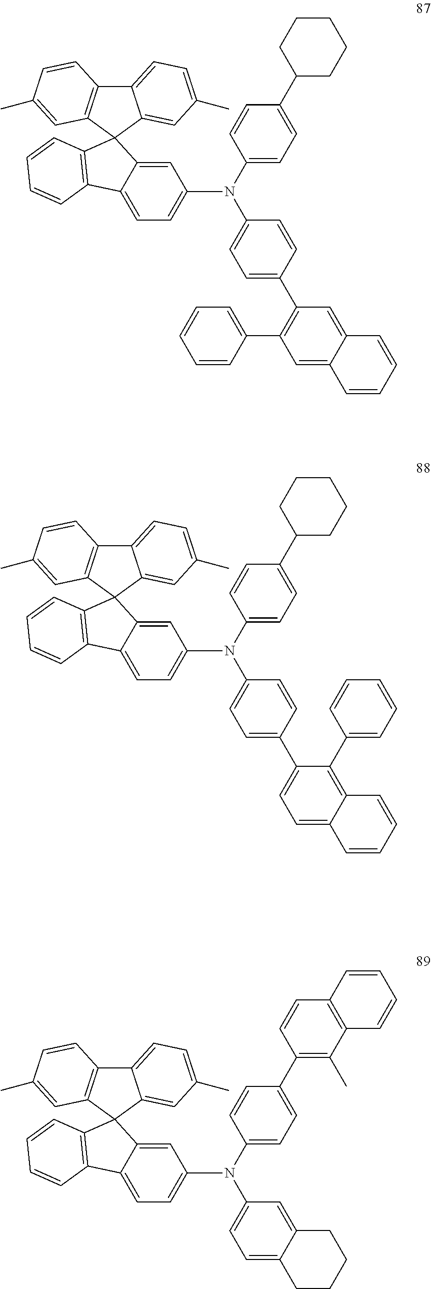 [SMILES: Cc1ccc2c(c1)C1(c3ccccc3-c3ccc(N(c4ccc(-c5cc6ccccc6cc5-c5ccccc5)cc4)c4ccc(C5CCCCC5)cc4)cc31)c1cc(C)ccc1-2 ; Cc1ccc2c(c1)C1(c3ccccc3-c3ccc(N(c4ccc(-c5ccc6ccccc6c5-c5ccccc5)cc4)c4ccc(C5CCCCC5)cc4)cc31)c1cc(C)ccc1-2 ; Cc1ccc2c(c1)C1(c3ccccc3-c3ccc(N(c4ccc(-c5ccc6ccccc6c5C)cc4)c4ccc5c(c4)CCCC5)cc31)c1cc(C)ccc1-2]

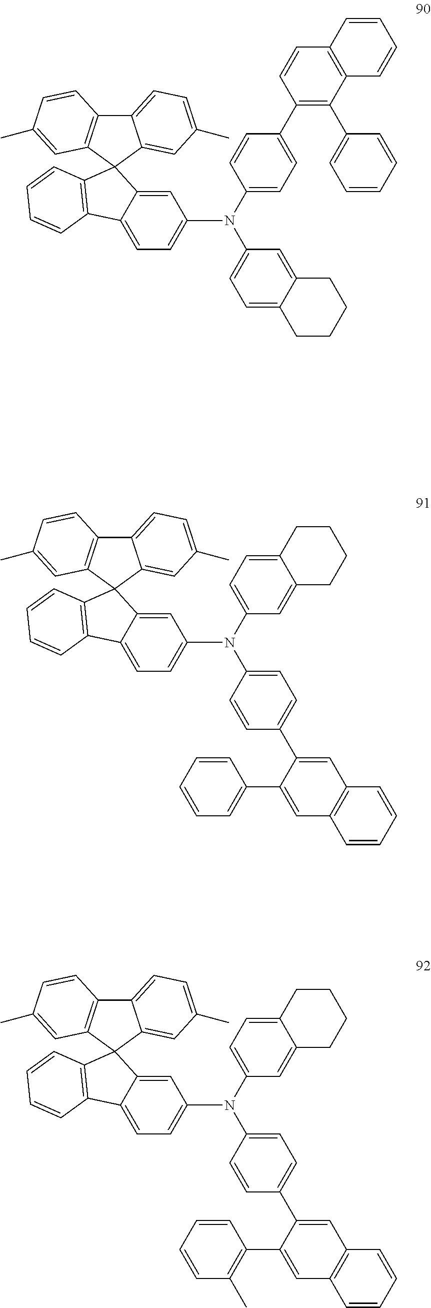 [SMILES: Cc1ccc2c(c1)C1(c3ccccc3-c3ccc(N(c4ccc(-c5ccc6ccccc6c5-c5ccccc5)cc4)c4ccc5c(c4)CCCC5)cc31)c1cc(C)ccc1-2 ; Cc1ccc2c(c1)C1(c3ccccc3-c3ccc(N(c4ccc(-c5cc6ccccc6cc5-c5ccccc5)cc4)c4ccc5c(c4)CCCC5)cc31)c1cc(C)ccc1-2 ; Cc1ccc2c(c1)C1(c3ccccc3-c3ccc(N(c4ccc(-c5cc6ccccc6cc5-c5ccccc5C)cc4)c4ccc5c(c4)CCCC5)cc31)c1cc(C)ccc1-2]

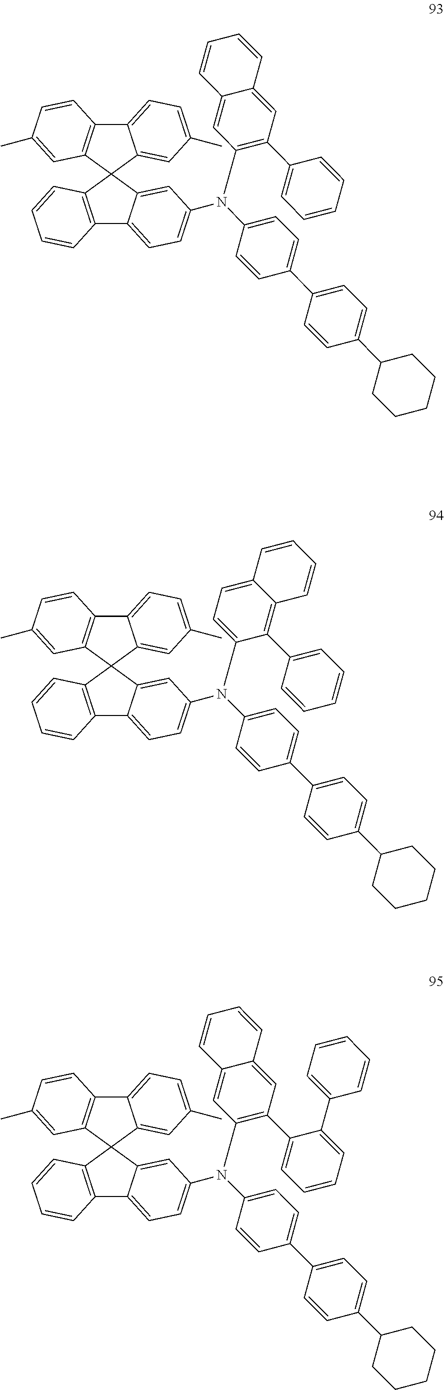 [SMILES: Cc1ccc2c(c1)C1(c3ccccc3-c3ccc(N(c4ccc(-c5ccc(C6CCCCC6)cc5)cc4)c4cc5ccccc5cc4-c4ccccc4)cc31)c1cc(C)ccc1-2 ; Cc1ccc2c(c1)C1(c3ccccc3-c3ccc(N(c4ccc(-c5ccc(C6CCCCC6)cc5)cc4)c4ccc5ccccc5c4-c4ccccc4)cc31)c1cc(C)ccc1-2 ; Cc1ccc2c(c1)C1(c3ccccc3-c3ccc(N(c4ccc(-c5ccc(C6CCCCC6)cc5)cc4)c4cc5ccccc5cc4-c4ccccc4-c4ccccc4)cc31)c1cc(C)ccc1-2]

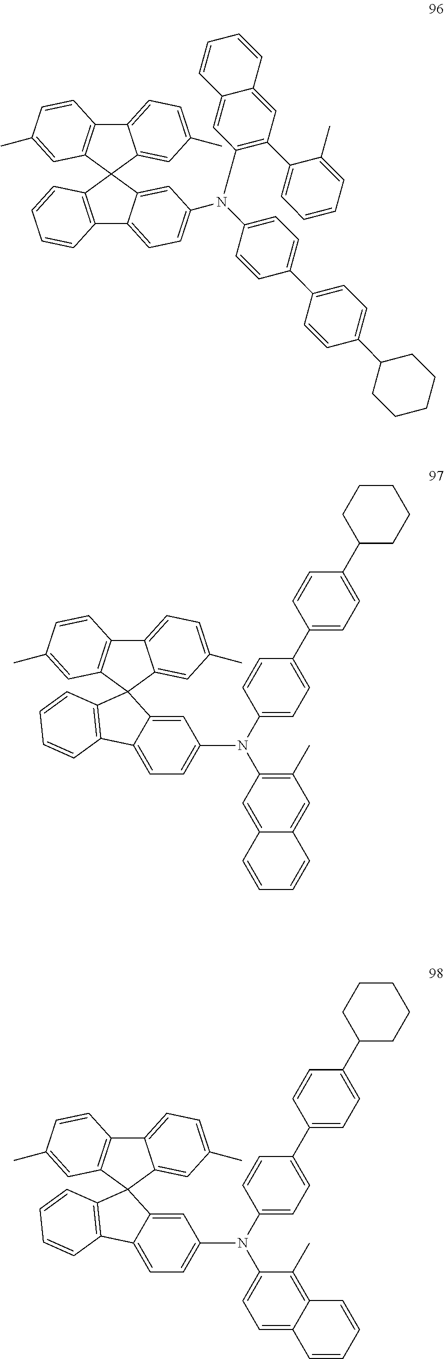 [SMILES: Cc1ccc2c(c1)C1(c3ccccc3-c3ccc(N(c4ccc(-c5ccc(C6CCCCC6)cc5)cc4)c4cc5ccccc5cc4-c4ccccc4C)cc31)c1cc(C)ccc1-2 ; Cc1ccc2c(c1)C1(c3ccccc3-c3ccc(N(c4ccc(-c5ccc(C6CCCCC6)cc5)cc4)c4cc5ccccc5cc4C)cc31)c1cc(C)ccc1-2 ; Cc1ccc2c(c1)C1(c3ccccc3-c3ccc(N(c4ccc(-c5ccc(C6CCCCC6)cc5)cc4)c4ccc5ccccc5c4C)cc31)c1cc(C)ccc1-2]

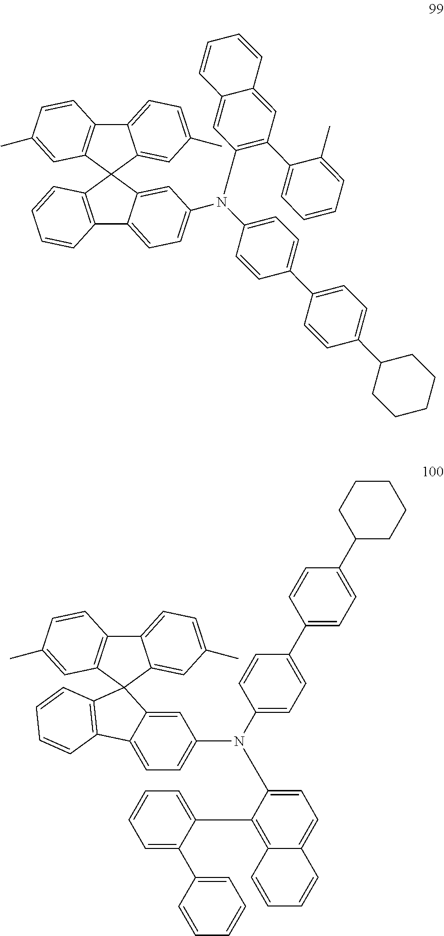 [SMILES: Cc1ccc2c(c1)C1(c3ccccc3-c3ccc(N(c4ccc(-c5ccc(C6CCCCC6)cc5)cc4)c4cc5ccccc5cc4-c4ccccc4C)cc31)c1cc(C)ccc1-2 ; Cc1ccc2c(c1)C1(c3ccccc3-c3ccc(N(c4ccc(-c5ccc(C6CCCCC6)cc5)cc4)c4ccc5ccccc5c4-c4ccccc4-c4ccccc4)cc31)c1cc(C)ccc1-2]

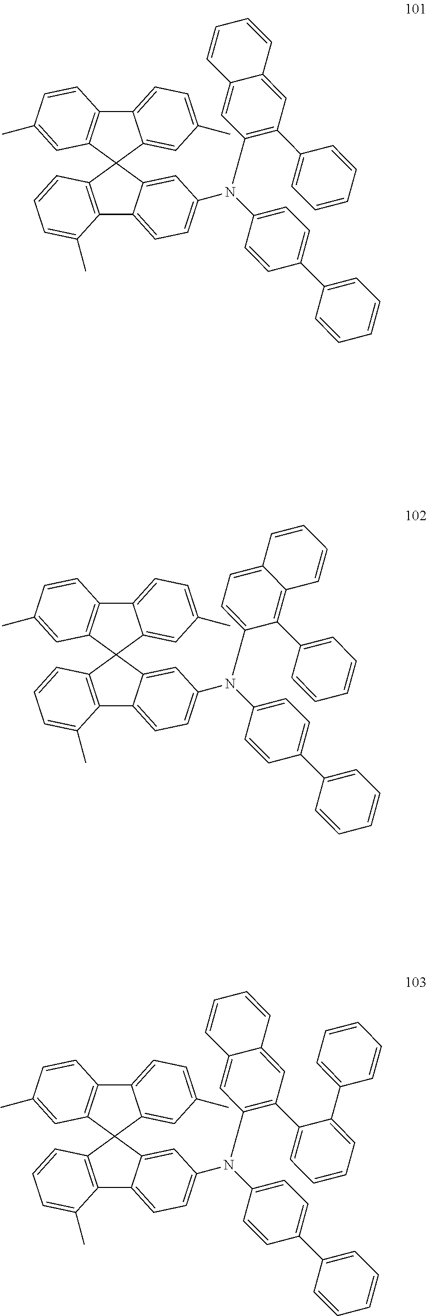 [SMILES: Cc1ccc2c(c1)C1(c3cc(C)ccc3-2)c2cc(N(c3ccc(-c4ccccc4)cc3)c3cc4ccccc4cc3-c3ccccc3)ccc2-c2c(C)cccc21 ; Cc1ccc2c(c1)C1(c3cc(C)ccc3-2)c2cc(N(c3ccc(-c4ccccc4)cc3)c3ccc4ccccc4c3-c3ccccc3)ccc2-c2c(C)cccc21 ; Cc1ccc2c(c1)C1(c3cc(C)ccc3-2)c2cc(N(c3ccc(-c4ccccc4)cc3)c3cc4ccccc4cc3-c3ccccc3-c3ccccc3)ccc2-c2c(C)cccc21]

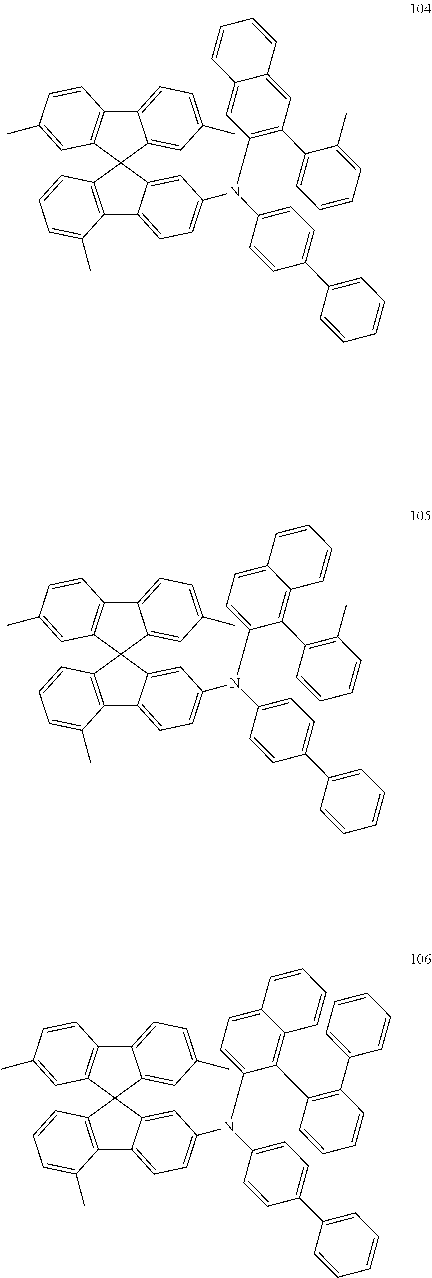 [SMILES: Cc1ccc2c(c1)C1(c3cc(C)ccc3-2)c2cc(N(c3ccc(-c4ccccc4)cc3)c3cc4ccccc4cc3-c3ccccc3C)ccc2-c2c(C)cccc21 ; Cc1ccc2c(c1)C1(c3cc(C)ccc3-2)c2cc(N(c3ccc(-c4ccccc4)cc3)c3ccc4ccccc4c3-c3ccccc3C)ccc2-c2c(C)cccc21 ; Cc1ccc2c(c1)C1(c3cc(C)ccc3-2)c2cc(N(c3ccc(-c4ccccc4)cc3)c3ccc4ccccc4c3-c3ccccc3-c3ccccc3)ccc2-c2c(C)cccc21]

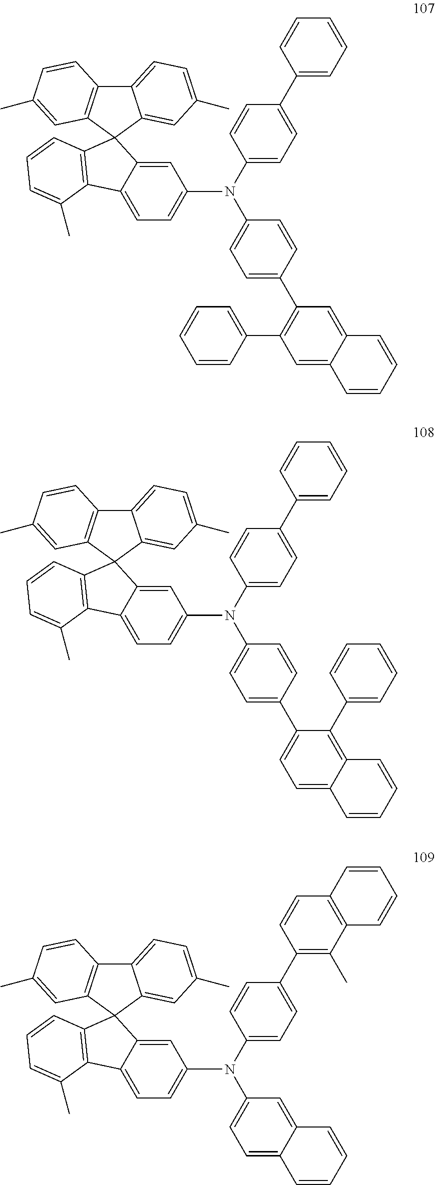 [SMILES: Cc1ccc2c(c1)C1(c3cc(C)ccc3-2)c2cc(N(c3ccc(-c4ccccc4)cc3)c3ccc(-c4cc5ccccc5cc4-c4ccccc4)cc3)ccc2-c2c(C)cccc21 ; Cc1ccc2c(c1)C1(c3cc(C)ccc3-2)c2cc(N(c3ccc(-c4ccccc4)cc3)c3ccc(-c4ccc5ccccc5c4-c4ccccc4)cc3)ccc2-c2c(C)cccc21 ; Cc1ccc2c(c1)C1(c3cc(C)ccc3-2)c2cc(N(c3ccc(-c4ccc5ccccc5c4C)cc3)c3ccc4ccccc4c3)ccc2-c2c(C)cccc21]

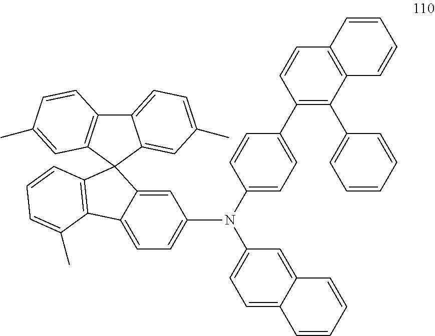 [SMILES: Cc1ccc2c(c1)C1(c3cc(C)ccc3-2)c2cc(N(c3ccc(-c4ccc5ccccc5c4-c4ccccc4)cc3)c3ccc4ccccc4c3)ccc2-c2c(C)cccc21]

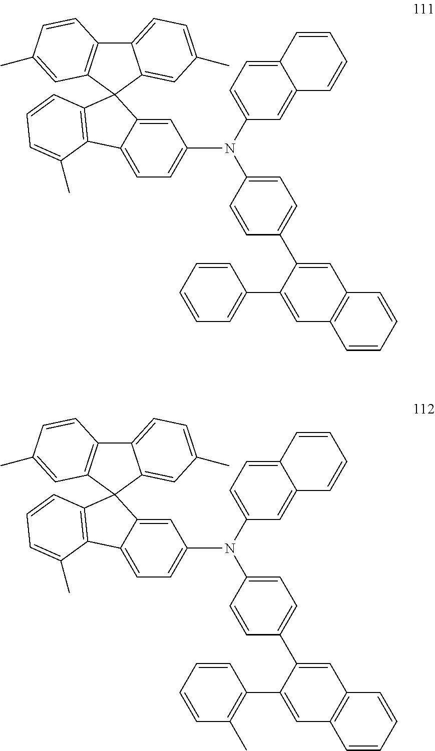 [SMILES: Cc1ccc2c(c1)C1(c3cc(C)ccc3-2)c2cc(N(c3ccc(-c4cc5ccccc5cc4-c4ccccc4)cc3)c3ccc4ccccc4c3)ccc2-c2c(C)cccc21 ; Cc1ccc2c(c1)C1(c3cc(C)ccc3-2)c2cc(N(c3ccc(-c4cc5ccccc5cc4-c4ccccc4C)cc3)c3ccc4ccccc4c3)ccc2-c2c(C)cccc21]

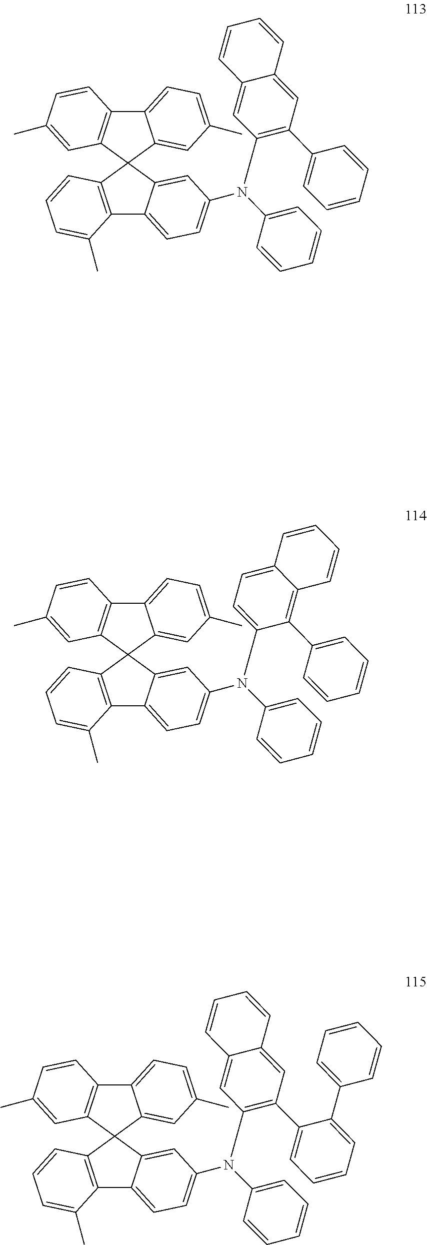 [SMILES: Cc1ccc2c(c1)C1(c3cc(C)ccc3-2)c2cc(N(c3ccccc3)c3cc4ccccc4cc3-c3ccccc3)ccc2-c2c(C)cccc21 ; Cc1ccc2c(c1)C1(c3cc(C)ccc3-2)c2cc(N(c3ccccc3)c3ccc4ccccc4c3-c3ccccc3)ccc2-c2c(C)cccc21 ; Cc1ccc2c(c1)C1(c3cc(C)ccc3-2)c2cc(N(c3ccccc3)c3cc4ccccc4cc3-c3ccccc3-c3ccccc3)ccc2-c2c(C)cccc21]

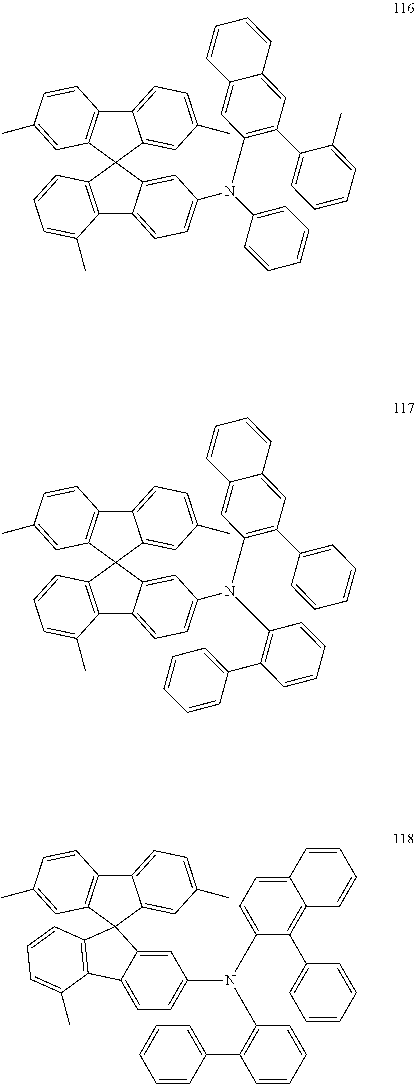 [SMILES: Cc1ccc2c(c1)C1(c3cc(C)ccc3-2)c2cc(N(c3ccccc3)c3cc4ccccc4cc3-c3ccccc3C)ccc2-c2c(C)cccc21 ; Cc1ccc2c(c1)C1(c3cc(C)ccc3-2)c2cc(N(c3ccccc3-c3ccccc3)c3cc4ccccc4cc3-c3ccccc3)ccc2-c2c(C)cccc21 ; Cc1ccc2c(c1)C1(c3cc(C)ccc3-2)c2cc(N(c3ccccc3-c3ccccc3)c3ccc4ccccc4c3-c3ccccc3)ccc2-c2c(C)cccc21]

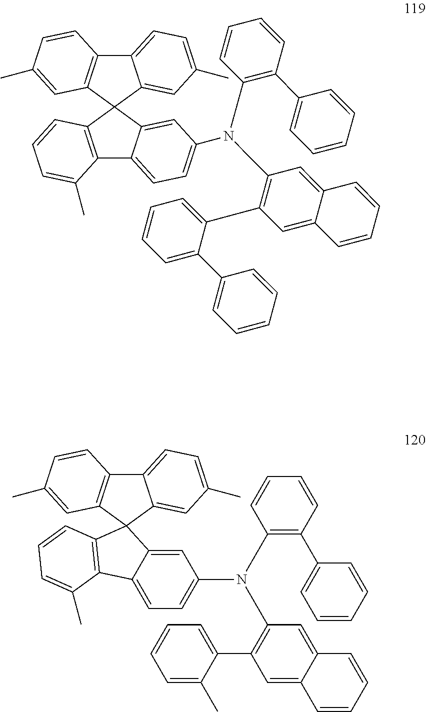 [SMILES: Cc1ccc2c(c1)C1(c3cc(C)ccc3-2)c2cc(N(c3ccccc3-c3ccccc3)c3cc4ccccc4cc3-c3ccccc3-c3ccccc3)ccc2-c2c(C)cccc21 ; Cc1ccc2c(c1)C1(c3cc(C)ccc3-2)c2cc(N(c3ccccc3-c3ccccc3)c3cc4ccccc4cc3-c3ccccc3C)ccc2-c2c(C)cccc21]

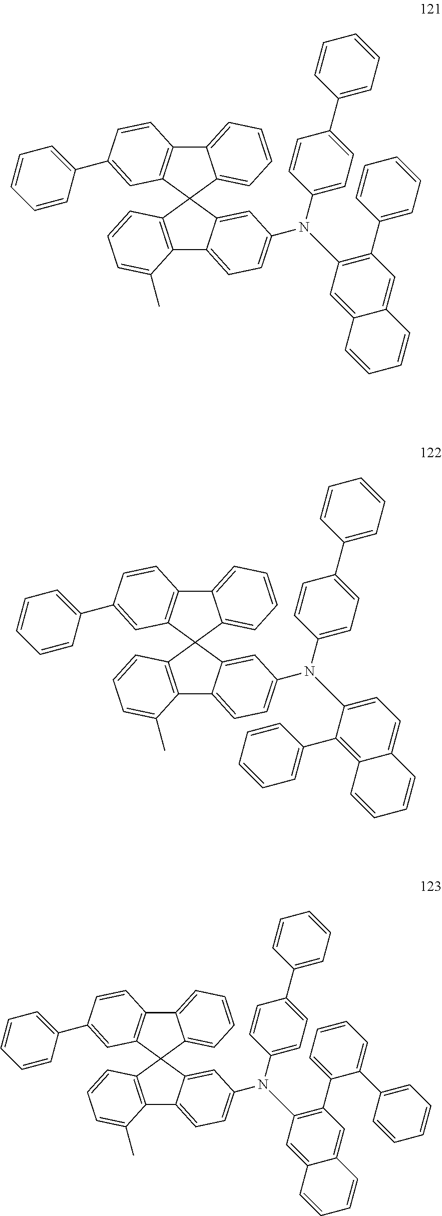 [SMILES: Cc1cccc2c1-c1ccc(N(c3ccc(-c4ccccc4)cc3)c3cc4ccccc4cc3-c3ccccc3)cc1C21c2ccccc2-c2ccc(-c3ccccc3)cc21 ; Cc1cccc2c1-c1ccc(N(c3ccc(-c4ccccc4)cc3)c3ccc4ccccc4c3-c3ccccc3)cc1C21c2ccccc2-c2ccc(-c3ccccc3)cc21 ; Cc1cccc2c1-c1ccc(N(c3ccc(-c4ccccc4)cc3)c3cc4ccccc4cc3-c3ccccc3-c3ccccc3)cc1C21c2ccccc2-c2ccc(-c3ccccc3)cc21]

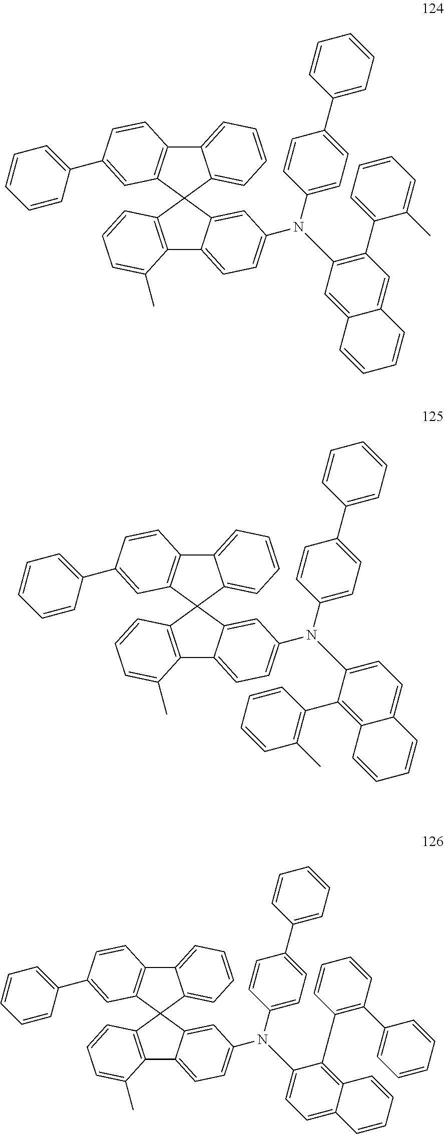 [SMILES: Cc1ccccc1-c1cc2ccccc2cc1N(c1ccc(-c2ccccc2)cc1)c1ccc2c(c1)C1(c3ccccc3-c3ccc(-c4ccccc4)cc31)c1cccc(C)c1-2 ; Cc1ccccc1-c1c(N(c2ccc(-c3ccccc3)cc2)c2ccc3c(c2)C2(c4ccccc4-c4ccc(-c5ccccc5)cc42)c2cccc(C)c2-3)ccc2ccccc12 ; Cc1cccc2c1-c1ccc(N(c3ccc(-c4ccccc4)cc3)c3ccc4ccccc4c3-c3ccccc3-c3ccccc3)cc1C21c2ccccc2-c2ccc(-c3ccccc3)cc21]

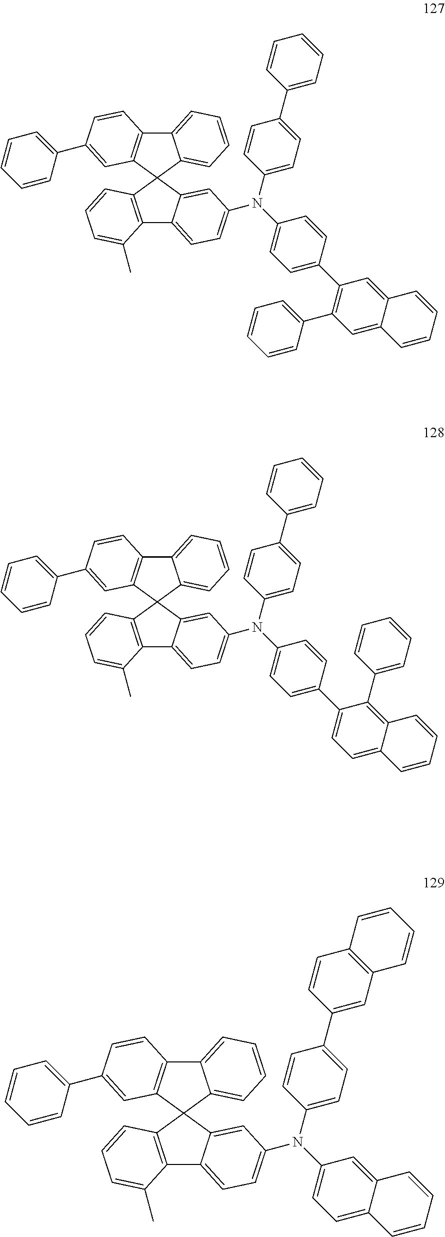 [SMILES: Cc1cccc2c1-c1ccc(N(c3ccc(-c4ccccc4)cc3)c3ccc(-c4cc5ccccc5cc4-c4ccccc4)cc3)cc1C21c2ccccc2-c2ccc(-c3ccccc3)cc21 ; Cc1cccc2c1-c1ccc(N(c3ccc(-c4ccccc4)cc3)c3ccc(-c4ccc5ccccc5c4-c4ccccc4)cc3)cc1C21c2ccccc2-c2ccc(-c3ccccc3)cc21 ; Cc1cccc2c1-c1ccc(N(c3ccc(-c4ccc5ccccc5c4)cc3)c3ccc4ccccc4c3)cc1C21c2ccccc2-c2ccc(-c3ccccc3)cc21]

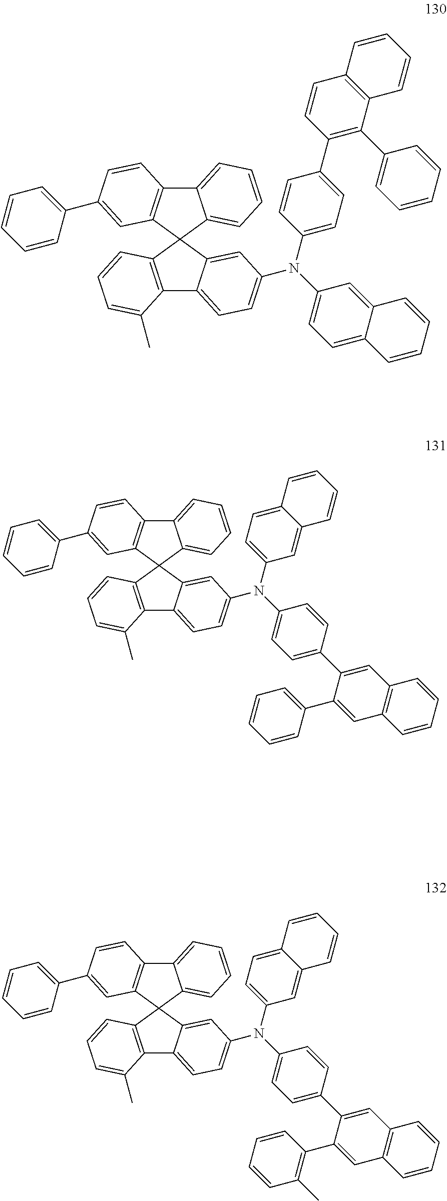 [SMILES: Cc1cccc2c1-c1ccc(N(c3ccc(-c4ccc5ccccc5c4-c4ccccc4)cc3)c3ccc4ccccc4c3)cc1C21c2ccccc2-c2ccc(-c3ccccc3)cc21 ; Cc1cccc2c1-c1ccc(N(c3ccc(-c4cc5ccccc5cc4-c4ccccc4)cc3)c3ccc4ccccc4c3)cc1C21c2ccccc2-c2ccc(-c3ccccc3)cc21 ; Cc1ccccc1-c1cc2ccccc2cc1-c1ccc(N(c2ccc3c(c2)C2(c4ccccc4-c4ccc(-c5ccccc5)cc42)c2cccc(C)c2-3)c2ccc3ccccc3c2)cc1]

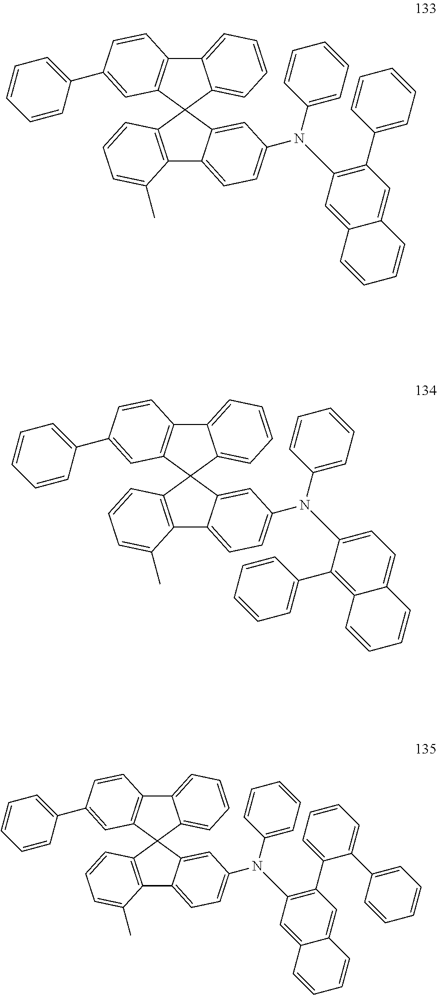 [SMILES: Cc1cccc2c1-c1ccc(N(c3ccccc3)c3cc4ccccc4cc3-c3ccccc3)cc1C21c2ccccc2-c2ccc(-c3ccccc3)cc21 ; Cc1cccc2c1-c1ccc(N(c3ccccc3)c3ccc4ccccc4c3-c3ccccc3)cc1C21c2ccccc2-c2ccc(-c3ccccc3)cc21 ; Cc1cccc2c1-c1ccc(N(c3ccccc3)c3cc4ccccc4cc3-c3ccccc3-c3ccccc3)cc1C21c2ccccc2-c2ccc(-c3ccccc3)cc21]

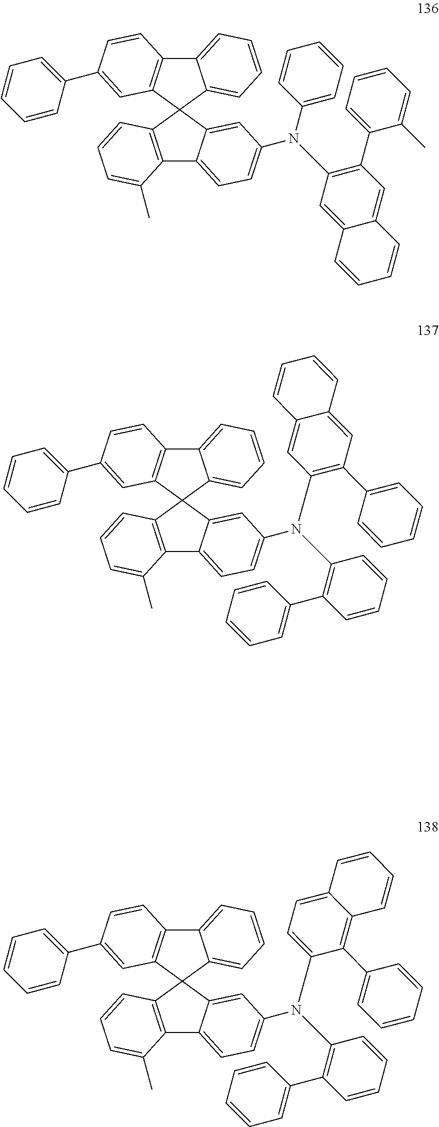 [SMILES: Cc1ccccc1-c1cc2ccccc2cc1N(c1ccccc1)c1ccc2c(c1)C1(c3ccccc3-c3ccc(-c4ccccc4)cc31)c1cccc(C)c1-2 ; Cc1cccc2c1-c1ccc(N(c3ccccc3-c3ccccc3)c3cc4ccccc4cc3-c3ccccc3)cc1C21c2ccccc2-c2ccc(-c3ccccc3)cc21 ; Cc1cccc2c1-c1ccc(N(c3ccccc3-c3ccccc3)c3ccc4ccccc4c3-c3ccccc3)cc1C21c2ccccc2-c2ccc(-c3ccccc3)cc21]

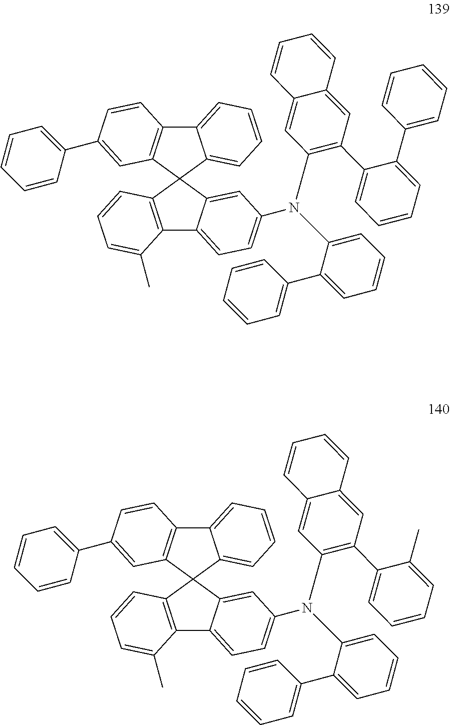 [SMILES: Cc1cccc2c1-c1ccc(N(c3ccccc3-c3ccccc3)c3cc4ccccc4cc3-c3ccccc3-c3ccccc3)cc1C21c2ccccc2-c2ccc(-c3ccccc3)cc21 ; Cc1ccccc1-c1cc2ccccc2cc1N(c1ccc2c(c1)C1(c3ccccc3-c3ccc(-c4ccccc4)cc31)c1cccc(C)c1-2)c1ccccc1-c1ccccc1]

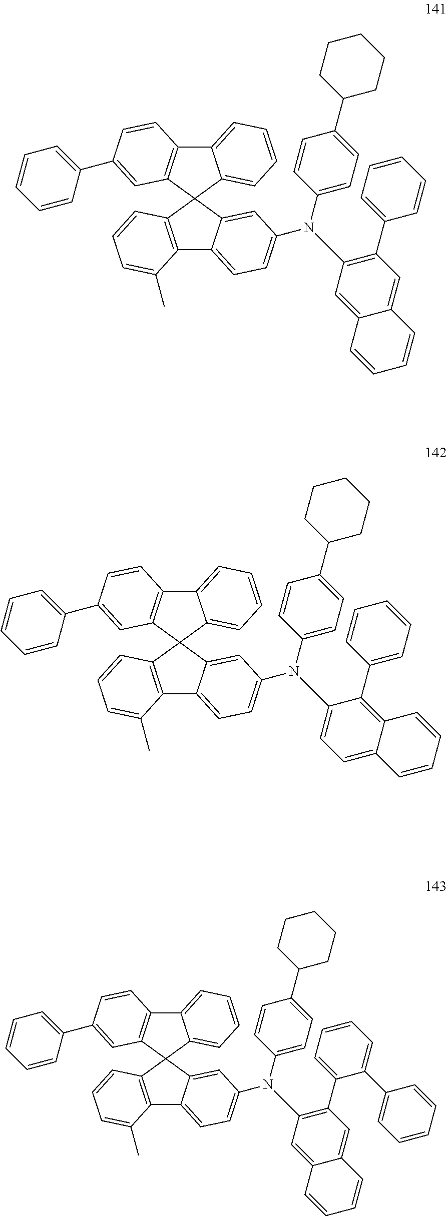 [SMILES: Cc1cccc2c1-c1ccc(N(c3ccc(C4CCCCC4)cc3)c3cc4ccccc4cc3-c3ccccc3)cc1C21c2ccccc2-c2ccc(-c3ccccc3)cc21 ; Cc1cccc2c1-c1ccc(N(c3ccc(C4CCCCC4)cc3)c3ccc4ccccc4c3-c3ccccc3)cc1C21c2ccccc2-c2ccc(-c3ccccc3)cc21 ; Cc1cccc2c1-c1ccc(N(c3ccc(C4CCCCC4)cc3)c3cc4ccccc4cc3-c3ccccc3-c3ccccc3)cc1C21c2ccccc2-c2ccc(-c3ccccc3)cc21]

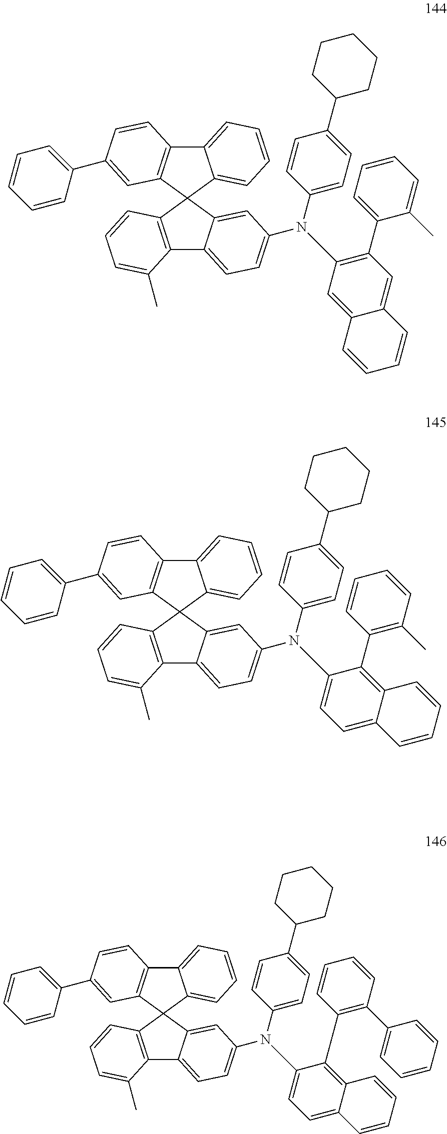 [SMILES: Cc1ccccc1-c1cc2ccccc2cc1N(c1ccc(C2CCCCC2)cc1)c1ccc2c(c1)C1(c3ccccc3-c3ccc(-c4ccccc4)cc31)c1cccc(C)c1-2 ; Cc1ccccc1-c1c(N(c2ccc(C3CCCCC3)cc2)c2ccc3c(c2)C2(c4ccccc4-c4ccc(-c5ccccc5)cc42)c2cccc(C)c2-3)ccc2ccccc12 ; Cc1cccc2c1-c1ccc(N(c3ccc(C4CCCCC4)cc3)c3ccc4ccccc4c3-c3ccccc3-c3ccccc3)cc1C21c2ccccc2-c2ccc(-c3ccccc3)cc21]

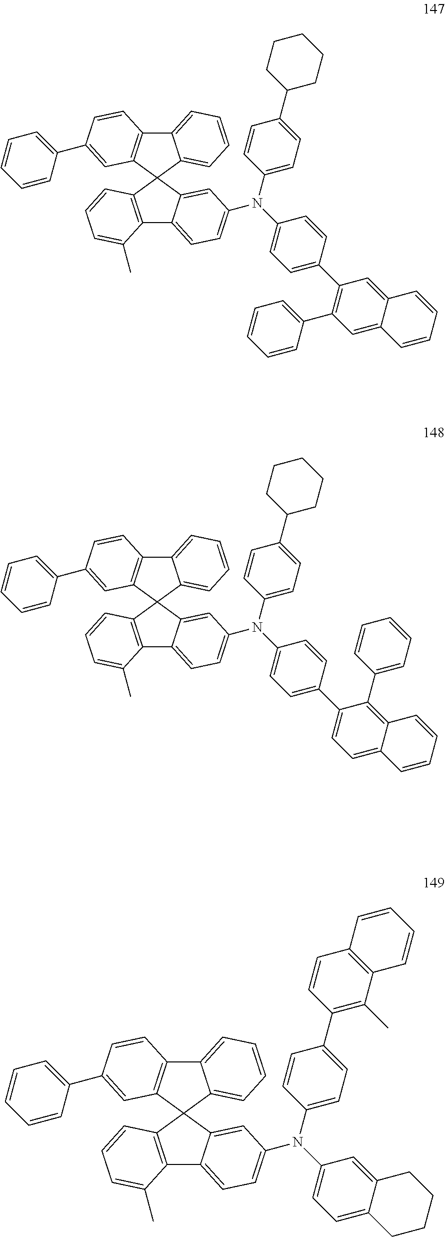 [SMILES: Cc1cccc2c1-c1ccc(N(c3ccc(-c4cc5ccccc5cc4-c4ccccc4)cc3)c3ccc(C4CCCCC4)cc3)cc1C21c2ccccc2-c2ccc(-c3ccccc3)cc21 ; Cc1cccc2c1-c1ccc(N(c3ccc(-c4ccc5ccccc5c4-c4ccccc4)cc3)c3ccc(C4CCCCC4)cc3)cc1C21c2ccccc2-c2ccc(-c3ccccc3)cc21 ; Cc1cccc2c1-c1ccc(N(c3ccc(-c4ccc5ccccc5c4C)cc3)c3ccc4c(c3)CCCC4)cc1C21c2ccccc2-c2ccc(-c3ccccc3)cc21]

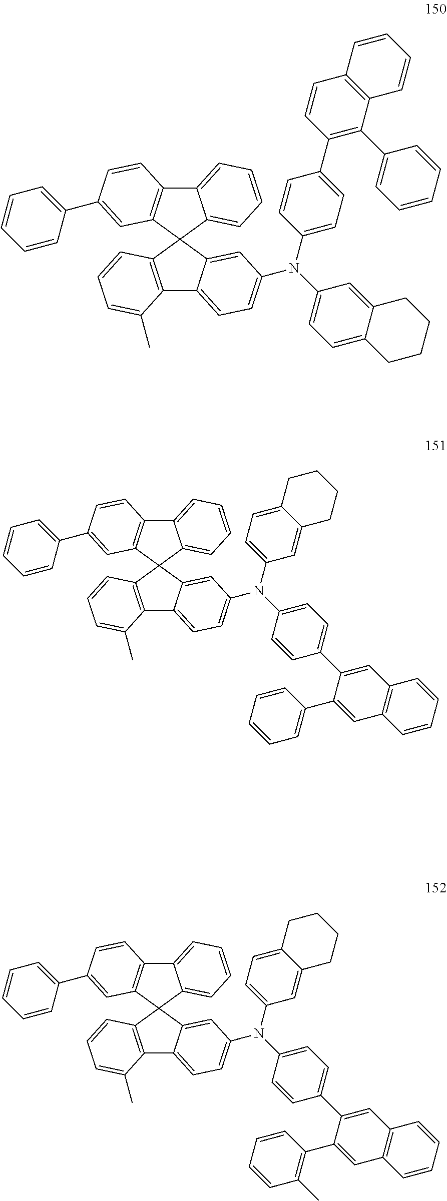 [SMILES: Cc1cccc2c1-c1ccc(N(c3ccc(-c4ccc5ccccc5c4-c4ccccc4)cc3)c3ccc4c(c3)CCCC4)cc1C21c2ccccc2-c2ccc(-c3ccccc3)cc21 ; Cc1cccc2c1-c1ccc(N(c3ccc(-c4cc5ccccc5cc4-c4ccccc4)cc3)c3ccc4c(c3)CCCC4)cc1C21c2ccccc2-c2ccc(-c3ccccc3)cc21 ; Cc1ccccc1-c1cc2ccccc2cc1-c1ccc(N(c2ccc3c(c2)CCCC3)c2ccc3c(c2)C2(c4ccccc4-c4ccc(-c5ccccc5)cc42)c2cccc(C)c2-3)cc1]

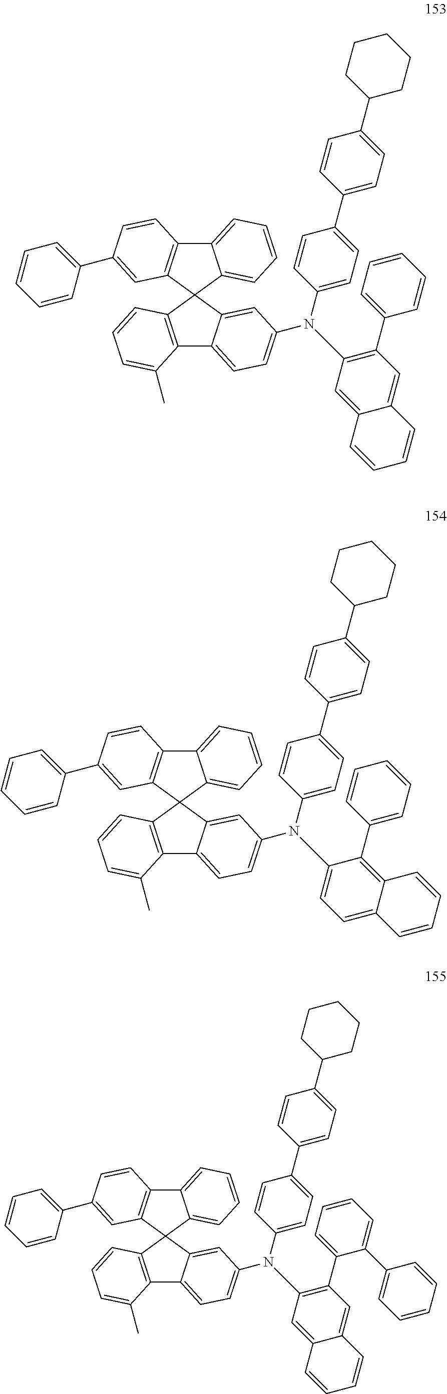 [SMILES: Cc1cccc2c1-c1ccc(N(c3ccc(-c4ccc(C5CCCCC5)cc4)cc3)c3cc4ccccc4cc3-c3ccccc3)cc1C21c2ccccc2-c2ccc(-c3ccccc3)cc21 ; Cc1cccc2c1-c1ccc(N(c3ccc(-c4ccc(C5CCCCC5)cc4)cc3)c3ccc4ccccc4c3-c3ccccc3)cc1C21c2ccccc2-c2ccc(-c3ccccc3)cc21 ; Cc1cccc2c1-c1ccc(N(c3ccc(-c4ccc(C5CCCCC5)cc4)cc3)c3cc4ccccc4cc3-c3ccccc3-c3ccccc3)cc1C21c2ccccc2-c2ccc(-c3ccccc3)cc21]

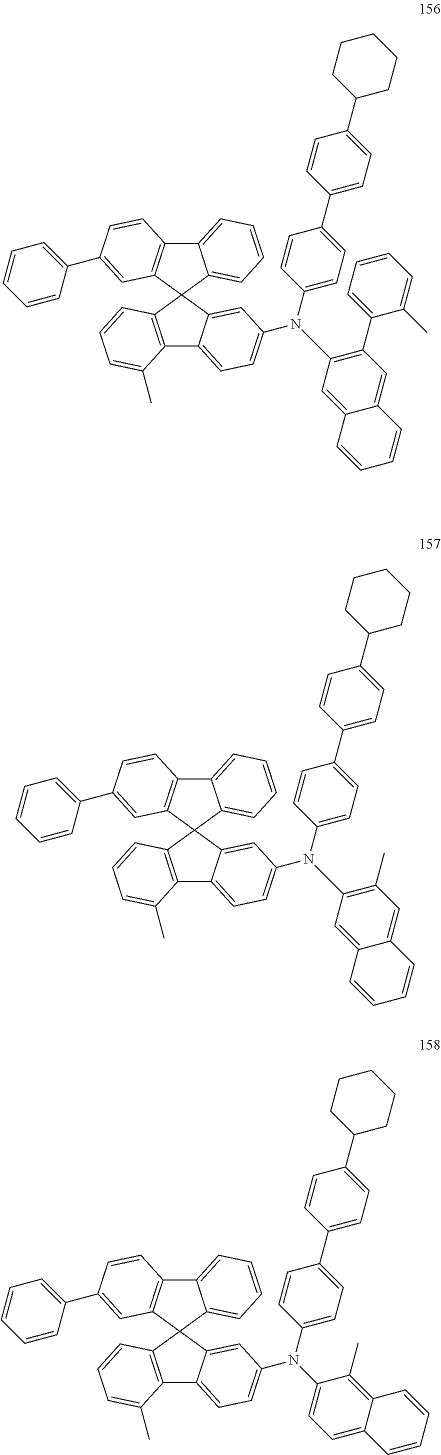 [SMILES: Cc1ccccc1-c1cc2ccccc2cc1N(c1ccc(-c2ccc(C3CCCCC3)cc2)cc1)c1ccc2c(c1)C1(c3ccccc3-c3ccc(-c4ccccc4)cc31)c1cccc(C)c1-2 ; Cc1cc2ccccc2cc1N(c1ccc(-c2ccc(C3CCCCC3)cc2)cc1)c1ccc2c(c1)C1(c3ccccc3-c3ccc(-c4ccccc4)cc31)c1cccc(C)c1-2 ; Cc1cccc2c1-c1ccc(N(c3ccc(-c4ccc(C5CCCCC5)cc4)cc3)c3ccc4ccccc4c3C)cc1C21c2ccccc2-c2ccc(-c3ccccc3)cc21]

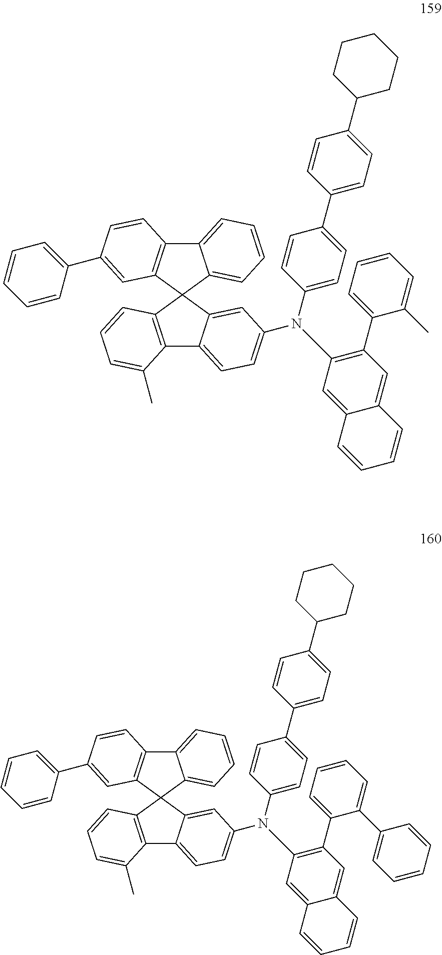 [SMILES: Cc1ccccc1-c1cc2ccccc2cc1N(c1ccc(-c2ccc(C3CCCCC3)cc2)cc1)c1ccc2c(c1)C1(c3ccccc3-c3ccc(-c4ccccc4)cc31)c1cccc(C)c1-2 ; Cc1cccc2c1-c1ccc(N(c3ccc(-c4ccc(C5CCCCC5)cc4)cc3)c3cc4ccccc4cc3-c3ccccc3-c3ccccc3)cc1C21c2ccccc2-c2ccc(-c3ccccc3)cc21]

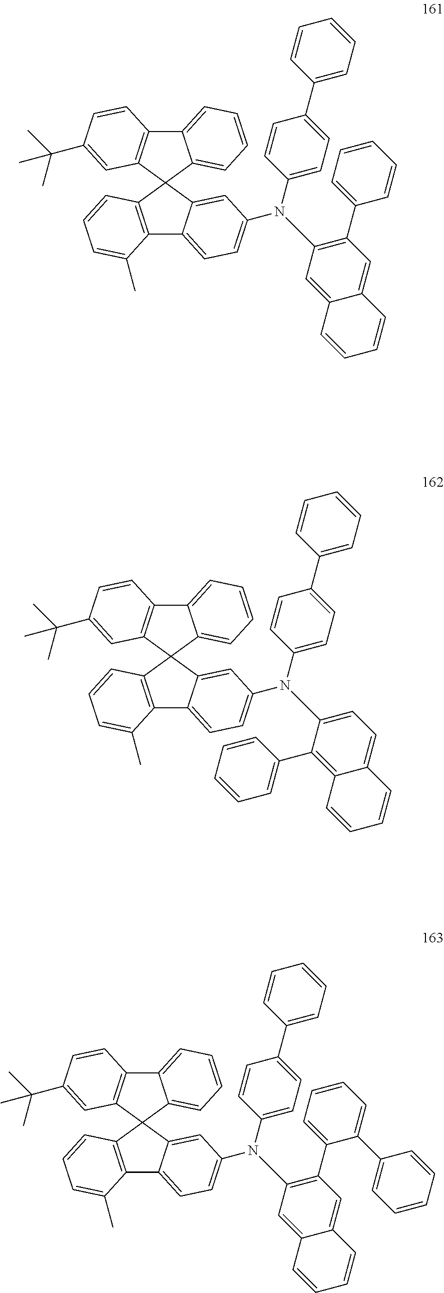 [SMILES: Cc1cccc2c1-c1ccc(N(c3ccc(-c4ccccc4)cc3)c3cc4ccccc4cc3-c3ccccc3)cc1C21c2ccccc2-c2ccc(C(C)(C)C)cc21 ; Cc1cccc2c1-c1ccc(N(c3ccc(-c4ccccc4)cc3)c3ccc4ccccc4c3-c3ccccc3)cc1C21c2ccccc2-c2ccc(C(C)(C)C)cc21 ; Cc1cccc2c1-c1ccc(N(c3ccc(-c4ccccc4)cc3)c3cc4ccccc4cc3-c3ccccc3-c3ccccc3)cc1C21c2ccccc2-c2ccc(C(C)(C)C)cc21]

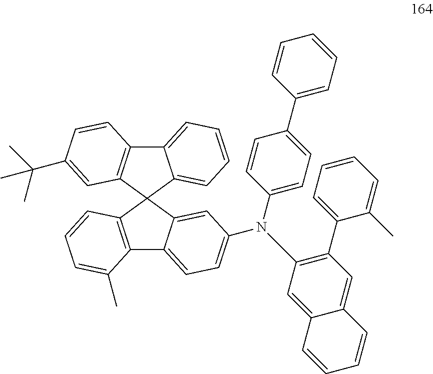 [SMILES: Cc1ccccc1-c1cc2ccccc2cc1N(c1ccc(-c2ccccc2)cc1)c1ccc2c(c1)C1(c3ccccc3-c3ccc(C(C)(C)C)cc31)c1cccc(C)c1-2]

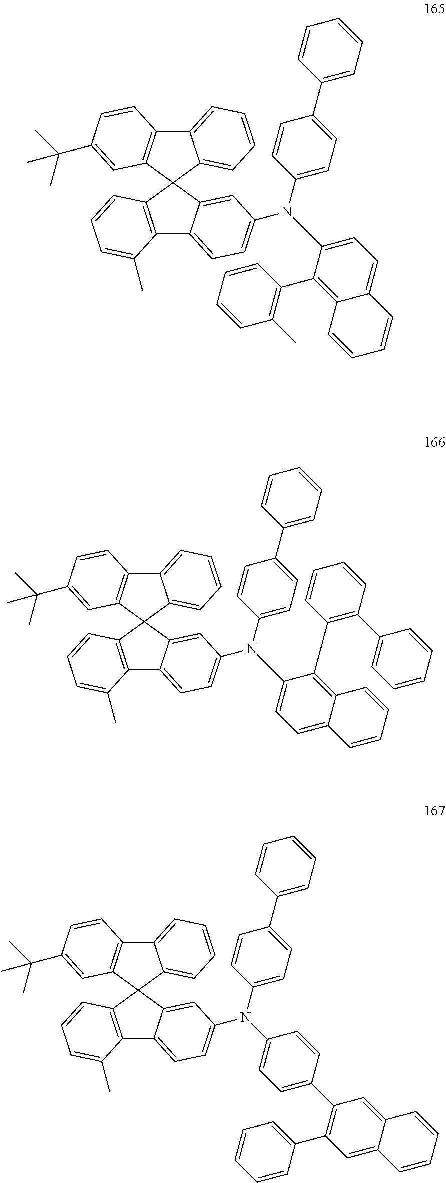 [SMILES: Cc1ccccc1-c1c(N(c2ccc(-c3ccccc3)cc2)c2ccc3c(c2)C2(c4ccccc4-c4ccc(C(C)(C)C)cc42)c2cccc(C)c2-3)ccc2ccccc12 ; Cc1cccc2c1-c1ccc(N(c3ccc(-c4ccccc4)cc3)c3ccc4ccccc4c3-c3ccccc3-c3ccccc3)cc1C21c2ccccc2-c2ccc(C(C)(C)C)cc21 ; Cc1cccc2c1-c1ccc(N(c3ccc(-c4ccccc4)cc3)c3ccc(-c4cc5ccccc5cc4-c4ccccc4)cc3)cc1C21c2ccccc2-c2ccc(C(C)(C)C)cc21]

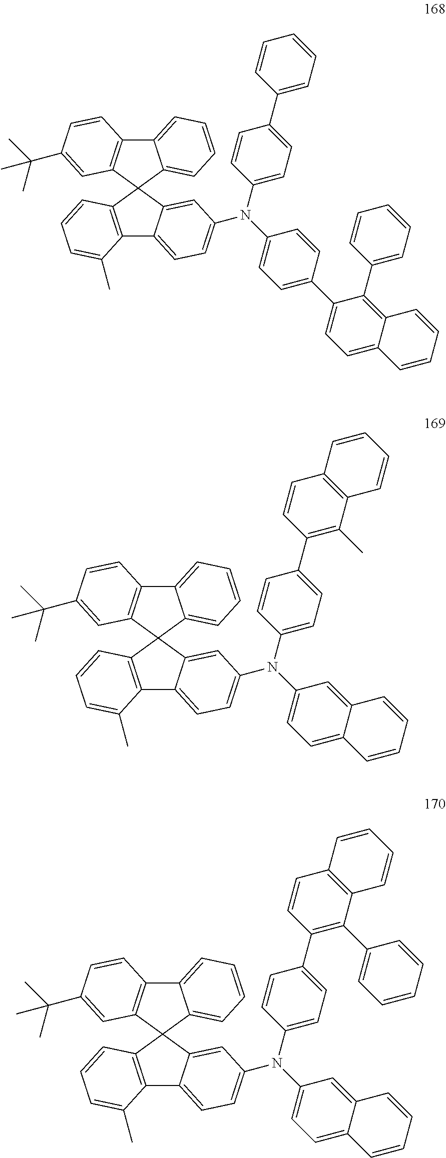 [SMILES: Cc1cccc2c1-c1ccc(N(c3ccc(-c4ccccc4)cc3)c3ccc(-c4ccc5ccccc5c4-c4ccccc4)cc3)cc1C21c2ccccc2-c2ccc(C(C)(C)C)cc21 ; Cc1cccc2c1-c1ccc(N(c3ccc(-c4ccc5ccccc5c4C)cc3)c3ccc4ccccc4c3)cc1C21c2ccccc2-c2ccc(C(C)(C)C)cc21 ; Cc1cccc2c1-c1ccc(N(c3ccc(-c4ccc5ccccc5c4-c4ccccc4)cc3)c3ccc4ccccc4c3)cc1C21c2ccccc2-c2ccc(C(C)(C)C)cc21]

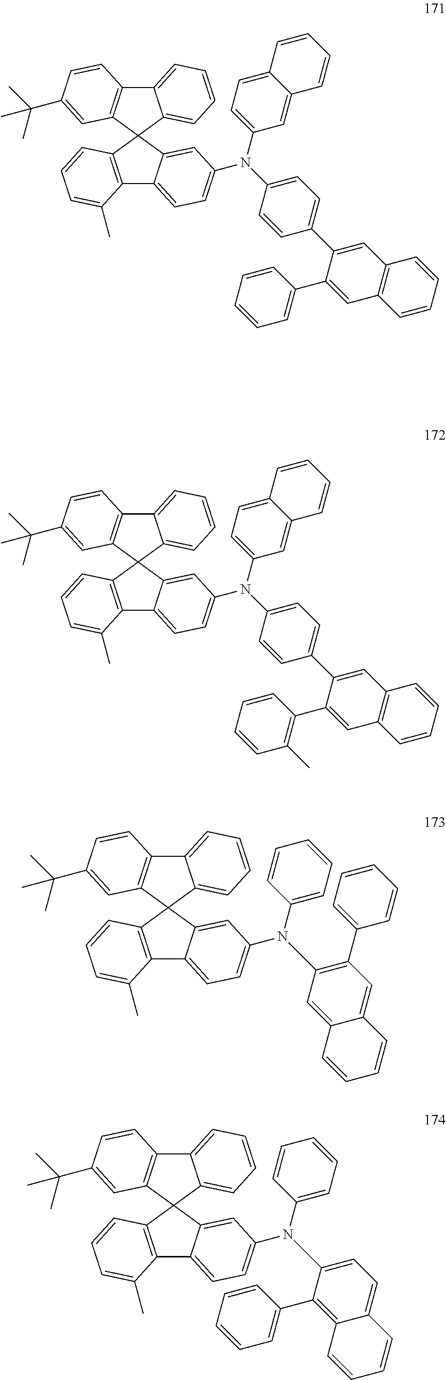 [SMILES: Cc1cccc2c1-c1ccc(N(c3ccc(-c4cc5ccccc5cc4-c4ccccc4)cc3)c3ccc4ccccc4c3)cc1C21c2ccccc2-c2ccc(C(C)(C)C)cc21 ; Cc1ccccc1-c1cc2ccccc2cc1-c1ccc(N(c2ccc3c(c2)C2(c4ccccc4-c4ccc(C(C)(C)C)cc42)c2cccc(C)c2-3)c2ccc3ccccc3c2)cc1 ; Cc1cccc2c1-c1ccc(N(c3ccccc3)c3cc4ccccc4cc3-c3ccccc3)cc1C21c2ccccc2-c2ccc(C(C)(C)C)cc21 ; Cc1cccc2c1-c1ccc(N(c3ccccc3)c3ccc4ccccc4c3-c3ccccc3)cc1C21c2ccccc2-c2ccc(C(C)(C)C)cc21]

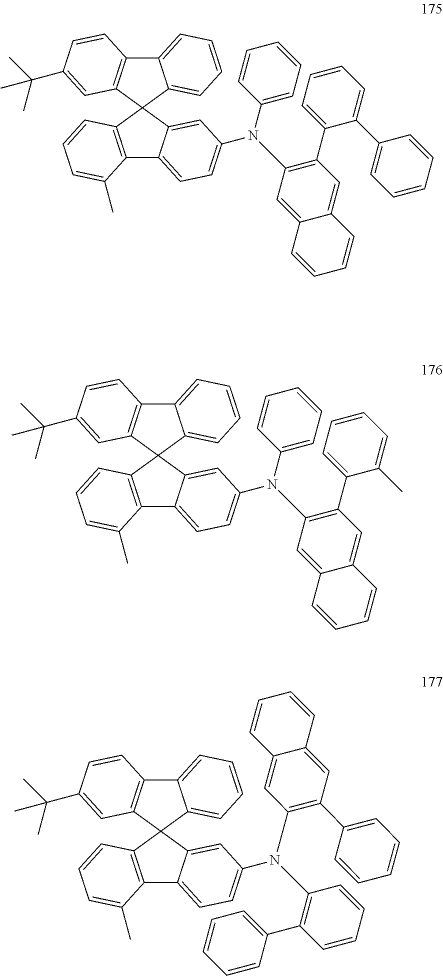

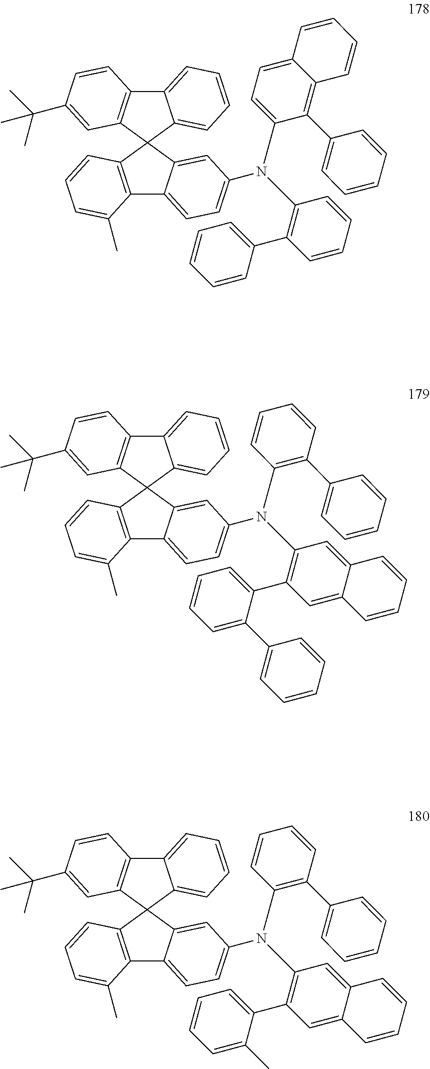 [SMILES: Cc1cccc2c1-c1ccc(N(c3ccccc3-c3ccccc3)c3ccc4ccccc4c3-c3ccccc3)cc1C21c2ccccc2-c2ccc(C(C)(C)C)cc21 ; Cc1cccc2c1-c1ccc(N(c3ccccc3-c3ccccc3)c3cc4ccccc4cc3-c3ccccc3-c3ccccc3)cc1C21c2ccccc2-c2ccc(C(C)(C)C)cc21 ; Cc1ccccc1-c1cc2ccccc2cc1N(c1ccc2c(c1)C1(c3ccccc3-c3ccc(C(C)(C)C)cc31)c1cccc(C)c1-2)c1ccccc1-c1ccccc1]

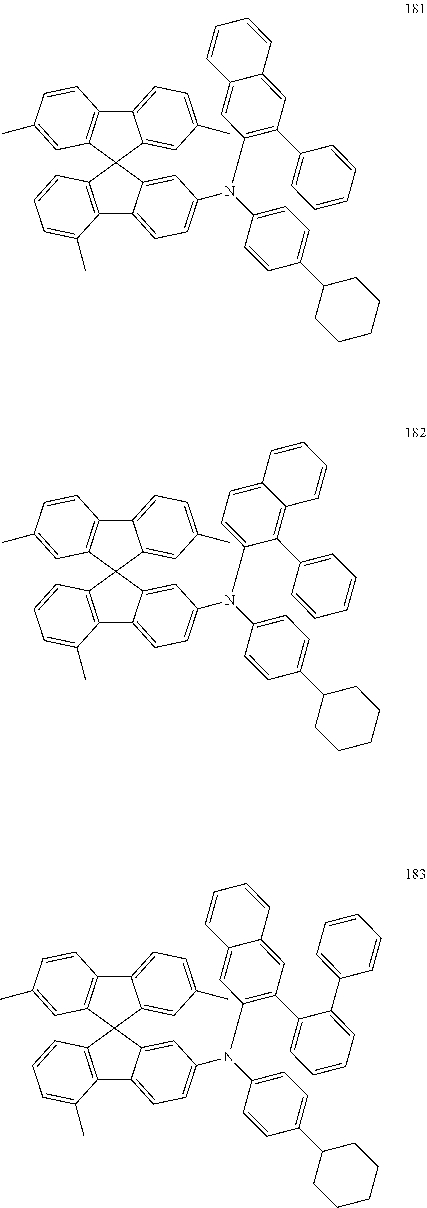 [SMILES: Cc1ccc2c(c1)C1(c3cc(C)ccc3-2)c2cc(N(c3ccc(C4CCCCC4)cc3)c3cc4ccccc4cc3-c3ccccc3)ccc2-c2c(C)cccc21 ; Cc1ccc2c(c1)C1(c3cc(C)ccc3-2)c2cc(N(c3ccc(C4CCCCC4)cc3)c3ccc4ccccc4c3-c3ccccc3)ccc2-c2c(C)cccc21 ; Cc1ccc2c(c1)C1(c3cc(C)ccc3-2)c2cc(N(c3ccc(C4CCCCC4)cc3)c3cc4ccccc4cc3-c3ccccc3-c3ccccc3)ccc2-c2c(C)cccc21]

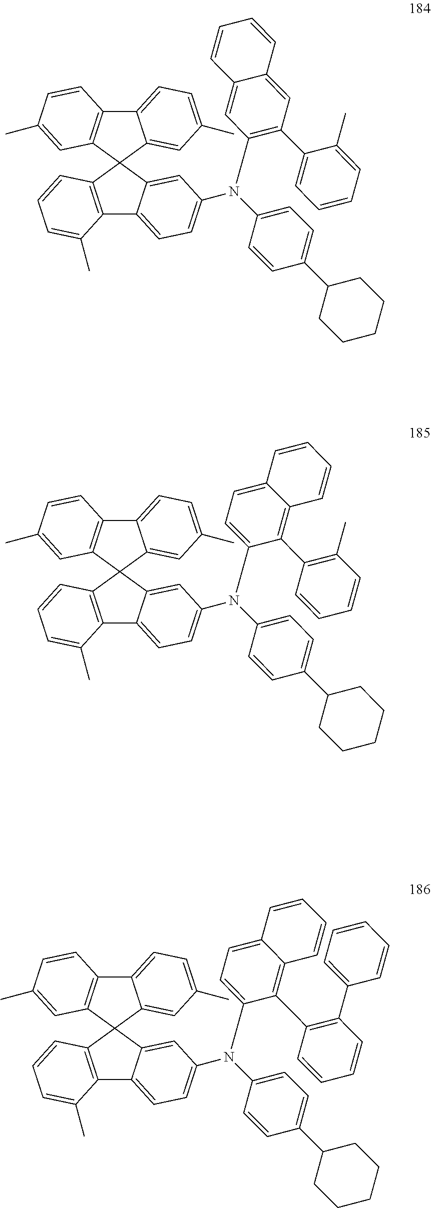 [SMILES: Cc1ccc2c(c1)C1(c3cc(C)ccc3-2)c2cc(N(c3ccc(C4CCCCC4)cc3)c3cc4ccccc4cc3-c3ccccc3C)ccc2-c2c(C)cccc21 ; Cc1ccc2c(c1)C1(c3cc(C)ccc3-2)c2cc(N(c3ccc(C4CCCCC4)cc3)c3ccc4ccccc4c3-c3ccccc3C)ccc2-c2c(C)cccc21 ; Cc1ccc2c(c1)C1(c3cc(C)ccc3-2)c2cc(N(c3ccc(C4CCCCC4)cc3)c3ccc4ccccc4c3-c3ccccc3-c3ccccc3)ccc2-c2c(C)cccc21]

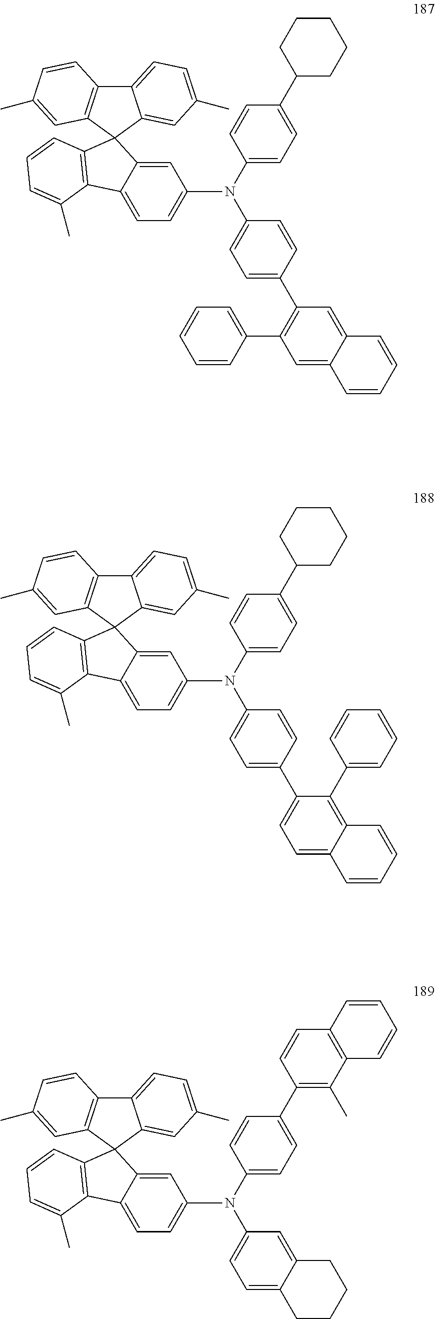 [SMILES: Cc1ccc2c(c1)C1(c3cc(C)ccc3-2)c2cc(N(c3ccc(-c4cc5ccccc5cc4-c4ccccc4)cc3)c3ccc(C4CCCCC4)cc3)ccc2-c2c(C)cccc21 ; Cc1ccc2c(c1)C1(c3cc(C)ccc3-2)c2cc(N(c3ccc(-c4ccc5ccccc5c4-c4ccccc4)cc3)c3ccc(C4CCCCC4)cc3)ccc2-c2c(C)cccc21 ; Cc1ccc2c(c1)C1(c3cc(C)ccc3-2)c2cc(N(c3ccc(-c4ccc5ccccc5c4C)cc3)c3ccc4c(c3)CCCC4)ccc2-c2c(C)cccc21]

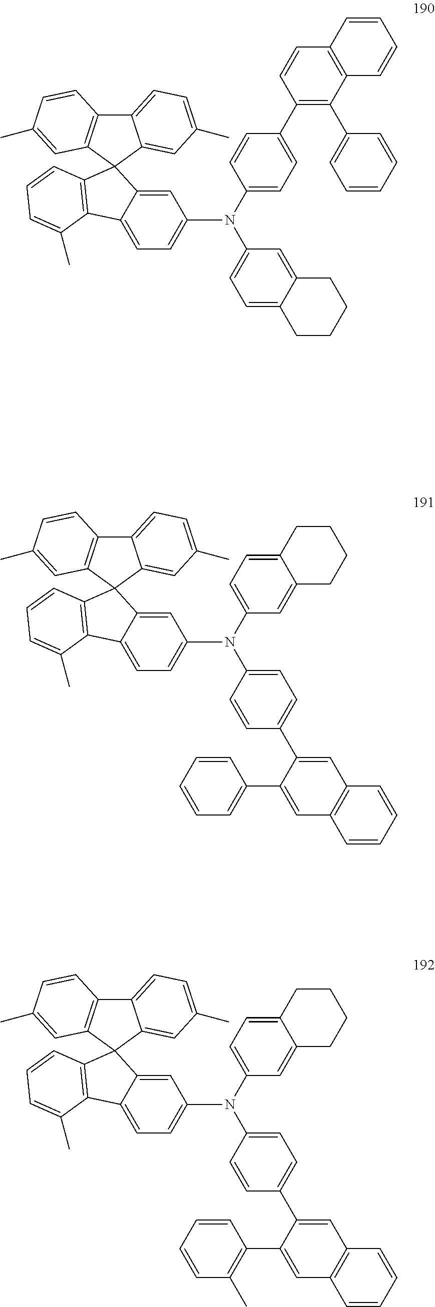 [SMILES: Cc1ccc2c(c1)C1(c3cc(C)ccc3-2)c2cc(N(c3ccc(-c4ccc5ccccc5c4-c4ccccc4)cc3)c3ccc4c(c3)CCCC4)ccc2-c2c(C)cccc21 ; Cc1ccc2c(c1)C1(c3cc(C)ccc3-2)c2cc(N(c3ccc(-c4cc5ccccc5cc4-c4ccccc4)cc3)c3ccc4c(c3)CCCC4)ccc2-c2c(C)cccc21 ; Cc1ccc2c(c1)C1(c3cc(C)ccc3-2)c2cc(N(c3ccc(-c4cc5ccccc5cc4-c4ccccc4C)cc3)c3ccc4c(c3)CCCC4)ccc2-c2c(C)cccc21]

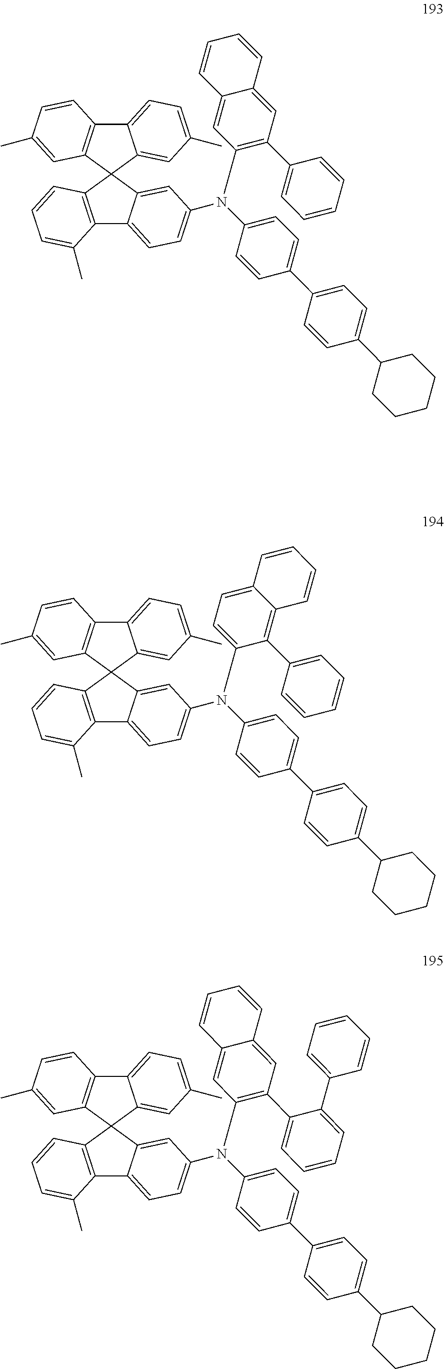 [SMILES: Cc1ccc2c(c1)C1(c3cc(C)ccc3-2)c2cc(N(c3ccc(-c4ccc(C5CCCCC5)cc4)cc3)c3cc4ccccc4cc3-c3ccccc3)ccc2-c2c(C)cccc21 ; Cc1ccc2c(c1)C1(c3cc(C)ccc3-2)c2cc(N(c3ccc(-c4ccc(C5CCCCC5)cc4)cc3)c3ccc4ccccc4c3-c3ccccc3)ccc2-c2c(C)cccc21 ; Cc1ccc2c(c1)C1(c3cc(C)ccc3-2)c2cc(N(c3ccc(-c4ccc(C5CCCCC5)cc4)cc3)c3cc4ccccc4cc3-c3ccccc3-c3ccccc3)ccc2-c2c(C)cccc21]

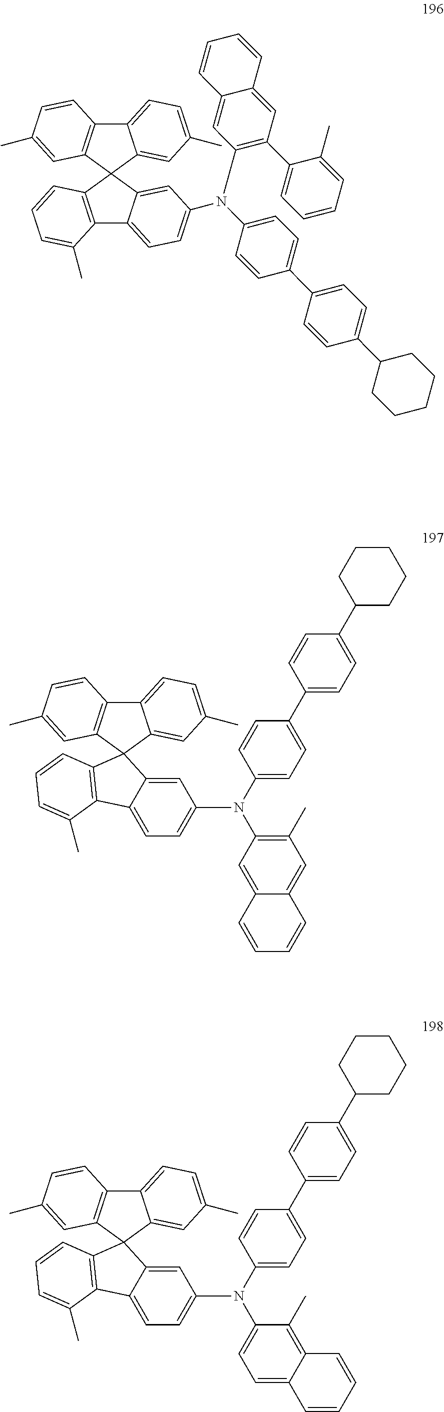 [SMILES: Cc1ccc2c(c1)C1(c3cc(C)ccc3-2)c2cc(N(c3ccc(-c4ccc(C5CCCCC5)cc4)cc3)c3cc4ccccc4cc3-c3ccccc3C)ccc2-c2c(C)cccc21 ; Cc1ccc2c(c1)C1(c3cc(C)ccc3-2)c2cc(N(c3ccc(-c4ccc(C5CCCCC5)cc4)cc3)c3cc4ccccc4cc3C)ccc2-c2c(C)cccc21 ; Cc1ccc2c(c1)C1(c3cc(C)ccc3-2)c2cc(N(c3ccc(-c4ccc(C5CCCCC5)cc4)cc3)c3ccc4ccccc4c3C)ccc2-c2c(C)cccc21]

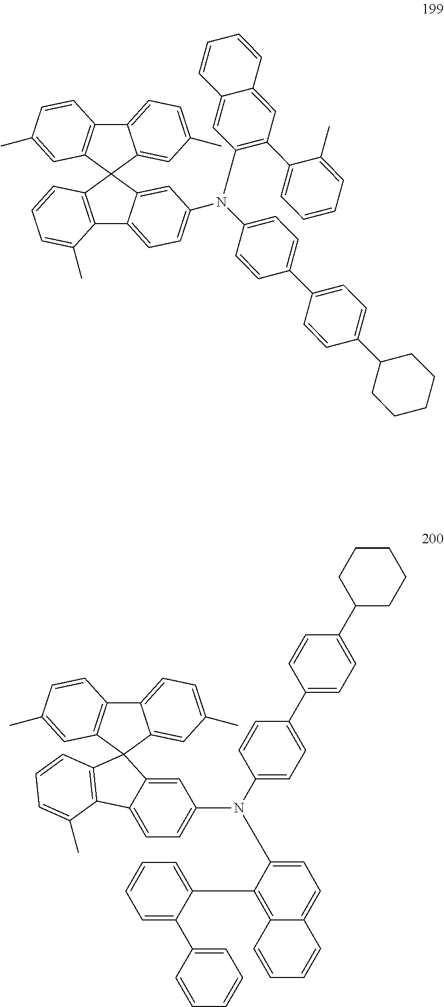 [SMILES: Cc1ccc2c(c1)C1(c3cc(C)ccc3-2)c2cc(N(c3ccc(-c4ccc(C5CCCCC5)cc4)cc3)c3cc4ccccc4cc3-c3ccccc3C)ccc2-c2c(C)cccc21 ; Cc1ccc2c(c1)C1(c3cc(C)ccc3-2)c2cc(N(c3ccc(-c4ccc(C5CCCCC5)cc4)cc3)c3ccc4ccccc4c3-c3ccccc3-c3ccccc3)ccc2-c2c(C)cccc21]

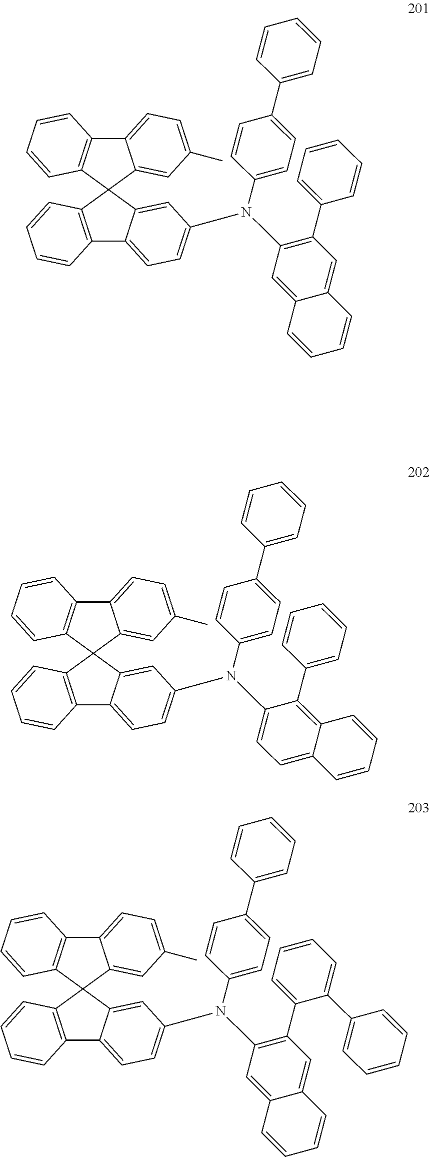 [SMILES: Cc1ccc2c(c1)C1(c3ccccc3-2)c2ccccc2-c2ccc(N(c3ccc(-c4ccccc4)cc3)c3cc4ccccc4cc3-c3ccccc3)cc21 ; Cc1ccc2c(c1)C1(c3ccccc3-2)c2ccccc2-c2ccc(N(c3ccc(-c4ccccc4)cc3)c3ccc4ccccc4c3-c3ccccc3)cc21 ; Cc1ccc2c(c1)C1(c3ccccc3-2)c2ccccc2-c2ccc(N(c3ccc(-c4ccccc4)cc3)c3cc4ccccc4cc3-c3ccccc3-c3ccccc3)cc21]

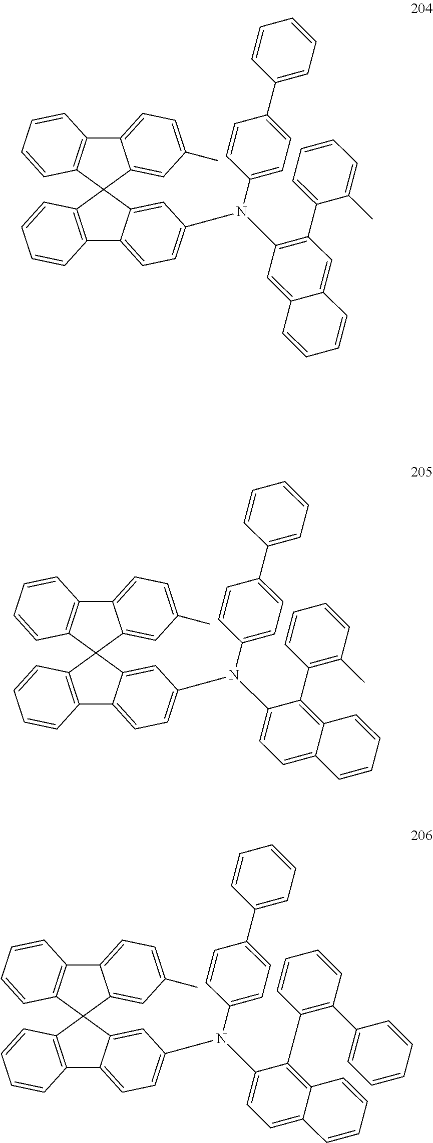 [SMILES: Cc1ccc2c(c1)C1(c3ccccc3-2)c2ccccc2-c2ccc(N(c3ccc(-c4ccccc4)cc3)c3cc4ccccc4cc3-c3ccccc3C)cc21 ; Cc1ccc2c(c1)C1(c3ccccc3-2)c2ccccc2-c2ccc(N(c3ccc(-c4ccccc4)cc3)c3ccc4ccccc4c3-c3ccccc3C)cc21 ; Cc1ccc2c(c1)C1(c3ccccc3-2)c2ccccc2-c2ccc(N(c3ccc(-c4ccccc4)cc3)c3ccc4ccccc4c3-c3ccccc3-c3ccccc3)cc21]

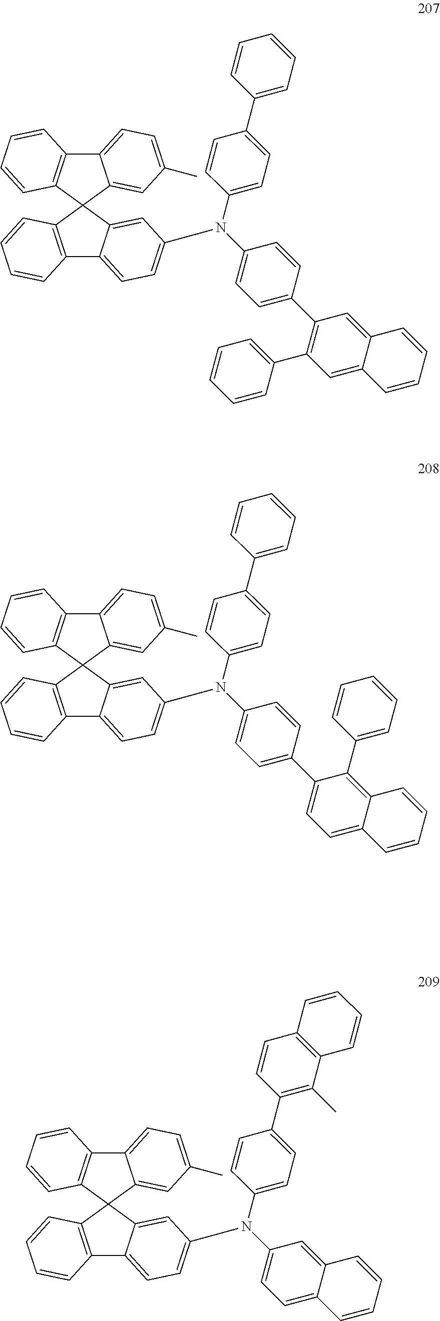 [SMILES: Cc1ccc2c(c1)C1(c3ccccc3-2)c2ccccc2-c2ccc(N(c3ccc(-c4ccccc4)cc3)c3ccc(-c4cc5ccccc5cc4-c4ccccc4)cc3)cc21 ; Cc1ccc2c(c1)C1(c3ccccc3-2)c2ccccc2-c2ccc(N(c3ccc(-c4ccccc4)cc3)c3ccc(-c4ccc5ccccc5c4-c4ccccc4)cc3)cc21 ; Cc1ccc2c(c1)C1(c3ccccc3-2)c2ccccc2-c2ccc(N(c3ccc(-c4ccc5ccccc5c4C)cc3)c3ccc4ccccc4c3)cc21]

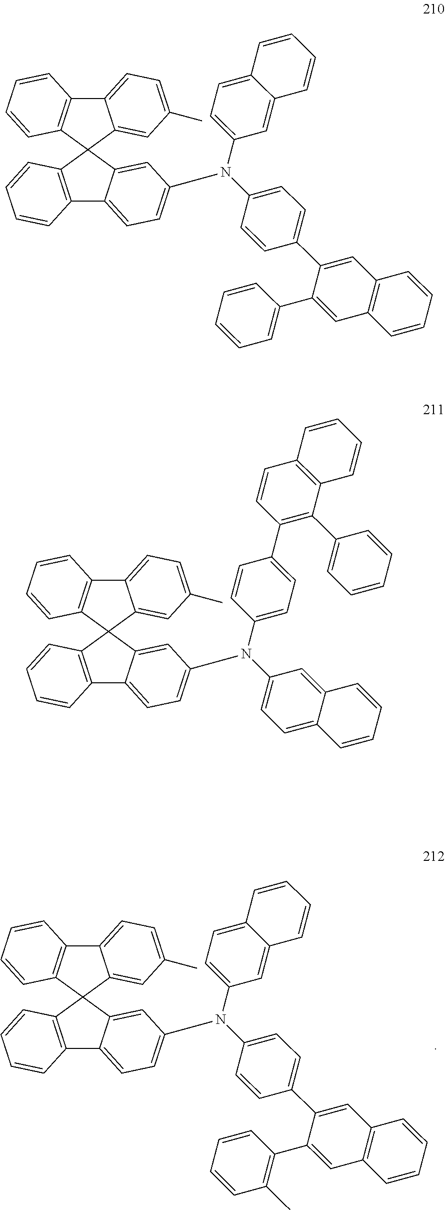 [SMILES: Cc1ccc2c(c1)C1(c3ccccc3-2)c2ccccc2-c2ccc(N(c3ccc(-c4cc5ccccc5cc4-c4ccccc4)cc3)c3ccc4ccccc4c3)cc21 ; Cc1ccc2c(c1)C1(c3ccccc3-2)c2ccccc2-c2ccc(N(c3ccc(-c4ccc5ccccc5c4-c4ccccc4)cc3)c3ccc4ccccc4c3)cc21 ; Cc1ccc2c(c1)C1(c3ccccc3-2)c2ccccc2-c2ccc(N(c3ccc(-c4cc5ccccc5cc4-c4ccccc4C)cc3)c3ccc4ccccc4c3)cc21]

The amine compound of one or more embodiments may include a spiro fluorene moiety substituted with at least one substituted or unsubstituted methyl group, and a naphthyl group, combined with the nitrogen atom of the amine. Consequently, the amine compound of one or more embodiments may have high glass transition temperature and may prevent or reduce crystallization. In some embodiments, because the amine compound of one or more embodiments includes a spiro fluorene moiety substituted with at least one substituted or unsubstituted methyl group, and a naphthyl group, combined with the nitrogen atom of the amine, low refractive index and excellent or suitable hole transport capacity may be shown. Accordingly, the light emitting element ED including the amine compound of one or more embodiments may show a reduced driving voltage, high luminance, high efficiency, and long-life characteristics. In the light emitting element ED of one or more embodiments, the hole transport region HTR may include the amine compound of one or more embodiments.

In one or more embodiments, the hole transport region HTR may be provided on the first electrode EL1. The thickness of the hole transport region HTR may be, for example, about 50 Å to about 15,000 Å.

In one or more embodiments, the hole transport region HTR may include at least one selected from among a hole injection layer HIL, a hole transport layer HTL, a buffer layer or an emission auxiliary layer, and an electron blocking layer EBL. At least one selected from among the hole injection layer HIL, the hole transport layer HTL, and the electron blocking layer EBL may include the amine compound of one or more embodiments. For example, in one or more embodiments, the hole transport layer HTL may include at least one amine compound of one or more embodiments.

The hole transport region HTR may have a single layer formed utilizing a single material, a single layer formed utilizing multiple different materials, or a multilayer structure including multiple layers formed utilizing multiple different materials.

For example, in some embodiments, the hole transport region HTR may have the structure of a single layer of a hole injection layer HIL or a hole transport layer HTL, and may have a structure of a single layer formed utilizing a hole injection material and a hole transport material. In some embodiments, the hole transport region HTR may have a structure of a single layer formed utilizing multiple different materials, or a structure stacked from the first electrode EL1 of hole injection layer HIL/hole transport layer HTL, hole injection layer HIL/hole transport layer HTL/buffer layer, hole injection layer HIL/buffer layer, hole transport layer HTL/buffer layer, or hole injection layer HIL/hole transport layer HTL/electron blocking layer EBL, without limitation.

The hole transport region HTR may be formed utilizing one or more suitable methods such as a vacuum deposition method, a spin coating method, a casting method, a Langmuir-Blodgett (LB) method, an inkjet printing method, a laser printing method, and/or a laser induced thermal imaging (LITI) method.

In one or more embodiments, the hole transport region HTR may further include the compounds explained herein. The hole transport region HTR may include a compound represented by Formula H-1.

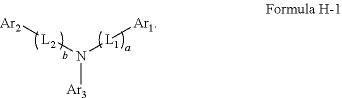

In Formula H-1, L₁ and L₂ may each independently be a direct linkage, a substituted or unsubstituted arylene group of 6 to 30 ring-forming carbon atoms, or a substituted or unsubstituted heteroarylene group of 2 to 30 ring-forming carbon atoms. “a” and “b” may each independently be an integer of 0 to 10. In some embodiments, when “a” or “b” is an integer of 2 or more, multiple L₁(s) and L₂(s) may each independently be a substituted or unsubstituted arylene group of 6 to 30 ring-forming carbon atoms, or a substituted or unsubstituted heteroarylene group of 2 to 30 ring-forming carbon atoms.

In Formula H-1, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. In some embodiments, in Formula H-1, Ar₃ may be a substituted or unsubstituted aryl group of 6 to 30 ring-forming carbon atoms.

The compound represented by Formula H-1 may be a monoamine compound. In some embodiments, the compound represented by Formula H-1 may be a diamine compound in which at least one selected from among Ar₁ to Ar₃ includes an amine group as a substituent. In some embodiments, the compound represented by Formula H-1 may be a carbazole-based compound in which at least one selected from among Ar₁ and Ar₂ includes a substituted or unsubstituted carbazole group, or a fluorene-based compound in which at least one selected from among Ar₁ and Ar₂ includes a substituted or unsubstituted fluorene group.

The compound represented by Formula H-1 may be represented by any one selected from among compounds in Compound Group H. However, the compounds listed in Compound Group H are only mere examples, and the compound represented by Formula H-1 is not limited to the compounds represented in Compound Group H.

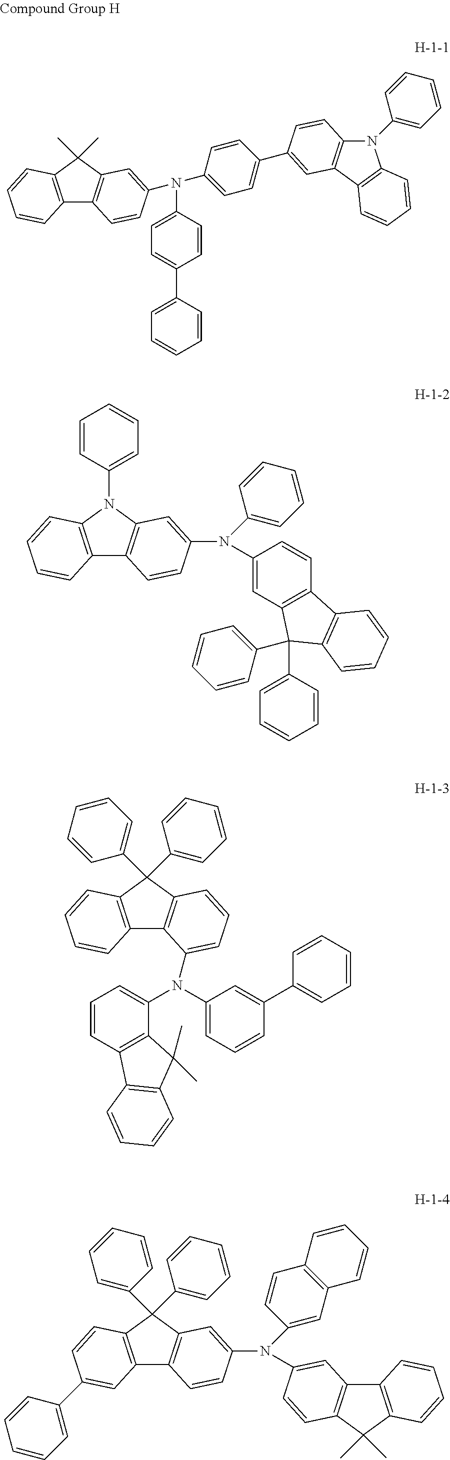 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

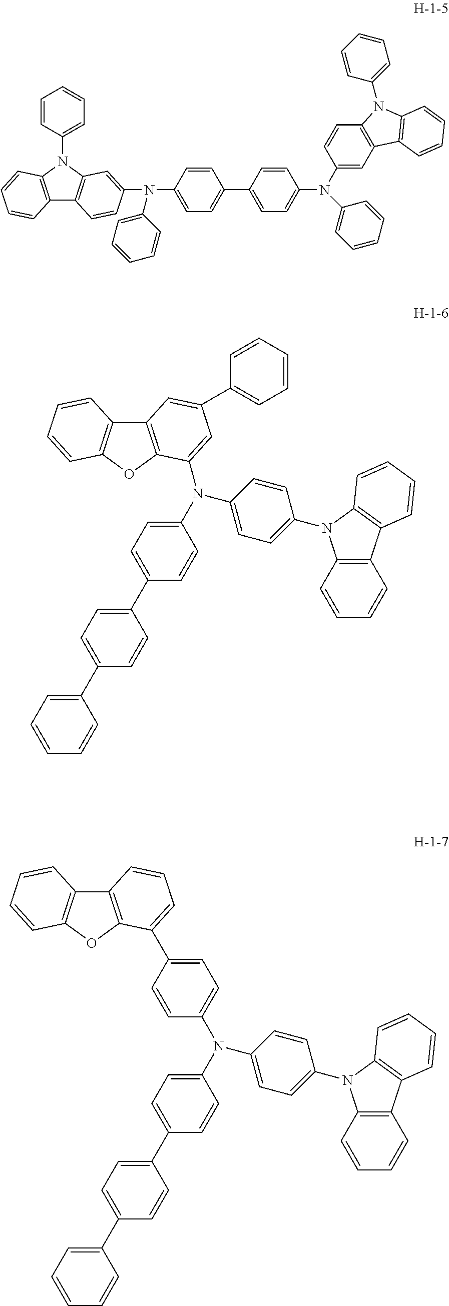 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

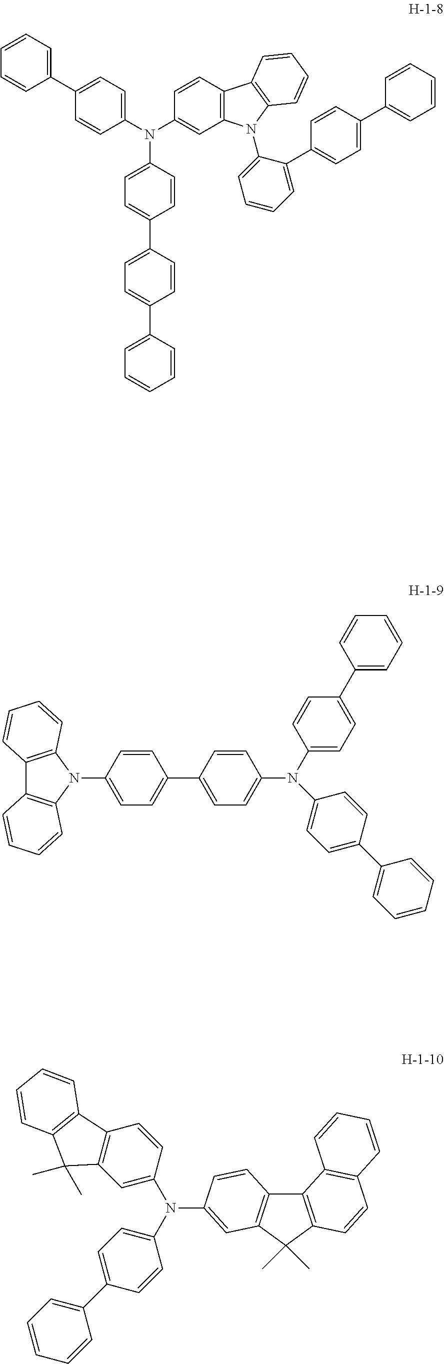 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

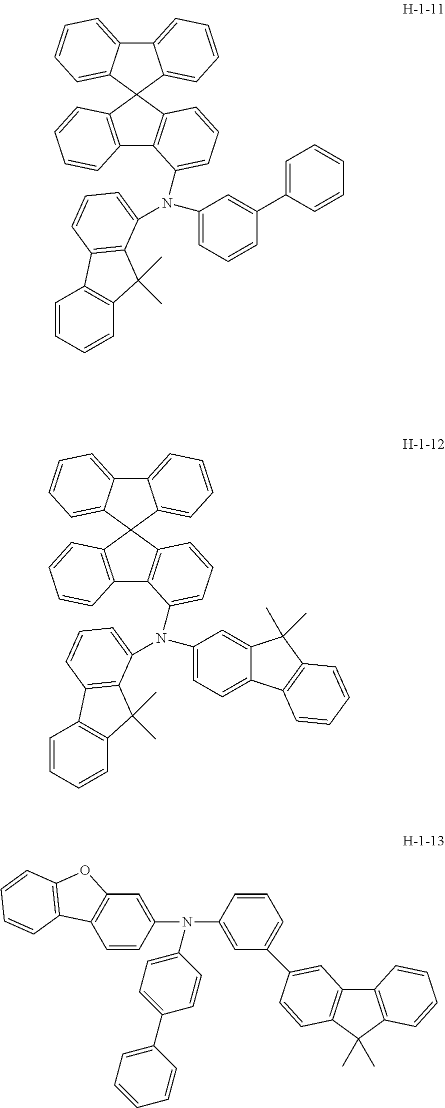 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21]

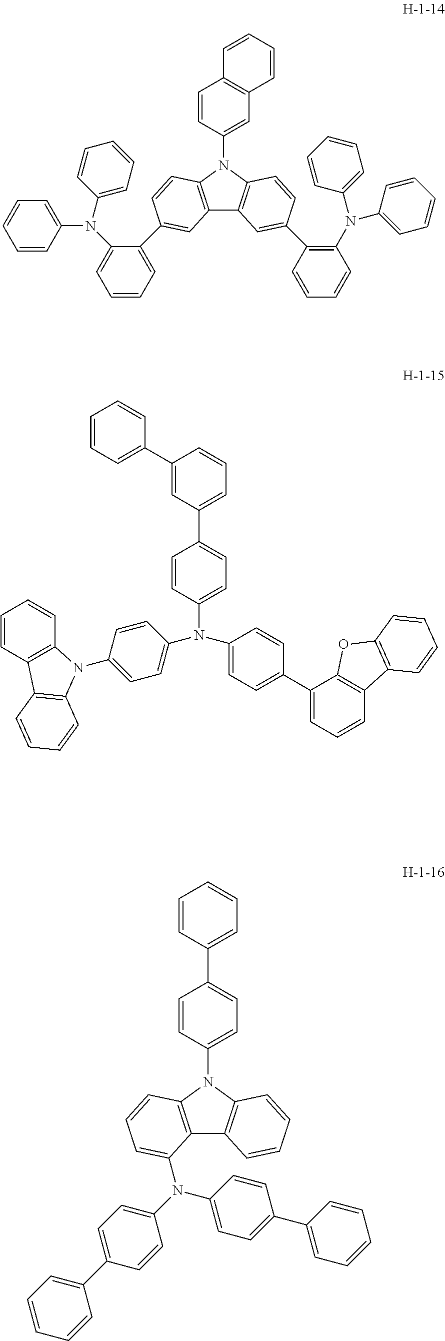 [SMILES: c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1 ; c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1]

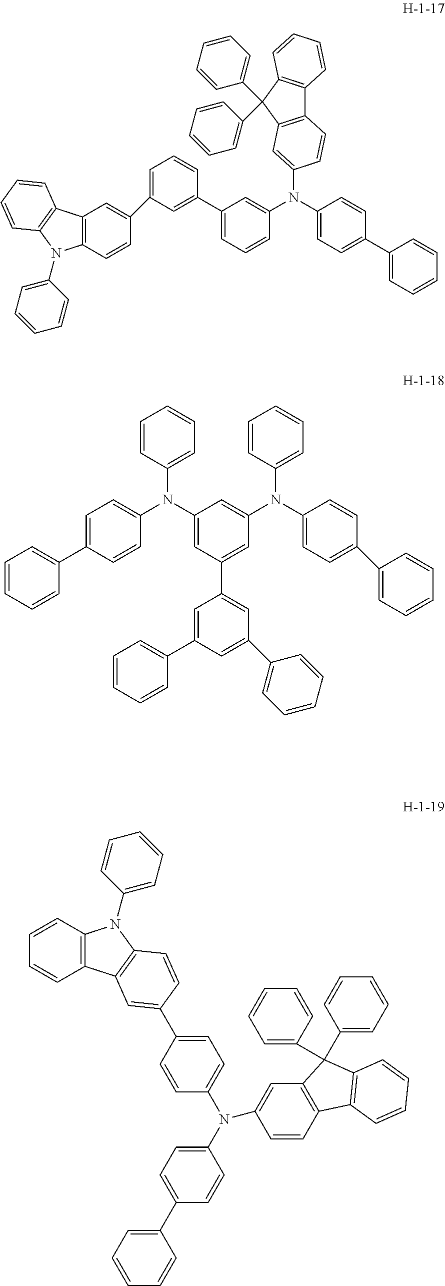 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

In one or more embodiments, the hole transport region HTR may include a phthalocyanine compound such as copper phthalocyanine, N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′,4″-tris(N,N-diphenylamino)triphenylamine (TDATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(1-naphthalen-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], and dipyrazino[2,3-f:2′,3′-h]quinoxaline-2,3,6,7,10,11-hexacarbonitrile (HAT-CN).

In one or more embodiments, the hole transport region HTR may include carbazole derivatives such as N-phenyl carbazole and polyvinyl carbazole, fluorene-based derivatives, N,N′-bis(3-methylphenyl)-N,N′-diphenyl-[1,1′-biphenyl]-4,4′-diamine (TPD), triphenylamine-based derivatives such as 4,4′,4″-tris(N-carbazolyl)triphenylamine (TCTA), N,N′-di(naphthalen-1-yl)-N,N′-diphenyl-benzidine (NPB), 4,4′-cyclohexylidene bis[N,N-bis(4-methylphenyl)benzenamine] (TAPC), 4,4′-bis[N,N′-(3-tolyl)amino]-3,3′-dimethylbiphenyl (HMTPD), 1,3-bis(N-carbazolyl)benzene, etc.

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the above-described compounds of the hole transport region in at least one selected from among a hole injection layer HIL, hole transport layer HTL, and electron blocking layer EBL. The thickness of the hole transport region HTR may be from about 100 Å to about 10,000 Å, for example, from about 100 Å to about 5,000 Å. When the hole transport region HTR includes a hole injection layer HIL, the thickness of the hole injection region HIL may be, for example, from about 30 Å to about 1,000 Å. When the hole transport region HTR includes a hole transport layer HTL, the thickness of the hole transport layer HTL may be from about 30 Å to about 1,000 Å. For example, when the hole transport region HTR includes an electron blocking layer EBL, the thickness of the electron blocking layer EBL may be from about 10 Å to about 1,000 Å. When the thicknesses of the hole transport region HTR, the hole injection layer HIL, the hole transport layer HTL, and the electron blocking layer EBL satisfy the above-described ranges, satisfactory hole transport properties may be achieved without a substantial increase in a driving voltage.

In one or more embodiments, the hole transport region HTR may further include a charge generating material to increase conductivity in addition to the above-described materials. The charge generating material may be dispersed uniformly or non-uniformly in the hole transport region HTR. The charge generating material may be, for example, a p-dopant. The p-dopant may include at least one of metal halide compounds, quinone derivatives, metal oxides, or cyano group-containing compounds, without limitation. For example, the p-dopant may include metal halide compounds such as CuI and RbI, quinone derivatives such as tetracyanoquinodimethane (TCNQ) and 2,3,5,6-tetrafluoro-7,7,8,8-tetracyanoquinodimethane (F4-TCNQ), metal oxides such as tungsten oxide and molybdenum oxide, cyano group-containing compounds such as dipyrazino[2,3-f:2′,3′-h]quinoxaline-2,3,6,7,10,11-hexacarbonitrile (HATCN) and 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., without limitation.

As described above, the hole transport region HTR may further include a buffer layer in addition to the hole injection layer HIL, the hole transport layer HTL, and the electron blocking layer EBL. The buffer layer may compensate resonance distance according to the wavelength of light emitted from an emission layer EML and may increase emission efficiency. The materials included in the buffer layer may utilize materials which may be included in the hole transport region HTR. The electron blocking layer EBL is a layer playing the role of preventing or reducing electron injection from the electron transport region ETR to the hole transport region HTR.

The first electrode EL1 has conductivity (e.g., is a conductor). The first electrode EL1 may be formed utilizing a metal material, a metal alloy, or a conductive compound. The first electrode EL1 may be an anode or a cathode. However, embodiments of the present disclosure are not limited thereto. In some embodiments, the first electrode EL1 may be a pixel electrode. The first electrode EL1 may be a transmissive electrode, a transflective electrode, or a reflective electrode. The first electrode EL1 may include at least one selected from among silver (Ag), magnesium (Mg), copper (Cu), aluminum (Al), platinum (Pt), palladium (Pd), gold (Au), nickel (Ni), neodymium (Nd), iridium (Ir), chromium (Cr), lithium (Li), calcium (Ca), lithium fluoride (LiF), molybdenum (Mo), titanium (Ti), tungsten (W), indium (In), tin (Sn), and zinc (Zn), compounds of two or more selected therefrom, mixtures of two or more selected therefrom, and/or oxides thereof.

When the first electrode EL1 is a transmissive electrode, the first electrode EL1 may include a transparent metal oxide such as indium tin oxide (ITO), indium zinc oxide (IZO), zinc oxide (ZnO), and/or indium tin zinc oxide (ITZO). When the first electrode EL1 is a transflective electrode or a reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, compounds thereof, or mixtures thereof (for example, a mixture of Ag and Mg). Also, the first electrode EL1 may have a structure including multiple layers including a reflective layer or a transflective layer formed utilizing the above materials, and a transmissive conductive layer formed utilizing ITO, IZO, ZnO, or ITZO. For example, in some embodiments, the first electrode EL1 may have a three-layer structure of ITO/Ag/ITO. However, embodiments of the present disclosure are not limited thereto. The first electrode EL1 may include the above-described metal materials, combinations of two or more metal materials selected from the above-described metal materials, and/or oxides of the above-described metal materials. The thickness of the first electrode EL1 may be from about 700 Å to about 10,000 Å. For example, in one or more embodiments, the thickness of the first electrode EL1 may be from about 1,000 Å to about 3,000 Å.

The emission layer EML may be provided on the hole transport region HTR. The emission layer EML may have a thickness of, for example, about 100 Å to about 1,000 Å, or about 100 Å to about 300 Å. The emission layer EML may have a single layer formed utilizing a single material, a single layer formed utilizing multiple different materials, or a multilayer structure having multiple layers formed utilizing multiple different materials.

In the light emitting element ED of one or more embodiments, the emission layer EML may include anthracene derivatives, pyrene derivatives, fluoranthene derivatives, chrysene derivatives, dihydrobenzanthracene derivatives, and/or triphenylene derivatives. For example, in one or more embodiments, the emission layer EML may include anthracene derivatives or pyrene derivatives.

In the light emitting elements ED of one or more embodiments, shown in FIG. 3 to FIG. 6, the emission layer EML may include a host and a dopant, and the emission layer EML may include a compound represented by Formula E-1. The compound represented by Formula E-1 may be utilized as a fluorescence host material.

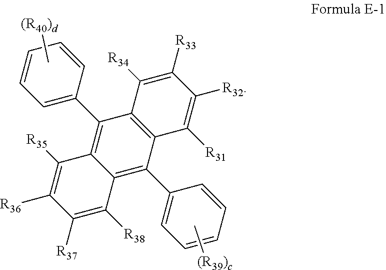

In Formula E-1, R₃₁ to R₄₀ may each independently be hydrogen, deuterium, a halogen, a substituted or unsubstituted silyl group, a substituted or unsubstituted thio group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In some embodiments, R₃₁ to R₄₀ may be combined with an adjacent group to form a saturated hydrocarbon ring, an unsaturated hydrocarbon ring, a saturated heterocycle, or an unsaturated heterocycle.

In Formula E-1, “c” and “d” may each independently be an integer of 0 to 5.

The compound represented by Formula E-1 may be any one selected from among Compound E1 to Compound E19.

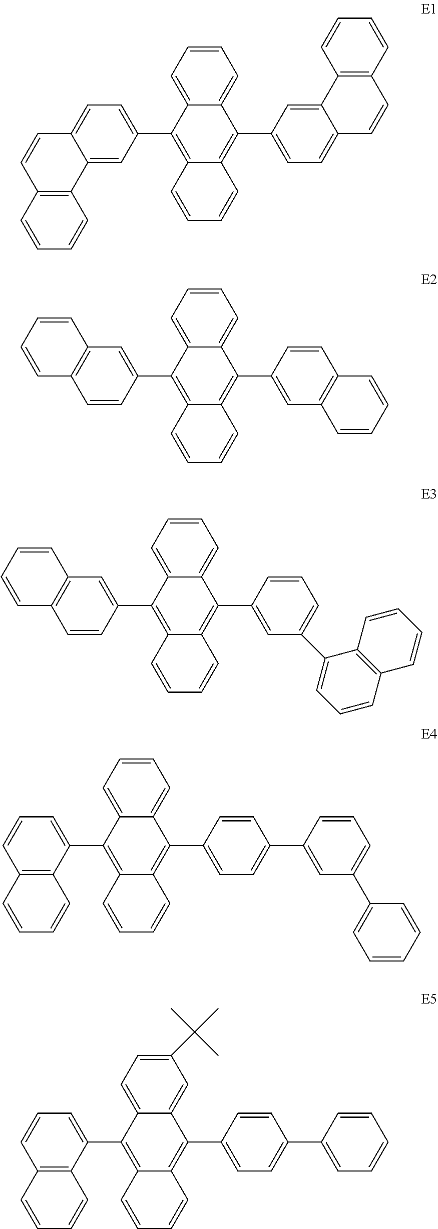

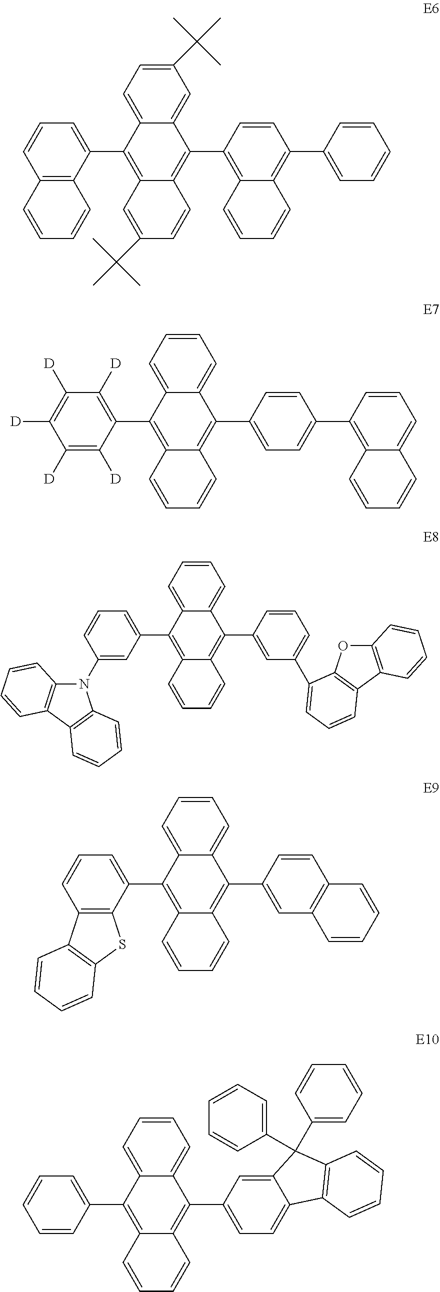

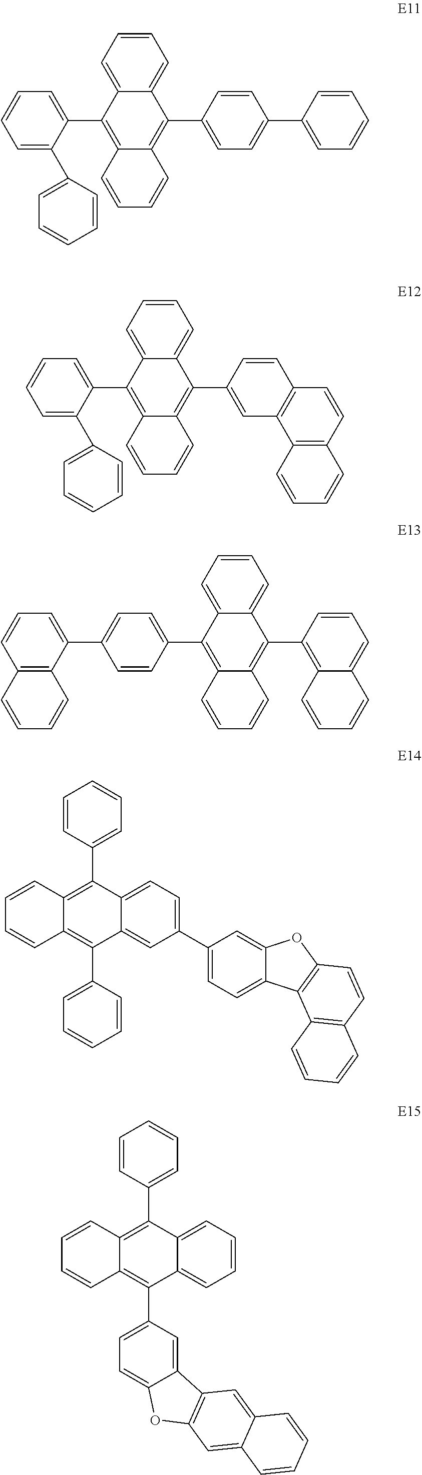

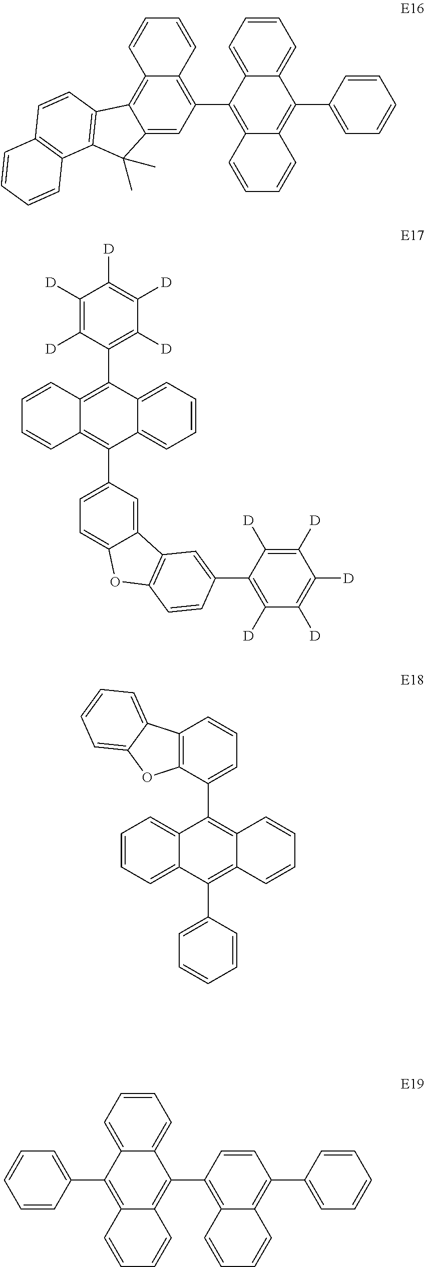

In one or more embodiments, the emission layer EML may include at least one selected from among a first compound represented by Formula E-1, a second compound represented by Formula HT-1, a third compound represented by Formula ET-1, and a fourth compound represented by Formula M-b.

In one or more embodiments, the second compound may be utilized as a hole transport host material of the emission layer EML.

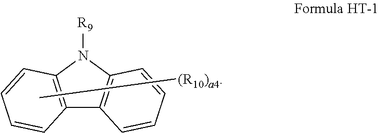

In Formula HT-1, a4 may be an integer of 0 to 8. When a4 is an integer of 2 or more, multiple R₁₀(s) may be the same, or at least one may be different. R₉ and R₁₀ may each independently be hydrogen, deuterium, a substituted or unsubstituted aryl group of 6 to 60 ring-forming carbon atoms, or a substituted or unsubstituted heteroaryl group of 2 to 60 ring-forming carbon atoms. For example, in one or more embodiments, R₉ may be a substituted phenyl group, an unsubstituted dibenzofuran group, or a substituted fluorenyl group. R₁₀ may be a substituted or unsubstituted carbazole group.

The second compound may be represented by any one selected from among compounds in Compound Group 2. In Compound Group 2, D is deuterium, “Ph” may refer to an unsubstituted phenyl group.

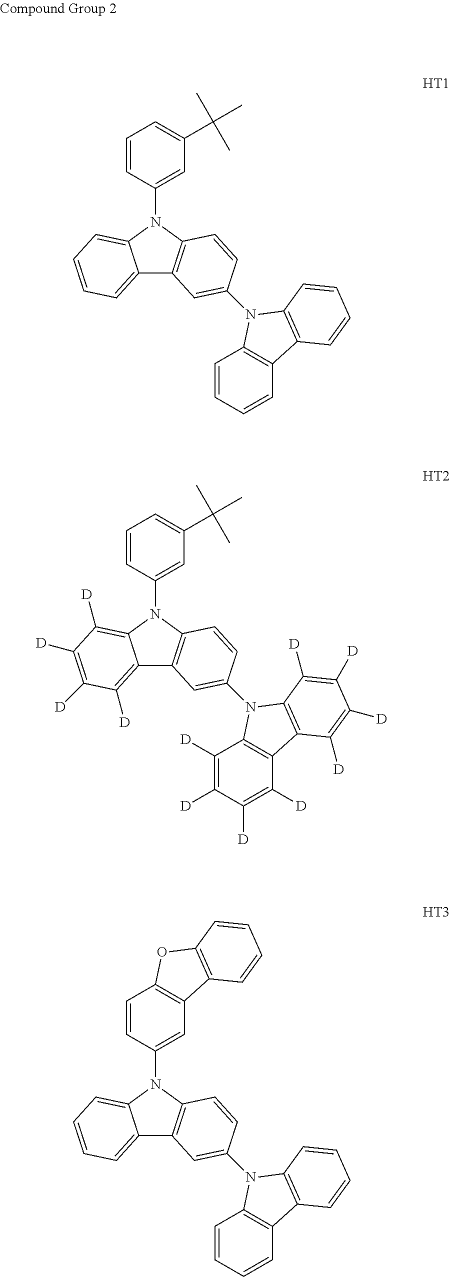 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

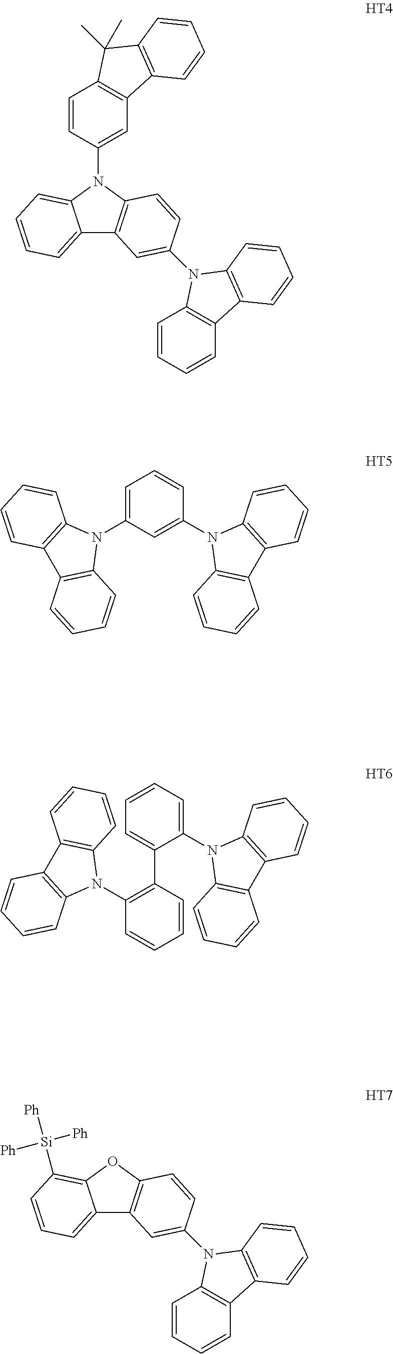 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1]

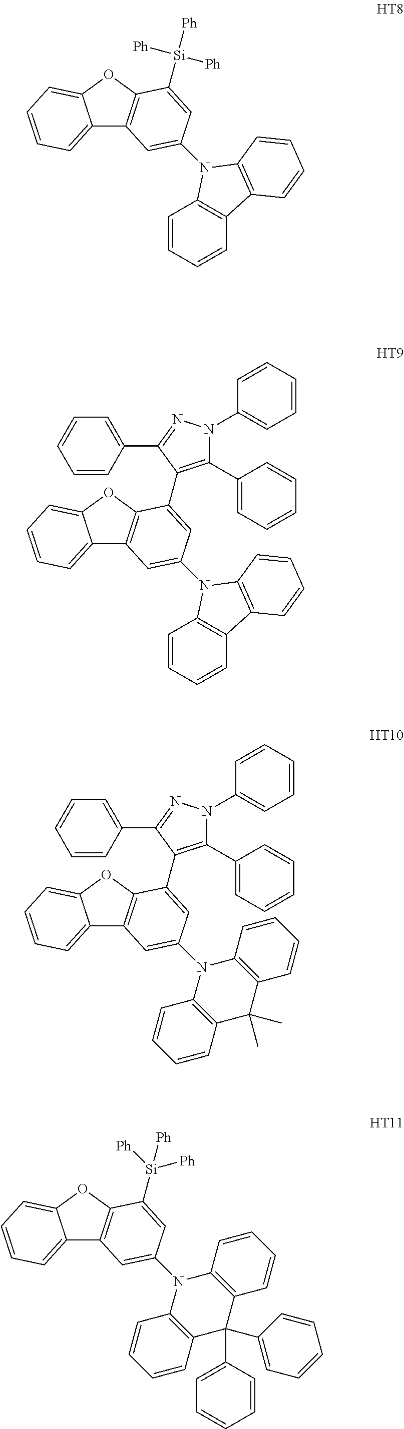 [SMILES: c1ccc([Si](c2ccccc2)(c2ccccc2)c2cc(-n3c4ccccc4c4ccccc43)cc3c2oc2ccccc23)cc1 ; c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1]

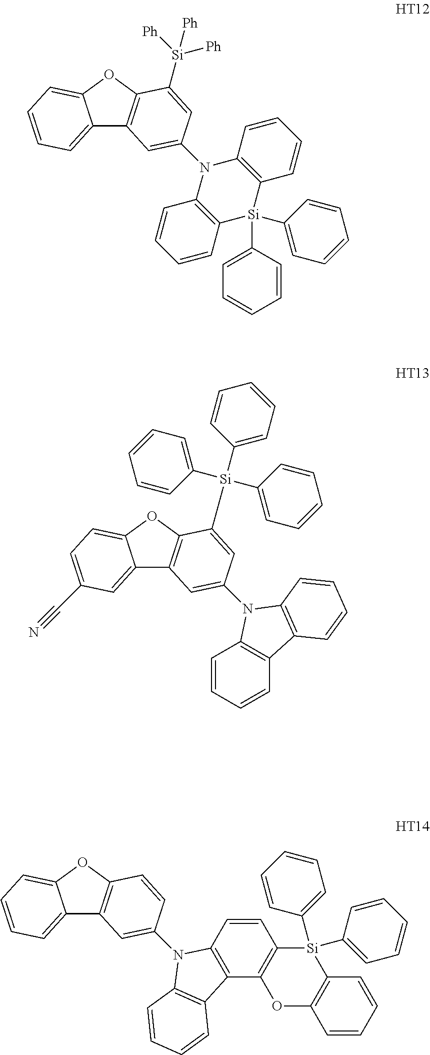 [SMILES: c1ccc([Si]2(c3ccccc3)c3ccccc3N(c3cc([Si](c4ccccc4)(c4ccccc4)c4ccccc4)c4oc5ccccc5c4c3)c3ccccc32)cc1 ; N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1]

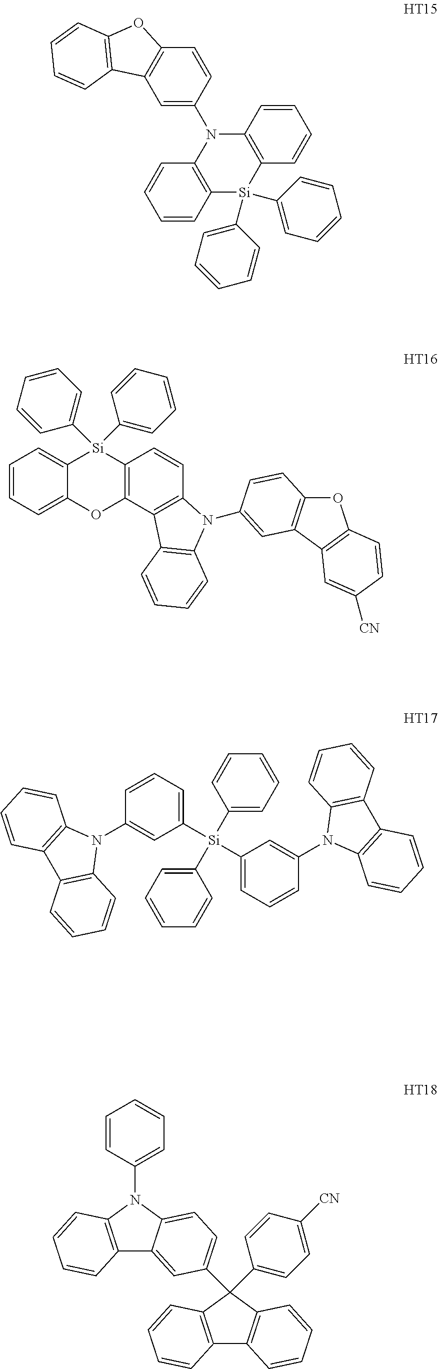 [SMILES: c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4O6)cc3c2c1 ; c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1]

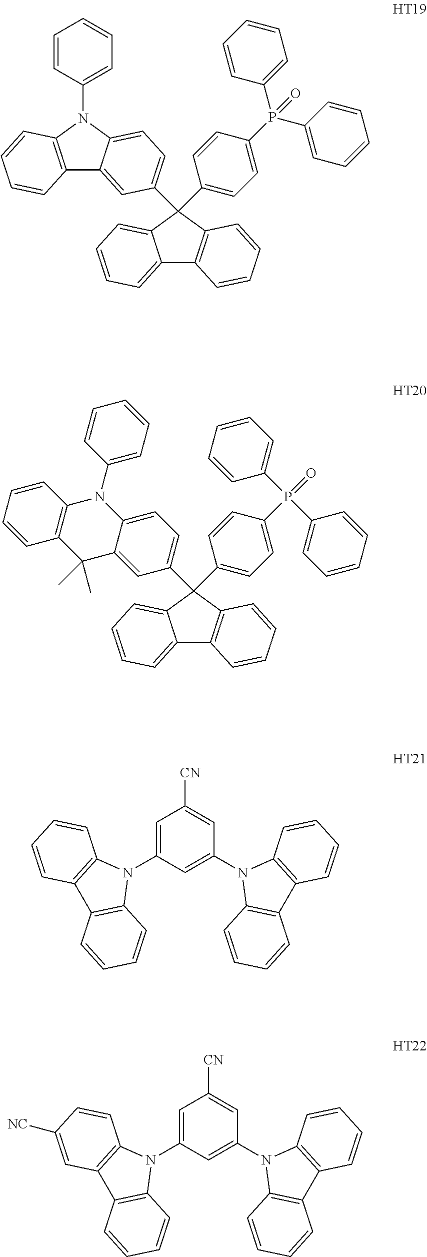 [SMILES: O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21 ; N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1]

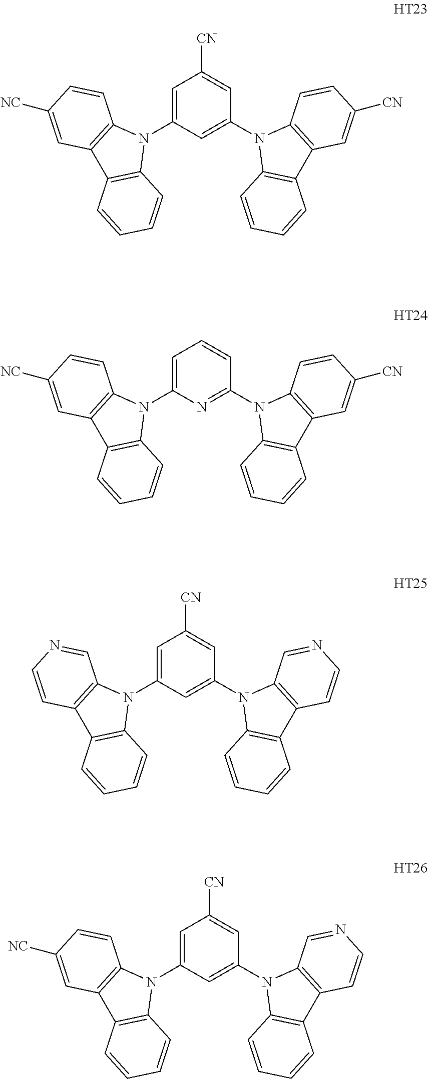 [SMILES: N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1 ; N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1]

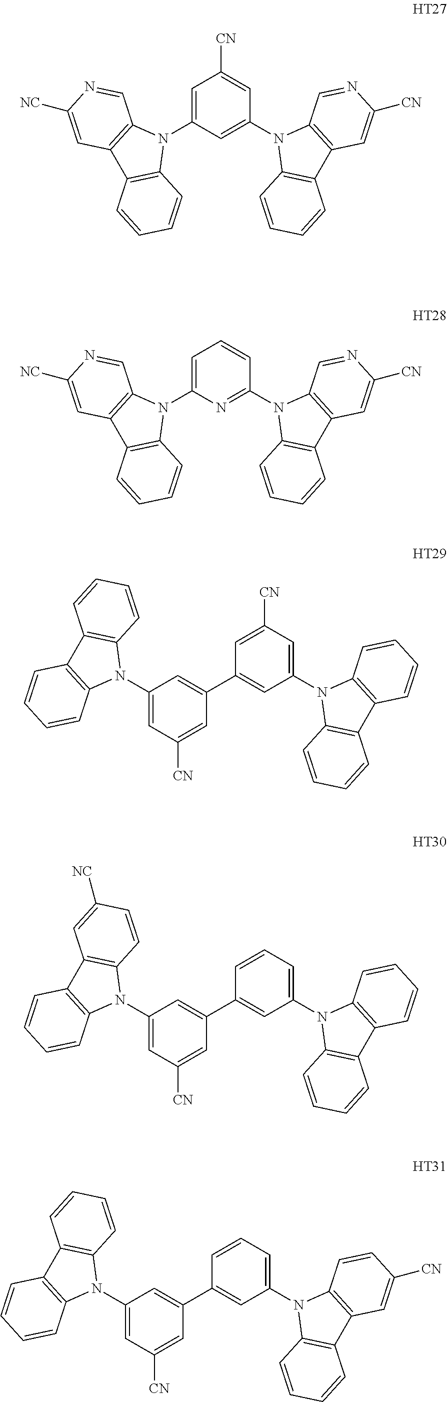 [SMILES: N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1 ; N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1]

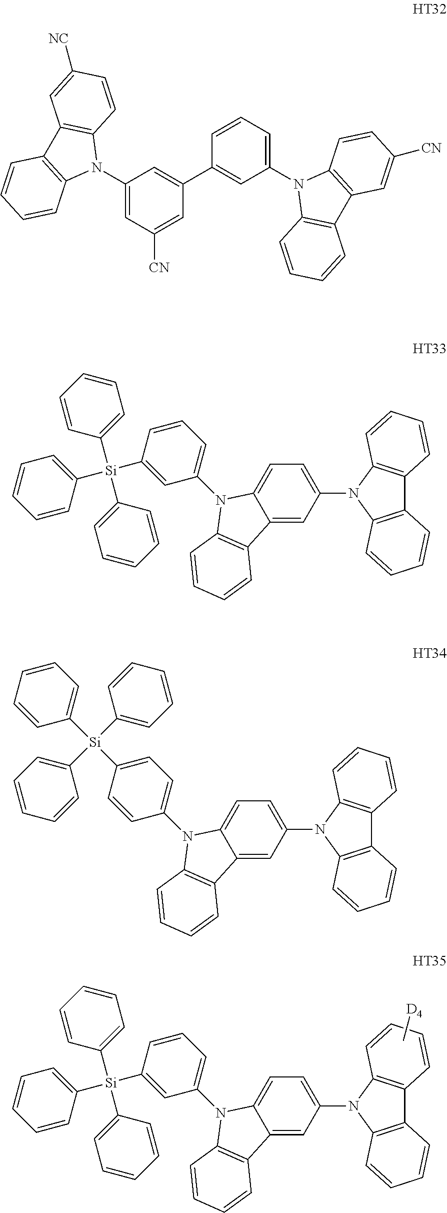

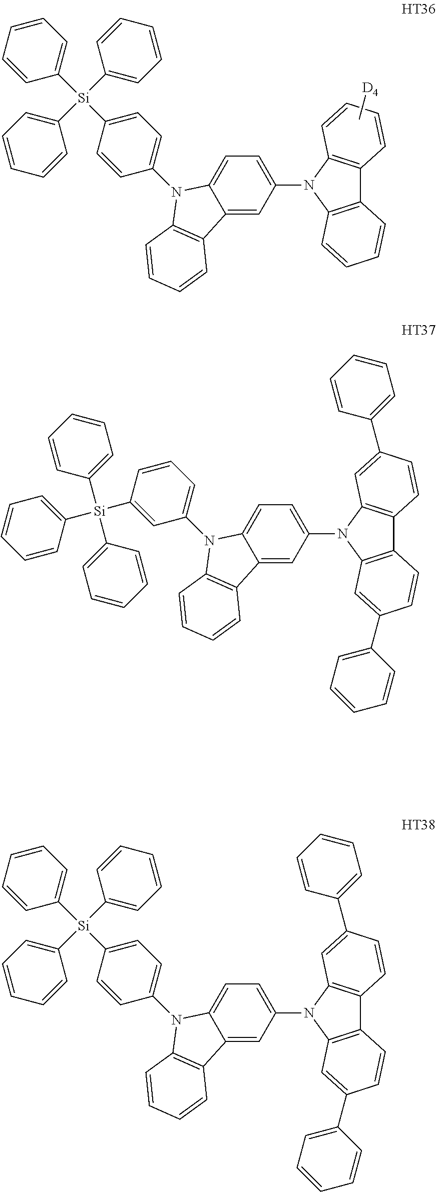

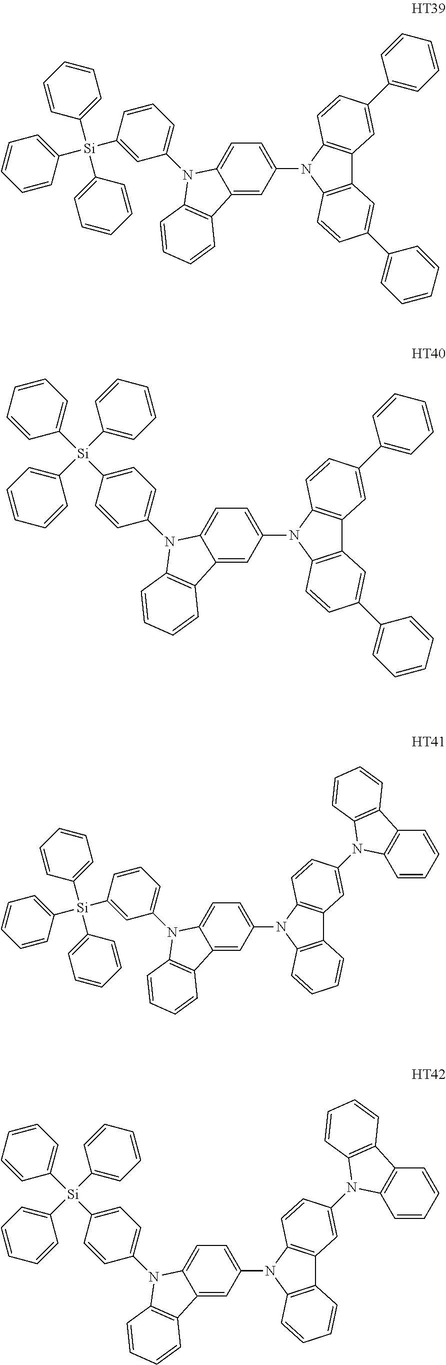 [SMILES: c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1 ; c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1]

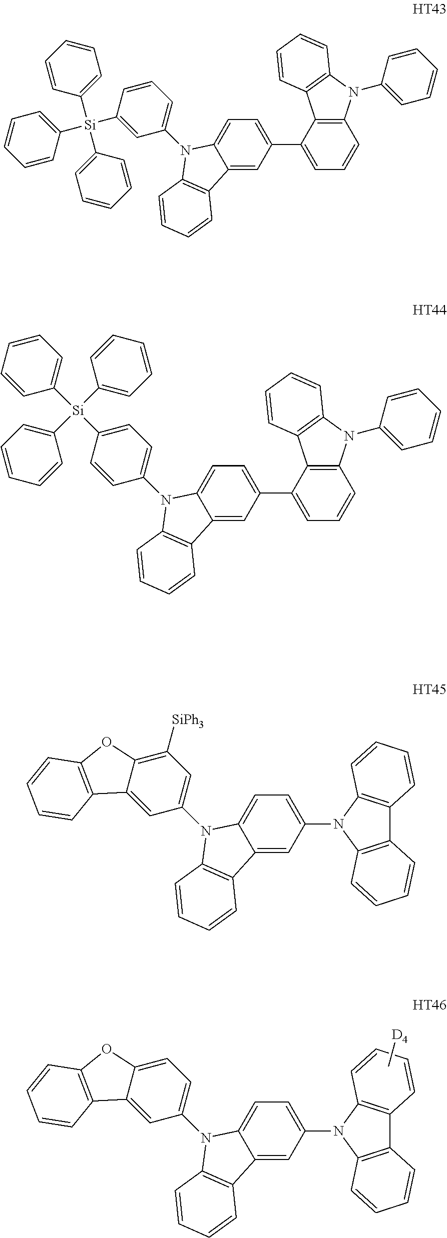

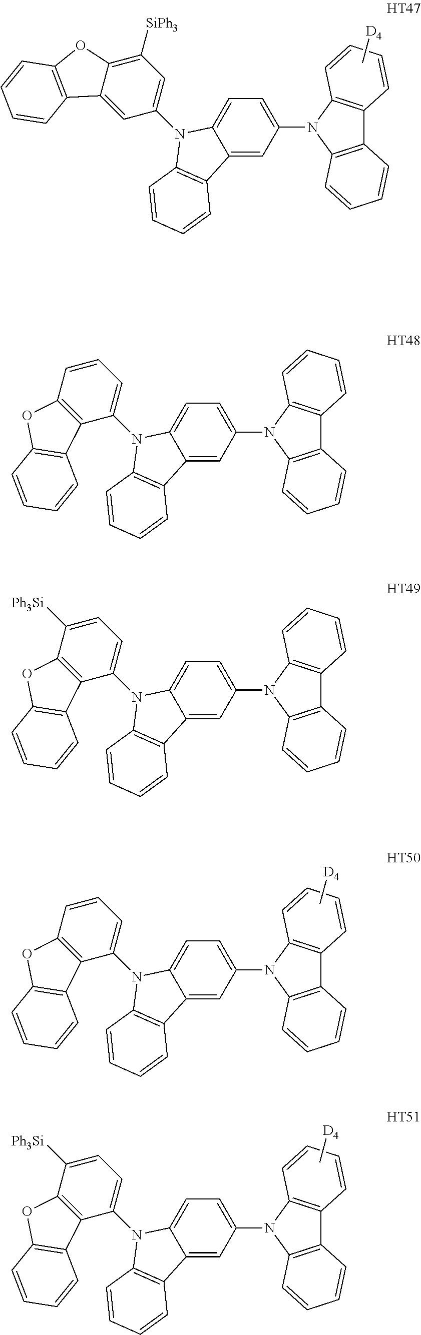

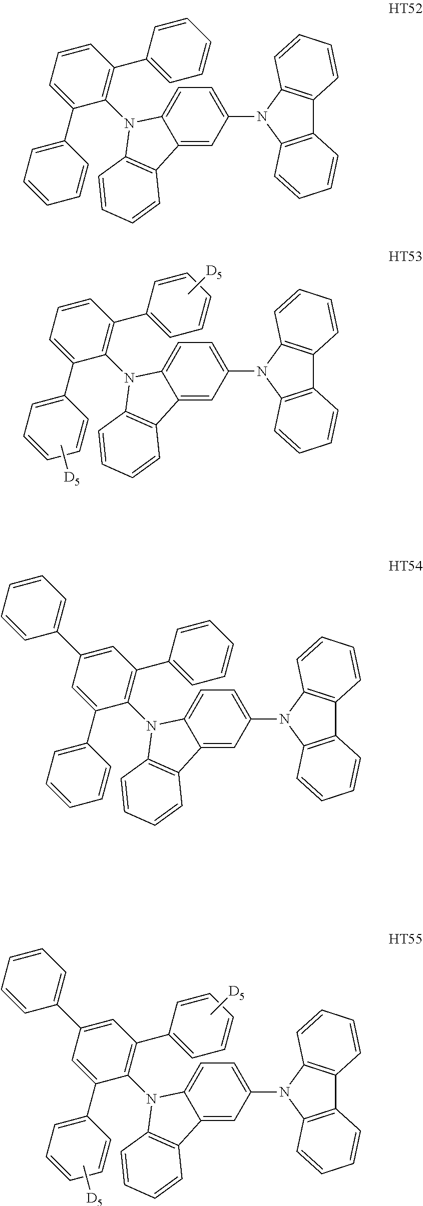

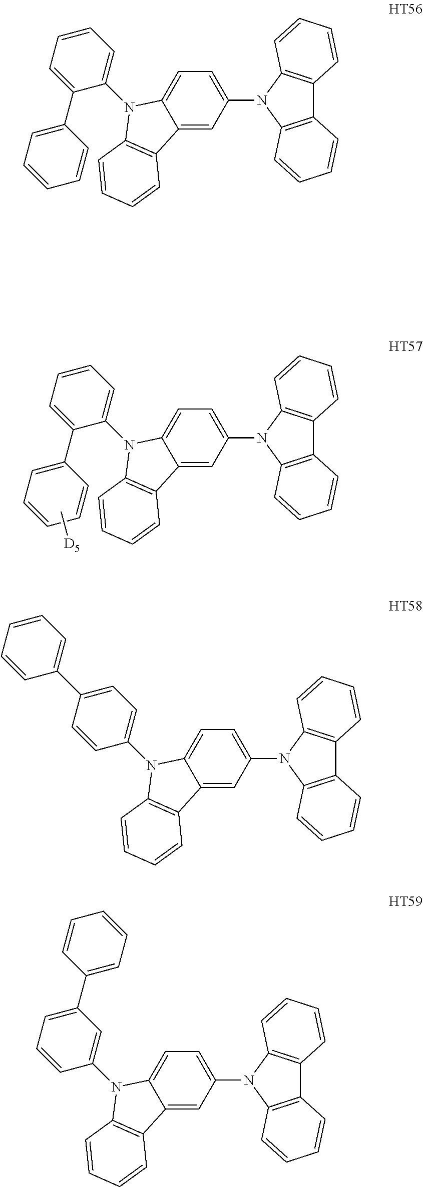

In one or more embodiments, the emission layer EML may include a third compound represented by Formula ET-1. For example, the third compound may be utilized as an electron transport host material of the emission layer EML.

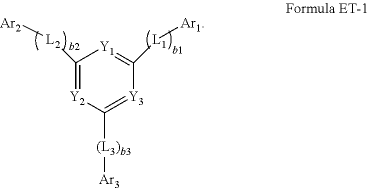

In Formula ET-1, at least one selected from among Y₁ to Y₃ may be N, and the remainder may be CRₐ, and Rₐ may be hydrogen, deuterium, a substituted or un substituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms.

b1 to b3 may each independently be an integer of 0 to 10. L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms.

Ar₁ to Ar₃ may each independently be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, in one or more embodiments, Ar₁ to Ar₃ may be substituted or unsubstituted phenyl groups, or substituted or unsubstituted carbazole groups.

The third compound may be represented by any one selected from among compounds in Compound Group 3. The light emitting element ED of one or more embodiments may include any one selected from among the compounds in Compound Group 3. In Compound Group 3, D is a deuterium atom.

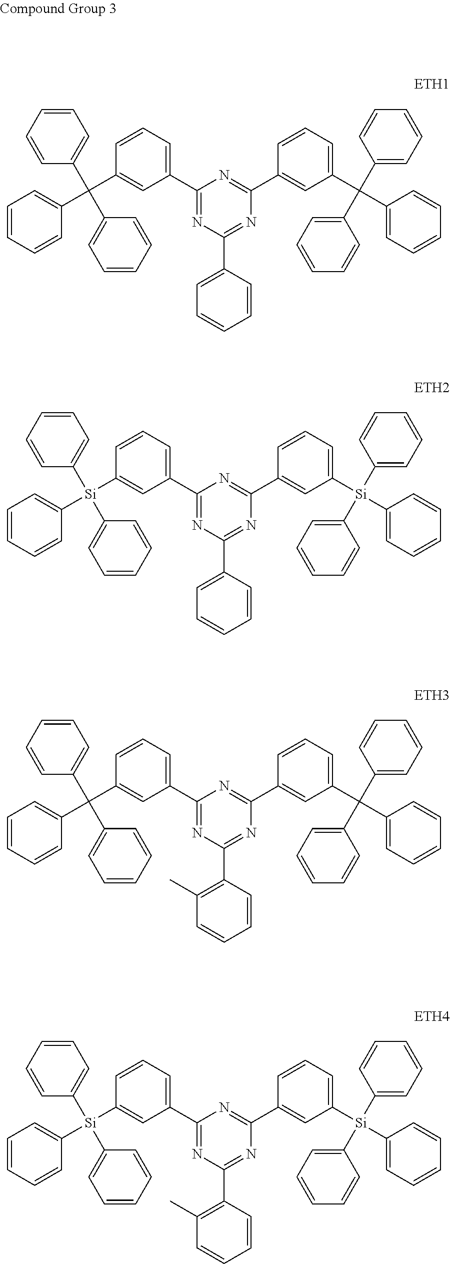 [SMILES: c1ccc(-c2nc(-c3cccc(C(c4ccccc4)(c4ccccc4)c4ccccc4)c3)nc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)nc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)nc(-c2cccc([Si](c3ccccc3)(c3ccccc3)c3ccccc3)c2)n1]

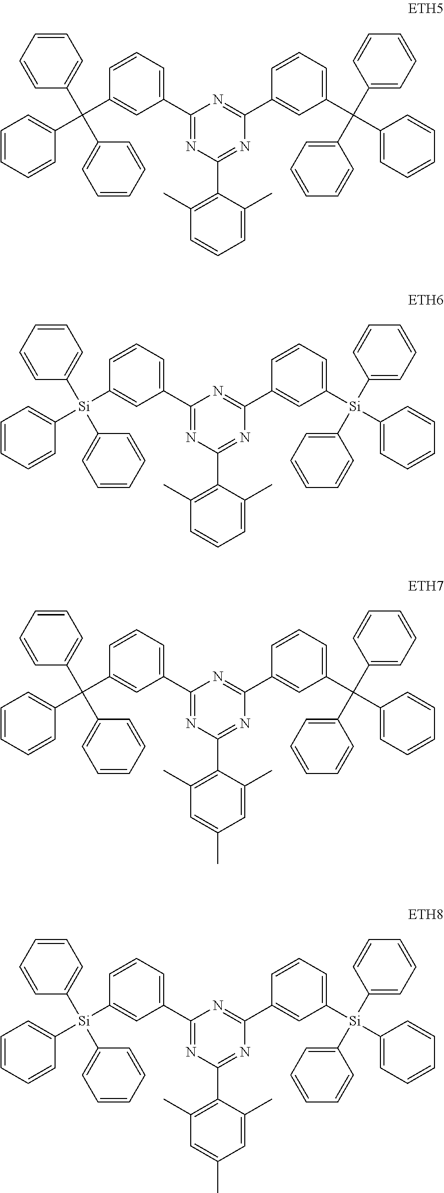 [SMILES: Cc1cccc(C)c1-c1nc(-c2cccc(C(c3ccccc3)(c3ccccc3)c3ccccc3)c2)nc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)nc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)nc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)nc(-c3cccc([Si](c4ccccc4)(c4ccccc4)c4ccccc4)c3)n2)c(C)c1]

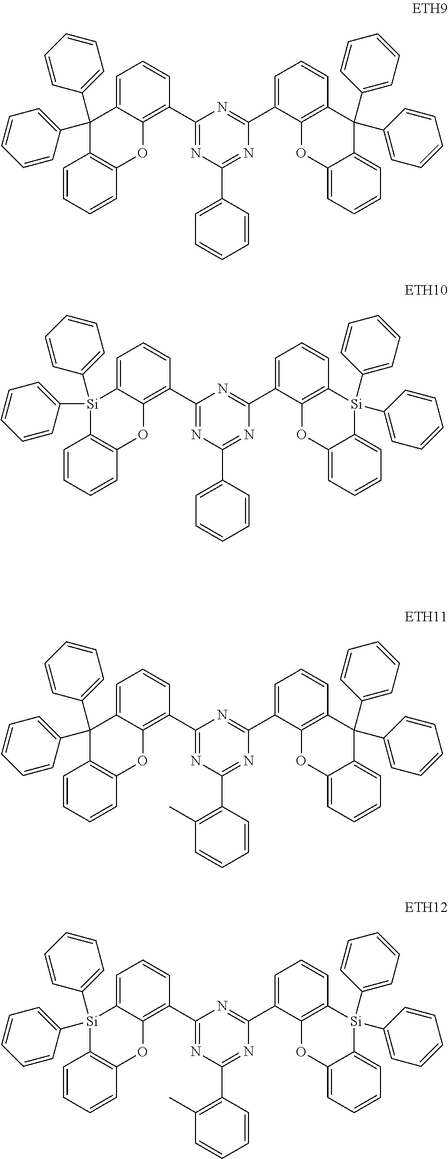 [SMILES: c1ccc(-c2nc(-c3cccc4c3Oc3ccccc3C4(c3ccccc3)c3ccccc3)nc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1]

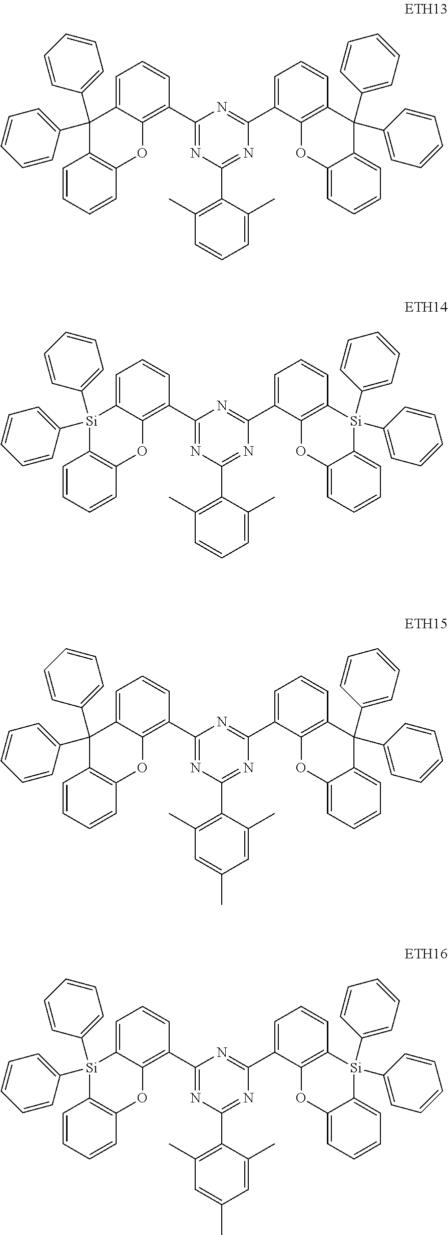 [SMILES: Cc1cccc(C)c1-c1nc(-c2cccc3c2Oc2ccccc2C3(c2ccccc2)c2ccccc2)nc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)nc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)nc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

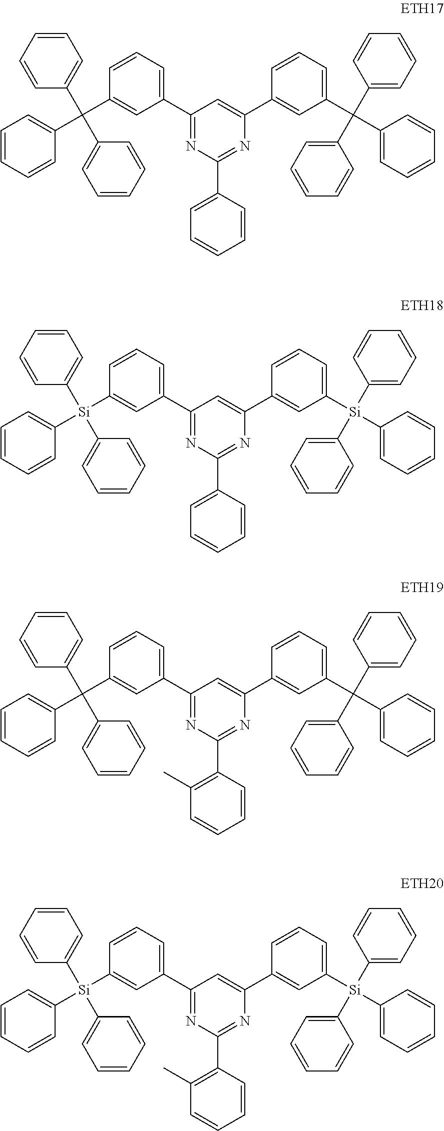 [SMILES: c1ccc(-c2nc(-c3cccc(C(c4ccccc4)(c4ccccc4)c4ccccc4)c3)cc(-c3cccc(C(c4ccccc4)(c4ccccc4)c4ccccc4)c3)n2)cc1 ; c1ccc(-c2nc(-c3cccc([Si](c4ccccc4)(c4ccccc4)c4ccccc4)c3)cc(-c3cccc([Si](c4ccccc4)(c4ccccc4)c4ccccc4)c3)n2)cc1 ; Cc1ccccc1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1ccccc1-c1nc(-c2cccc([Si](c3ccccc3)(c3ccccc3)c3ccccc3)c2)cc(-c2cccc([Si](c3ccccc3)(c3ccccc3)c3ccccc3)c2)n1]

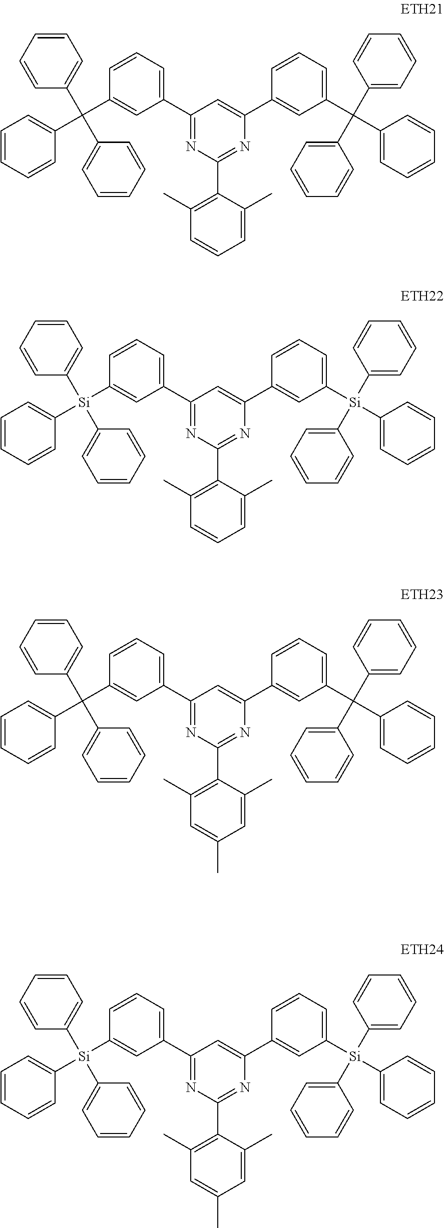 [SMILES: Cc1cccc(C)c1-c1nc(-c2cccc(C(c3ccccc3)(c3ccccc3)c3ccccc3)c2)cc(-c2cccc(C(c3ccccc3)(c3ccccc3)c3ccccc3)c2)n1 ; Cc1cccc(C)c1-c1nc(-c2cccc([Si](c3ccccc3)(c3ccccc3)c3ccccc3)c2)cc(-c2cccc([Si](c3ccccc3)(c3ccccc3)c3ccccc3)c2)n1 ; Cc1cc(C)c(-c2nc(-c3cccc(C(c4ccccc4)(c4ccccc4)c4ccccc4)c3)cc(-c3cccc(C(c4ccccc4)(c4ccccc4)c4ccccc4)c3)n2)c(C)c1 ; Cc1cc(C)c(-c2nc(-c3cccc([Si](c4ccccc4)(c4ccccc4)c4ccccc4)c3)cc(-c3cccc([Si](c4ccccc4)(c4ccccc4)c4ccccc4)c3)n2)c(C)c1]

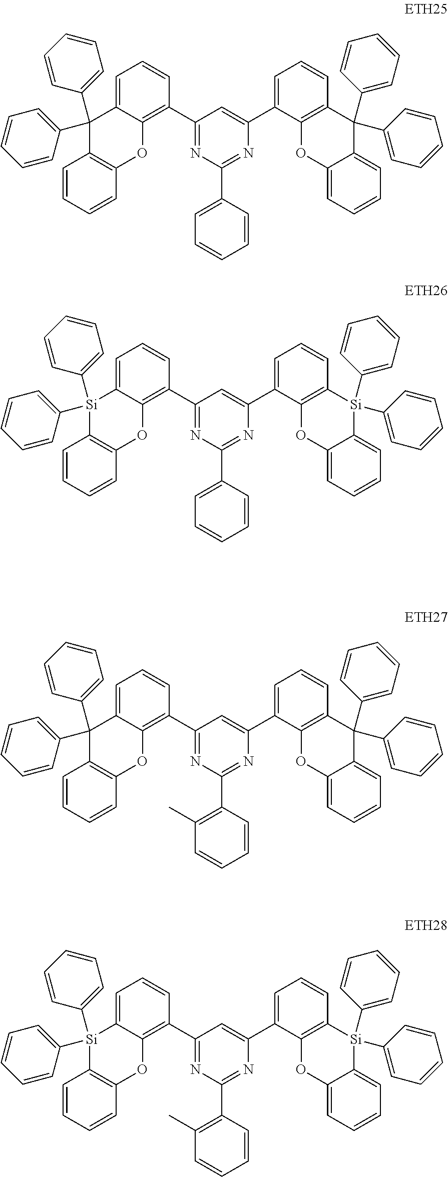 [SMILES: c1ccc(-c2nc(-c3cccc4c3Oc3ccccc3C4(c3ccccc3)c3ccccc3)cc(-c3cccc4c3Oc3ccccc3C4(c3ccccc3)c3ccccc3)n2)cc1 ; c1ccc(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)cc1 ; Cc1ccccc1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1ccccc1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1]

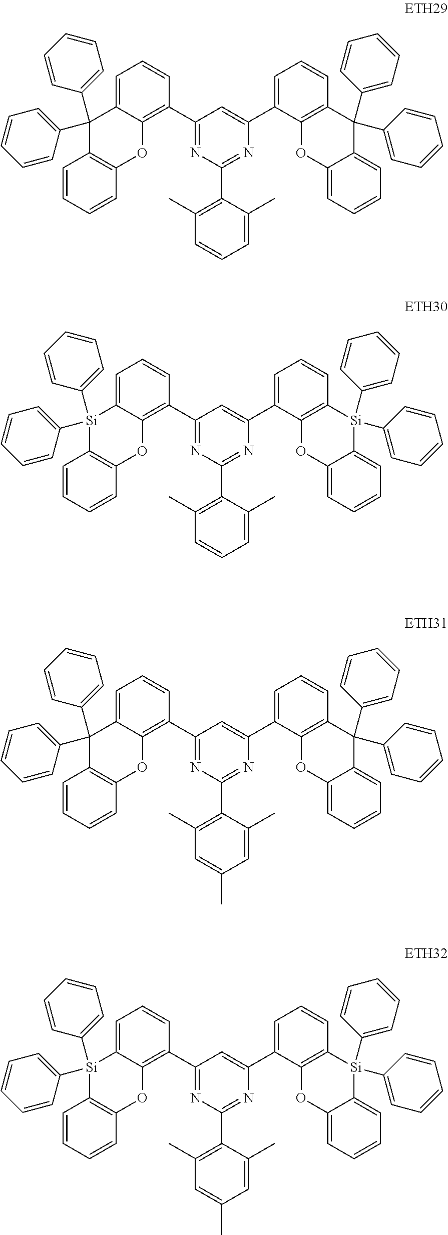 [SMILES: Cc1cccc(C)c1-c1nc(-c2cccc3c2Oc2ccccc2C3(c2ccccc2)c2ccccc2)cc(-c2cccc3c2Oc2ccccc2C3(c2ccccc2)c2ccccc2)n1 ; Cc1cccc(C)c1-c1nc(-c2cccc3c2Oc2ccccc2[Si]3(c2ccccc2)c2ccccc2)cc(-c2cccc3c2Oc2ccccc2[Si]3(c2ccccc2)c2ccccc2)n1 ; Cc1cc(C)c(-c2nc(-c3cccc4c3Oc3ccccc3C4(c3ccccc3)c3ccccc3)cc(-c3cccc4c3Oc3ccccc3C4(c3ccccc3)c3ccccc3)n2)c(C)c1 ; Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1]

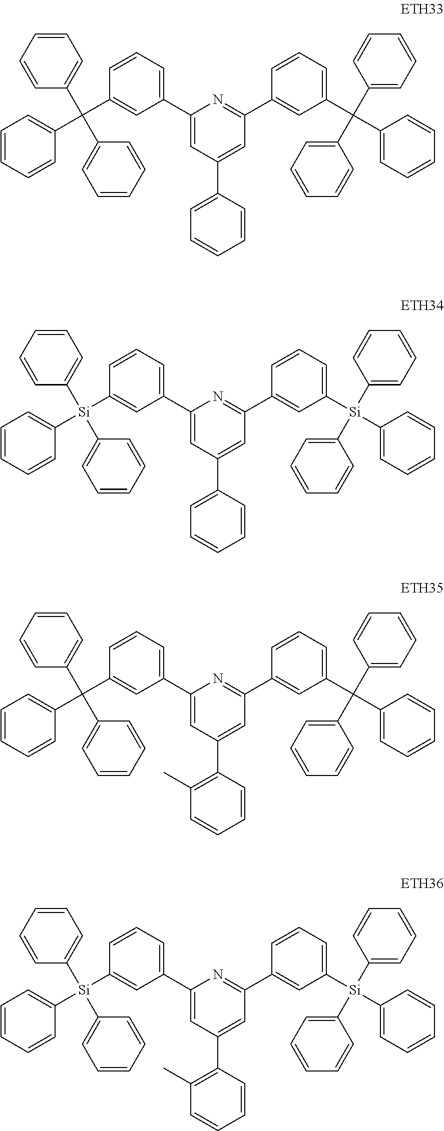 [SMILES: c1ccc(-c2cc(-c3cccc(C(c4ccccc4)(c4ccccc4)c4ccccc4)c3)nc(-c3cccc(C(c4ccccc4)(c4ccccc4)c4ccccc4)c3)c2)cc1 ; c1ccc(-c2cc(-c3cccc([Si](c4ccccc4)(c4ccccc4)c4ccccc4)c3)nc(-c3cccc([Si](c4ccccc4)(c4ccccc4)c4ccccc4)c3)c2)cc1 ; Cc1ccccc1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1ccccc1-c1cc(-c2cccc([Si](c3ccccc3)(c3ccccc3)c3ccccc3)c2)nc(-c2cccc([Si](c3ccccc3)(c3ccccc3)c3ccccc3)c2)c1]

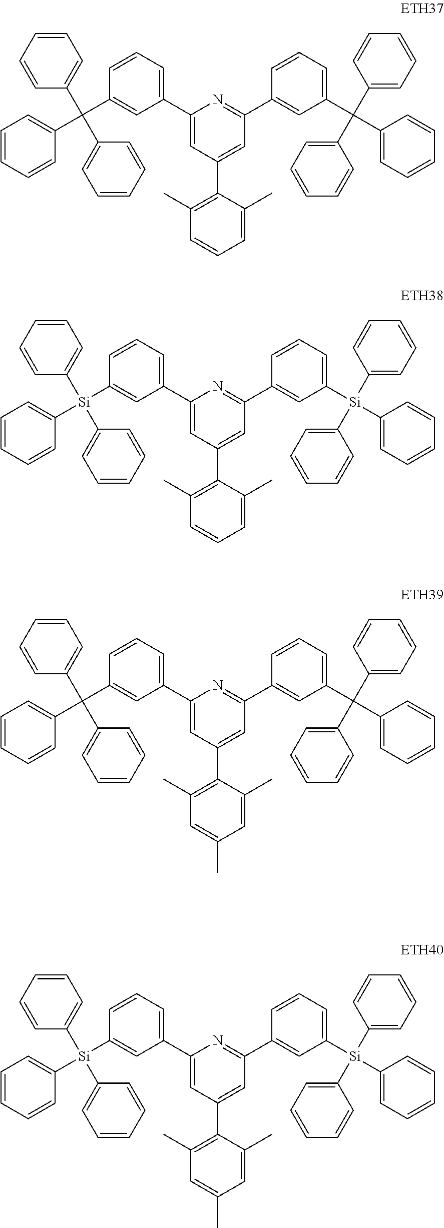 [SMILES: Cc1cccc(C)c1-c1cc(-c2cccc(C(c3ccccc3)(c3ccccc3)c3ccccc3)c2)nc(-c2cccc(C(c3ccccc3)(c3ccccc3)c3ccccc3)c2)c1 ; Cc1cccc(C)c1-c1cc(-c2cccc([Si](c3ccccc3)(c3ccccc3)c3ccccc3)c2)nc(-c2cccc([Si](c3ccccc3)(c3ccccc3)c3ccccc3)c2)c1 ; Cc1cc(C)c(-c2cc(-c3cccc(C(c4ccccc4)(c4ccccc4)c4ccccc4)c3)nc(-c3cccc(C(c4ccccc4)(c4ccccc4)c4ccccc4)c3)c2)c(C)c1 ; Cc1cc(C)c(-c2cc(-c3cccc([Si](c4ccccc4)(c4ccccc4)c4ccccc4)c3)nc(-c3cccc([Si](c4ccccc4)(c4ccccc4)c4ccccc4)c3)c2)c(C)c1]

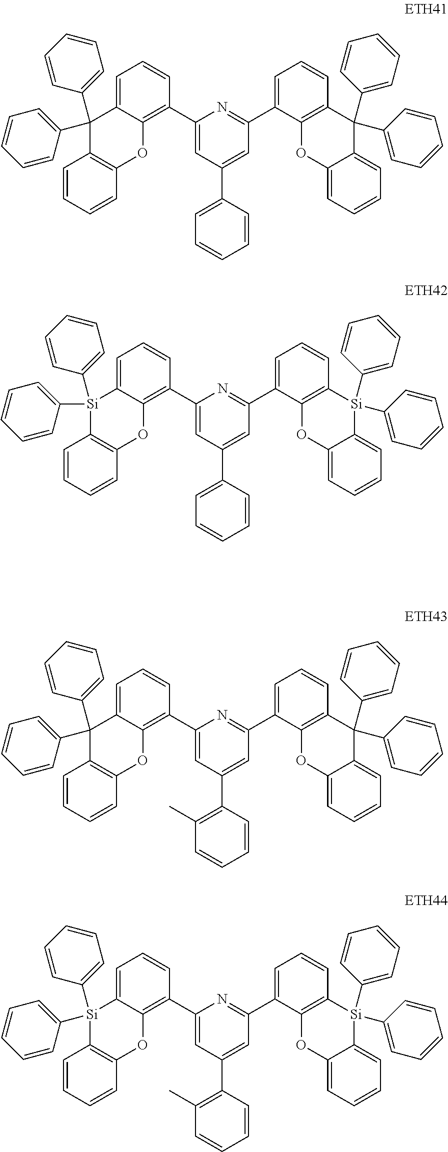 [SMILES: c1ccc(-c2cc(-c3cccc4c3Oc3ccccc3C4(c3ccccc3)c3ccccc3)nc(-c3cccc4c3Oc3ccccc3C4(c3ccccc3)c3ccccc3)c2)cc1 ; c1ccc(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)cc1 ; Cc1ccccc1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1ccccc1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1]

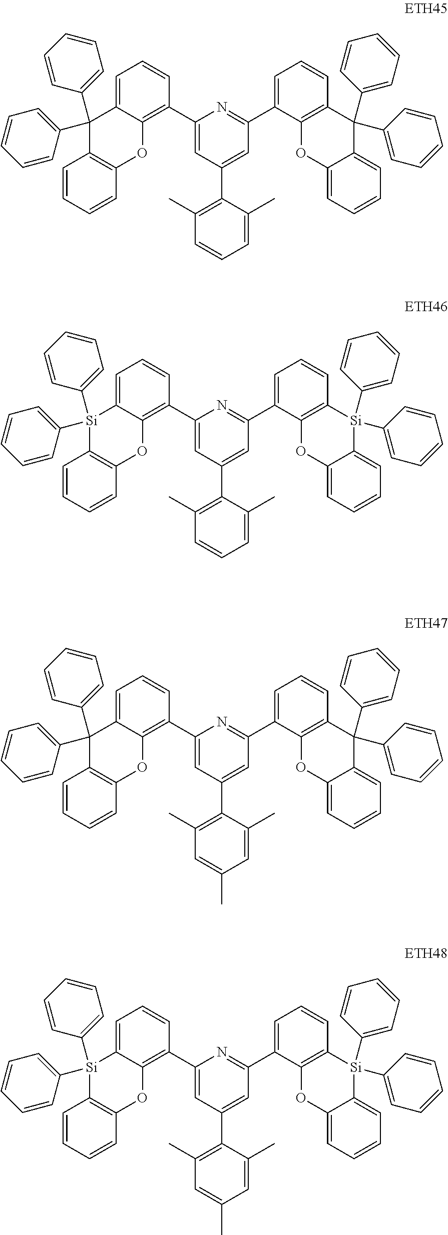 [SMILES: Cc1cccc(C)c1-c1cc(-c2cccc3c2Oc2ccccc2C3(c2ccccc2)c2ccccc2)nc(-c2cccc3c2Oc2ccccc2C3(c2ccccc2)c2ccccc2)c1 ; Cc1cccc(C)c1-c1cc(-c2cccc3c2Oc2ccccc2[Si]3(c2ccccc2)c2ccccc2)nc(-c2cccc3c2Oc2ccccc2[Si]3(c2ccccc2)c2ccccc2)c1 ; Cc1cc(C)c(-c2cc(-c3cccc4c3Oc3ccccc3C4(c3ccccc3)c3ccccc3)nc(-c3cccc4c3Oc3ccccc3C4(c3ccccc3)c3ccccc3)c2)c(C)c1 ; Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

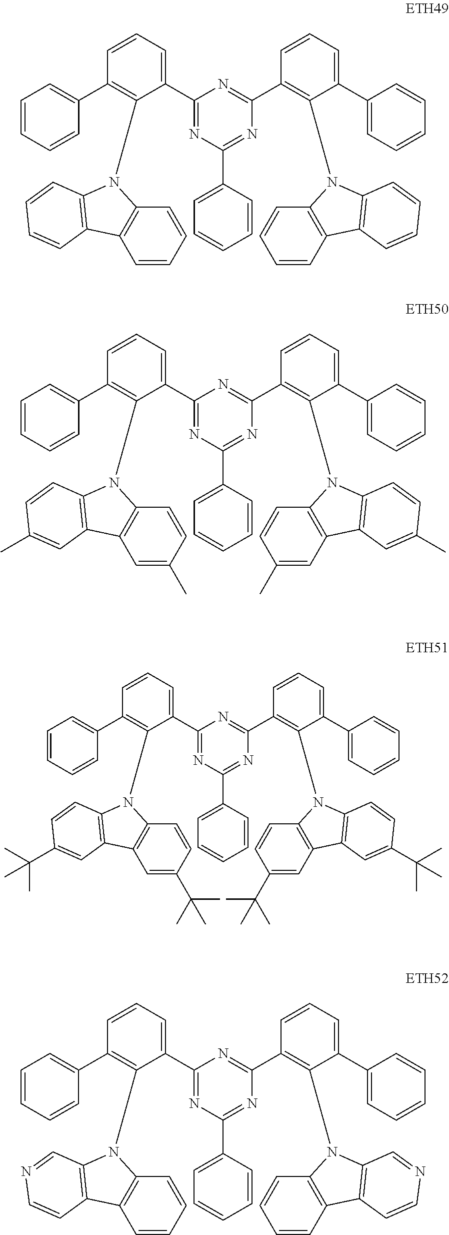

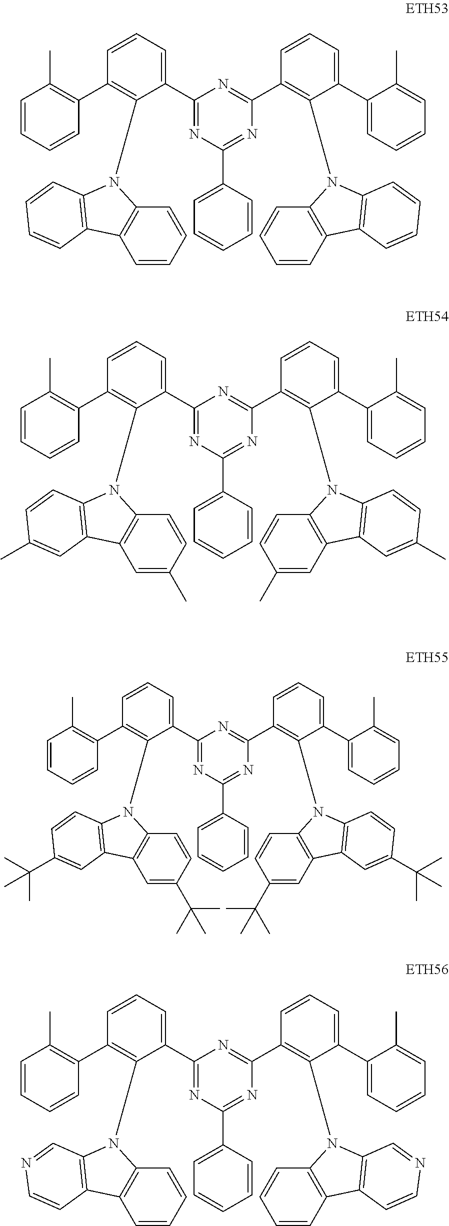

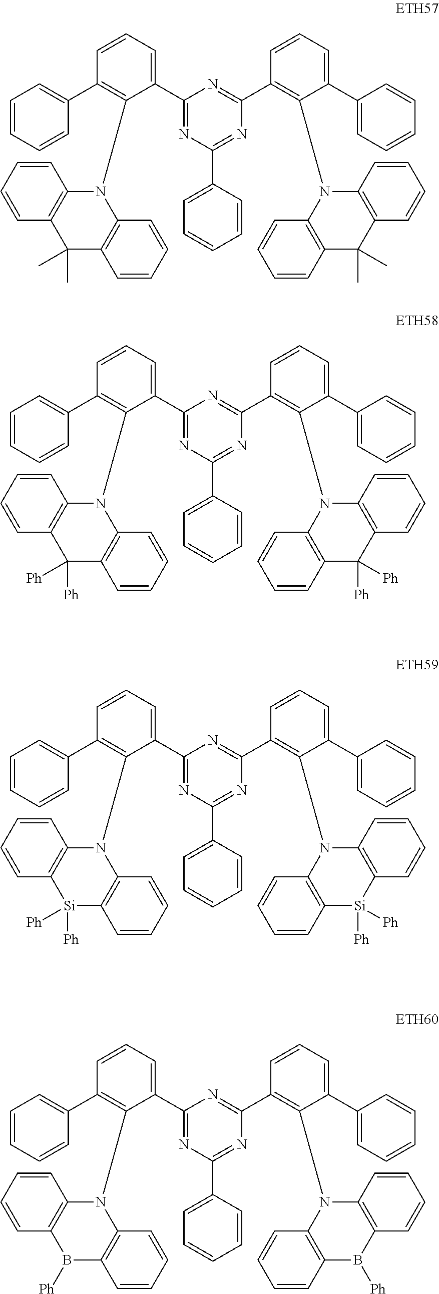

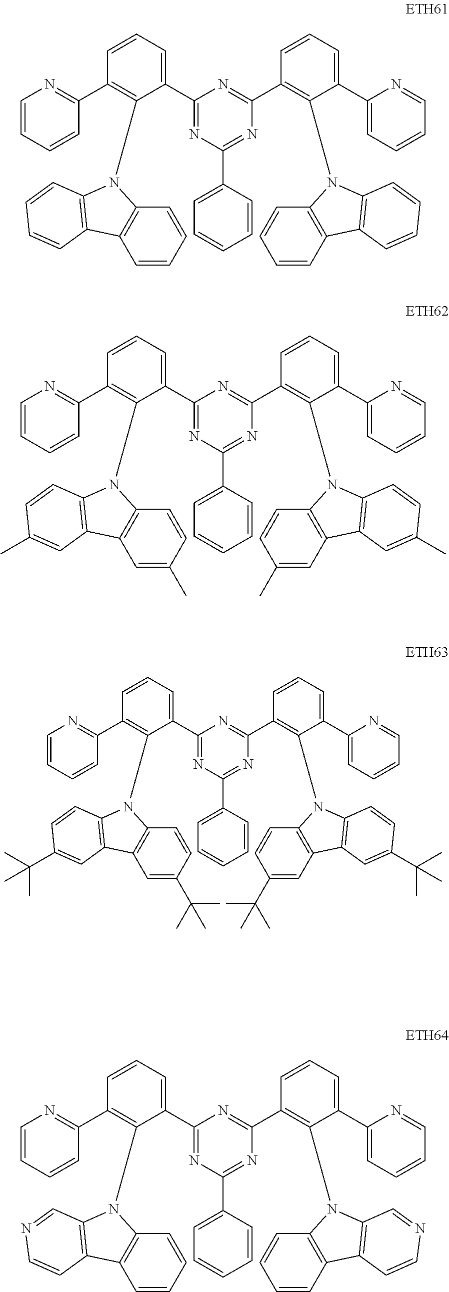

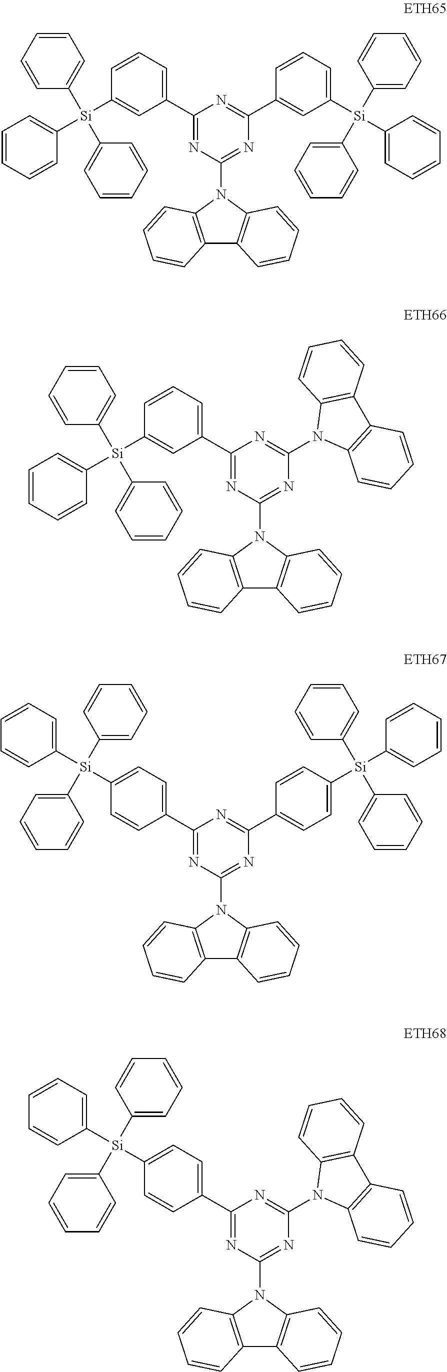 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

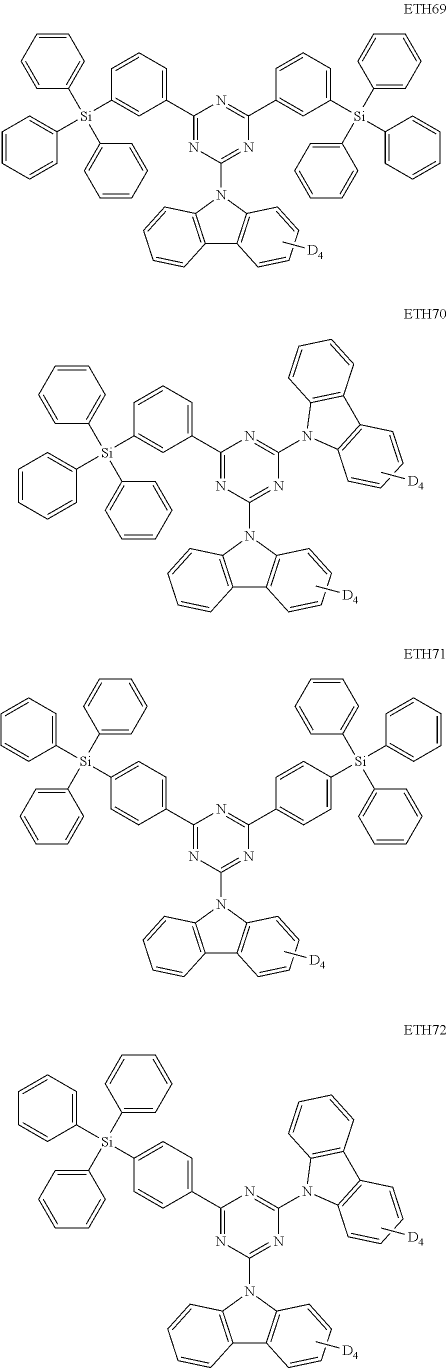

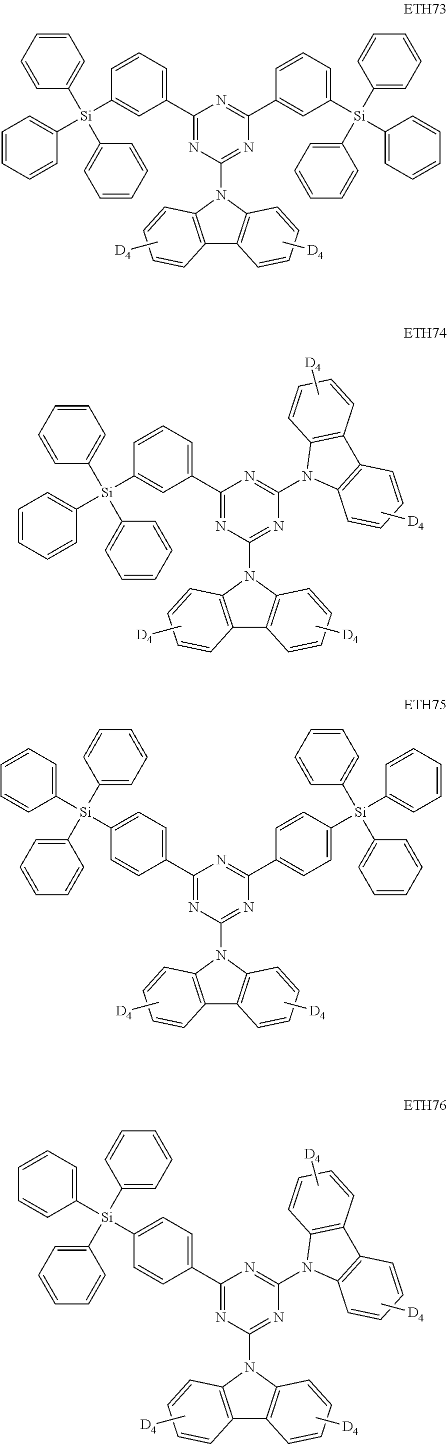

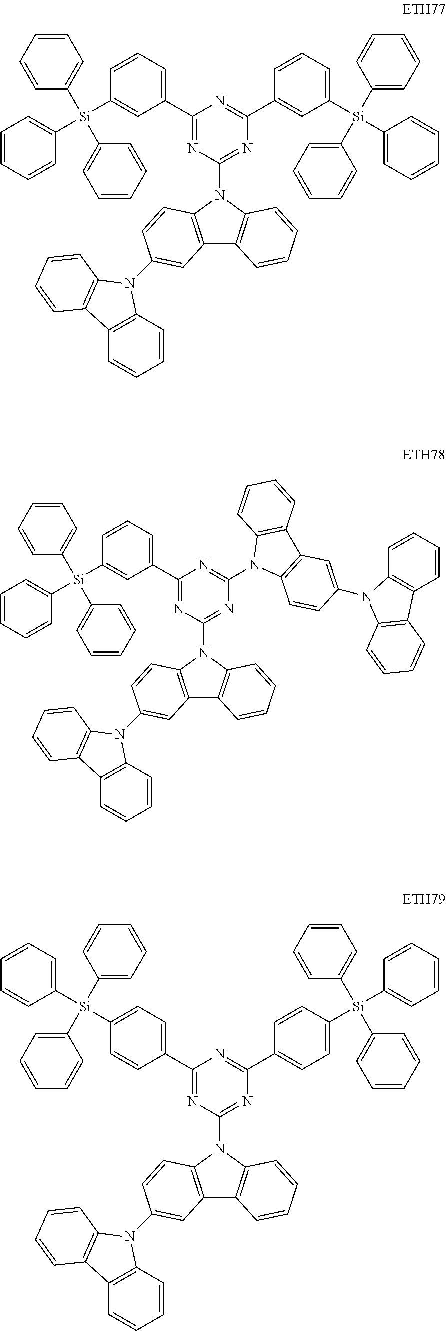 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5cc(-n6c7ccccc7c7ccccc76)ccc54)n3)c2)cc1 ; c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1]

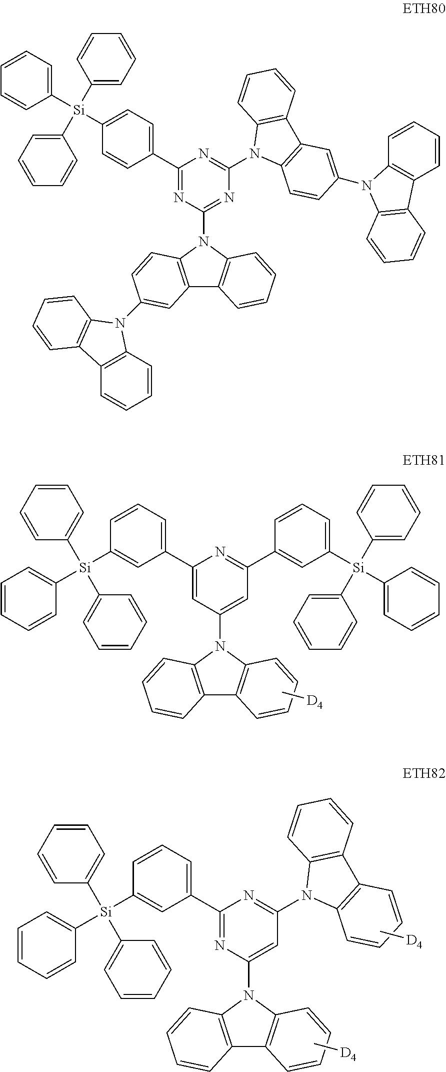

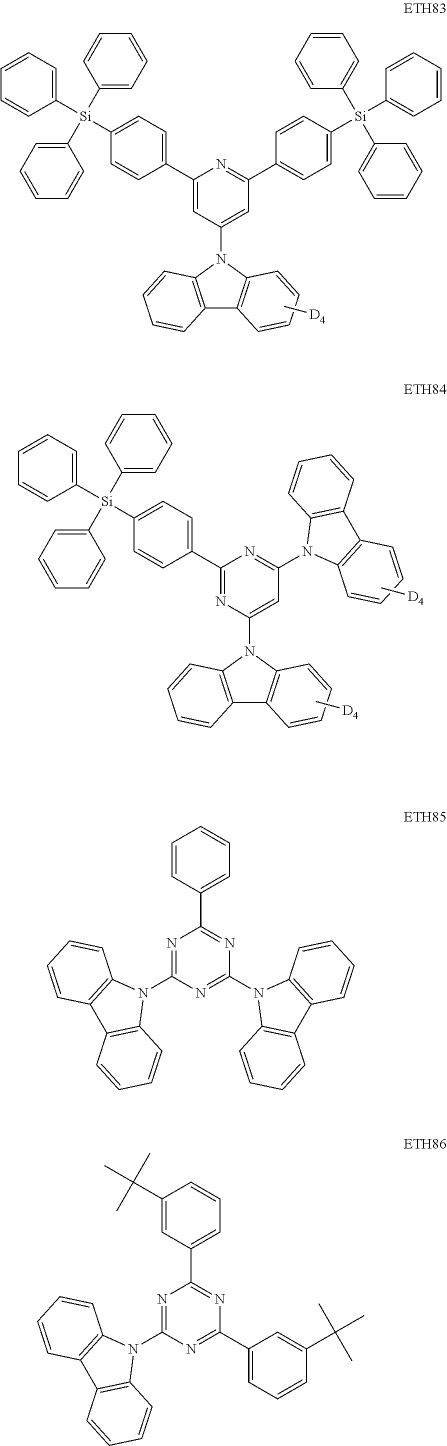

In one or more embodiments, the emission layer EML may include a compound represented by Formula E-2a or Formula E-2b. The compound represented by Formula E-2a or Formula E-2b may be utilized as a phosphorescence host material.

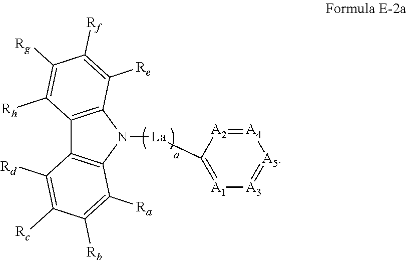

In Formula E-2a, “a” may be an integer of 0 to 10, La may be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when “a” is an integer of 2 or more, multiple La(s) may each independently be a substituted or unsubstituted arylene group of 6 to 30 ring-forming carbon atoms, or a substituted or unsubstituted heteroarylene group of 2 to 30 ring-forming carbon atoms.

In some embodiments, in Formula E-2a, A₁ to A₅ may each independently be N or CRᵢ. Rₐ to Rᵢ may each independently be hydrogen, deuterium, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. Rₐ to Rᵢ may be combined with an adjacent group to form a hydrocarbon ring or a heterocycle including N, O, S, etc. as a ring-forming atom.

In some embodiments, in Formula E-2a, two or three selected from A₁ to A₅ may be N, and the remainder may be CRᵢ.

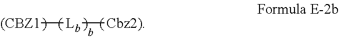

In Formula E-2b, Cbz1 and Cbz2 may each independently be an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” is an integer of 0 to 10, and when “b” is an integer of 2 or more, multiple L_{b}(s) may each independently be a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one selected from among compounds in Compound Group E-2. However, the compounds listed in Compound Group E-2 are mere examples, and the compound represented by Formula E-2a or Formula E-2b is not limited to the compounds represented in Compound Group E-2.

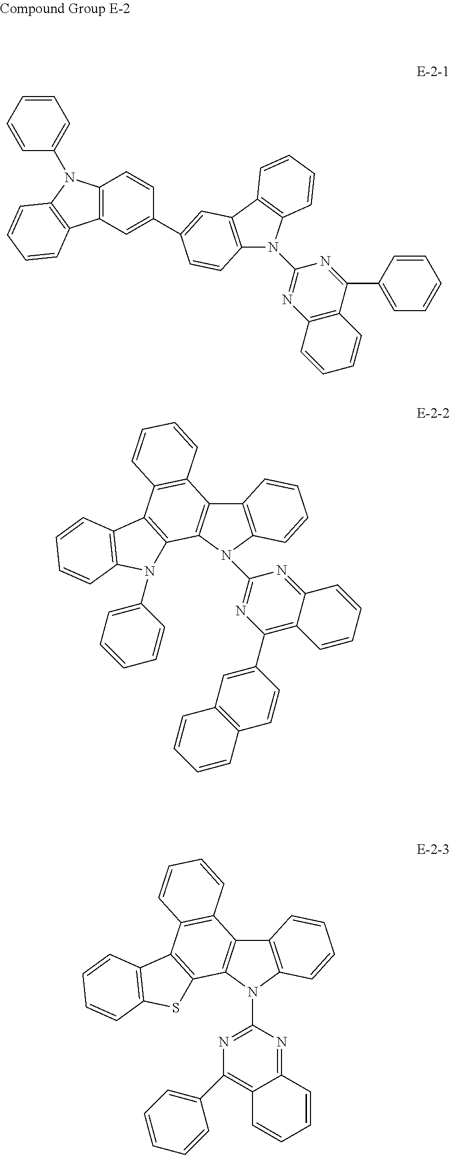 [SMILES: c1ccc(-c2nc(-n3c4ccccc4c4cc(-c5ccc6c(c5)c5ccccc5n6-c5ccccc5)ccc43)nc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

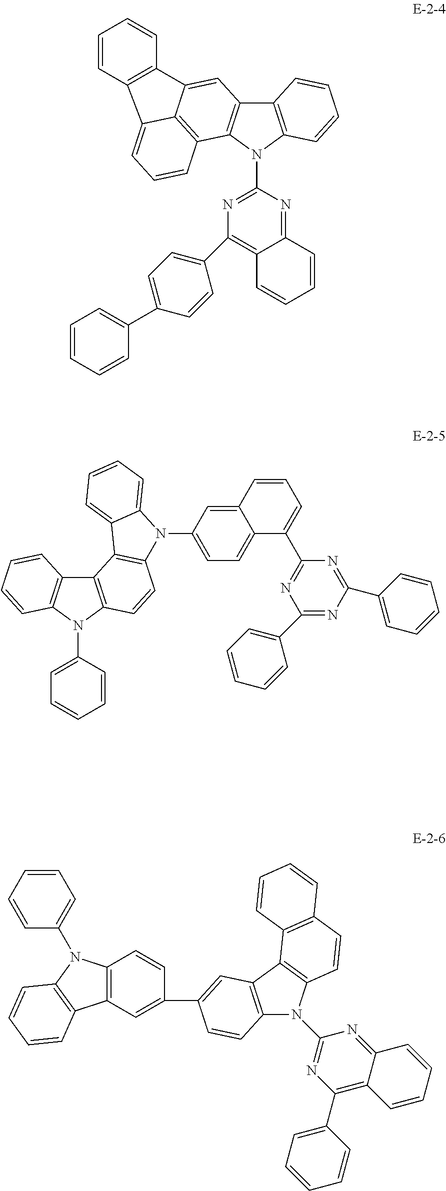 [SMILES: c1ccc(-c2ccc(-c3nc(-n4c5ccccc5c5cc6c7c(cccc7c54)-c4ccccc4-6)nc4ccccc34)cc2)cc1 ; c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1]

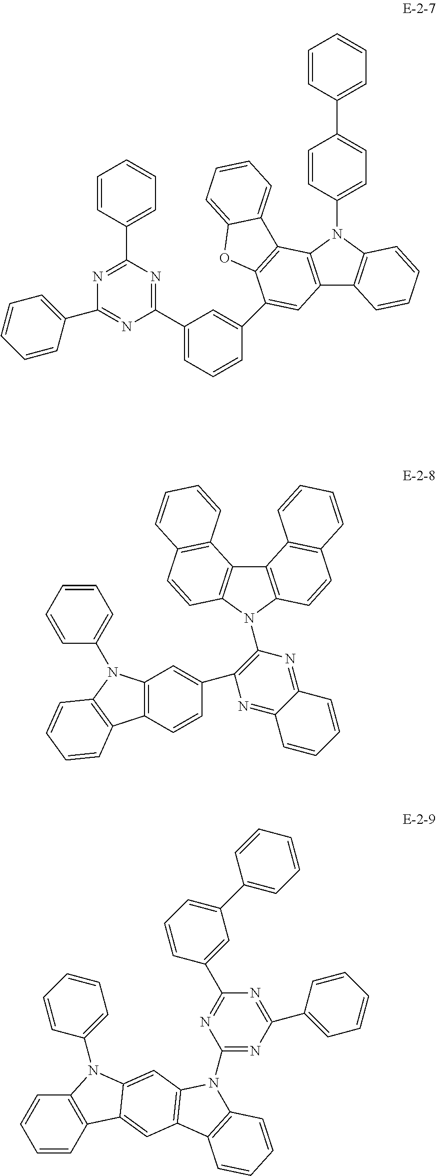 [SMILES: c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1]

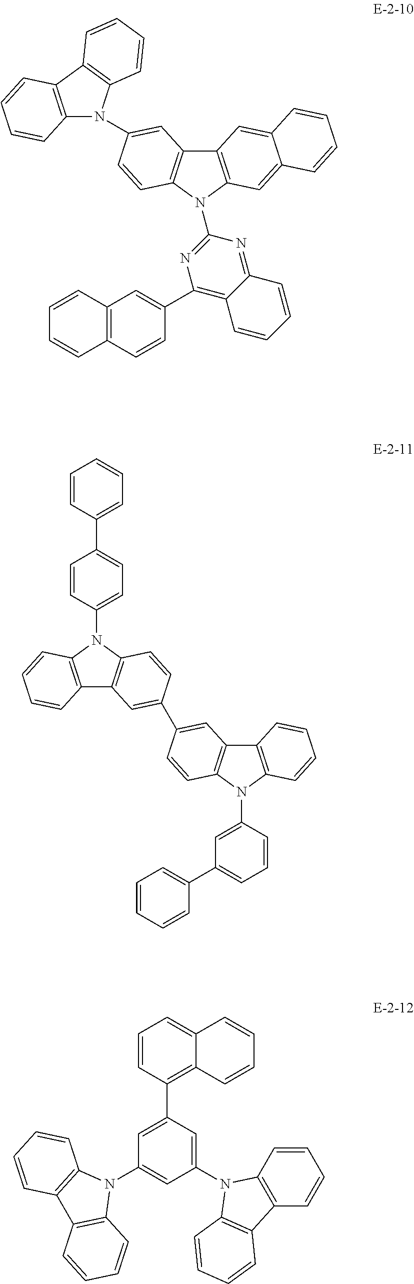 [SMILES: c1ccc2cc(-c3nc(-n4c5ccc(-n6c7ccccc7c7ccccc76)cc5c5cc6ccccc6cc54)nc4ccccc34)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1]

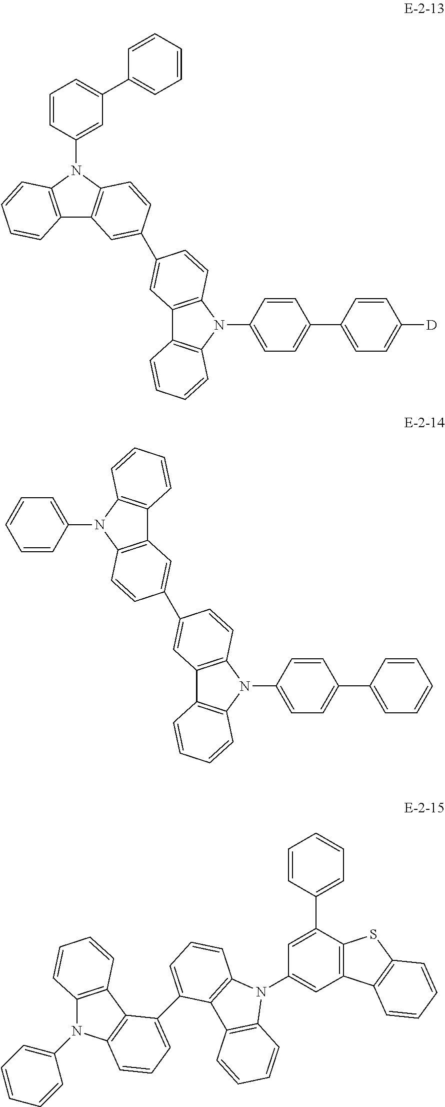 [SMILES: [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

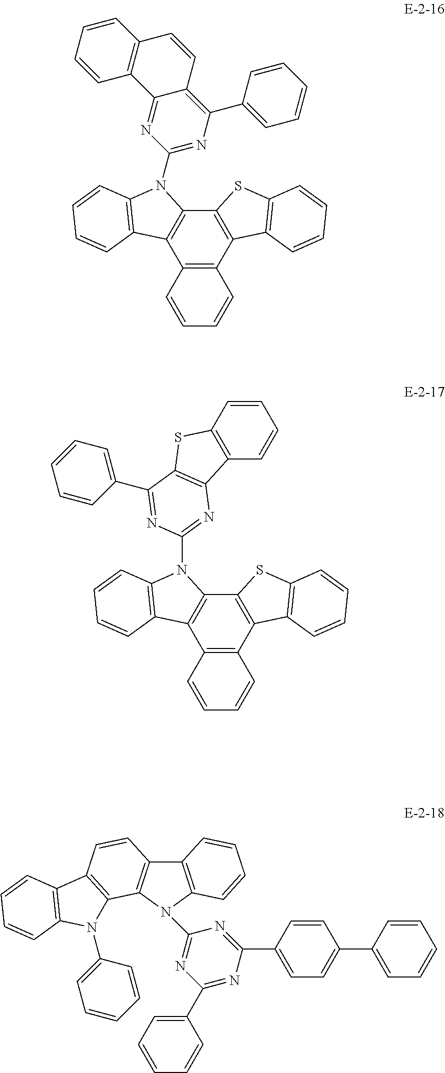 [SMILES: c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1 ; c1ccc(-c2ccc(-c3nc(-c4ccccc4)nc(-n4c5ccccc5c5ccc6c7ccccc7n(-c7ccccc7)c6c54)n3)cc2)cc1]

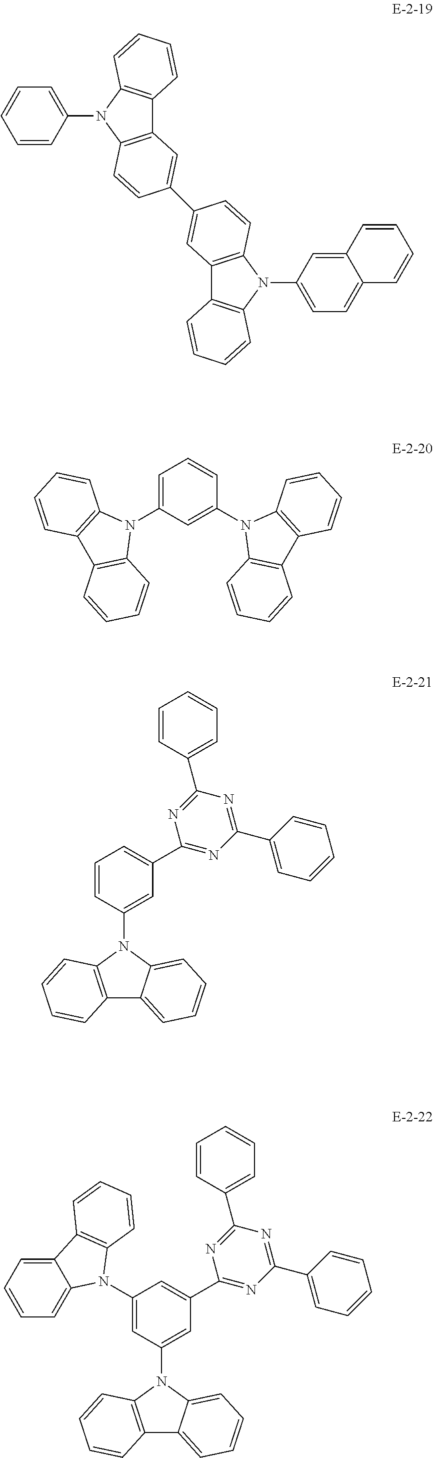 [SMILES: c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1]

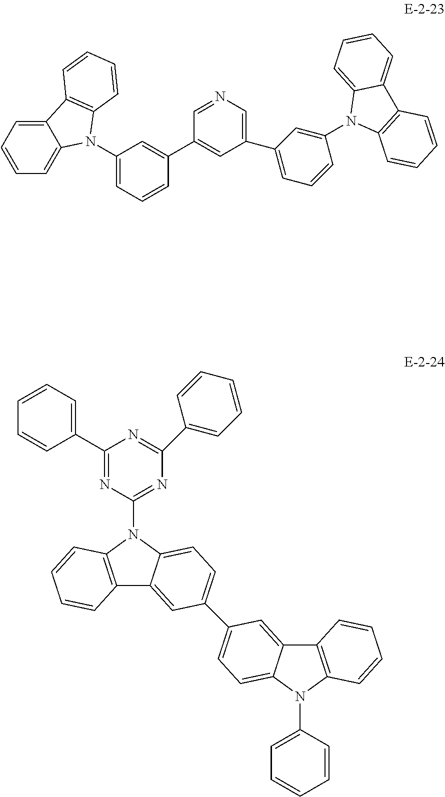 [SMILES: c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

In one or more embodiments, The emission layer EML may further include a material well-suitable in the art as a host material. For example, the emission layer EML may include as a host material, at least one of bis(4-(9H-carbazol-9-yl)phenyl) diphenylsilane (BCPDS), (4-(1-(4-(diphenylamino)phenyl)cyclohexyl)phenyl) diphenyl-phosphine oxide (POPCPA), bis[2-(diphenylphosphino)phenyl] ether oxide (DPEPO), 4,4′-bis(carbazol-9-yl)biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, embodiments of the present disclosure are not limited thereto. For example, tris(8-hydroxyquinolinato)aluminum (Alq₃), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be utilized as the host material.

In one or more embodiments, the emission layer EML may include a compound represented by Formula M-a or Formula M-b. The compound represented by Formula M-a or Formula M-b may be utilized as a phosphorescence dopant material.

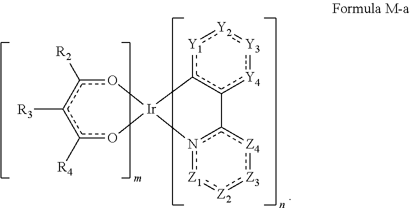

In Formula M-a, Y₁ to Y₄ and Z₁ to Z₄ may each independently be CR₁ or N, and R₁ to R₄ may each independently be hydrogen, deuterium, a substituted or unsubstituted amine group, a substituted or unsubstituted thio group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. In Formula M-a, “m” is 0 or 1, and “n” is 2 or 3. In Formula M-a, when “m” is 0, “n” is 3, and when “m” is 1, “n” is 2.

The compound represented by Formula M-a may be utilized as a phosphorescence dopant.

The compound represented by Formula M-a may be any one selected from among Compounds M-a1 to M-a25. However, Compounds M-a1 to M-a25 are mere examples, and the compound represented by Formula M-a is not limited to the compounds represented by Compounds M-a1 to M-a25.

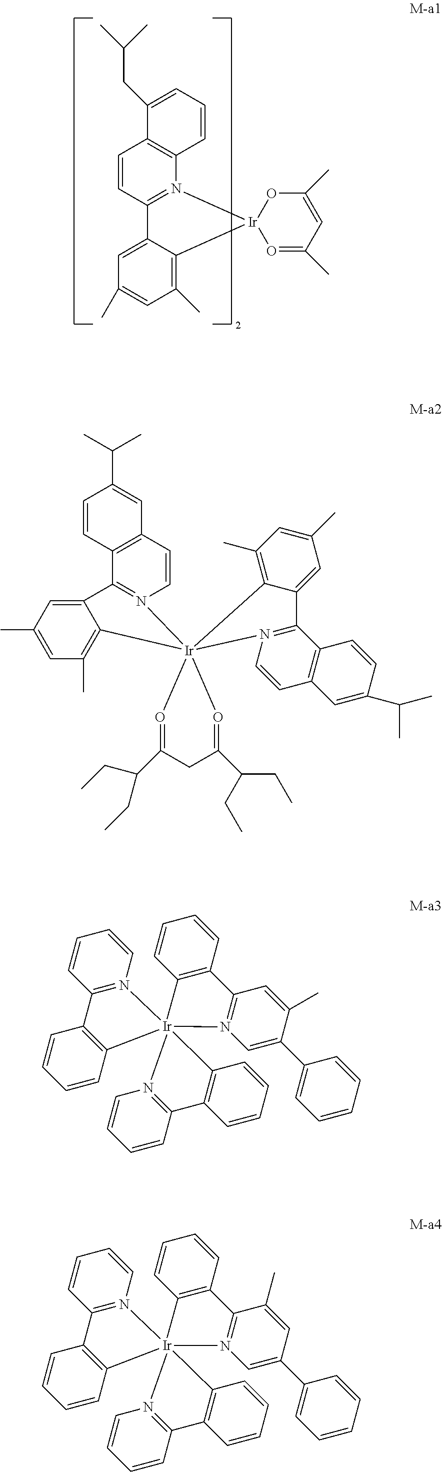

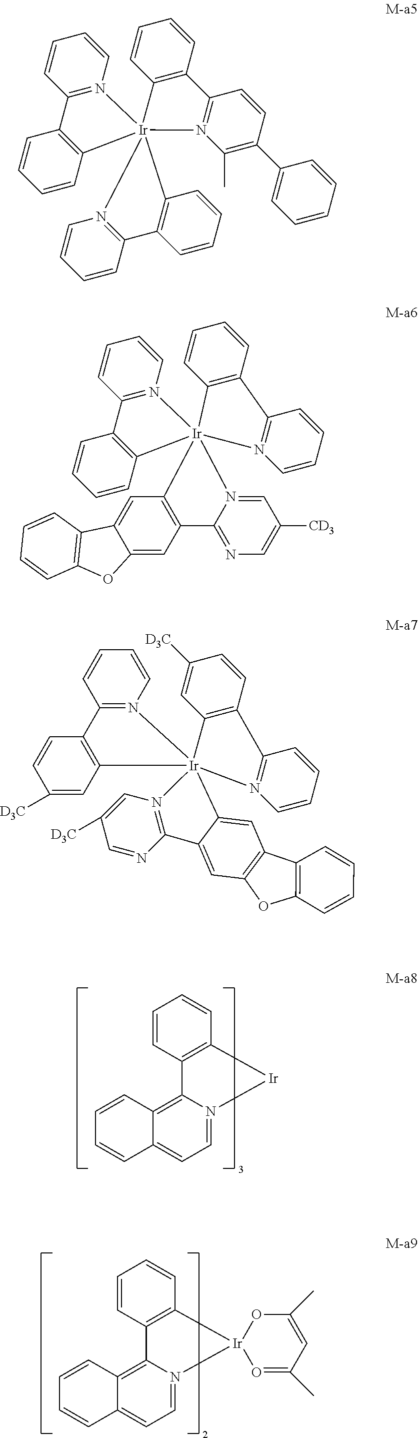

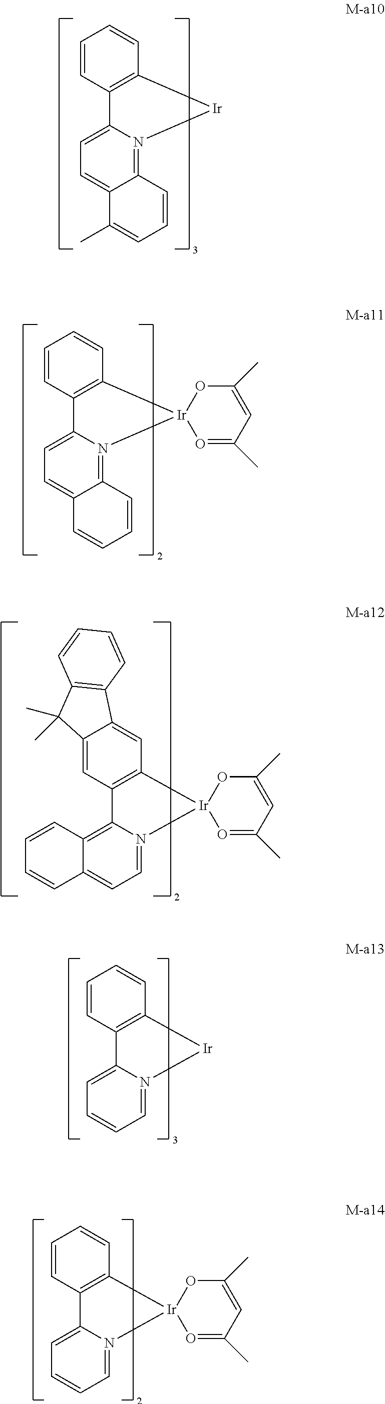

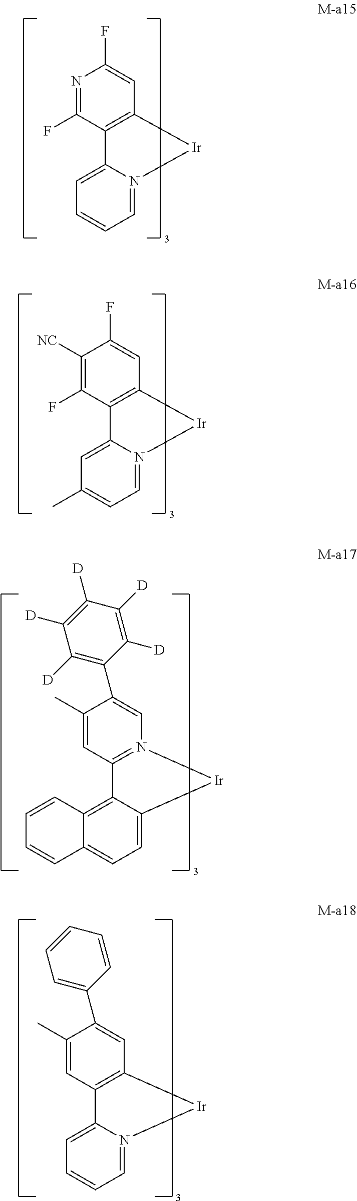

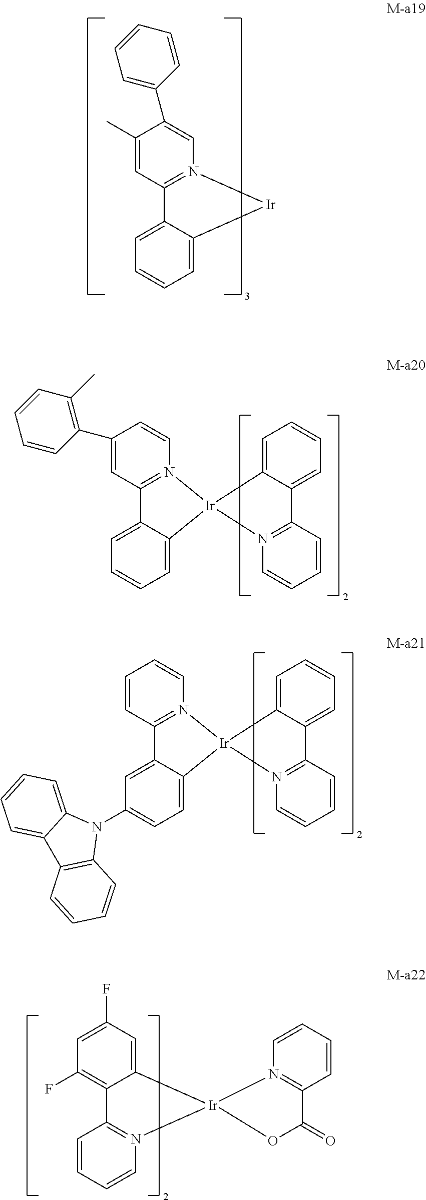

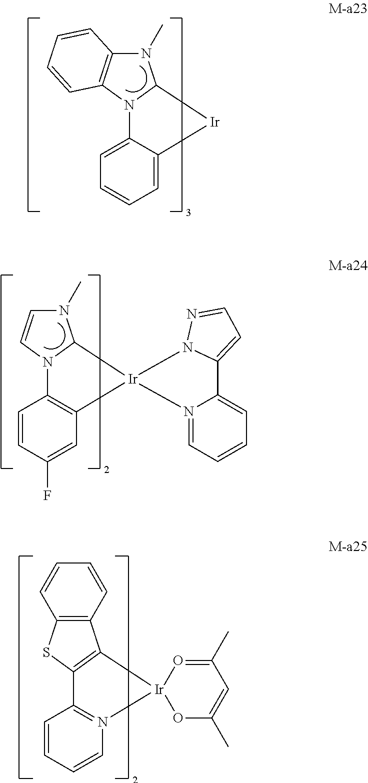

In one or more embodiments, Compound M-a1 and Compound M-a2 may be utilized as red dopant materials, and Compound M-a3 to Compound M-a7 may be utilized as green dopant materials.

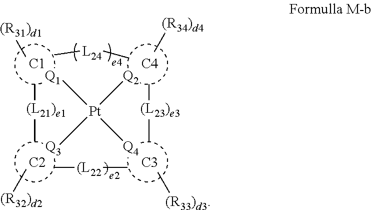

In Formula M-b, Q₁ to Q₄ may each independently be C or N, C1 to C4 may each independently be a substituted or unsubstituted hydrocarbon ring of 5 to 30 ring-forming carbon atoms, or a substituted or unsubstituted heterocycle of 2 to 30 ring-forming carbon atoms. L₂₁ to L₂₄ may each independently be a direct linkage,

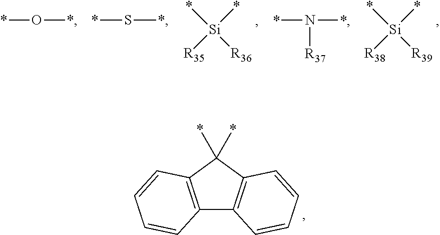

a substituted or unsubstituted alkylene group of 1 to 20 carbon atoms, a substituted or unsubstituted arylene group of 6 to 30 ring-forming carbon atoms, or a substituted or unsubstituted heteroarylene group of 2 to 30 ring-forming carbon atoms, and e1 to e4 may each independently be 0 or 1. R₃₁ to R₃₉ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring, and d1 to d4 may each independently be an integer of 0 to 4.

The compound represented by Formula M-b may be utilized as a blue phosphorescence dopant or a green phosphorescence dopant.

The compound represented by Formula M-b may be any one selected from among Compound M-b-1 to Compound M-b-11. However, the compounds are mere examples, and the compound represented by Formula M-b is not limited to the compounds.

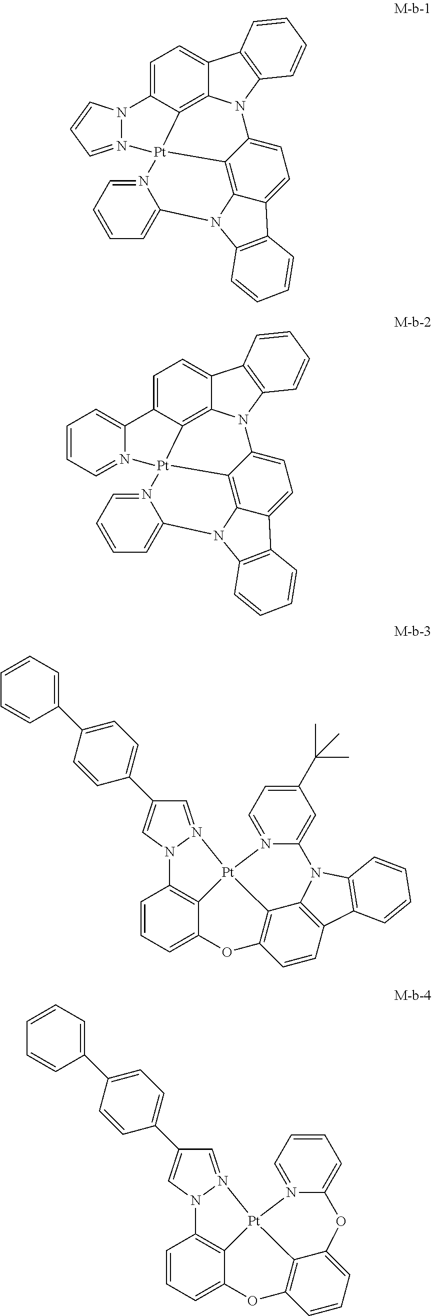

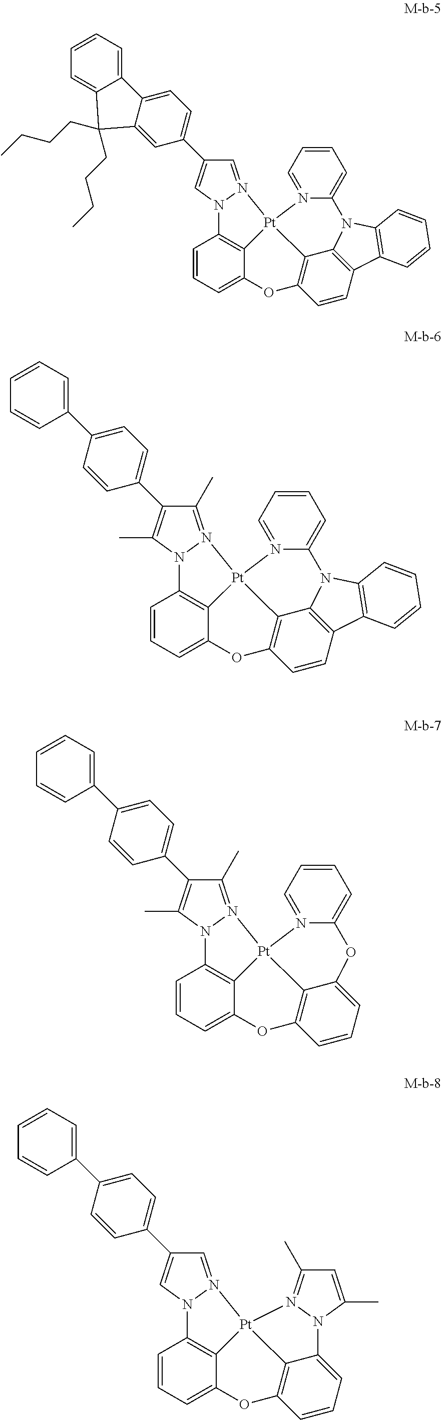

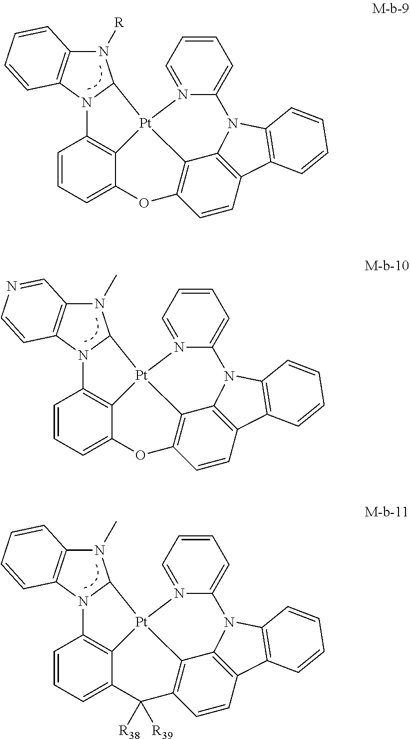

In the compounds above, R, R₃₈, and R₃₉ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In one or more embodiments, the emission layer EML may include a compound represented by any one selected from among Formula F-a to Formula F-c. The compounds represented by Formula F-a to Formula F-c may be utilized as fluorescence dopant materials.

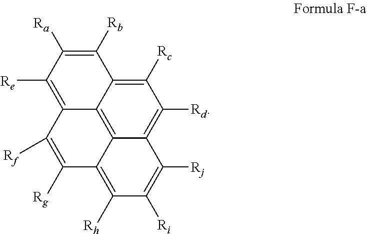

In Formula F-a, two selected from Rₐ to R; may each independently be substituted with *-NA₁Ar₂. The remainder not substituted with *—NAr₁Ar₂ among Rₐ to Rⱼ may each independently be hydrogen, deuterium, a halogen, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In *—NAr₁Ar₂, Ar₁ and Ar₂ may each independently be a substituted or unsubstituted aryl group of 6 to 30 ring-forming carbon atoms, or a substituted or unsubstituted heteroaryl group of 2 to 30 ring-forming carbon atoms. For example, in some embodiments, at least one selected from among Ar₁ and Ar₂ may be a heteroaryl group including O or S as a ring-forming atom.

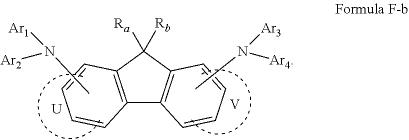

In Formula F-b, Rₐ and R_{b} may each independently be hydrogen, deuterium, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, and/or may be combined with an adjacent group to form a ring. Ar₁ to Ar₄ may each independently be a substituted or unsubstituted aryl group of 6 to 30 ring-forming carbon atoms, or a substituted or unsubstituted heteroaryl group of 2 to 30 ring-forming carbon atoms.

In Formula F-b, U and V may each independently be a substituted or unsubstituted hydrocarbon ring of 5 to 30 ring-forming carbon atoms, or a substituted or unsubstituted heterocycle of 2 to 30 ring-forming carbon atoms.

In Formula F-b, the number of rings represented by U and V may each independently be 0 or 1. For example, in Formula F-b, when the number of U or V is 1, one ring forms a fused ring at the designated part by U or V, and when the number of U or V is 0, a ring is not present at the designated part by U or V. For example, when the number of U is 0, and the number of V is 1, or when the number of U is 1, and the number of V is 0, a fused ring having the fluorene core of Formula F-b may be a ring compound with four rings. In some embodiments, when the number of both (e.g., simultaneously) U and V is 0, the fused ring of Formula F-b may be a ring compound with three rings. In some embodiments, when the number of both (e.g., simultaneously) U and V is 1, a fused ring having the fluorene core of Formula F-b may be a ring compound with five rings.

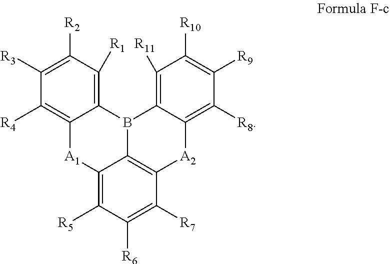

In Formula F-c, A₁ and A₂ may each independently be O, S, Se, or NRₘ, and Rₘ may be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ may each independently be hydrogen, deuterium, a halogen, a cyano group, a substituted or unsubstituted amine group, a substituted or unsubstituted boryl group, a substituted or unsubstituted oxy group, a substituted or unsubstituted thio group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring.

In Formula F-c, A₁ and A₂ may each independently be combined with the substituents of an adjacent ring to form a fused ring. For example, in some embodiments, when A₁ and A₂ may each independently be NRₘ, A₁ may be combined with R₄ or R₅ to form a ring. In some embodiments, A₂ may be combined with R₇ or R₈ to form a ring.

In one or more embodiments, the emission layer EML may include, as a suitable dopant material, one or more selected from styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino)pyrene), etc. In one or more embodiments, the emission layer EML may include a suitable phosphorescence dopant material. For example, the phosphorescence dopant material may utilize a metal complex including iridium (Ir), platinum (Pt), osmium (Os), gold (Au), titanium (Ti), zirconium (Zr), hafnium (Hf), europium (Eu), terbium (Tb), or thulium (Tm). For example, iridium(III) bis(4,6-difluorophenylpyridinato-N,C2′) (Firpic), bis(2,4-difluorophenylpyridinato)-tetrakis(1-pyrazolyl)borate iridium(III) (Fir6), or platinum octaethyl porphyrin (PtOEP) may be utilized as the phosphorescence dopant material. However, embodiments of the present disclosure are not limited thereto.

In one or more embodiments, the emission layer EML may include a quantum dot material. The core of the quantum dot may be selected from II-VI group compounds, III-VI group compounds, I-III-VI group compounds, III-V group compounds, III-II-V group compounds, IV-VI group compounds, IV group elements, IV group compounds, and combinations thereof.

The II-VI group compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, and mixtures thereof.

The III-VI group compound may include a binary compound such as In₂S₃ and/or In₂Se₃, a ternary compound such as InGaS₃ and/or InGaSe₃, or one or more suitable or optional combinations thereof.

The I-III-VI group compound may be selected from a ternary compound selected from the group consisting of AgInS, AgInS₂, CuInS, CuInS₂, AgGaS₂, CuGaS₂, CuGaO₂, AgGaO₂, AgAlO₂, and mixtures thereof, and/or a quaternary compound such as AgInGaS₂, and CuInGaS₂.

The III-V group compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and mixtures thereof. In some embodiments, the III-V group compound may further include a II group metal. For example, InZnP, etc. may be selected as a III-II-V group compound.

The IV-VI group compound may be selected from the group consisting of a binary compound selected from the group consisting of SnS, SnSe, SnTe, PbS, PbSe, PbTe, and mixtures thereof, a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The IV group element may be selected from the group consisting of Si, Ge, and a mixture thereof. The IV group compound may be a binary compound selected from the group consisting of SiC, SiGe, and a mixture thereof.

In one or more embodiments, the binary compound, the ternary compound, or the quaternary compound may be present at substantially uniform concentration distribution in a particle or may be present at a partially different concentration distribution within substantially the same particle. In some embodiments, a core/shell structure in which one quantum dot wraps another quantum dot may be desirable. The interface of the core and the shell may have a concentration gradient in which the concentration of an element present in the shell is decreased toward the center.

In some embodiments, the quantum dot may have the above-described core-shell structure including a core including a nanocrystal and a shell wrapping the core. The shell of the quantum dot may play the role of a protection layer for preventing or reducing the chemical deformation of the core to maintain semiconductor properties and/or a charging layer for imparting the quantum dot with electrophoretic properties. The shell may have a single layer or a multilayer. Examples of the shell of the quantum dot may include a metal or non-metal oxide, a semiconductor compound, or combinations thereof.

For example, the metal or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, CO₃O₄ and NiO, and/or a ternary compound such as MgAl₂O₄, CoFe₂O₄, NiFe₂O₄ and CoMn₂O₄, but embodiments of the present disclosure are not limited thereto.

Also, the semiconductor compound suitable as a shell may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but embodiments of the present disclosure are not limited thereto.

The quantum dot may have a full width of half maximum (FWHM) of emission spectrum of about 45 nm or less, about 40 nm or less, or, about 30 nm or less. Within this range, the color purity or color reproducibility of the light emitting element may be improved. In some embodiments, light emitted via such quantum dots is emitted in all directions, and light view angle properties may be improved.

In some embodiments, the shape of the quantum dot may be generally utilized shapes in the art, without specific limitation. In one or more embodiments, the shape of substantially spherical, pyramidal, multi-arm, or cubic nanoparticle, nanotube, nanowire, nanofiber, nanoplate particle, etc. may be utilized.

The quantum dot may control the color of light emitted according to the particle size, and accordingly, the quantum dot may have one or more suitable emission colors such as blue, red, and green.

In the light emitting elements ED of one or more embodiments, as shown in FIG. 3 to FIG. 6, the electron transport region ETR may be provided on the emission layer EML. The electron transport region ETR may include at least one of a hole blocking layer HBL, an electron transport layer ETL, or an electron injection layer EIL. However, embodiments of the present disclosure are not limited thereto.

The electron transport region ETR may have a single layer formed utilizing a single material, a single layer formed utilizing multiple different materials, or a multilayer structure having multiple layers formed utilizing multiple different materials.

For example, in some embodiments, the electron transport region ETR may have a single layer structure of an electron injection layer EIL or an electron transport layer ETL, or a single layer structure formed utilizing an electron injection material and an electron transport material. Further, in some embodiments, the electron transport region ETR may have a single layer structure formed utilizing multiple different materials, or a structure stacked from the emission layer EML of electron transport layer ETL/electron injection layer EIL, hole blocking layer HBL/electron transport layer ETL/electron injection layer EIL, without limitation. The thickness of the electron transport region ETR may be, for example, from about 1,000 Å to about 1,500 Å.

The electron transport region ETR may be formed utilizing one or more suitable methods such as a vacuum deposition method, a spin coating method, a casting method, a Langmuir-Blodgett (LB) method, an inkjet printing method, a laser printing method, and/or a laser induced thermal imaging (LITI) method.

In one or more embodiments, the electron transport region ETR may include a compound represented by Formula ET-2.

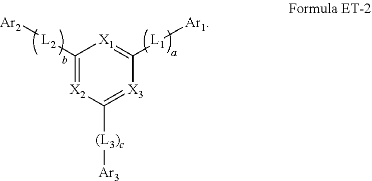

In Formula ET-2, at least one selected from among X₁ to X₃ is N, and the remainder are CRₐ. Rₐ may be hydrogen, deuterium, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may each independently be hydrogen, deuterium, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-2, “a” to “c” may each independently be an integer of 0 to 10. In Formula ET-2, L₁ to L₃ may each independently be a direct linkage, a substituted or unsubstituted arylene group of 6 to 30 ring-forming carbon atoms, or a substituted or unsubstituted heteroarylene group of 2 to 30 ring-forming carbon atoms. In some embodiments, when “a” to “c” are integers of 2 or more, L₁ to L₃ may each independently be a substituted or unsubstituted arylene group of 6 to 30 ring-forming carbon atoms, or a substituted or unsubstituted heteroarylene group of 2 to 30 ring-forming carbon atoms.

In one or more embodiments, the electron transport region ETR may include an anthracene-based compound. However, embodiments of the present disclosure are not limited thereto, and the electron transport region ETR may include, for example, at least one selected from among tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzimidazolyl-1-ylphenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(biphenyl-4-yl)-4-phenyl-5-tert-butylphenyl-1,2,4-triazole (TAZ), 4-(naphthalen-1-yl)-3,5-diphenyl-4H-1,2,4-triazole (NTAZ), 2-(biphenyl-4-yl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), diphenyl(4-(triphenylsilyl)phenyl)phosphine oxide (TSPO1), and mixtures thereof, without limitation.

In one or more embodiments, the electron transport region ETR may include at least one selected from among Compounds ET1 to ET36.

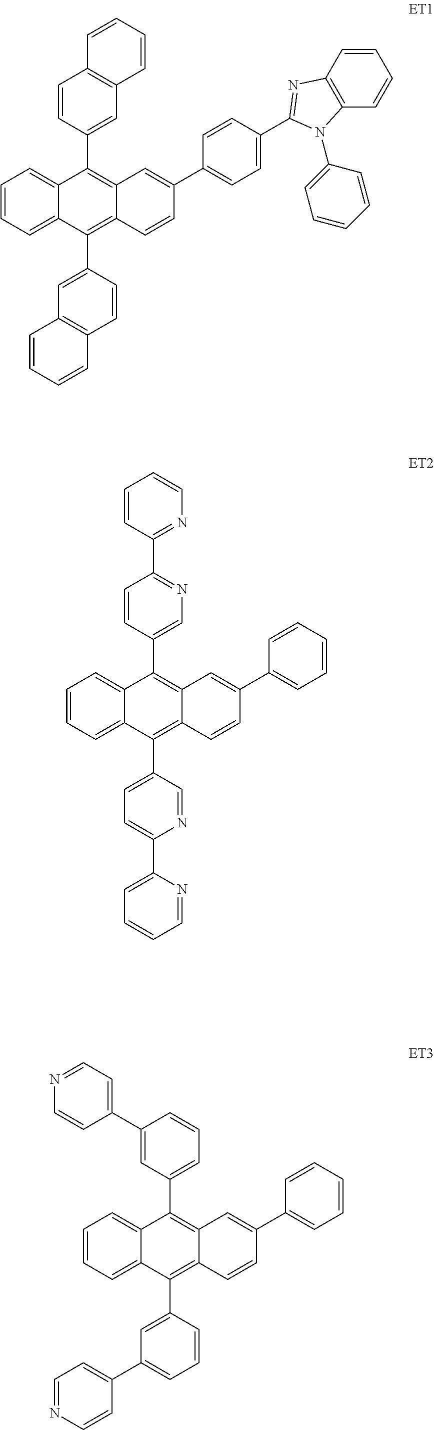 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

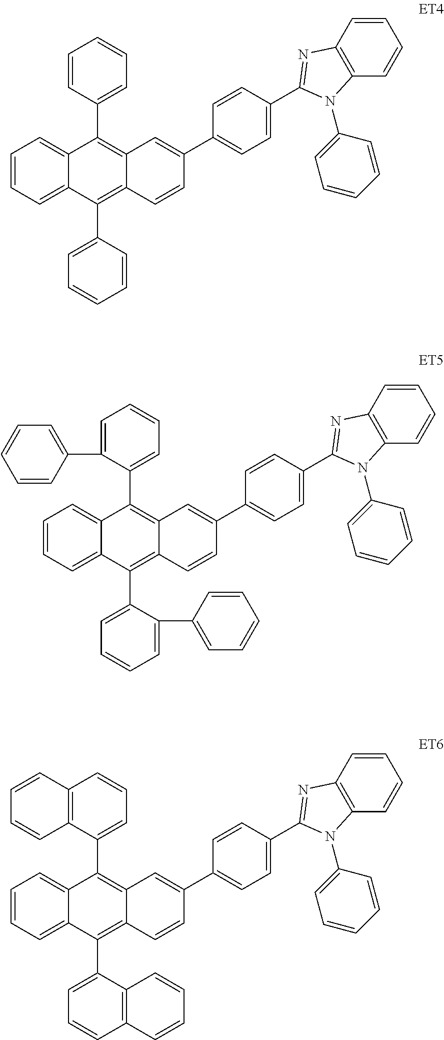 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

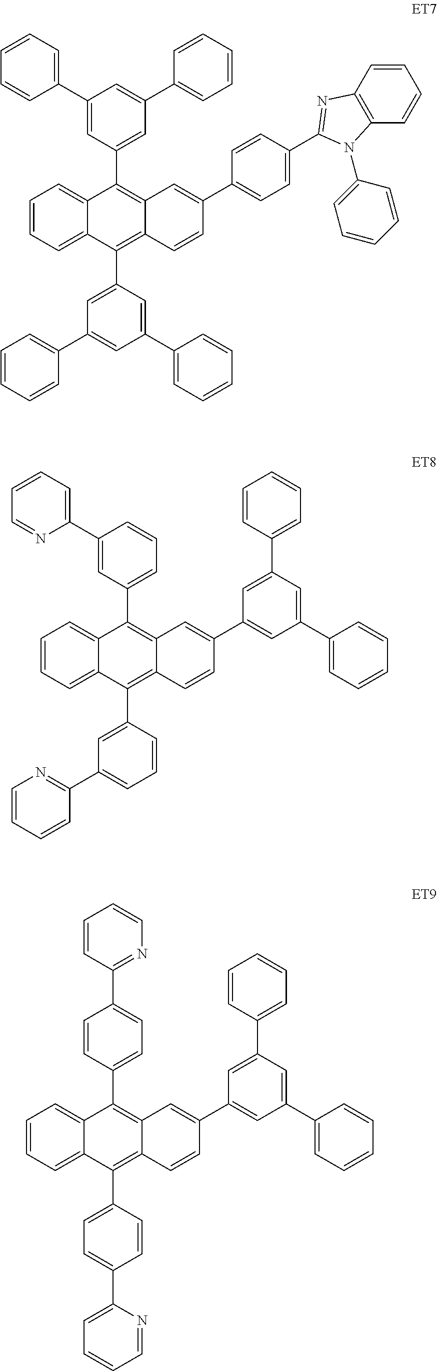 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

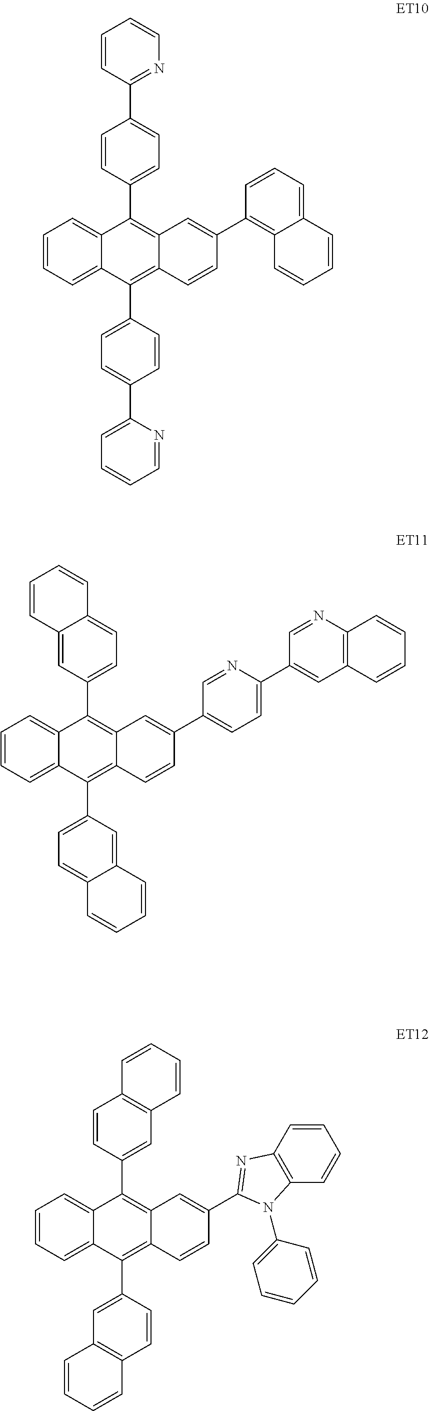 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc4c(-c5ccc6ccccc6c5)c5ccccc5c(-c5ccc6ccccc6c5)c4c3)nc3ccccc32)cc1]

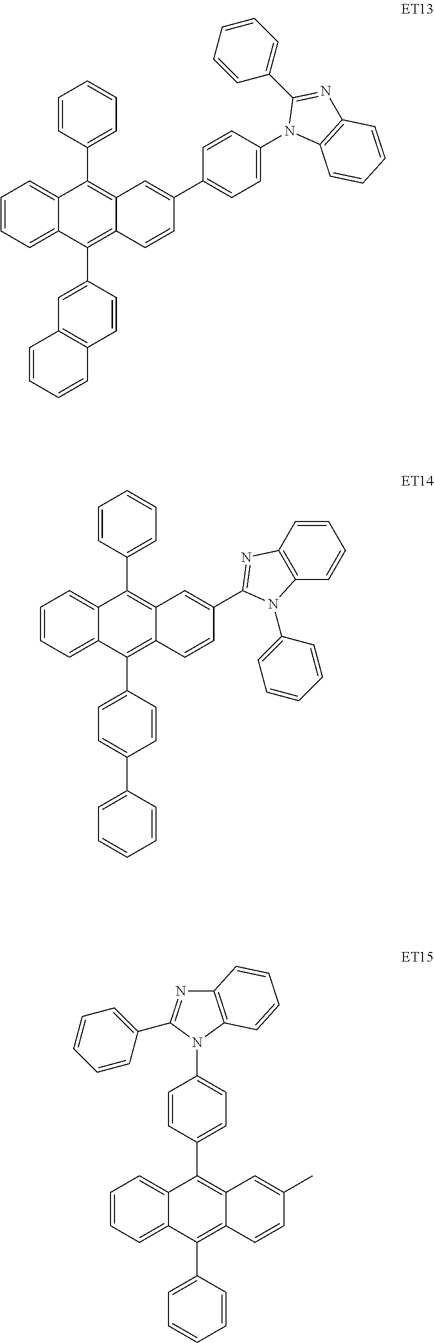 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1]

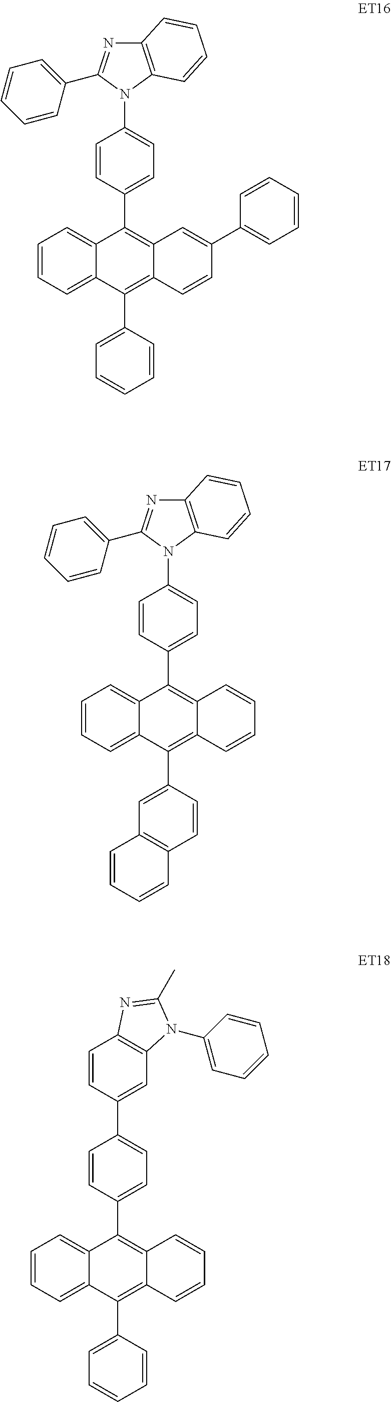 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

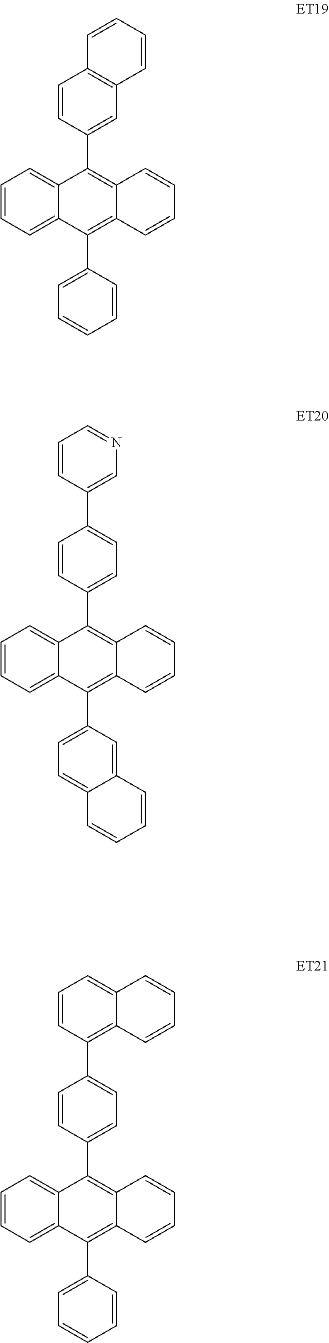

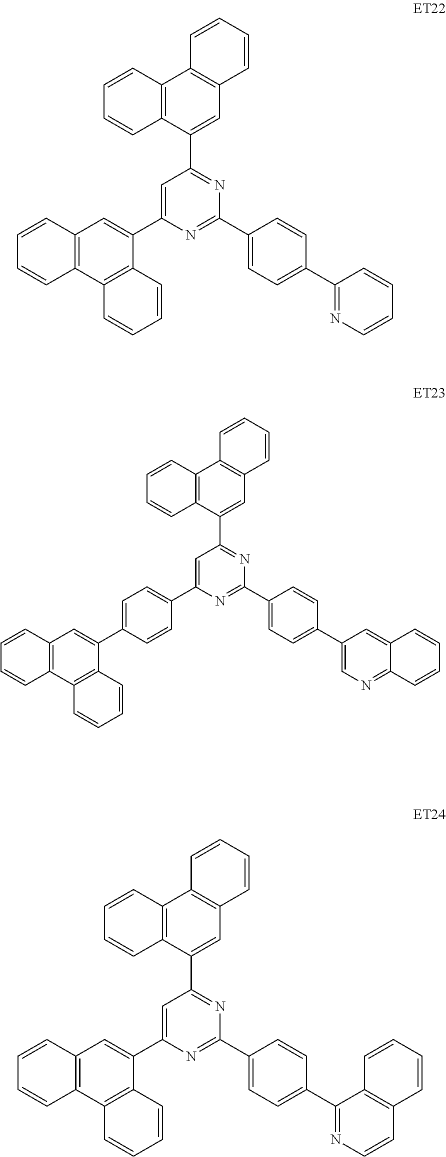 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

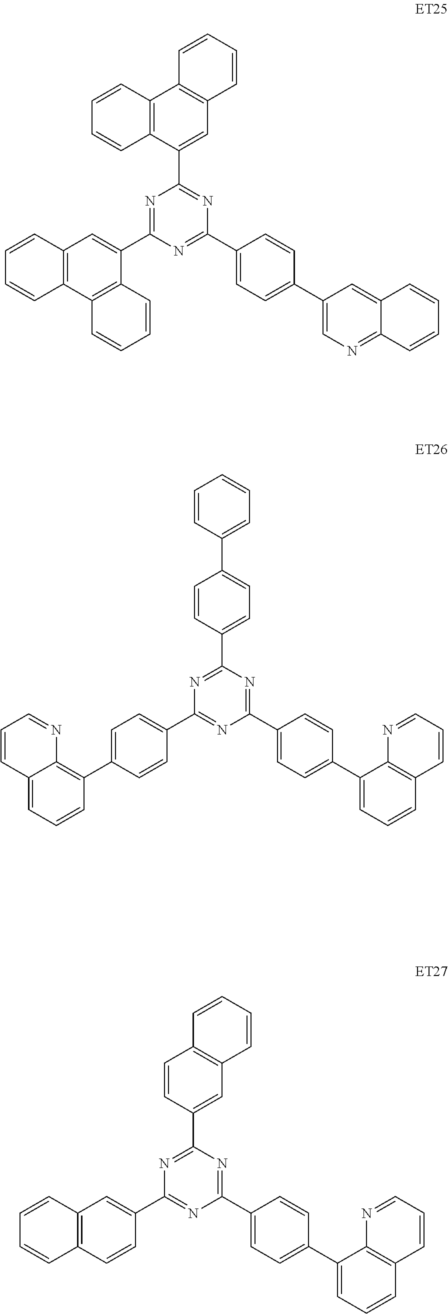 [SMILES: c1ccc2ncc(-c3ccc(-c4nc(-c5cc6ccccc6c6ccccc56)nc(-c5cc6ccccc6c6ccccc56)n4)cc3)cc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

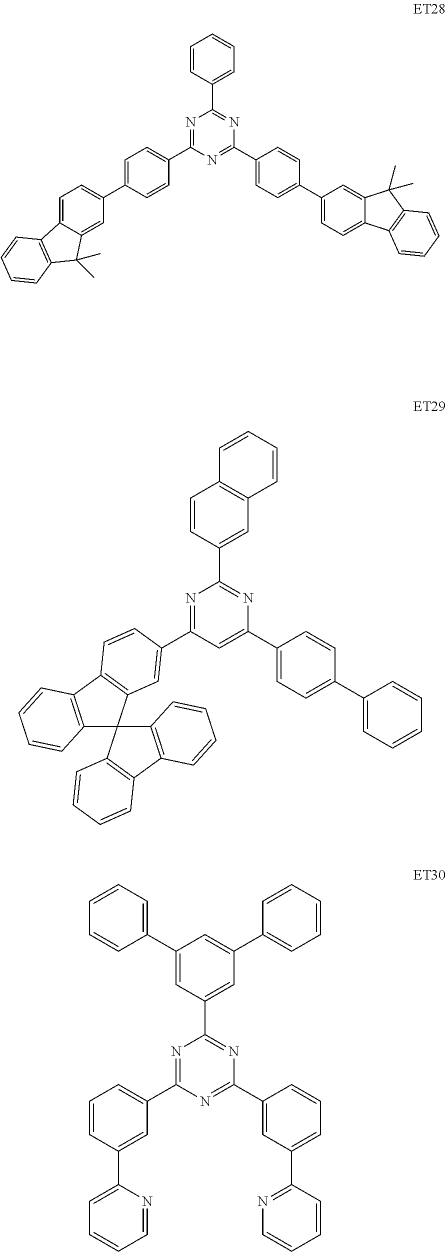 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

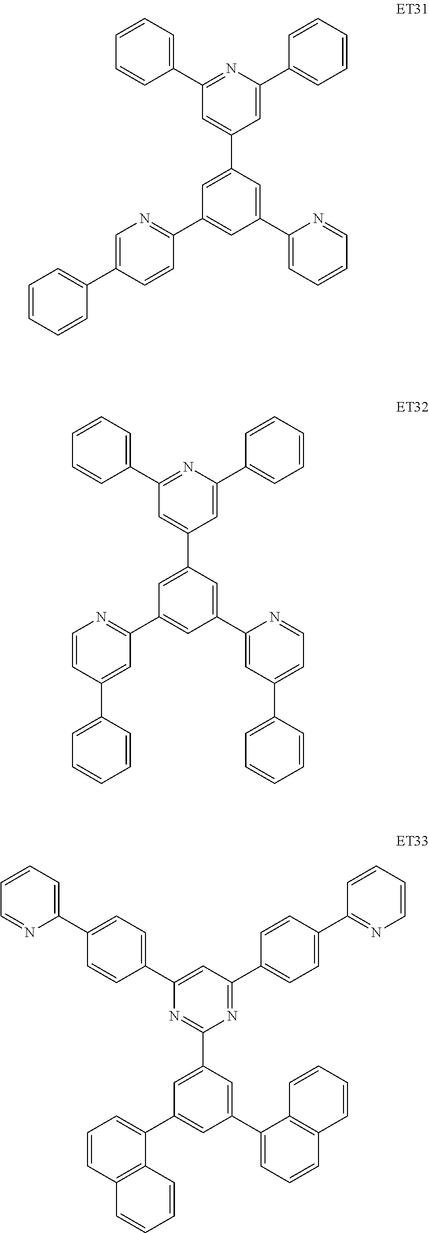 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

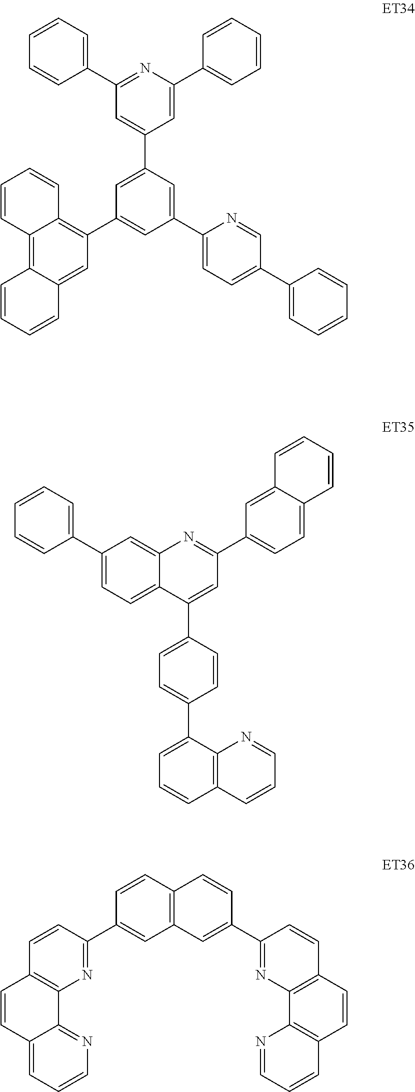 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI and KI, a lanthanide metal such as Yb, or a co-depositing material of the metal halide and the lanthanide metal. For example, the electron transport region ETR may include KI:Yb, RbI:Yb, LiF:Yb, etc., as the co-depositing material. In some embodiments, the electron transport region ETR may utilize a metal oxide such as Li₂O and BaO, or 8-hydroxy-lithium quinolate (Liq). However, embodiments of the present disclosure are not limited thereto. In some embodiments, the electron transport region ETR may also be formed utilizing a mixture material of an electron transport material and an insulating organo metal salt. The organo metal salt may be a material having an energy band gap of about 4 eV or more. For example, the organo metal salt may include, for example, metal acetates, metal benzoates, metal acetoacetates, metal acetylacetonates, and/or metal stearates.

In one or more embodiments, The electron transport region ETR may include at least one of 2,9-dimethyl-4,7-diphenyl-1,10-phenanthroline (BCP), diphenyl(4-(triphenylsilyl)phenyl)phosphine oxide (TSPO1) or 4,7-diphenyl-1,10-phenanthroline (Bphen) in addition to the aforementioned materials. However, embodiments of the present disclosure are not limited thereto.

The electron transport region ETR may include the above-described compounds of the electron transport region in at least one selected from among an electron injection layer EIL, an electron transport layer ETL, and a hole blocking layer HBL.

When the electron transport region ETR includes an electron transport layer ETL, the thickness of the electron transport layer ETL may be from about 100 Å to about 1,000 Å, for example, from about 150 Å to about 500 Å. When the thickness of the electron transport layer ETL satisfies the above-described range, satisfactory electron transport properties may be obtained without a substantial increase in a driving voltage. When the electron transport region ETR includes an electron injection layer EIL, the thickness of the electron injection layer EIL may be from about 1 Å to about 100 Å, or from about 3 Å to about 90 Å. When the thickness of the electron injection layer EIL satisfies the above described range, satisfactory electron injection properties may be obtained without inducing a substantial increase in a driving voltage.

The second electrode EL2 may be provided on the electron transport region ETR. The second electrode EL2 may be a common electrode. The second electrode EL2 may be a cathode or an anode, but embodiments of the present disclosure are not limited thereto. For example, when the first electrode EL1 is an anode, the second cathode EL2 may be a cathode, and when the first electrode EL1 is a cathode, the second electrode EL2 may be an anode. The second electrode EL2 may include at least one selected from among Ag, Mg, Cu, Al, Pt, Pd, Au, Ni, Nd, Ir, Cr, Li, Ca, LiF, Mo, Ti, W, In, Sn, and Zn, one or more compounds of two or more selected therefrom, one or more mixtures of two or more selected therefrom, and/or one or more oxides thereof.

The second electrode EL2 may be a transmissive electrode, a transflective electrode, or a reflective electrode. When the second electrode EL2 is a transmissive electrode, the second electrode EL2 may include a transparent metal oxide, for example, ITO, IZO, ZnO, ITZO, etc.

When the second electrode EL2 is a transflective electrode or a reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, compounds thereof, or mixtures thereof (for example, AgMg, AgYb, or MgAg). In some embodiments, the second electrode EL2 may have a multilayered structure including a reflective layer or a transflective layer formed utilizing one or more selected from the above-described materials and a transparent conductive layer formed utilizing ITO, IZO, ZnO, ITZO, etc. For example, the second electrode EL2 may include one or more selected from the aforementioned metal materials, combinations of two or more metal materials selected from the aforementioned metal materials, and/or oxides of the aforementioned metal materials.

In some embodiments, the second electrode EL2 may be connected with an auxiliary electrode. When the second electrode EL2 is connected with the auxiliary electrode, the resistance of the second electrode EL2 may decrease.

In one or more embodiments, on the second electrode EL2 in the light emitting element ED, a capping layer CPL may be further disposed. The capping layer CPL may include a multilayer or a single layer.

In one or more embodiments, the capping layer CPL may be an organic layer or an inorganic layer. For example, in some embodiments, when the capping layer CPL includes an inorganic material, the inorganic material may include an alkali metal compound such as LiF, an alkaline earth metal compound such as SiON, SiNx, SiOy, etc.

For example, in some embodiments, when the capping layer CPL includes an organic material, the organic material may include 2,2′-dimethyl-N,N′-di-[(1-naphthyl)-N,N′-diphenyl]-1,1′-biphenyl-4,4′-diamine (α-NPD), NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol-9-yl) triphenylamine (TCTA), etc., or includes an epoxy resin and/or acrylate such as methacrylate. In some embodiments, the capping layer CPL may include at least one selected from among Compounds P1 to P5, but embodiments of the present disclosure are not limited thereto.

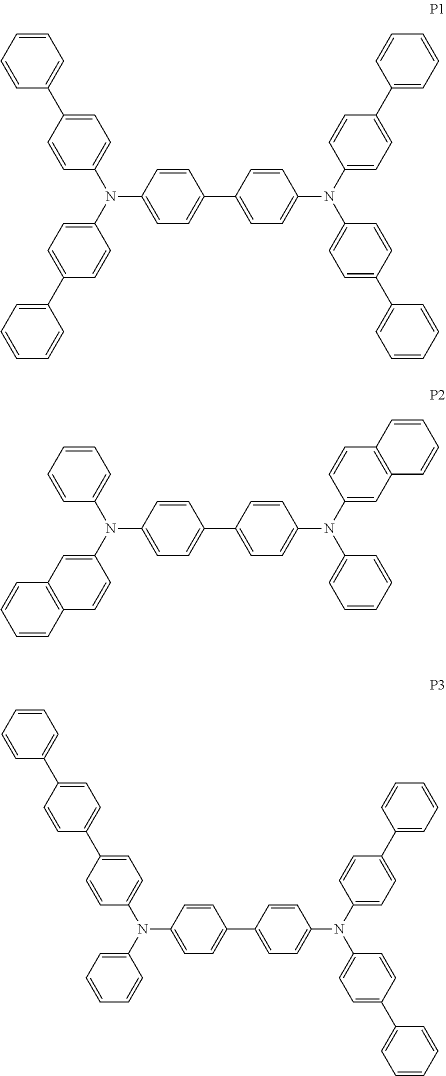 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

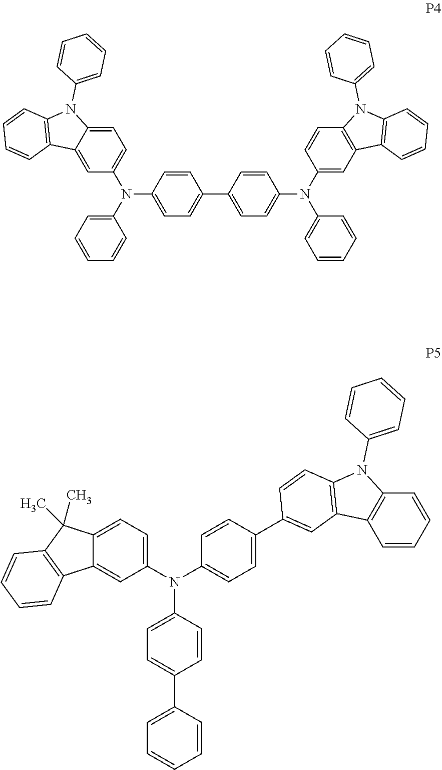 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In some embodiments, the refractive index of the capping layer CPL may be about 1.6 or more. For example, in some embodiments, the refractive index of the capping layer CPL with respect to light in a wavelength range of about 550 nm to about 660 nm may be about 1.6 or more.

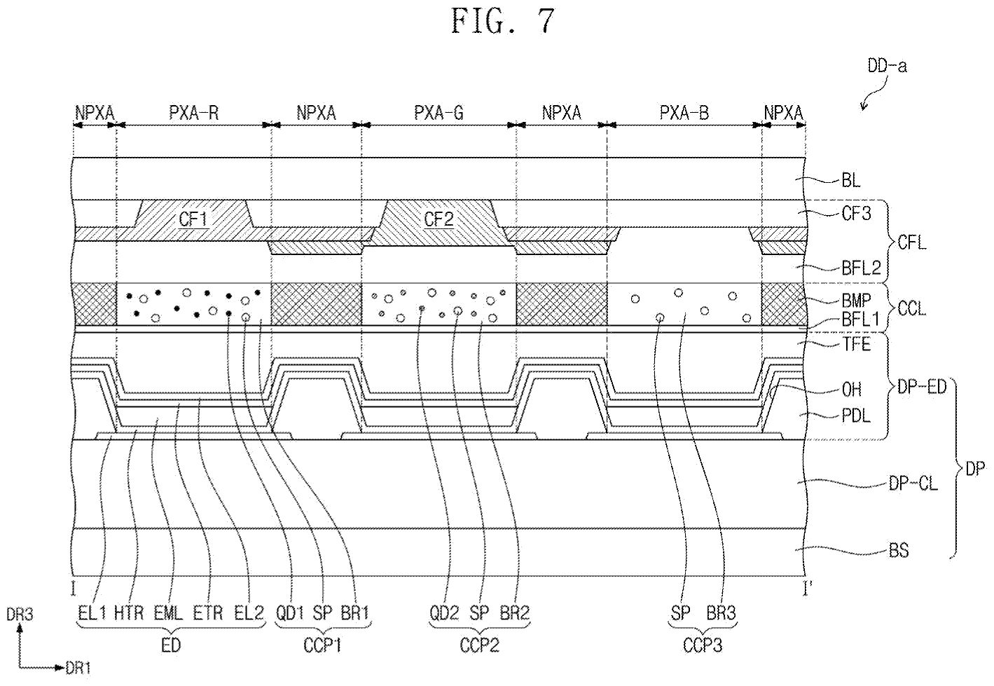

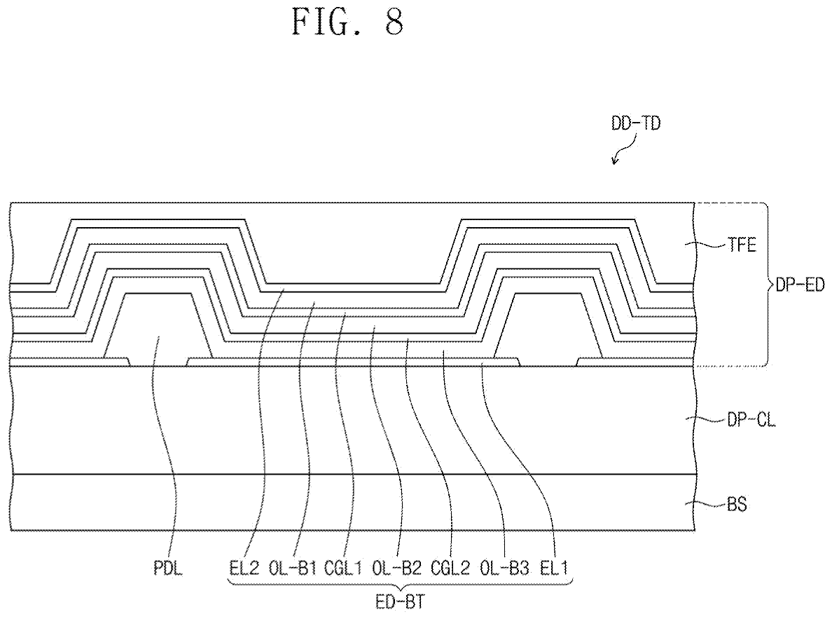

FIG. 7 and FIG. 8 are cross-sectional views on display devices according to one or more embodiments of the present disclosure. Hereinafter, in the explanation on the display devices of embodiments, by referring to FIG. 7 and FIG. 8, the overlapping parts with the explanation on FIG. 1 to FIG. 6 will not be explained/described again for conciseness, and only different features will be explained chiefly and mainly.

Referring to FIG. 7, a display device DD according to one or more embodiments may include a display panel DP including a display element layer DP-ED, a light controlling layer CCL disposed on the display panel DP, and a color filter layer CFL.

In one or more embodiments shown in FIG. 7, the display panel DP may include a base layer BS, a circuit layer DP-CL provided on the base layer BS, and a display element layer DP-ED, and the display element layer DP-ED may include a light emitting element ED.

The light emitting element ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. In one or more embodiments, the structures of the light emitting elements of FIG. 3 to FIG. 6 may be applied to the structure of the light emitting element ED shown in FIG. 7.

Referring to FIG. 7, the emission layer EML may be disposed in an opening part OH defined in a pixel definition layer PDL. For example, the emission layer EML divided by the pixel definition layer PDL and correspondingly provided to each of luminous areas PXA-R, PXA-G, and PXA-B may be to emit light in substantially the same wavelength region. In the display device DD-a of one or more embodiments, the emission layer EML may be to emit blue light. In some embodiments, the emission layer EML may be provided as a common layer for all luminous areas PXA-R, PXA-G and PXA-B.

The light controlling layer CCL may be disposed on the display panel DP. The light controlling layer CCL may include a light converter. The light converter may be a quantum dot or a phosphor. The light converter may transform the wavelength of light provided and then emit. For example, the light controlling layer CCL may be a layer including a quantum dot or a layer including a phosphor.

The light controlling layer CCL may include multiple light controlling parts CCP1, CCP2, and CCP3. The light controlling parts CCP1, CCP2, and CCP3 may be separated and apart from one another.

Referring to FIG. 7, a partition pattern BMP may be disposed between the separated light controlling parts CCP1, CCP2, and CCP3, but embodiments of the present disclosure are not limited thereto. In FIG. 7, the partition pattern BMP is shown not to be overlapped with the light controlling parts CCP1, CCP2 and CCP3, but, in some embodiments, at least a portion of the edges of the light controlling parts CCP1, CCP2, and CCP3 may be overlapped with the partition pattern BMP.

The light controlling layer CCL may include a first light controlling part CCP1 including a first quantum dot QD1 converting first color light provided from the light emitting element ED into second color light, a second light controlling part CCP2 including a second quantum dot QD2 converting first color light into third color light, and a third light controlling part CCP3 transmitting first color light.

In one or more embodiments, the first light controlling part CCP1 may provide red light which is the second color light, and the second light controlling part CCP2 may provide green light which is the third color light. The third color controlling part CCP3 may be to transmit and provide blue light which is the first color light provided from the light emitting element ED. For example, the first quantum dot QD1 may be a red quantum dot to emit red light, and the second quantum dot QD2 may be a green quantum dot to emit green light. On the quantum dots QD1 and QD2, the same content as those described above may be applied.

In some embodiments, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include (e.g., may exclude) a quantum dot (e.g., not include any quantum dot) but include the scatterer SP.

The scatterer SP may be an inorganic particle. For example, the scatterer SP may include at least one selected from among TiO₂, ZnO, Al₂O₃, SiO₂, and hollow silica. In one or more embodiments, the scatterer SP may include at least one selected from among TiO₂, ZnO, Al₂O₃, SiO₂, and hollow silica, or may be a mixture of two or more materials selected from among TiO₂, ZnO, Al₂O₃, SiO₂, and hollow silica.

The first light controlling part CCP1, the second light controlling part CCP2, and the third light controlling part CCP3 may respectively include base resins BR1, BR2, and BR3 dispersing the quantum dots QD1 and QD2 and the scatterer SP. In one or more embodiments, the first light controlling part CCP1 may include the first quantum dot QD1 and the scatterer SP dispersed in a first base resin BR1, the second light controlling part CCP2 may include the second quantum dot QD2 and the scatterer SP dispersed in a second base resin BR2, and the third light controlling part CCP3 may include the scatterer particle SP dispersed in a third base resin BR3. The base resins BR1, BR2 and BR3 are mediums in which the quantum dots QD1 and QD2 and the scatterer SP are dispersed, and may be composed of one or more suitable resin compositions which may be generally referred to as a binder. For example, the base resins BR1, BR2 and BR3 may independently be acrylic resins, urethane-based resins, silicone-based resins, epoxy-based resins, etc. The base resins BR1, BR2 and BR3 may be transparent resins. In one or more embodiments, the first base resin BR1, the second base resin BR2, and the third base resin BR3 may be the same or different from each other.

In one or more embodiments, the light controlling layer CCL may include a barrier layer BFL1. The barrier layer BFL1 may play a role of blocking the penetration of moisture and/or oxygen (hereinafter, will be referred to as “humidity/oxygen”). The barrier layer BFL1 may be disposed on the light controlling parts CCP1, CCP2, and CCP3 and may block or reduce the exposure of the light controlling parts CCP1, CCP2 and CCP3 to humidity/oxygen. In some embodiments, the barrier layer BFL1 may cover the light controlling parts CCP1, CCP2, and CCP3. In some embodiments, a barrier layer BFL2 may be provided between the light controlling parts CCP1, CCP2, and CCP3 and a color filter layer CFL.

The barrier layers BFL1 and BFL2 may include at least one inorganic layer. For example, in some embodiments, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide, silicon oxynitride, and/or a thin metal film to secure light transmittance. In some embodiments, the barrier layers BFL1 and BFL2 may further include an organic layer. The barrier layers BFL1 and BFL2 may be composed of a single layer or multiple layers.

In one or more embodiments, in the display device DD, the color filter layer CFL may be disposed on the light controlling layer CCL. For example, in some embodiments, the color filter layer CFL may be disposed directly on the light controlling layer CCL. In these embodiments, the barrier layer BFL2 may not be provided.

The color filter layer CFL may include a light blocking part BM and filters CF1, CF2, and CF3. The color filter layer CFL may include a first filter CF1 transmitting second color light, a second filter CF2 transmitting third color light, and a third filter CF3 transmitting first color light. For example, in some embodiments, the first filter CF1 may be a red filter, the second filter CF2 may be a green filter, and the third filter CF3 may be a blue filter. Each of the filters CF1, CF2 and CF3 may include a polymer photosensitive resin and a pigment and/or dye. The first filter CF1 may include a red pigment and/or dye, the second filter CF2 may include a green pigment and/or dye, and the third filter CF3 may include a blue pigment and/or dye. However, embodiments of the present disclosure are not limited thereto, for example, in some embodiments, the third filter CF3 may not include (e.g., may exclude) the pigment and/or dye. The third filter CF3 may include a polymer photosensitive resin and not include a (e.g., any) pigment and/or dye. The third filter CF3 may be transparent. The third filter CF3 may be formed utilizing a transparent photosensitive resin.

In one or more embodiments, the first filter CF1 and the second filter CF2 may be yellow filters. The first filter CF1 and the second filter CF2 may be provided in one body without distinction.

The light blocking part BM may be a black matrix. The light blocking part BM may be formed by including an organic light blocking material and/or an inorganic light blocking material, including a black pigment and/or black dye. The light blocking part BM may prevent or reduce light leakage and may divide the boundaries among adjacent filters CF1, CF2, and CF3. In some embodiments, the light blocking part BM may be formed as a blue filter.

The first to third filters CF1, CF2, and CF3 may be disposed corresponding to the red luminous area PXA-R, the green luminous area PXA-G, and the blue luminous area PXA-B, respectively.

On the color filter layer CFL, a base substrate BL may be disposed. The base substrate BL may be a member providing a base surface on which the color filter layer CFL, the light controlling layer CCL, etc. are disposed. The base substrate BL may be a glass substrate, a metal substrate, a plastic substrate, etc. However, embodiments of the present disclosure are not limited thereto, and the base substrate BL may be an inorganic layer, an organic layer, or a composite material layer. In some embodiments, the base substrate BL may not be provided.

FIG. 8 is a cross-sectional view showing a part of the display device according to one or more embodiments of the present disclosure. In FIG. 8, the cross-sectional view of a part corresponding to the display panel DP of FIG. 7 is shown. In a display device DD-TD of one or more embodiments, the light emitting element ED-BT may include multiple light emitting structures OL-B1, OL-B2, and OL-B3. The light emitting element ED-BT may include oppositely disposed first electrode EL1 and second electrode EL2, and the multiple light emitting structures OL-B1, OL-B2, and OL-B3 stacked in order in a thickness direction and provided between the first electrode EL1 and the second electrode EL2. Each of the light emitting structures OL-B1, OL-B2, and OL-B3 may include an emission layer EML (FIG. 7), and a hole transport region HTR and an electron transport region ETR, disposed with the emission layer (FIG. 7) therebetween.

For example, in some embodiments, the light emitting element ED-BT included in the display device DD-TD may be a light emitting element of a tandem structure including multiple emission layers.

In one or more embodiments shown in FIG. 8, light emitted from the light emitting structures OL-B1, OL-B2, and OL-B3 may be all blue light. However, embodiments of the present disclosure are not limited thereto, and the wavelength regions of light emitted from the light emitting structures OL-B1, OL-B2, and OL-B3 may be different from each other. For example, in one or more embodiments, the light emitting element ED-BT including the multiple light emitting structures OL-B1, OL-B2, and OL-B3 emitting light in different wavelength regions may be to emit white light (e.g., combined white light).

Between neighboring light emitting structures OL-B1, OL-B2, and OL-B3, charge generating layers CGL1 and CGL2 may be disposed. The charge generating layers CGL1 and CGL2 may include a p-type or kind charge (e.g., P-charge) generating layer and/or an n-type or kind charge (e.g., N-charge) generating layer.

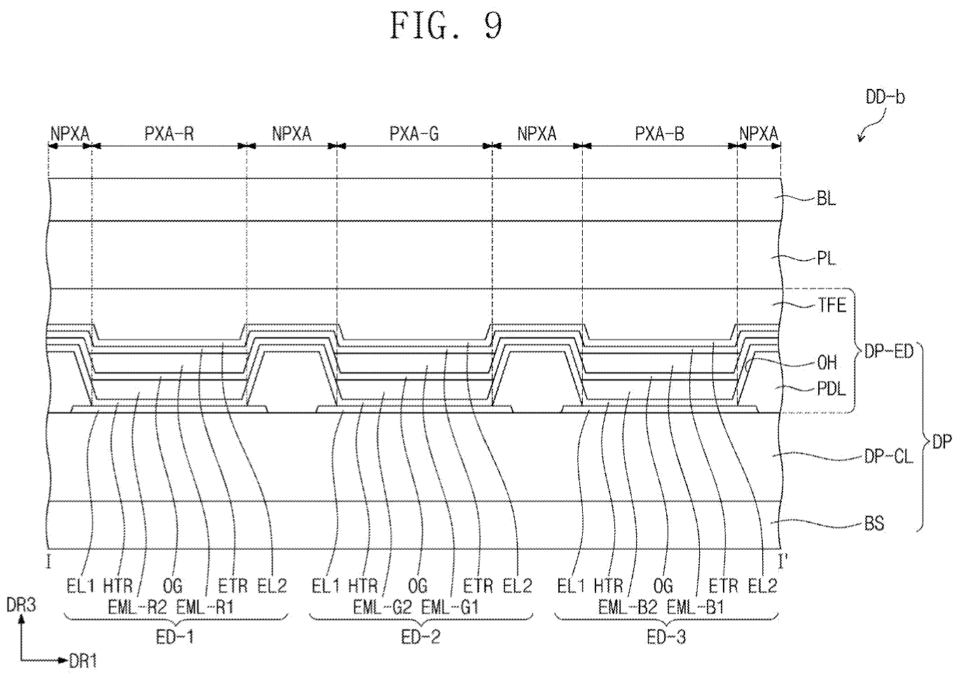

Referring to FIG. 9, a display device DD-b according to one or more embodiments of the present disclosure may include light emitting elements ED-1, ED-2, and ED-3, each formed by stacking two emission layers. Compared to the display device DD shown in FIG. 2, the display device DD-b shown in FIG. 9 is different in that first to third light emitting elements ED-1, ED-2, and ED-3 each include two emission layers stacked in a thickness direction. In each of the first to third light emitting elements ED-1, ED-2, and ED-3, the two emission layers may be to emit light in substantially the same wavelength region.

In one or more embodiments, the first light emitting element ED-1 may include a first red emission layer EML-R1 and a second red emission layer EML-R2. The second light emitting element ED-2 may include a first green emission layer EML-G1 and a second green emission layer EML-G2. In addition, the third light emitting element ED-3 may include a first blue emission layer EML-B1 and a second blue emission layer EML-B2. Between the first red emission layer EML-R1 and the second red emission layer EML-R2, between the first green emission layer EML-G1 and the second green emission layer EML-G2, and between the first blue emission layer EML-B1 and the second blue emission layer EML-B2, an emission auxiliary part OG may be disposed.

The emission auxiliary part OG may include a single layer or a multilayer. The emission auxiliary part OG may include a charge generating layer. In one or more embodiments, the emission auxiliary part OG may include an electron transport region, a charge generating layer, and a hole transport region stacked in order. The emission auxiliary part OG may be provided as a common layer in all of the first to third light emitting elements ED-1, ED-2, and ED-3. However, embodiments of the present disclosure are not limited thereto, and the emission auxiliary part OG may be patterned and provided in an opening part OH defined in a pixel definition layer PDL.

The first red emission layer EML-R1, the first green emission layer EML-G1 and the first blue emission layer EML-B1 may be disposed between the electron transport region ETR and the emission auxiliary part OG. The second red emission layer EML-R2, the second green emission layer EML-G2 and the second blue emission layer EML-B2 may be disposed between the emission auxiliary part OG and the hole transport region HTR.

For example, in some embodiments, the first light emitting element ED-1 may include a first electrode EL1, a hole transport region HTR, a second red emission layer EML-R2, an emission auxiliary part OG, a first red emission layer EML-R1, an electron transport region ETR, and a second electrode EL2, stacked in order (e.g., in the stated order). The second light emitting element ED-2 may include a first electrode EL1, a hole transport region HTR, a second green emission layer EML-G2, an emission auxiliary part OG, a first green emission layer EML-G1, an electron transport region ETR, and a second electrode EL2, stacked in order (e.g., in the stated order). The third light emitting element ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in order (e.g., in the stated order).

In some embodiments, an optical auxiliary layer PL may be disposed on a display element layer DP-ED. The optical auxiliary layer PL may include a polarization layer. The optical auxiliary layer PL may be disposed on a display panel DP and may control reflected light at the display panel DP by external light. In some embodiments, the optical auxiliary layer PL may not be provided in the display device.

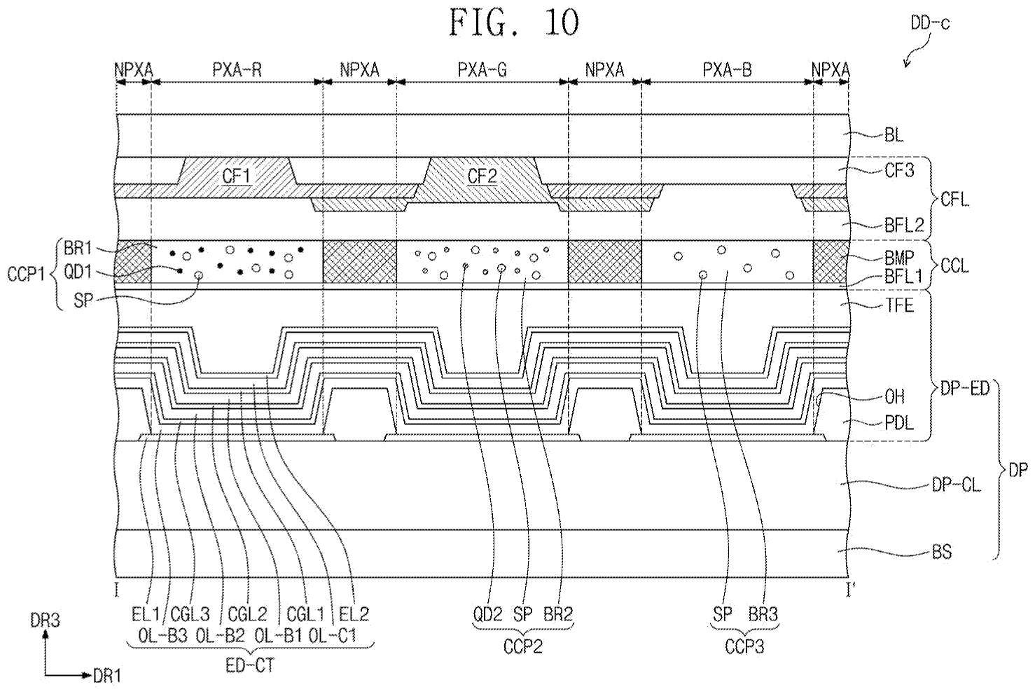

Different from FIG. 8 and FIG. 9, a display device DD-c in FIG. 10 is shown to include four light emitting structures OL-B1, OL-B2, OL-B3, and OL-C1. A light emitting element ED-CT may include oppositely disposed first electrode EL1 and second electrode EL2, and first to fourth light emitting structures OL-B1, OL-B2, OL-B3, and OL-C1 stacked in a thickness direction between the first electrode EL1 and the second electrode EL2. Between the first to fourth light emitting structures OL-B1, OL-B2, OL-B3, and OL-C1, charge generating layers CGL1, CGL2, and CGL3 may be disposed. In some embodiments, among the four light emitting structures, the first to third light emitting structures OL-B1, OL-B2, and OL-B3 may be to emit blue light, and the fourth light emitting structure OL-C1 may be to emit green light. However, embodiments of the present disclosure are not limited thereto, and the first to fourth light emitting structures OL-B1, OL-B2, OL-B3, and OL-C1 may be to emit different wavelengths of light.

Charge generating layers CGL1, CGL2, and CGL3 disposed among neighboring light emitting structures OL-B1, OL-B2, OL-B3, and OL-C1 may include a p-type or kind charge (e.g., P-charge) generating layer and/or an n-type or kind charge (e.g., N-charge) generating layer.

Hereinafter, referring to embodiments and comparative embodiments, the amine compound according to one or more embodiments and the light emitting element according to one or more embodiments of the present disclosure will be explained in detail. In addition, the embodiments described are mere illustrations to assist the understanding of the present disclosure, but the scope of the present disclosure is not limited thereto.

### Examples

### 1. Synthesis of Amine Compounds of Embodiments

The synthetic methods of amine compounds according to embodiments will be explained in detail by illustrating the synthetic methods of Compounds 1, 2, 7, 8, 10, 11, 41, 42, 47, 48, 61, 62, 67, 68, 70, 71, 81, 82, 87, 88, 121, 122, 141, and 142. In addition, the synthetic methods of the amine compounds explained hereinafter are embodiments and examples, and the synthetic method of the amine compound according to one or more embodiments of the present disclosure is not limited to the embodiments described.

### (1) Synthesis of Compound C1

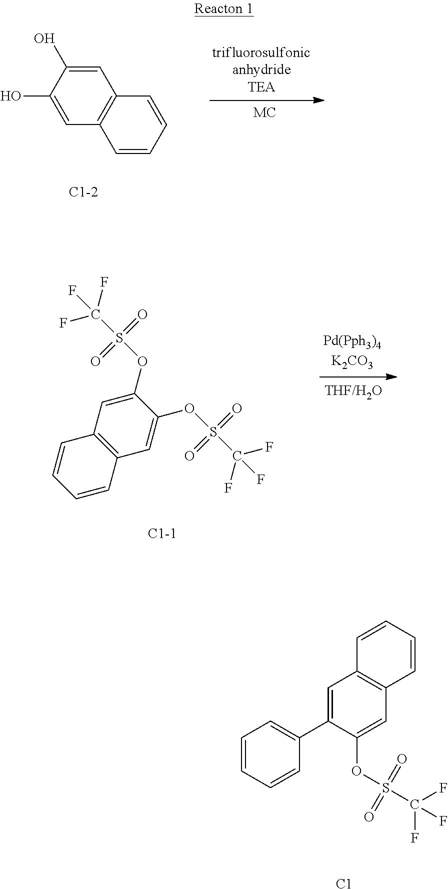 [SMILES: Oc1cc2ccccc2cc1O ; O=S(=O)(Oc1cc2ccccc2cc1OS(=O)(=O)C(F)(F)F)C(F)(F)F ; O=S(=O)(Oc1cc2ccccc2cc1-c1ccccc1)C(F)(F)F ; "C1"]

Compound 01-2 (20 mmol, 1 eq), trifluorosulfonic anhydride (80 mmol, 4 eq), triethylamine (60 mmol, 3 eq), and dichloromethane (200 mL) were put in a 1-neck-round flask and stirred at about 0° C. for about 2 hours.

After finishing the reaction, the resultant was worked-up with ether/H₂O, and separated by column chromatography utilizing only hexane as an eluent to obtain 18 mmol of Compound C1-1 (yield=90%).

Compound C1-1 (18 mmol, 1 eq), 1-amino-4-bromobenzene (18 mmol, 1 eq), Pd(PPh₃)₄ (0.9 mmol, 0.05 eq), K₂CO₃ (54 mmol, 3 eq), and THF/H₂O (200/40 mL) were stirred at about 80° C. (for about 24 hours). After finishing the reaction, the resultant was worked-up with ether/H₂O, and separated by column chromatography utilizing only hexane as an eluent to obtain 9 mmol of Compound C1 (yield=50%).

### (2) Synthesis of Compound C2

Compound C2 is synthesized by Reaction 2.

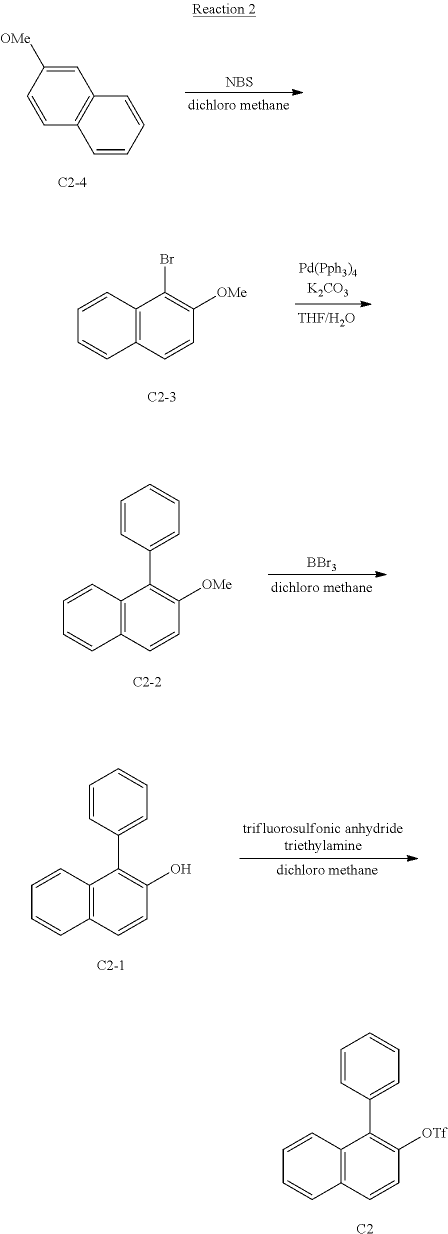

Compound C2-4 (50 mmol, 1 eq), NBS (50 mmol, 1 eq), and dichloromethane (400 mL) were put in a 1-neck-round flask and stirred at about 70° C. (for about 4 hours). After finishing the reaction, the resultant was worked-up with ether/H₂O, and separated by column chromatography utilizing only hexane as an eluent to obtain 45 mmol of Compound C2-3 (yield=90%).

Compound C2-3 (45 mmol, 1 eq), phenyl boronic acid (45 mmol, 1 eq), Pd(PPh₃)₄ (2.25 mmol, 0.05 eq), K₂CO₃ (135 mmol, 3 eq), and THF/H₂O (400/80 ml mL) were stirred at about 80° C. (for about 24 hours).

After finishing the reaction, the resultant was worked-up with ether/H₂O, and separated by column chromatography utilizing only hexane as an eluent to obtain 36 mmol of Compound C2-2 (yield=80%).

Compound C2-2 (36 mmol, 1 eq), BBr₃ (36 mmol, 1 eq), and dichloromethane (400 mL) were stirred at about 100° C. (for about 4 hours). After finishing the reaction, the resultant was worked-up with ether/H₂O, and separated by column chromatography utilizing dichloromethane/hexane=1/3 as an eluent to obtain 31.5 mmol of Compound C2-1 (yield=90%).

Compound C2-1 (31.5 mmol, 1 eq), trifluorosulfonic anhydride (63 mmol, 2 eq), triethylamine (63 mmol, 2 eq), and dichloromethane (400 ml mL) were put in a 1-neck-round flask and stirred at about 0° C. (for about 2 hours).

After finishing the reaction, the resultant was worked-up with ether/H₂O, and separated by column chromatography utilizing only hexane as an eluent to obtain 26.8 mmol of Compound C2 (yield=85%).

### (3) Synthesis of Amine Compound 1

Amine Compound 1 according to one or more embodiments may be synthesized by, for example, the steps of Reaction 3.

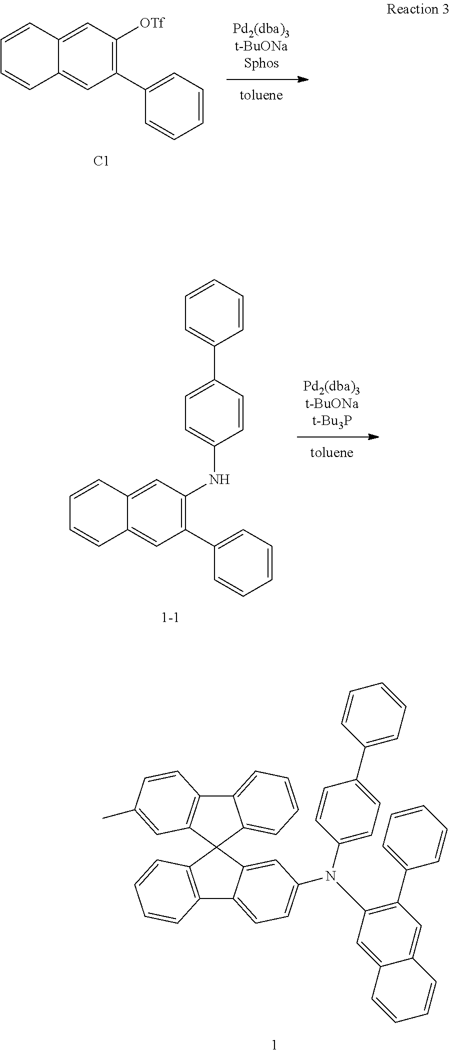 [SMILES: O=S(=O)(Oc1cc2ccccc2cc1-c1ccccc1)C(F)(F)F ; "C1" ; c1ccc(-c2ccc(Nc3cc4ccccc4cc3-c3ccccc3)cc2)cc1 ; "1-1" ; Cc1ccc2c(c1)C1(c3ccccc3-2)c2ccccc2-c2ccc(N(c3ccc(-c4ccccc4)cc3)c3cc4ccccc4cc3-c3ccccc3)cc21]

4-Aminobiphenyl (26 mmol, 1.3 eq), Compound C1 (20 mmol, 1 eq), Pd₂(dba)₃ (1 mmol, 0.05 eq), t-BuONa (40 mmol, 2 eq), SPhos (2 mmol, 0.1 eq), and 300 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/5 as an eluent to obtain 12 mmol of Compound 1-1 (yield=60%).

Compound 1-1 (12 mmol, 1.2 eq), 2-bromo-2′-methyl-9,9′-spirobi[fluorene](10 mmol, 1 eq), Pd₂(dba)₃ (0.5 mmol, 0.05 eq), t-BuONa (20 mmol, 2 eq), t-Bu₃P (0.1 mmol, 0.1 eq), 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 6.3 mmol of Compound 1 (yield=63%).

### (4) Synthesis of Amine Compound 2

Amine Compound 2 according to one or more embodiments may be synthesized by, for example, the steps of Reaction 4.

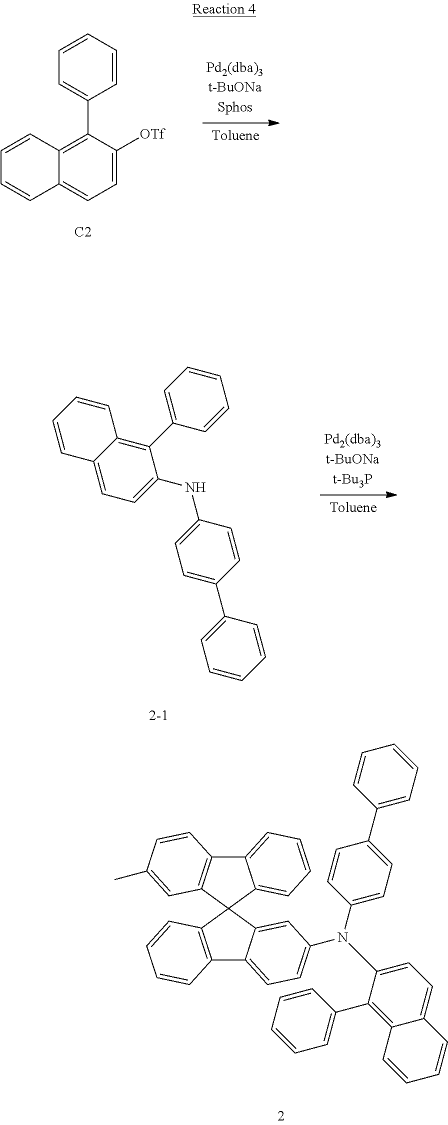

4-Aminobiphenyl (26 mmol, 1.3 eq), Compound C2 (20 mmol, 1 eq), Pd₂(dba)₃ (1 mmol, 0.05 eq), t-BuONa (40 mmol, 2 eq), SPhos (2 mmol, 0.1 eq), and 300 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/5 as an eluent to obtain 10 mmol of Compound 2-1 (yield=50%).

Compound 2-1 (10 mmol, 1.2 eq), 2-bromo-2′-methyl-9,9′-spirobi[fluorene] (8 mmol, 1 eq), Pd₂(dba)₃ (0.4 mmol, 0.05 eq), t-BuONa (16 mmol, 2 eq), t-Bu₃P (0.8 mmol, 0.1 eq), 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 6.1 mmol of Compound 2 (yield=76%).

### (5) Synthesis of Amine Compound 7

Amine Compound 7 according to one or more embodiments may be synthesized by, for example, the steps of Reaction 5.

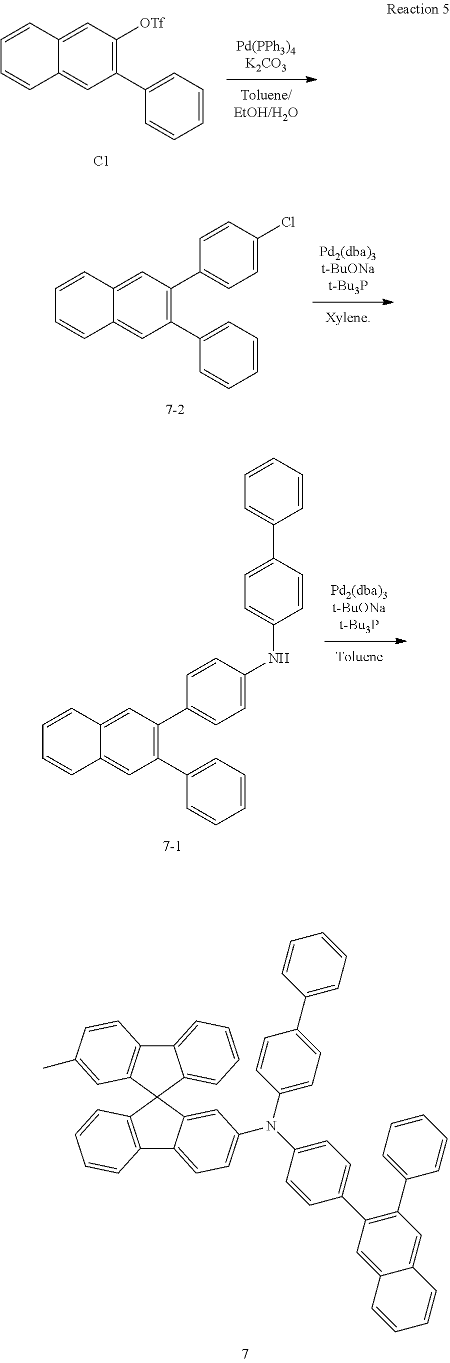

Compound C1 (40 mmol, 1 eq), (4-chlorophenyl)boronic acid (44 mmol, 1.1 eq), Pd(PPh₃)₄ (2 mmol, 0.05 eq), K₂CO₃ (120 mmol, 3 eq), and toluene/EtOH/H₂O (200/40/40 mL) were put in 1-neck-round flask and stirred at about 120° C. (for about 4 hours). OTf in Compound C1 is trifluoromethanesulfonate.

After finishing the reaction, the resultant was worked-up with ether/H₂O, and separated by column chromatography utilizing dichloromethane/hexane=1/20 as an eluent to obtain 28 mmol of Compound 7-2 (yield=70%).

4-Aminobiphenyl (26 mmol, 1.3 eq), Compound 7-2 (20 mmol, 1 eq), Pd₂(dba)₃ (1 mmol, 0.05 eq), t-BuONa (40 mmol, 2 eq), t-Bu₃P (2 mmol, 0.1 eq), and 300 mL of xylene were put in a 1-neck-round flask and stirred at about 140° C. (for about 2 hours).

After finishing the reaction, the resultant was worked-up with ether/H₂O, and separated by column chromatography utilizing dichloromethane/hexane=1/5 as an eluent to obtain 16 mmol of Compound 7-1 (yield=80%).

Compound 7-1 (12 mmol, 1.2 eq), 2-bromo-2′-methyl-9,9′-spirobi[fluorene](10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 6.8 mmol of Compound 7 (yield=68%).

### (6) Synthesis of Amine Compound 8

Amine Compound 8 according to one or more embodiments may be synthesized by, for example, the steps of Reaction 6.

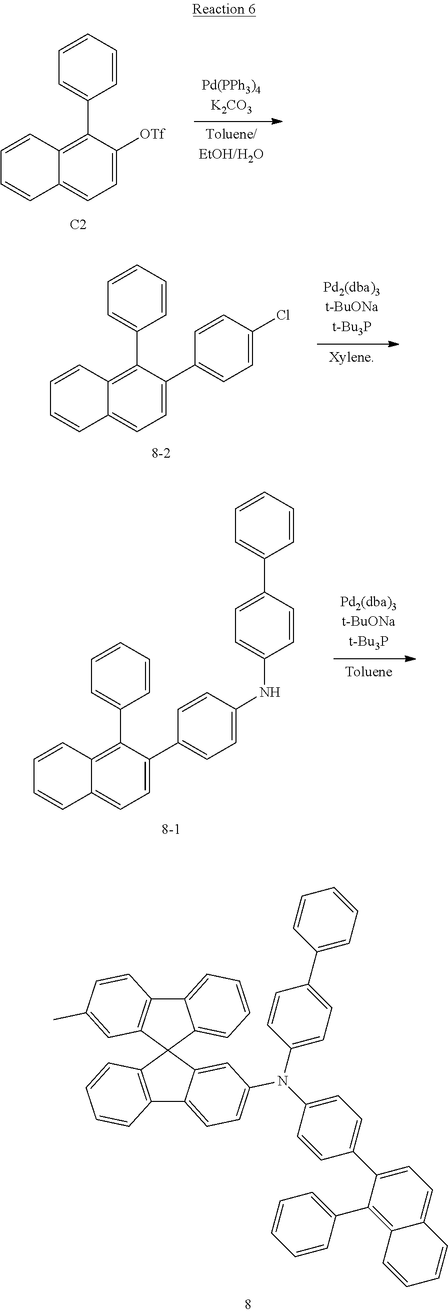

Compound C2 (40 mmol, 1 eq), (4-chlorophenyl)boronic acid (44 mmol, 1.1 eq), Pd(PPh₃)₄ (2 mmol, 0.05 eq), K₂CO₃ (120 mmol, 3 eq), and toluene/EtOH/H₂O (200/40/40 mL) were put in a 1-neck-round flask and stirred at about 120° C. (for about 4 hours).

After finishing the reaction, the resultant was worked-up with ether/H₂O, and separated by column chromatography utilizing dichloromethane/hexane=1/20 as an eluent to obtain 28.8 mmol of Compound 8-2 (yield=72%).

4-Aminobiphenyl (26 mmol, 1.3 eq), Compound 8-2 (20 mmol, 1 eq), Pd₂(dba)₃ (1 mmol, 0.05 eq), t-BuONa (40 mmol, 2 eq), t-Bu₃P (2 mmol, 0.1 eq), and 300 mL of xylene were put in a 1-neck-round flask and stirred at about 140° C. (for about 2 hours).

After finishing the reaction, the resultant was worked-up with ether/H₂O, and separated by column chromatography utilizing dichloromethane/hexane=1/5 as an eluent to obtain 16.6 mmol of Compound 8-1 (yield=83%).

Compound 8-1 (12 mmol, 1.2 eq), 2-bromo-2′-methyl-9,9′-spirobi[fluorene](10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 7.1 mmol of Compound 8 (yield=71%).

### (7) Synthesis of Amine Compound 10

Amine Compound 10 according to an embodiment may be synthesized by, for example, the steps of Reaction 7.

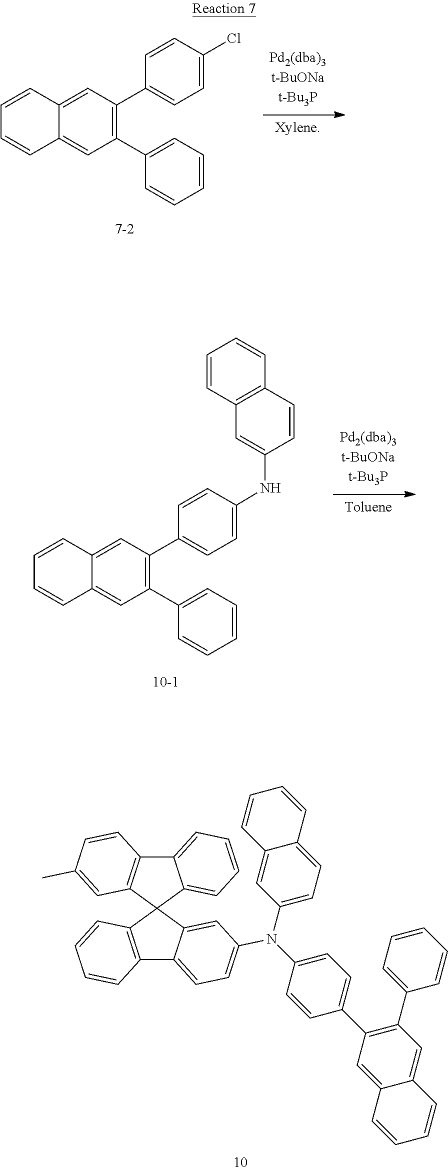 [SMILES: Clc1ccc(-c2cc3ccccc3cc2-c2ccccc2)cc1 ; c1ccc(-c2cc3ccccc3cc2-c2ccc(Nc3ccc4ccccc4c3)cc2)cc1 ; Cc1ccc2c(c1)C1(c3ccccc3-2)c2ccccc2-c2ccc(N(c3ccc(-c4cc5ccccc5cc4-c4ccccc4)cc3)c3ccc4ccccc4c3)cc21]

2-Aminonaphthalene (26 mmol, 1.3 eq), Compound 7-2 (20 mmol, 1 eq), Pd₂(dba)₃ (1 mmol, 0.05 eq), t-BuONa (40 mmol, 2 eq), t-Bu₃P (2 mmol, 0.1 eq), and 300 mL of xylene were put in a 1-neck-round flask and stirred at about 140° C. (for about 2 hours).

After finishing the reaction, the resultant was worked-up with ether/H₂O, and separated by column chromatography utilizing dichloromethane/hexane=1/5 as an eluent to obtain 17 mmol of Compound 10-1 (yield=85%).

Compound 10-1 (12 mmol, 1.2 eq), 2-bromo-2′-methyl-9,9′-spirobi[fluorene](10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 7.0 mmol of Compound 10 (yield=70%).

### (8) Synthesis of Amine Compound 11

Amine Compound 11 according to one or more embodiments may be synthesized by, for example, the steps of Reaction 8.

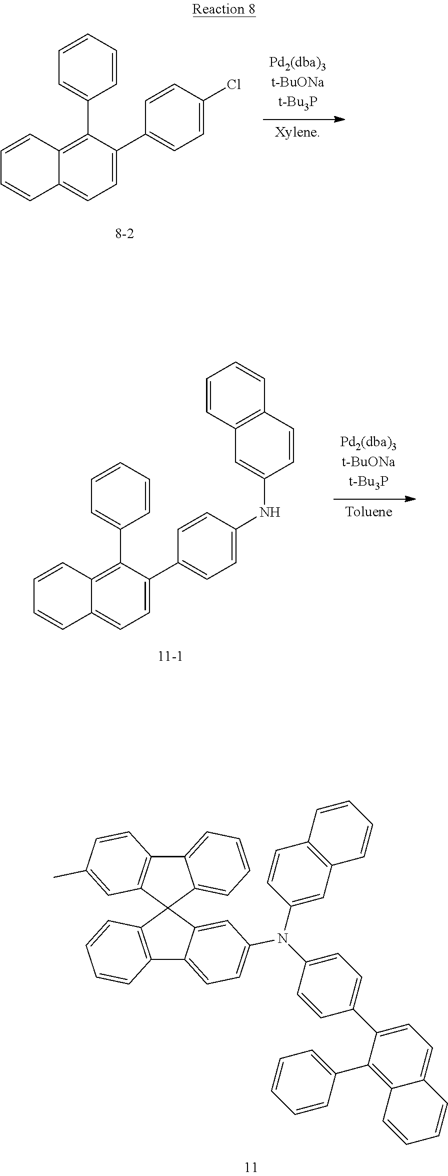 [SMILES: Clc1ccc(-c2ccc3ccccc3c2-c2ccccc2)cc1 ; c1ccc(-c2c(-c3ccc(Nc4ccc5ccccc5c4)cc3)ccc3ccccc23)cc1 ; Cc1ccc2c(c1)C1(c3ccccc3-2)c2ccccc2-c2ccc(N(c3ccc(-c4ccc5ccccc5c4-c4ccccc4)cc3)c3ccc4ccccc4c3)cc21]

2-Aminonaphthalene (26 mmol, 1.3 eq), Compound 8-2 (20 mmol, 1 eq), Pd₂(dba)₃ (1 mmol, 0.05 eq), t-BuONa (40 mmol, 2 eq), t-Bu₃P (2 mmol, 0.1 eq), and 300 mL of xylene were put in a 1-neck-round flask and stirred at about 140° C. (for about 2 hours).

After finishing the reaction, the resultant was worked-up with ether/H₂O, and separated by column chromatography utilizing dichloromethane/hexane=1/5 as an eluent to obtain 15.8 mmol of Compound 11-1 (yield=79%).

Compound 11-1 (12 mmol, 1.2 eq), 2-bromo-2′-methyl-9,9′-spirobi[fluorene](10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 7.4 mmol of Compound 11 (yield=74%).

### (9) Synthesis of Amine Compound 41

Amine Compound 41 according to one or more embodiments may be synthesized by, for example, the steps of Reaction 9.

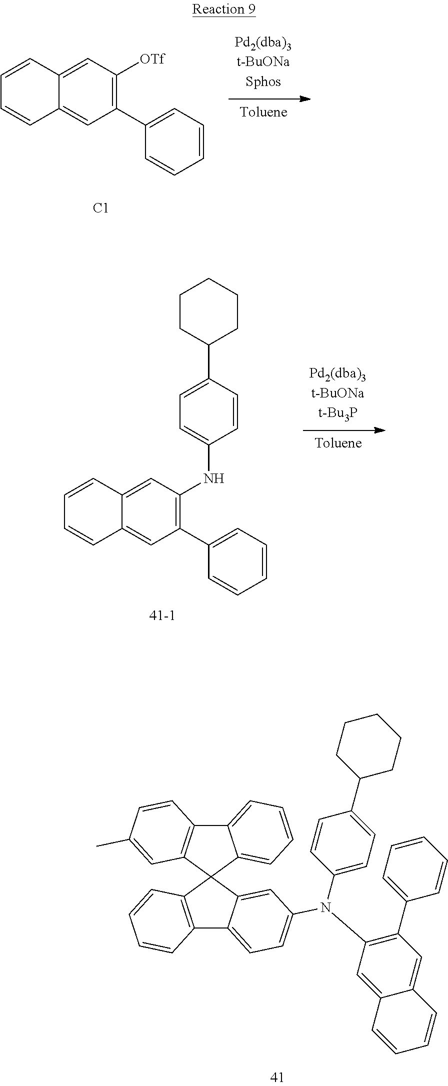 [SMILES: O=S(=O)(Oc1cc2ccccc2cc1-c1ccccc1)C(F)(F)F ; "C1" ; c1ccc(-c2cc3ccccc3cc2Nc2ccc(C3CCCCC3)cc2)cc1 ; Cc1ccc2c(c1)C1(c3ccccc3-2)c2ccccc2-c2ccc(N(c3ccc(C4CCCCC4)cc3)c3cc4ccccc4cc3-c3ccccc3)cc21]

4-Cyclohexylaniline (26 mmol, 1.3 eq), Compound C1 (20 mmol, 1 eq), Pd₂(dba)₃ (1 mmol, 0.05 eq), t-BuONa (40 mmol, 2 eq), SPhos (2 mmol, 0.1 eq), and 300 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/5 as an eluent to obtain 13.2 mmol of Compound 41-1 (yield=66%).

Compound 41-1 (12 mmol, 1.2 eq), 2-bromo-2′-methyl-9,9′-spirobi[fluorene](10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 6.0 mmol of Compound 41 (yield=60%).

### (10) Synthesis of Amine Compound 42

Amine Compound 42 according to one or more embodiments may be synthesized by, for example, the steps of Reaction 10.

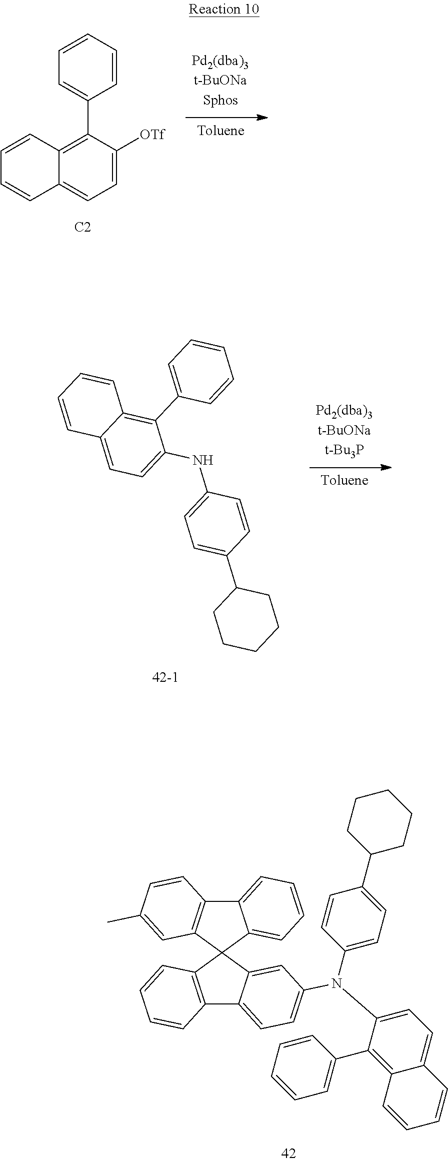

4-Cyclohexylaniline (26 mmol, 1.3 eq), Compound C2 (20 mmol, 1 eq), Pd₂(dba)₃ (1 mmol, 0.05 eq), t-BuONa (40 mmol, 2 eq), SPhos (2 mmol, 0.1 eq), and 300 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/5 as an eluent to obtain 10 mmol of Compound 42-1 (yield=50%).

Compound 42-1 (10 mmol, 1.2 eq), 2-bromo-2′-methyl-9,9′-spirobi[fluorene](8 mmol, 1 eq), Pd₂(dba)₃ (0.4 mmol, 0.05 eq), t-BuONa (16 mmol, 2 eq), t-Bu₃P (0.8 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 6.4 mmol of Compound 42 (yield=80%).

### (11) Synthesis of Amine Compound 47

Amine Compound 47 according to one or more embodiments may be synthesized by, for example, the steps of Reaction 11.

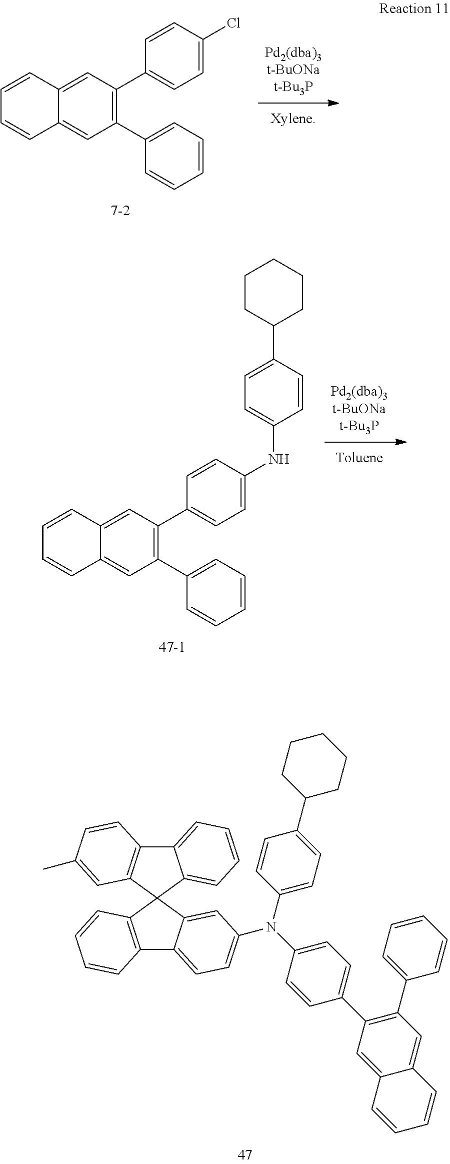

4-Cyclohexylaniline (26 mmol, 1.3 eq), Compound 7-2 (20 mmol, 1 eq), Pd₂(dba)₃ (1 mmol, 0.05 eq), t-BuONa (40 mmol, 2 eq), t-Bu₃P (2 mmol, 0.1 eq), and 300 mL of xylene were put in a 1-neck-round flask and stirred at about 140° C. (for about 2 hours).

After finishing the reaction, the resultant was worked-up with ether/H₂O, and separated by column chromatography utilizing dichloromethane/hexane=1/5 as an eluent to obtain 15 mmol of Compound 47-1 (yield=75%).

Compound 47-1 (12 mmol, 1.2 eq), 2-bromo-2′-methyl-9,9′-spirobi[fluorene](10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 7.2 mmol of Compound 47 (yield=72%).

### (12) Synthesis of Amine Compound 48

Amine Compound 48 according to one or more embodiments may be synthesized by, for example, the steps of Reaction 12.

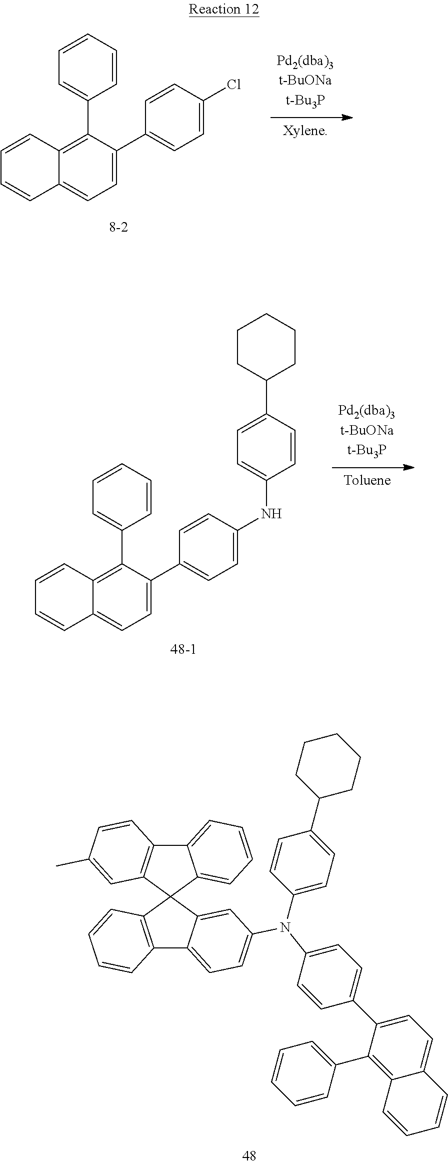

4-Cyclohexylaniline (26 mmol, 1.3 eq), Compound 8-2 (20 mmol, 1 eq), Pd₂(dba)₃ (1 mmol, 0.05 eq), t-BuONa (40 mmol, 2 eq), t-Bu₃P (2 mmol, 0.1 eq), and 300 mL of xylene were put in a 1-neck-round flask and stirred at about 140° C. (for about 2 hours).

After finishing the reaction, the resultant was worked-up with ether/H₂O, and separated by column chromatography utilizing dichloromethane/hexane=1/5 as an eluent to obtain 14.2 mmol of Compound 48-1 (yield=71%).

Compound 48-1 (12 mmol, 1.2 eq), 2-bromo-2′-methyl-9,9′-spirobi[fluorene](10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 7.4 mmol of Compound 48 (yield=74%).

### (13) Synthesis of Amine Compound 61

Amine Compound 61 according to one or more embodiments may be synthesized by, for example, the step of Reaction 13.

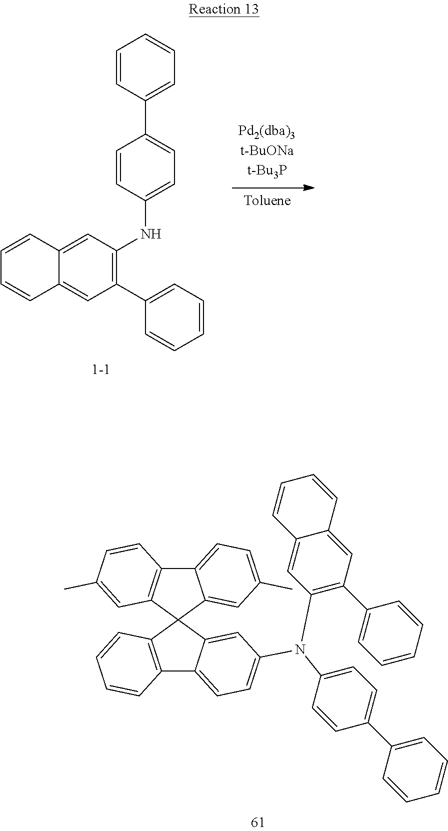 [SMILES: c1ccc(-c2ccc(Nc3cc4ccccc4cc3-c3ccccc3)cc2)cc1 ; Cc1ccc2c(c1)C1(c3ccccc3-c3ccc(N(c4ccc(-c5ccccc5)cc4)c4cc5ccccc5cc4-c4ccccc4)cc31)c1cc(C)ccc1-2]

Compound 1-1 (12 mmol, 1.2 eq), 2′-bromo-2,7-dimethyl-9,9′-spirobi[fluorene] (10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 5.3 mmol of Compound 61 (yield=53%).

### (14) Synthesis of Amine Compound 62

Amine Compound 62 according to one or more embodiments may be synthesized by, for example, the step of Reaction 14.

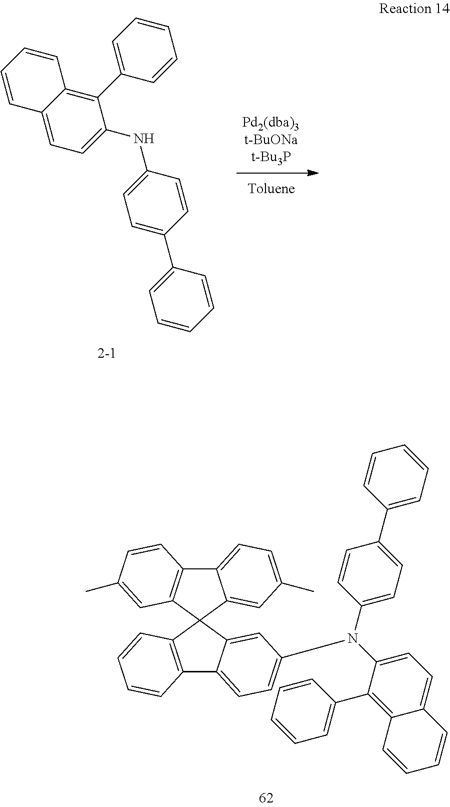 [SMILES: c1ccc(-c2ccc(Nc3ccc4ccccc4c3-c3ccccc3)cc2)cc1 ; "2-1" ; Cc1ccc2c(c1)C1(c3ccccc3-c3ccc(N(c4ccc(-c5ccccc5)cc4)c4ccc5ccccc5c4-c4ccccc4)cc31)c1cc(C)ccc1-2]

Compound 2-1 (12 mmol, 1.2 eq), 2′-bromo-2,7-dimethyl-9,9′-spirobi[fluorene] (10 mmol, 1 eq), Pd₂(dba)₃ (0.5 mmol, 0.05 eq), t-BuONa (20 mmol, 2 eq), t-Bu₃P (1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 6.1 mmol of Compound 62 (yield=61%).

### (15) Synthesis of Amine Compound 67

Amine Compound 67 according to one or more embodiments may be synthesized by, for example, the step of Reaction 15.

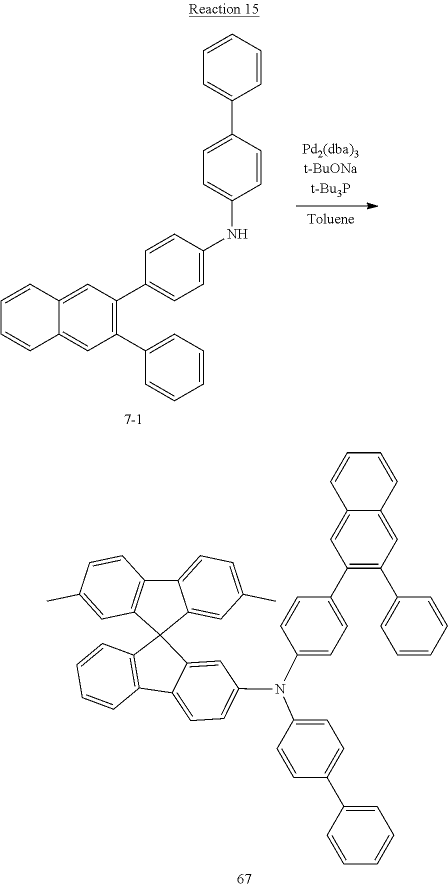 [SMILES: c1ccc(-c2ccc(Nc3ccc(-c4cc5ccccc5cc4-c4ccccc4)cc3)cc2)cc1 ; Cc1ccc2c(c1)C1(c3ccccc3-c3ccc(N(c4ccc(-c5ccccc5)cc4)c4ccc(-c5cc6ccccc6cc5-c5ccccc5)cc4)cc31)c1cc(C)ccc1-2]

Compound 7-1 (12 mmol, 1.2 eq), 2′-bromo-2,7-dimethyl-9,9′-spirobi[fluorene] (10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 5.0 mmol of Compound 67 (yield=50%).

### (16) Synthesis of Amine Compound 68

Amine Compound 68 according to one or more embodiments may be synthesized by, for example, the step of Reaction 16.

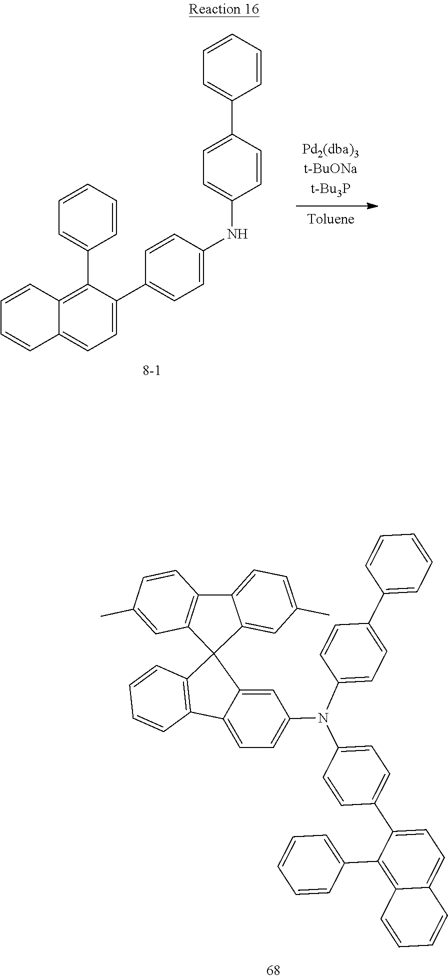 [SMILES: c1ccc(-c2ccc(Nc3ccc(-c4ccc5ccccc5c4-c4ccccc4)cc3)cc2)cc1 ; Cc1ccc2c(c1)C1(c3ccccc3-c3ccc(N(c4ccc(-c5ccccc5)cc4)c4ccc(-c5ccc6ccccc6c5-c5ccccc5)cc4)cc31)c1cc(C)ccc1-2]

Compound 8-1 (12 mmol, 1.2 eq), 2′-bromo-2,7-dimethyl-9,9′-spirobi[fluorene] (10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 5.1 mmol of Compound 68 (yield=51%).

### (17) Synthesis of Amine Compound 70

Amine Compound 70 according to one or more embodiments may be synthesized by, for example, the step of Reaction 17.

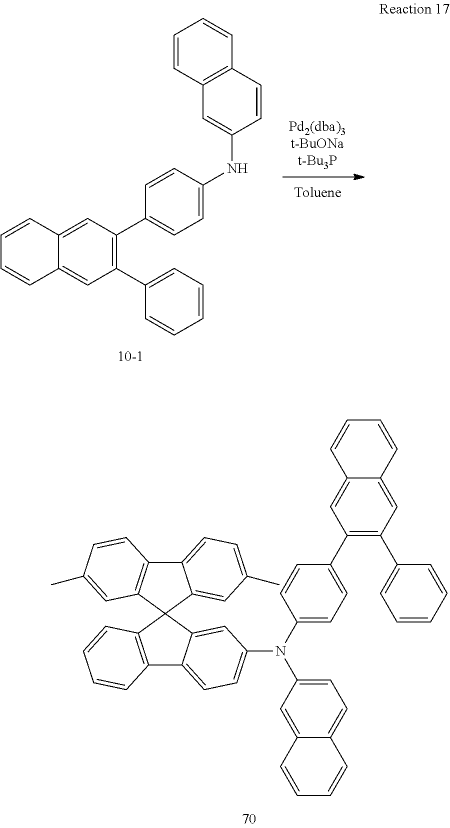

Compound 10-1 (12 mmol, 1.2 eq), 2′-bromo-2,7-dimethyl-9,9′-spirobi[fluorene] (10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 5.0 mmol of Compound 70 (yield=50%).

### (18) Synthesis of Amine Compound 71

Amine Compound 71 according to one or more embodiments may be synthesized by, for example, the step of Reaction 18.

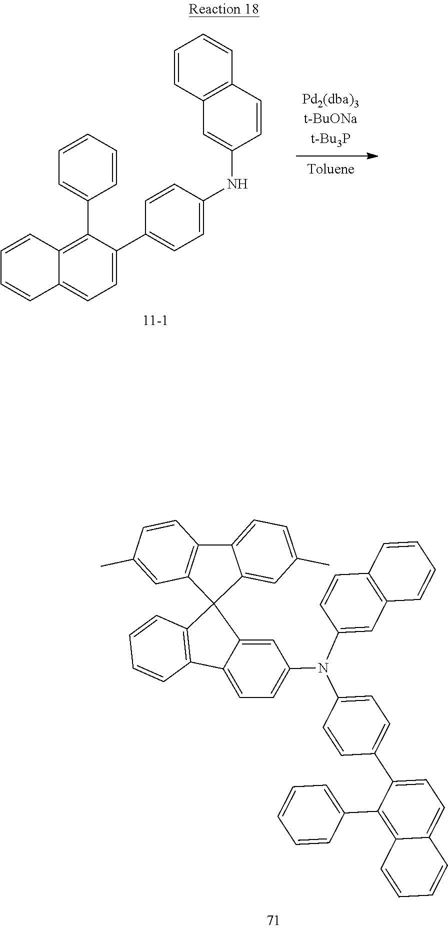 [SMILES: c1ccc(-c2c(-c3ccc(Nc4ccc5ccccc5c4)cc3)ccc3ccccc23)cc1 ; Cc1ccc2c(c1)C1(c3ccccc3-c3ccc(N(c4ccc(-c5ccc6ccccc6c5-c5ccccc5)cc4)c4ccc5ccccc5c4)cc31)c1cc(C)ccc1-2]

Compound 11-1 (12 mmol, 1.2 eq), 2′-bromo-2,7-dimethyl-9,9′-spirobi[fluorene] (10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 5.4 mmol of Compound 71 (yield=54%).

### (19) Synthesis of Amine Compound 81

Amine Compound 81 according to one or more embodiments may be synthesized by, for example, the step of Reaction 19.

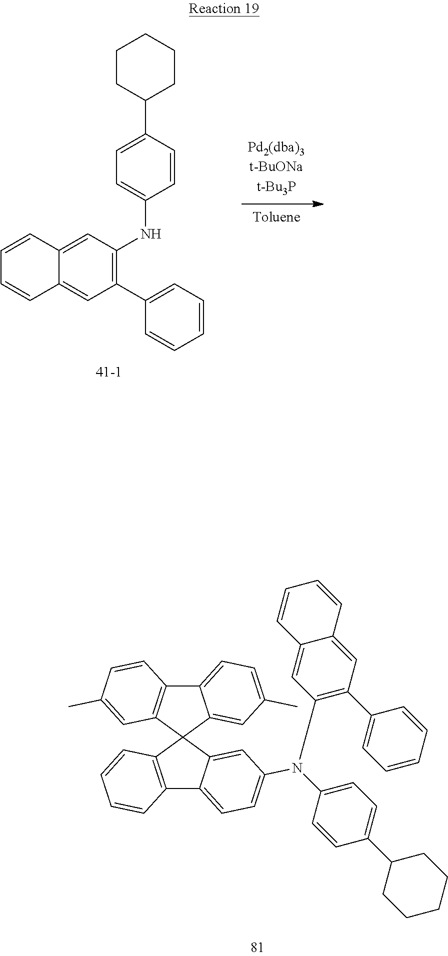 [SMILES: c1ccc(-c2cc3ccccc3cc2Nc2ccc(C3CCCCC3)cc2)cc1 ; Cc1ccc2c(c1)C1(c3ccccc3-c3ccc(N(c4ccc(C5CCCCC5)cc4)c4cc5ccccc5cc4-c4ccccc4)cc31)c1cc(C)ccc1-2]

Compound 41-1 (12 mmol, 1.2 eq), 2′-bromo-2,7-dimethyl-9,9′-spirobi[fluorene] (10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 5.3 mmol of Compound 81 (yield=53%).

### (20) Synthesis of Amine Compound 82

Amine Compound 82 according to one or more embodiments may be synthesized by, for example, the step of Reaction 20.

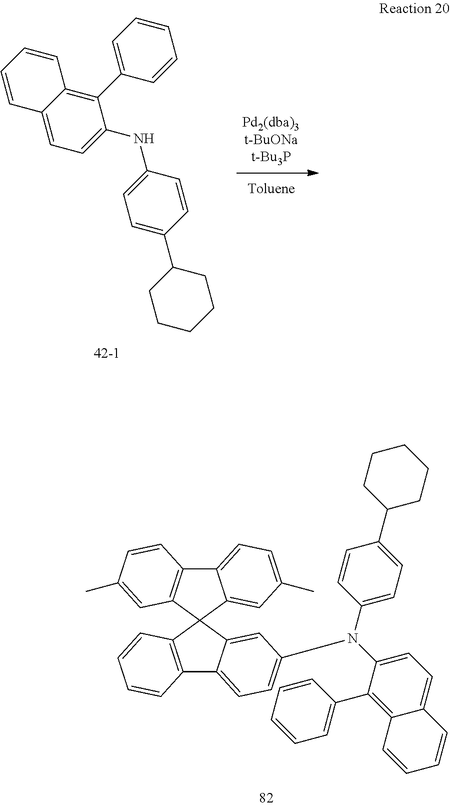 [SMILES: c1ccc(-c2c(Nc3ccc(C4CCCCC4)cc3)ccc3ccccc23)cc1 ; "42-1" ; Cc1ccc2c(c1)C1(c3ccccc3-c3ccc(N(c4ccc(C5CCCCC5)cc4)c4ccc5ccccc5c4-c4ccccc4)cc31)c1cc(C)ccc1-2]

Compound 42-1 (10 mmol, 1.2 eq), 2′-bromo-2,7-dimethyl-9,9′-spirobi[fluorene] (8 mmol, 1 eq), Pd₂(dba)₃ (0.4 mmol, 0.05 eq), t-BuONa (16 mmol, 2 eq), t-Bu₃P (0.8 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 5.4 mmol of Compound 82 (yield=54%).

### (21) Synthesis of Amine Compound 87

Amine Compound 87 according to one or more embodiments may be synthesized by, for example, the step of Reaction 21.

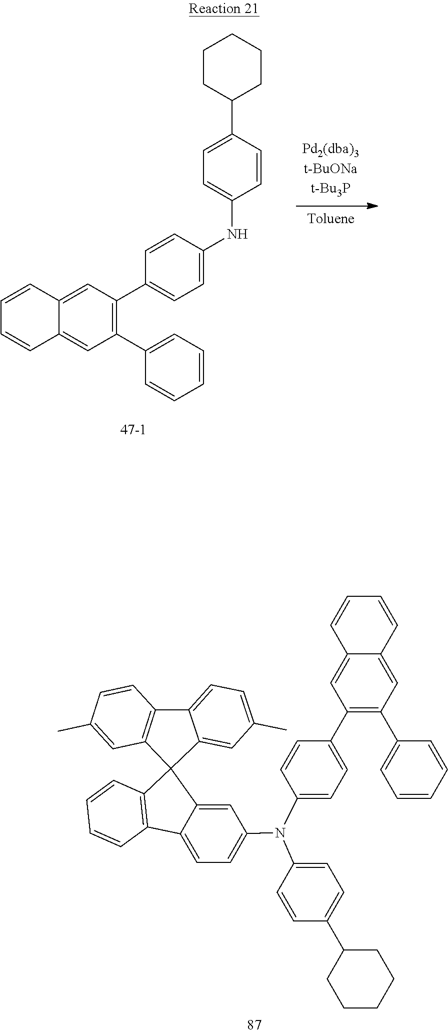 [SMILES: c1ccc(-c2cc3ccccc3cc2-c2ccc(Nc3ccc(C4CCCCC4)cc3)cc2)cc1 ; Cc1ccc2c(c1)C1(c3ccccc3-c3ccc(N(c4ccc(-c5cc6ccccc6cc5-c5ccccc5)cc4)c4ccc(C5CCCCC5)cc4)cc31)c1cc(C)ccc1-2]

Compound 47-1 (12 mmol, 1.2 eq), 2′-bromo-2,7-dimethyl-9,9′-spirobi[fluorene] (10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 5.2 mmol of Compound 87 (yield=52%).

### (22) Synthesis of Amine Compound 88

Amine Compound 88 according to one or more embodiments may be synthesized by, for example, the step of Reaction 22.

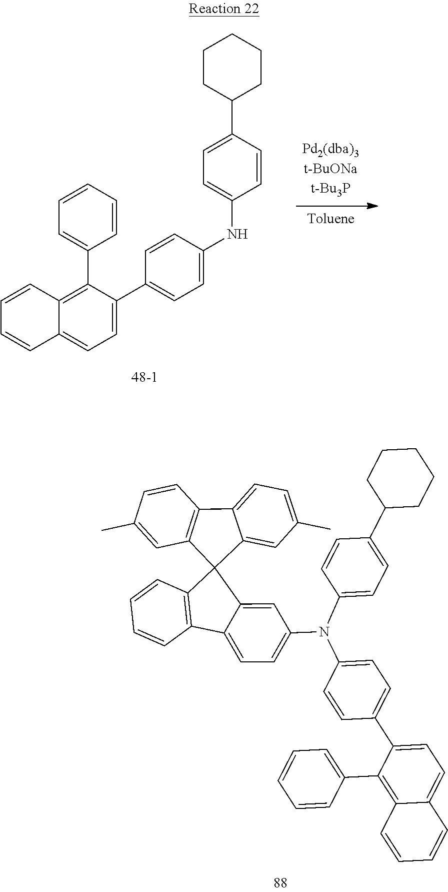 [SMILES: c1ccc(-c2c(-c3ccc(Nc4ccc(C5CCCCC5)cc4)cc3)ccc3ccccc23)cc1 ; Cc1ccc2c(c1)C1(c3ccccc3-c3ccc(N(c4ccc(-c5ccc6ccccc6c5-c5ccccc5)cc4)c4ccc(C5CCCCC5)cc4)cc31)c1cc(C)ccc1-2]

Compound 48-1 (12 mmol, 1.2 eq), 2′-bromo-2,7-dimethyl-9,9′-spirobi[fluorene] (10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 5.4 mmol of Compound 88 (yield=54%).

### (23) Synthesis of Amine Compound 121

Amine Compound 121 according to one or more embodiments may be synthesized by, for example, the step of Reaction 23.

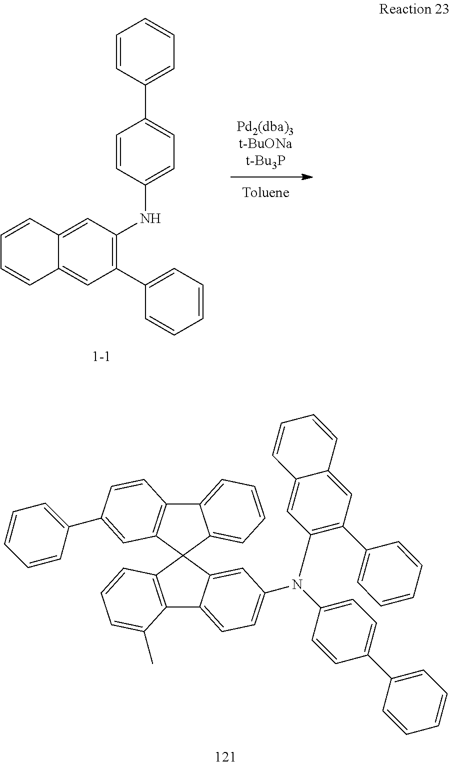 [SMILES: c1ccc(-c2ccc(Nc3cc4ccccc4cc3-c3ccccc3)cc2)cc1 ; "1-1" ; Cc1cccc2c1-c1ccc(N(c3ccc(-c4ccccc4)cc3)c3cc4ccccc4cc3-c3ccccc3)cc1C21c2ccccc2-c2ccc(-c3ccccc3)cc21]

Compound 1-1 (12 mmol, 1.2 eq), 2-bromo-5-methyl-2′-phenyl-9,9′-spirobi[fluorene] (10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 4.3 mmol of Compound 121 (yield=43%).

### (24) Synthesis of Amine Compound 122

Amine Compound 122 according to one or more embodiments may be synthesized by, for example, the step of Reaction 24.

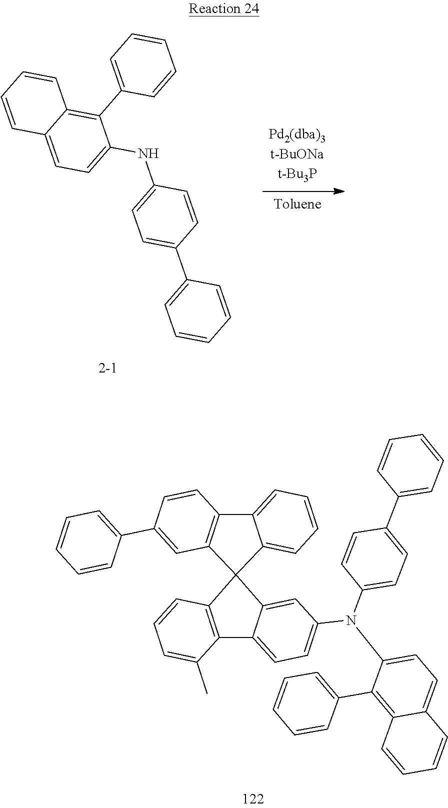 [SMILES: c1ccc(-c2ccc(Nc3ccc4ccccc4c3-c3ccccc3)cc2)cc1 ; Cc1cccc2c1-c1ccc(N(c3ccc(-c4ccccc4)cc3)c3ccc4ccccc4c3-c3ccccc3)cc1C21c2ccccc2-c2ccc(-c3ccccc3)cc21]

Compound 2-1 (12 mmol, 1.2 eq), 2-bromo-5-methyl-2′-phenyl-9,9′-spirobi[fluorene] (10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 4.1 mmol of Compound 122 (yield=41%).

### (25) Synthesis of Amine Compound 141

Amine Compound 141 according to one or more embodiments may be synthesized by, for example, the step of Reaction 25.

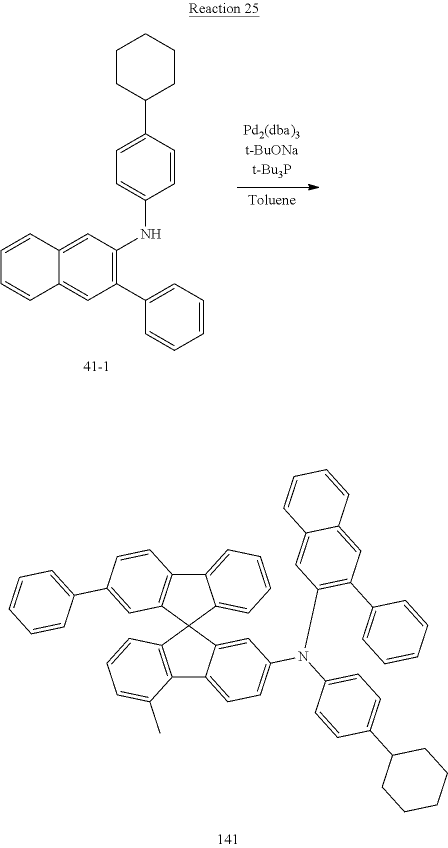 [SMILES: c1ccc(-c2cc3ccccc3cc2Nc2ccc(C3CCCCC3)cc2)cc1 ; Cc1cccc2c1-c1ccc(N(c3ccc(C4CCCCC4)cc3)c3cc4ccccc4cc3-c3ccccc3)cc1C21c2ccccc2-c2ccc(-c3ccccc3)cc21]

Compound 41-1 (12 mmol, 1.2 eq), 2-bromo-5-methyl-2′-phenyl-9,9′-spirobi[fluorene] (10 mmol, 1 eq), Pd₂(dba)₃ (0.5 mmol, 0.05 eq), t-BuONa (20 mmol, 2 eq), t-Bu₃P (0.1 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 4.3 mmol of Compound 141 (yield=43%).

### (26) Synthesis of Amine Compound 142

Amine Compound 142 according to one or more embodiments may be synthesized by, for example, the step of Reaction 26.

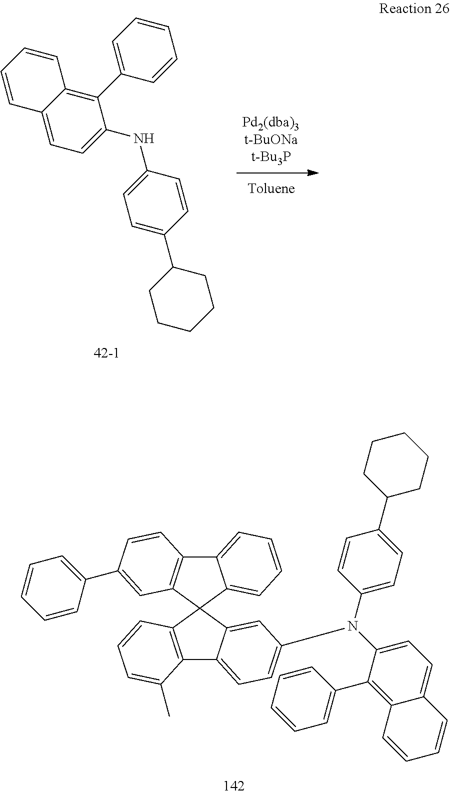 [SMILES: c1ccc(-c2c(Nc3ccc(C4CCCCC4)cc3)ccc3ccccc23)cc1 ; "42-1" ; Cc1cccc2c1-c1ccc(N(c3ccc(C4CCCCC4)cc3)c3ccc4ccccc4c3-c3ccccc3)cc1C21c2ccccc2-c2ccc(-c3ccccc3)cc21]

Compound 42-1 (10 mmol, 1.2 eq), 2-bromo-5-methyl-2′-phenyl-9,9′-spirobi[fluorene] (8 mmol, 1 eq), Pd₂(dba)₃ (0.4 mmol, 0.05 eq), t-BuONa (16 mmol, 2 eq), t-Bu₃P (0.8 mmol, 0.1 eq), and 150 mL of toluene were put in a 1-neck-round flask and stirred at about 110° C. (for about 1 hour).

After finishing the reaction, the resultant was worked-up with ether/H₂O, and separated by column chromatography utilizing dichloromethane/hexane=1/8 as an eluent to obtain 4.4 mmol of Compound 142 (yield=44%).

### 2. Manufacture and Evaluation of Light Emitting Elements

### (1) Manufacture of Light Emitting Element

A light emitting element including the amine compound of one or more embodiments or a comparative compound in a hole transport layer was manufactured by a method described herein. Light emitting elements of Example 1 to Example 24 were manufactured utilizing Compounds 1, 2, 7, 8, 10, 11, 41, 42, 47, 48, 61, 62, 67, 68, 70, 71, 81, 82, 87, 88, 121, 122, 141, and 142, which are the amine compounds of embodiments, as the materials of a hole transport layer. The light emitting element of Comparative Example 1 was manufactured utilizing a suitable material of NPB as the material of a hole transport layer. The light emitting element of Comparative Example 2 was manufactured utilizing Comparative Compound HT1 as the material of a hole transport layer. The light emitting element of Comparative Example 3 was manufactured utilizing Comparative Compound HT2 as the material of a hole transport layer. The light emitting element of Comparative Example 4 was manufactured utilizing Comparative Compound HT3 as the material of a hole transport layer. The light emitting element of Comparative Example 5 was manufactured utilizing Comparative Compound HT4 as the material of a hole transport layer.

An ITO glass substrate with about 15 Ω/cm² (about 1200 Å) of Corning Co. was cut into a size of 50 mm×50 mm×0.7 mm, washed with isopropyl alcohol and pure water, cleansed utilizing ultrasonic waves for about 5 minutes, exposed to UV for about 30 minutes and treated with ozone to form an anode. The ITO glass substrate was installed in a vacuum deposition apparatus.

On the substrate, 2-TNATA was vacuum deposited to a thickness of about 600 Å to form a hole injection layer, and then, the Example Compound or Comparative Compound was vacuum deposited to a thickness of about 300 Å to form a hole transport layer. On the hole transport layer, a blue fluorescence host of 9,10-di(naphthalen-2-yl)anthracene (hereinafter, DNA) and a blue fluorescence dopant of 4,4′-bis[2-(4-(N,N-diphenylamino)phenyl)vinyl]biphenyl (hereinafter, DPAVBi) were co-deposited in a ratio (e.g., amount) of about 98:2 to form an emission layer with a thickness of about 300 Å. On the emission layer, an electron transport layer was formed to a thickness of about 300 Å utilizing Alq₃, and then, an electron injection layer was formed to a thickness of about 10 Å by depositing an alkali metal halide of LiF. On the electron injection layer, Al was vacuum deposited to a thickness of about 3000 Å to form a LiF/Al electrode, thereby manufacturing a light emitting element.

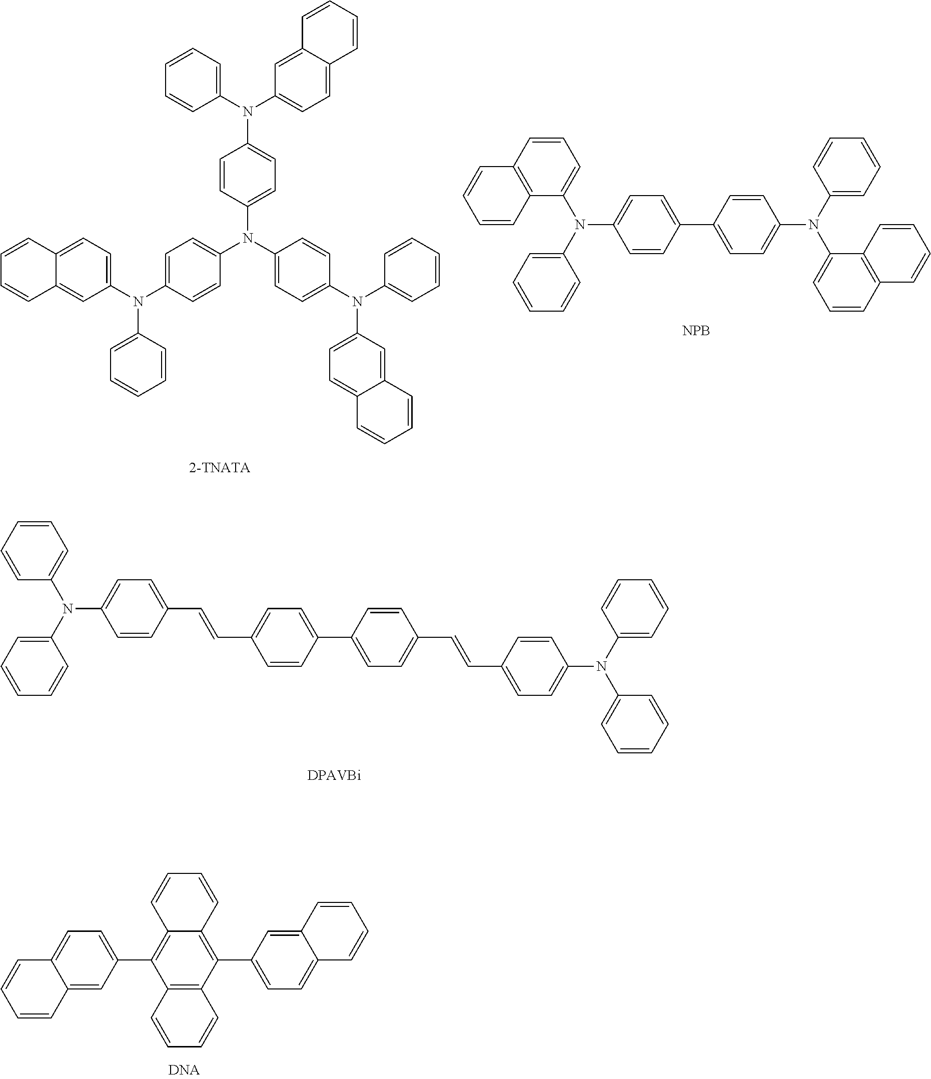

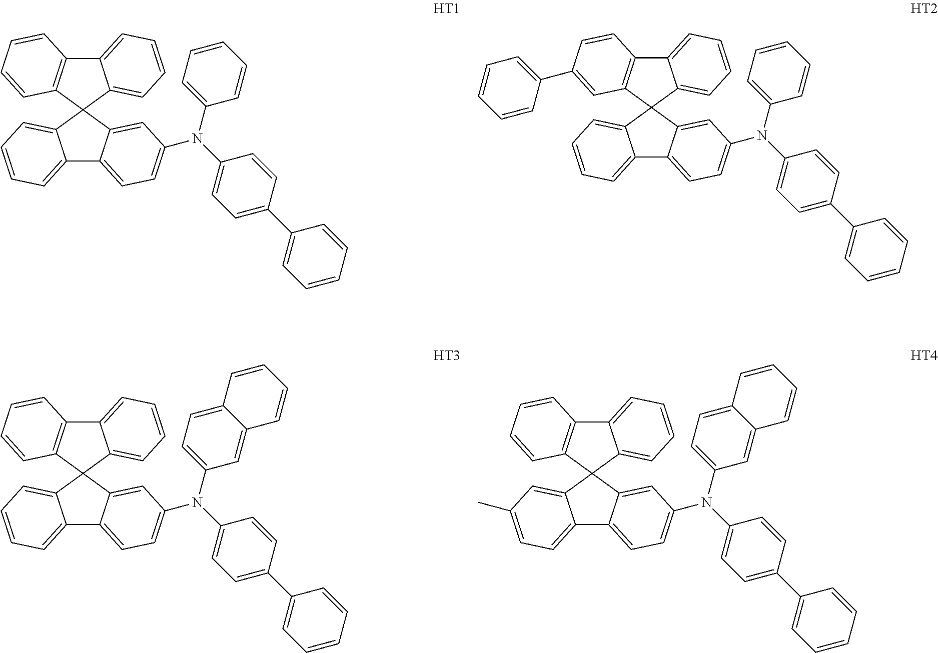 [SMILES: c1ccc(-c2ccc(N(c3ccccc3)c3ccc4c(c3)C3(c5ccccc5-c5ccccc53)c3ccccc3-4)cc2)cc1 ; c1ccc(-c2ccc(N(c3ccccc3)c3ccc4c(c3)C3(c5ccccc5-c5ccc(-c6ccccc6)cc53)c3ccccc3-4)cc2)cc1 ; c1ccc(-c2ccc(N(c3ccc4c(c3)C3(c5ccccc5-c5ccccc53)c3ccccc3-4)c3ccc4ccccc4c3)cc2)cc1 ; Cc1ccc2c(c1)C1(c3ccccc3-c3ccccc31)c1cc(N(c3ccc(-c4ccccc4)cc3)c3ccc4ccccc4c3)ccc1-2]

The Example Compounds and Comparative Compounds utilized in Examples 1 to 24, and Comparative Examples 1 to 5 are shown in Table 1.

TABLE 1

| | |
|---|---|
| Compound 1 | 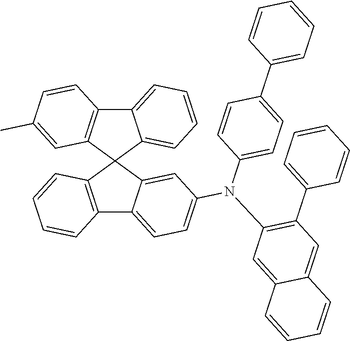 [SMILES: Cc1ccc2c(c1)C1(c3ccccc3-2)c2ccccc2-c2ccc(N(c3ccc(-c4ccccc4)cc3)c3cc4ccccc4cc3-c3ccccc3)cc21] |
| Compound 2 | 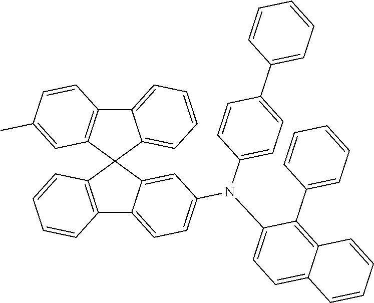 [SMILES: Cc1ccc2c(c1)C1(c3ccccc3-2)c2ccccc2-c2ccc(N(c3ccc(-c4ccccc4)cc3)c3ccc4ccccc4c3-c3ccccc3)cc21] |
| Compound 7 | 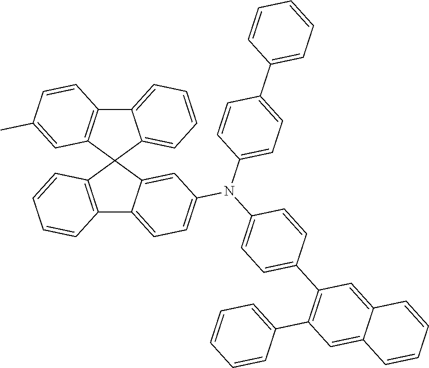 [SMILES: Cc1ccc2c(c1)C1(c3ccccc3-2)c2ccccc2-c2ccc(N(c3ccc(-c4ccccc4)cc3)c3ccc(-c4cc5ccccc5cc4-c4ccccc4)cc3)cc21] |
| Compound 8 | 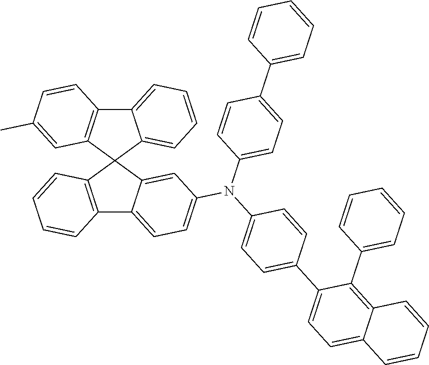 [SMILES: Cc1ccc2c(c1)C1(c3ccccc3-2)c2ccccc2-c2ccc(N(c3ccc(-c4ccccc4)cc3)c3ccc(-c4ccc5ccccc5c4-c4ccccc4)cc3)cc21] |
| Compound 10 | 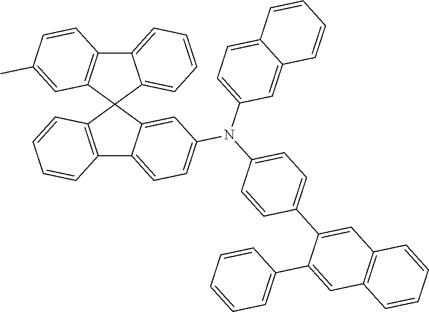 [SMILES: Cc1ccc2c(c1)C1(c3ccccc3-2)c2ccccc2-c2ccc(N(c3ccc(-c4cc5ccccc5cc4-c4ccccc4)cc3)c3ccc4ccccc4c3)cc21] |
| Compound 11 | 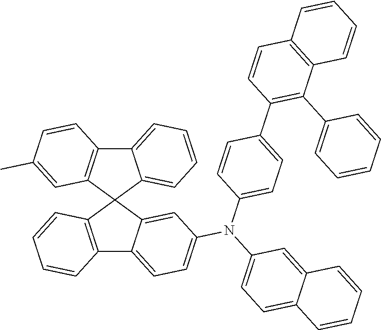 [SMILES: Cc1ccc2c(c1)C1(c3ccccc3-2)c2ccccc2-c2ccc(N(c3ccc(-c4ccc5ccccc5c4-c4ccccc4)cc3)c3ccc4ccccc4c3)cc21] |
| Compound 41 | 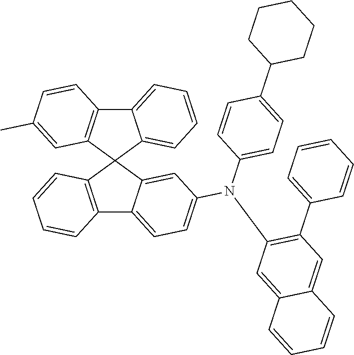 [SMILES: Cc1ccc2c(c1)C1(c3ccccc3-2)c2ccccc2-c2ccc(N(c3ccc(C4CCCCC4)cc3)c3cc4ccccc4cc3-c3ccccc3)cc21] |
| Compound 42 | 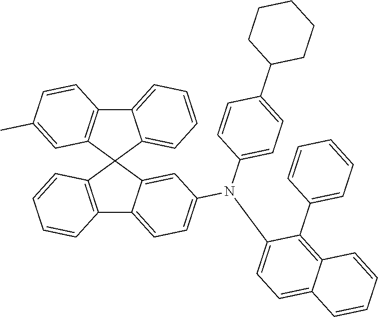 [SMILES: Cc1ccc2c(c1)C1(c3ccccc3-2)c2ccccc2-c2ccc(N(c3ccc(C4CCCCC4)cc3)c3ccc4ccccc4c3-c3ccccc3)cc21] |
| Compound 47 | 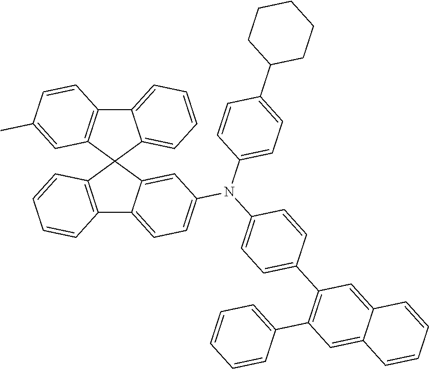 [SMILES: Cc1ccc2c(c1)C1(c3ccccc3-2)c2ccccc2-c2ccc(N(c3ccc(-c4cc5ccccc5cc4-c4ccccc4)cc3)c3ccc(C4CCCCC4)cc3)cc21] |
| Compound 48 | 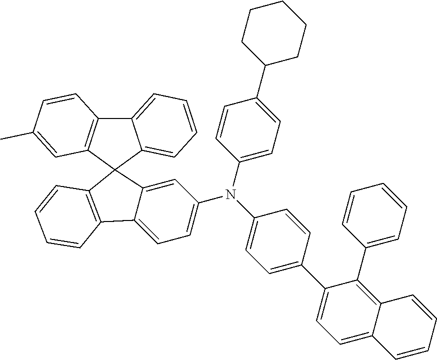 [SMILES: Cc1ccc2c(c1)C1(c3ccccc3-2)c2ccccc2-c2ccc(N(c3ccc(-c4ccc5ccccc5c4-c4ccccc4)cc3)c3ccc(C4CCCCC4)cc3)cc21] |
| Compound 61 | 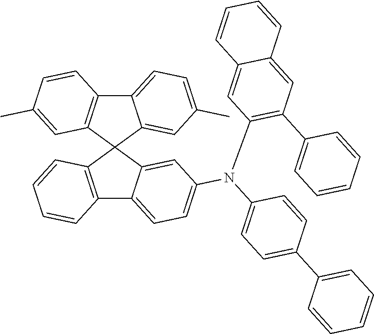 [SMILES: Cc1ccc2c(c1)C1(c3ccccc3-c3ccc(N(c4ccc(-c5ccccc5)cc4)c4cc5ccccc5cc4-c4ccccc4)cc31)c1cc(C)ccc1-2] |
| Compound 62 | 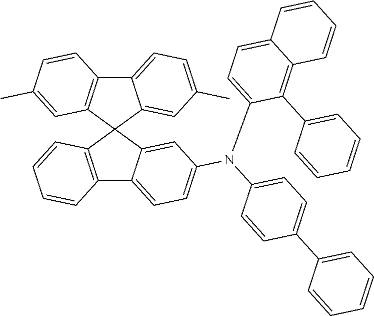 [SMILES: Cc1ccc2c(c1)C1(c3ccccc3-c3ccc(N(c4ccc(-c5ccccc5)cc4)c4ccc5ccccc5c4-c4ccccc4)cc31)c1cc(C)ccc1-2] |
| Compound 67 | 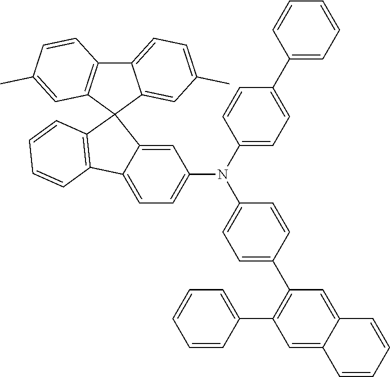 [SMILES: Cc1ccc2c(c1)C1(c3ccccc3-c3ccc(N(c4ccc(-c5ccccc5)cc4)c4ccc(-c5cc6ccccc6cc5-c5ccccc5)cc4)cc31)c1cc(C)ccc1-2] |
| Compound 68 | 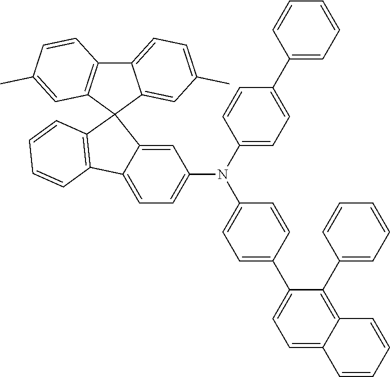 [SMILES: Cc1ccc2c(c1)C1(c3ccccc3-c3ccc(N(c4ccc(-c5ccccc5)cc4)c4ccc(-c5ccc6ccccc6c5-c5ccccc5)cc4)cc31)c1cc(C)ccc1-2] |
| Compound 70 | 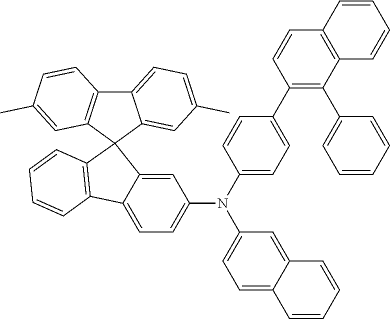 [SMILES: Cc1ccc2c(c1)C1(c3ccccc3-c3ccc(N(c4ccc(-c5ccc6ccccc6c5-c5ccccc5)cc4)c4ccc5ccccc5c4)cc31)c1cc(C)ccc1-2] |
| Compound 71 | 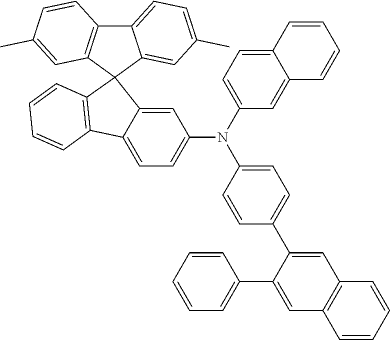 [SMILES: Cc1ccc2c(c1)C1(c3ccccc3-c3ccc(N(c4ccc(-c5cc6ccccc6cc5-c5ccccc5)cc4)c4ccc5ccccc5c4)cc31)c1cc(C)ccc1-2] |
| Compound 81 | 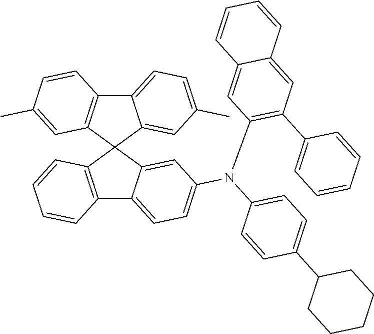 [SMILES: Cc1ccc2c(c1)C1(c3ccccc3-c3ccc(N(c4ccc(C5CCCCC5)cc4)c4cc5ccccc5cc4-c4ccccc4)cc31)c1cc(C)ccc1-2] |
| Compound 82 | 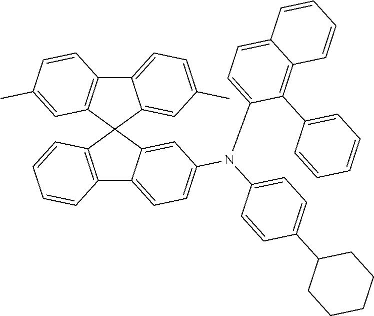 [SMILES: Cc1ccc2c(c1)C1(c3ccccc3-c3ccc(N(c4ccc(C5CCCCC5)cc4)c4ccc5ccccc5c4-c4ccccc4)cc31)c1cc(C)ccc1-2] |
| Compound 87 | 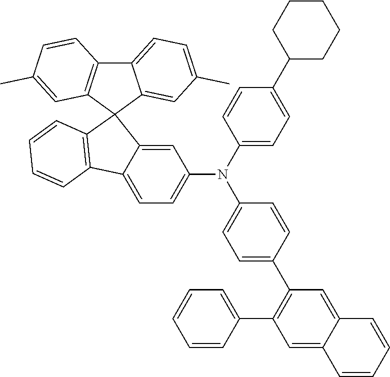 [SMILES: Cc1ccc2c(c1)C1(c3ccccc3-c3ccc(N(c4ccc(-c5cc6ccccc6cc5-c5ccccc5)cc4)c4ccc(C5CCCCC5)cc4)cc31)c1cc(C)ccc1-2] |
| Compound 88 | 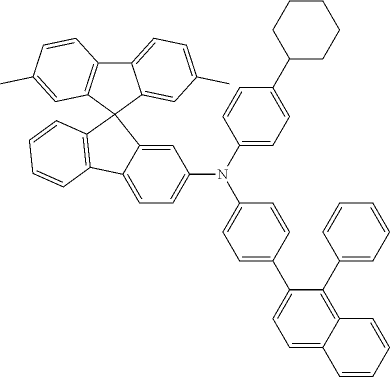 [SMILES: Cc1ccc2c(c1)C1(c3ccccc3-c3ccc(N(c4ccc(-c5ccc6ccccc6c5-c5ccccc5)cc4)c4ccc(C5CCCCC5)cc4)cc31)c1cc(C)ccc1-2] |
| Compound 121 | 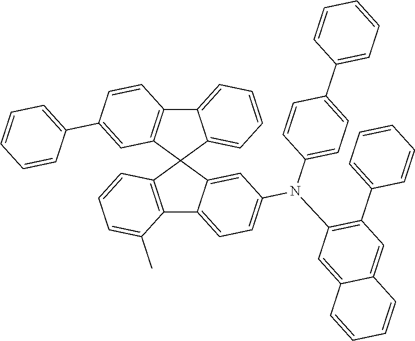 [SMILES: Cc1cccc2c1-c1ccc(N(c3ccc(-c4ccccc4)cc3)c3cc4ccccc4cc3-c3ccccc3)cc1C21c2ccccc2-c2ccc(-c3ccccc3)cc21] |
| Compound 122 | 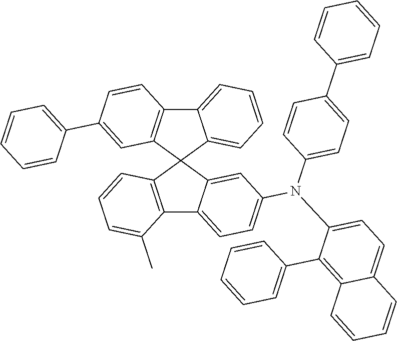 [SMILES: Cc1cccc2c1-c1ccc(N(c3ccc(-c4ccccc4)cc3)c3ccc4ccccc4c3-c3ccccc3)cc1C21c2ccccc2-c2ccc(-c3ccccc3)cc21] |
| Compound 141 | 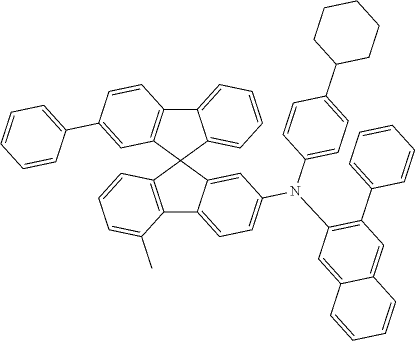 [SMILES: Cc1cccc2c1-c1ccc(N(c3ccc(C4CCCCC4)cc3)c3cc4ccccc4cc3-c3ccccc3)cc1C21c2ccccc2-c2ccc(-c3ccccc3)cc21] |
| Compound 142 | 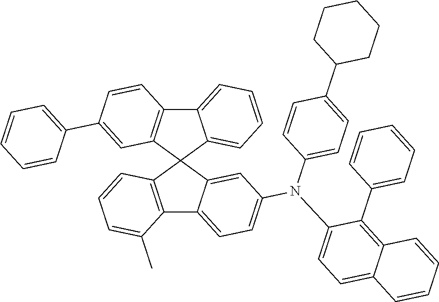 [SMILES: Cc1cccc2c1-c1ccc(N(c3ccc(C4CCCCC4)cc3)c3ccc4ccccc4c3-c3ccccc3)cc1C21c2ccccc2-c2ccc(-c3ccccc3)cc21] |
| NPB | 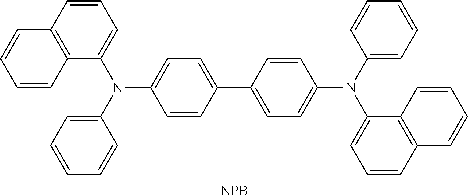 [SMILES: c1ccc(N(c2ccc(-c3ccc(N(c4ccccc4)c4cccc5ccccc45)cc3)cc2)c2cccc3ccccc23)cc1 ; "NPB"] |
| HT1 | 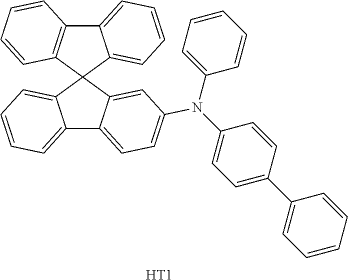 [SMILES: c1ccc(-c2ccc(N(c3ccccc3)c3ccc4c(c3)C3(c5ccccc5-c5ccccc53)c3ccccc3-4)cc2)cc1 ; "HT1"] |
| HT2 | 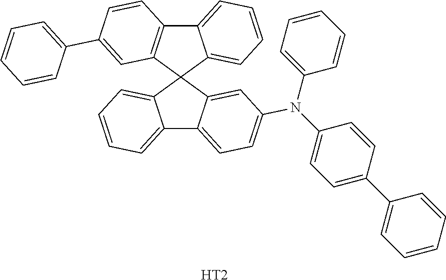 [SMILES: c1ccc(-c2ccc(N(c3ccccc3)c3ccc4c(c3)C3(c5ccccc5-c5ccc(-c6ccccc6)cc53)c3ccccc3-4)cc2)cc1 ; "HT2"] |
| HT3 | 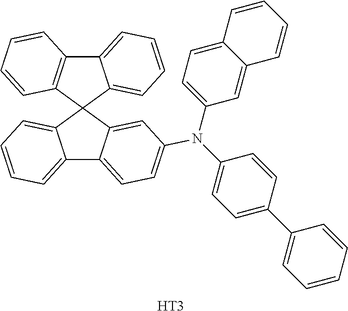 [SMILES: c1ccc(-c2ccc(N(c3ccc4c(c3)C3(c5ccccc5-c5ccccc53)c3ccccc3-4)c3ccc4ccccc4c3)cc2)cc1 ; "HT3"] |
| HT4 | 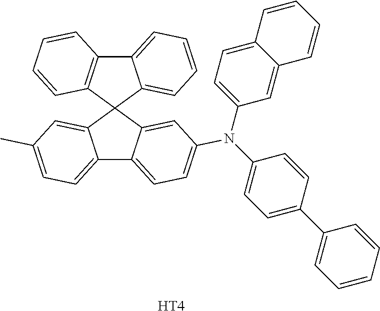 [SMILES: Cc1ccc2c(c1)C1(c3ccccc3-c3ccccc31)c1cc(N(c3ccc(-c4ccccc4)cc3)c3ccc4ccccc4c3)ccc1-2 ; "HT4"] |

### (2) Evaluation of Light Emitting Element

Table 2 shows evaluation results of driving voltage, luminance, efficiency, and half life of the light emitting elements of Examples and Comparative Examples. An evaluation equipment was I-V-L Test System Polaronix V7000 (manufacturer: DichloromethaneSience Inc.). The voltages, luminance (characteristics), and efficiency (characteristics) were evaluated based on a current density of about 50 mA/cm². The half life was evaluated based on a current density of about 100 mA/cm².

TABLE 2

| Element | | Driving | Current | | | | Half |
|---|---|---|---|---|---|---|---|
| manufacturing | Hole transport | voltage | density | Luminance | Efficiency | Emission | life |
| example | layer | (V) | (mA/cm²) | (cd/m²) | (cd/A) | color | (time) |
| Comparative | NPB | 7.01 | 50 | 2645 | 5.29 | Blue | 258 |
| Example 1 | | | | | | | |
| Comparative | HT1 | 5.18 | 50 | 2050 | 4.12 | Blue | 320 |
| Example 2 | | | | | | | |
| Comparative | HT2 | 5.00 | 50 | 2250 | 4.50 | Blue | 380 |
| Example 3 | | | | | | | |
| Comparative | HT3 | 5.18 | 50 | 2040 | 4.10 | Blue | 360 |
| Example 4 | | | | | | | |
| Comparative | HT4 | 5.80 | 50 | 2050 | 4.10 | Blue | 220 |
| Example 5 | | | | | | | |
| Example 1 | Compound 1 | 4.71 | 50 | 3240 | 6.48 | Blue | 520 |
| Example 2 | Compound 2 | 4.61 | 50 | 3260 | 6.52 | Blue | 550 |
| Example 3 | Compound 7 | 4.65 | 50 | 3210 | 6.42 | Blue | 560 |
| Example 4 | Compound 8 | 4.65 | 50 | 3200 | 6.40 | Blue | 540 |
| Example 5 | Compound 10 | 4.57 | 50 | 3300 | 6.60 | Blue | 560 |
| Example 6 | Compound 11 | 4.53 | 50 | 3200 | 6.40 | Blue | 570 |
| Example 7 | Compound 41 | 4.54 | 50 | 3250 | 6.50 | Blue | 560 |
| Example 8 | Compound 42 | 4.61 | 50 | 3250 | 6.50 | Blue | 560 |
| Example 9 | Compound 47 | 4.79 | 50 | 3300 | 6.60 | Blue | 550 |
| Example 10 | Compound 48 | 4.59 | 50 | 3280 | 6.56 | Blue | 550 |
| Example 11 | Compound 61 | 4.48 | 50 | 3340 | 6.68 | Blue | 630 |
| Example 12 | Compound 62 | 4.52 | 50 | 3400 | 6.70 | Blue | 645 |
| Example 13 | Compound 67 | 4.52 | 50 | 3320 | 6.64 | Blue | 630 |
| Example 14 | Compound 68 | 4.55 | 50 | 3370 | 6.74 | Blue | 610 |
| Example 15 | Compound 70 | 4.51 | 50 | 3290 | 6.58 | Blue | 600 |
| Example 16 | Compound 71 | 4.52 | 50 | 3320 | 6.64 | Blue | 590 |
| Example 17 | Compound 81 | 4.65 | 50 | 3270 | 6.54 | Blue | 560 |
| Example 18 | Compound 82 | 4.55 | 50 | 3290 | 6.58 | Blue | 575 |
| Example 19 | Compound 87 | 4.53 | 50 | 3300 | 6.60 | Blue | 560 |
| Example 20 | Compound 88 | 4.60 | 50 | 3330 | 6.66 | Blue | 570 |
| Example 21 | Compound 121 | 4.65 | 50 | 3340 | 6.68 | Blue | 530 |
| Example 22 | Compound 122 | 4.63 | 50 | 3290 | 6.58 | Blue | 560 |
| Example 23 | Compound 141 | 4.61 | 50 | 3200 | 6.40 | Blue | 520 |
| Example 24 | Compound 142 | 4.67 | 50 | 3240 | 6.48 | Blue | 550 |

Referring to the results of Table 2, it could be found that the light emitting elements of Example 1 to Example 24 showed reduced driving voltages and improved luminance when compared to the light emitting elements of Comparative Examples 1 to 5. In addition, it could be found that the light emitting elements of Examples 1 to 24 each showed improved efficiency and increased lifetime.

The light emitting elements of Examples 1 to 24 include Compounds 1, 2, 7, 8, 10, 11, 41, 42, 47, 48, 61, 62, 67, 68, 70, 71, 81, 82, 87, 88, 121, 122, 141, and 142, respectively, and Compounds 1, 2, 7, 8, 10, 11, 41, 42, 47, 48, 61, 62, 67, 68, 70, 71, 81, 82, 87, 88, 121, 122, 141, and 142 are amine compounds of embodiments of the present disclosure. The amine compound of one or more embodiments includes a naphthyl group, and a spiro fluorene moiety substituted with at least one methyl group, and the spiro fluorene moiety is directly bonded to the nitrogen atom of the amine group of the amine compound, and the naphthyl group may be directly or indirectly combined with the nitrogen atom of the amine group. As a result, the light emitting element of one or more embodiments, including the amine compound of one or more embodiments may show a low driving voltage, high luminance, high efficiency, and long-life characteristics.

The light emitting element of Comparative Example 1 includes Comparative Compound NPB, and Comparative Compound NPB includes two phenyl groups and a naphthyl group and is a diamine compound. It could be found that the light emitting elements of Examples 1 to 24 have low driving voltages, high luminance and efficiency characteristics, and long lifetime characteristics when compared to the light emitting element of Comparative Example 1 including Comparative Compound NPB.

The light emitting element of Comparative Example 2 includes Comparative Compound HT1, and Comparative Compound HT1 includes two phenyl groups and a spiro fluorenyl group which is unsubstituted with a methyl group. It could be found that the light emitting elements of Examples 1 to 24 have low driving voltages, high luminance and efficiency characteristics, and long lifetime characteristics when compared to the light emitting element of Comparative Example 2 including Comparative Compound HT1.

The light emitting element of Comparative Example 3 includes Comparative Compound HT2, and Comparative Compound HT2 includes two phenyl groups and a spiro fluorenyl group substituted with a phenyl group instead of a methyl group. It could be found that the light emitting elements of Examples 1 to 24 have low driving voltages, high luminance and efficiency characteristics, and long lifetime characteristics when compared to the light emitting element of Comparative Example 3 including Comparative Compound HT2.

The light emitting element of Comparative Example 4 includes Comparative Compound HT3, and the Comparative Compound HT3 includes a phenyl group, a naphthyl group and an unsubstituted spiro fluorenyl group. It could be found that the light emitting elements of Examples 1 to 24 have low driving voltages, high luminance and efficiency characteristics, and long lifetime characteristics when compared to the light emitting element of Comparative Example 4 including Comparative Compound HT3.

The light emitting element of Comparative Example 5 includes Comparative Compound HT4, and Comparative Compound HT4 includes a phenyl group, a naphthyl group and a spiro fluorenyl group in which a methyl group is substituted at a R₃₂ position based on Formula 1. It could be found that the light emitting elements of Examples 1 to 24 have low driving voltages, high luminance and efficiency characteristics, and long lifetime characteristics when compared to the light emitting element of Comparative Example 5 including Comparative Compound HT4.

When comparing Comparative Examples 1 to 4, it could be found that the light emitting elements of Examples 1 to 24 include an amine compound including a naphthyl group and a spiro fluorene moiety substituted with at least one methyl group, in a hole transport layer, and low driving voltages, high luminance, high efficiency and long-life characteristics are shown. In addition, when comparing the light emitting elements of Examples 1 to 24 with Comparative Example 5, it could be found that the case of including an amine compound in which methyl groups are substituted at R₁₂, R₂₃, and/or R₃₄ positions based on Formula 1, in a hole transport layer, showed a low driving voltage, high luminance, high efficiency and long-life characteristics when compared to the case of including an amine compound in which a methyl group is substituted at a R₃₂ position.

The amine compound of one or more embodiments of present disclosure includes a naphthyl group and a spiro fluorene moiety in which at least one methyl group is substituted at R₁₂, R₂₃, and/or R₃₄ positions of the spiro fluorene moiety, and shows excellent or suitable hole transport properties. Accordingly, it is considered that the light emitting element of one or more embodiments, including the amine compound of one or more embodiments shows a low driving voltage, high luminance and efficiency and long lifetime when compared to the light emitting element of the Comparative Example.

Based on Formula 1, when at least one selected from among R₁₂, R₂₃ and R₃₄ of a spiro fluorenyl group is a methyl group, a driving voltage may be reduced, and lifetime may be increased. In addition, a naphthyl group substituted with an aryl or alkyl group may increase carrier mobility in a molecule to play the role of reducing a driving voltage. Accordingly, a light emitting element including a hole transport region including an amine compound including a spiro fluorenyl group including at least one methyl group, and a naphthyl group, may show a low driving voltage, high luminance, high efficiency, and long-life characteristics.

The light emitting element of one or more embodiments may include a first electrode, a second electrode disposed on the first electrode, and at least one functional layer disposed between the first electrode and the second electrode. The at least one functional layer may include the amine compound of one or more embodiments of the present disclosure. The light emitting element of one or more embodiments including the amine compound of one or more embodiments may show a low driving voltage, high luminance, high efficiency, and long-life characteristics.

The amine compound of one or more embodiments may include a spiro fluorene moiety substituted with at least one methyl group, and a naphthyl group. Consequently, the amine compound of one or more embodiments may show a high glass transition temperature and may prevent or reduce crystallization. In addition, the amine compound of one or more embodiments may show low refraction index properties and excellent or suitable hole transport properties.

The light emitting element of one or more embodiments may include the amine compound of one or more embodiments and may show a low driving voltage, high luminance, high efficiency, and long-life characteristics.

The amine compound of one or more embodiments may contribute to the reduction of the driving voltage, the improvement of the luminance and efficiency, and the increase of the lifetime of a light emitting element.

Throughout the present disclosure, when a component such as a layer, a film, a region, or a plate is mentioned to be placed “on” another component, it will be understood that it may be directly on another component or that another component may be interposed therebetween. In some embodiments, “directly on” may refer to that there are no additional layers, films, regions, plates, etc., between a layer, a film, a region, a plate, etc. and the other part. For example, “directly on” may refer to two layers or two members are disposed without utilizing an additional member such as an adhesive member therebetween.

As utilized herein, the singular forms “a,” “an,” “one,” and “the” are intended to include the plural forms as well, unless the context clearly indicates otherwise. Further, the use of “may” when describing embodiments of the present disclosure refers to “one or more embodiments of the present disclosure”.

In the present disclosure, when particles are spherical, “diameter” indicates a particle diameter or an average particle diameter, and when the particles are non-spherical, the “diameter” indicates a major axis length or an average major axis length. The diameter (or size) of the particles may be measured utilizing a scanning electron microscope or a particle size analyzer. As the particle size analyzer, for example, HORIBA, LA-950 laser particle size analyzer, may be utilized. When the size of the particles is measured utilizing a particle size analyzer, the average particle diameter (or size) is referred to as D50. D50 refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size.

As utilized herein, the terms “substantially,” “about,” or similar terms are used as terms of approximation and not as terms of degree, and are intended to account for the inherent deviations in measured or calculated values that would be recognized by those of ordinary skill in the art. “About” as used herein, is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” may mean within one or more standard deviations, or within ±30%, 20%, 10%, 5% of the stated value.

Any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The light-emitting element, the display device, or any other relevant devices or components according to embodiments of the present disclosure described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

Although the embodiments of the present disclosure have been described, it is understood that the present disclosure should not be limited to these embodiments, but one or more suitable changes and modifications can be made by one ordinary skilled in the art within the spirit and scope of the present disclosure as hereinafter claimed and equivalent thereof.

What is claimed is:

1. A light emitting element, comprising:
a first electrode;
a second electrode on the first electrode;
an emission layer between the first electrode and the second electrode; and
a hole transport region between the first electrode and the emission layer, and comprising an amine compound represented by Formula 1: 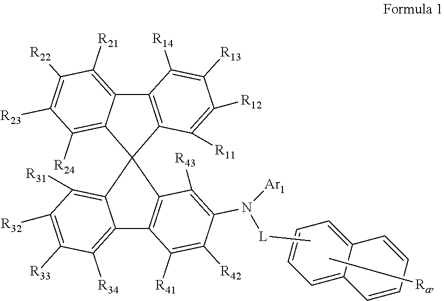
wherein, in Formula 1,
at least one selected from among R₁₁ to R₁₄, R₂₁ to R₂₄, R₃₁ to R₃₄, and R₄₁ to R₄₃ is a substituted or unsubstituted methyl group, and the remainder are each independently hydrogen, deuterium, a halogen, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring,
Ar₁ is a substituted or unsubstituted aryl group of 6 to 30 ring-forming carbon atoms, or a substituted or unsubstituted heteroaryl group of 2 to 30 ring-forming carbon atoms,
L is a direct linkage, a substituted or unsubstituted arylene group of 6 to 30 ring-forming carbon atoms, or a substituted or unsubstituted heteroarylene group of 2 to 30 ring-forming carbon atoms, and
Rₐ is hydrogen, deuterium, a halogen, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group from each other to form a ring.

2. The light emitting element of claim 1, wherein, in Formula 1, at least one selected from among R₁₂, R₂₃, and R₃₄ is an unsubstituted methyl group.

3. The light emitting element of claim 2, wherein Formula 1 is represented by any one selected from among Formula 1-1 to Formula 1-4: 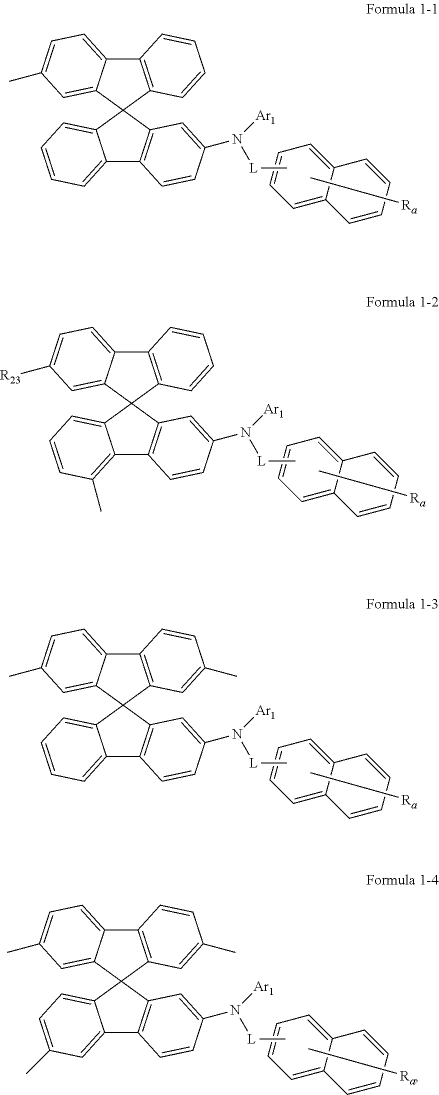
wherein, in Formula 1-1 to Formula 1-4, R₂₃, Ar₁, L, and Rₐ are the same as defined in Formula 1.

4. The light emitting element of claim 3, wherein, in Formula 1-2, R₂₃ is hydrogen, an unsubstituted methyl group, an unsubstituted phenyl group, or an unsubstituted t-butyl group.

5. The light emitting element of claim 1, wherein Formula 1 is represented by Formula 2: 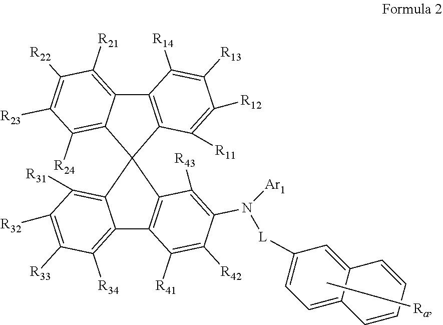
wherein, in Formula 2, R₁₁ to R₁₄, R₂₁ to R₂₄, R₃₁ to R₃₄, R₄₁ to R₄₃, Ar₁, L, and Rₐ are the same as defined in Formula 1.

6. The light emitting element of claim 5, wherein Formula 2 is represented by Formula 3-1 or Formula 3-2: 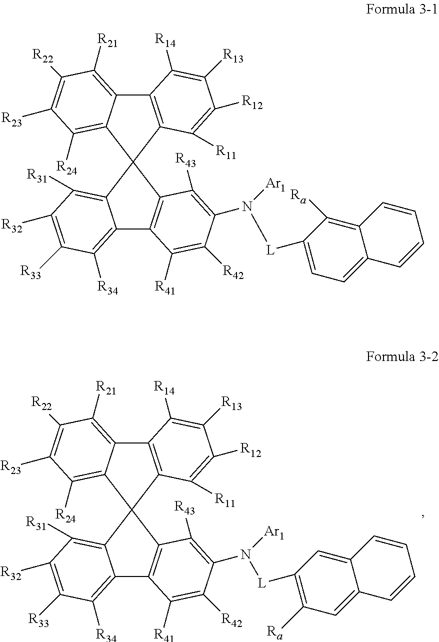
and
wherein, in Formula 3-1 and Formula 3-2, R₁₁ to R₁₄, R₂₁ to R₂₄, R₃₁ to R₃₄, R₄₁ to R₄₃, Ar₁, L, and Rₐ are the same as defined in Formula 1.

7. The light emitting element of claim 1, wherein, in Formula 1, Rₐ is a substituted or unsubstituted phenyl group, or a substituted or unsubstituted methyl group.

8. The light emitting element of claim 1, wherein Formula 1 is represented by Formula 4-1 or Formula 4-2: 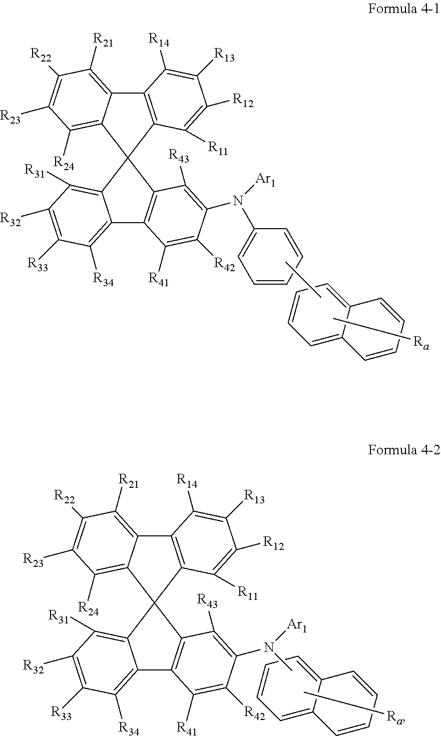
and
wherein, in Formula 4-1 and Formula 4-2, R₁₁ to R₁₄, R₂₁ to R₂₄, R₃₁ to R₃₄, R₄₁ to R₄₃, Ar¹, and Rₐ are the same as defined in Formula 1.

9. The light emitting element of claim 8, wherein Formula 4-1 is represented by Formula 5: 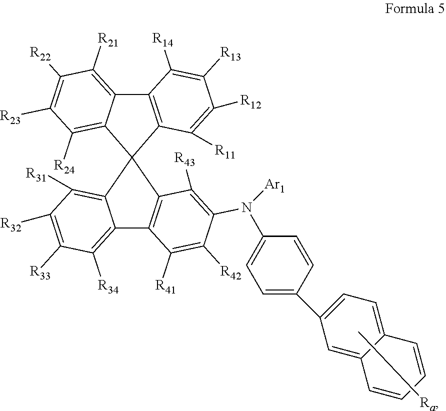
and
wherein, in Formula 5, R₁₁ to R₁₄, R₂₁ to R₂₄, R₃₁ to R₃₄, R₄₁ to R₄₃, Ar₁, and Rₐ are the same as defined in Formula 1.

10. The light emitting element of claim 1, wherein the amine compound represented by Formula 1 is a monoamine compound.

11. The light emitting element of claim 1, wherein the hole transport region comprises:
a hole injection layer on the first electrode; and
a hole transport layer between the hole injection layer and the emission layer, and
the hole transport layer comprises the amine compound represented by Formula 1.

12. The light emitting element of claim 1, wherein the emission layer is to emit blue light.

13. The light emitting element of claim 1, wherein the amine compound comprises at least one selected from among compounds in Compound Group 1:

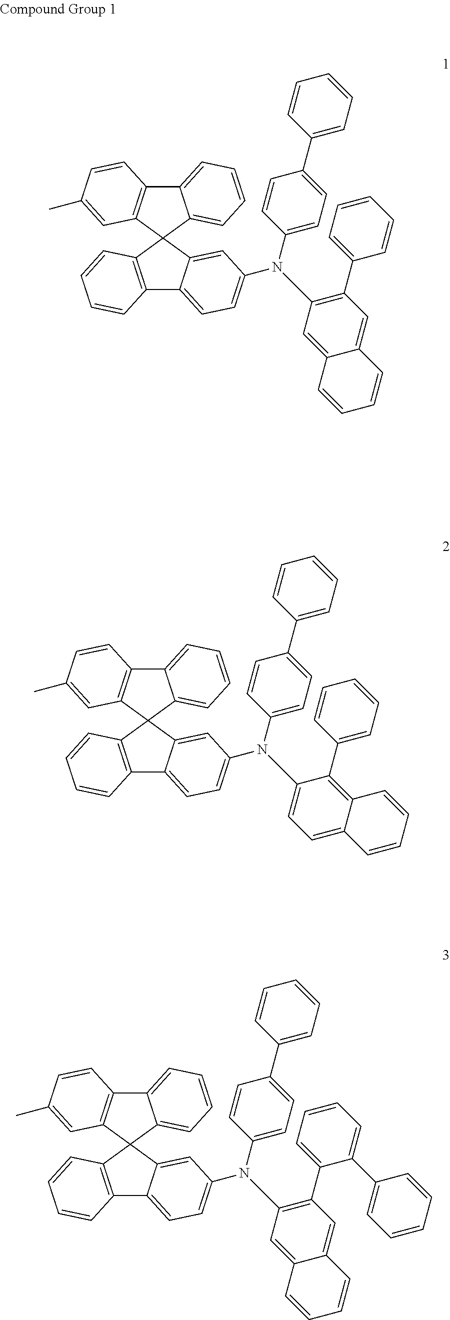 [SMILES: Cc1ccc2c(c1)C1(c3ccccc3-2)c2ccccc2-c2ccc(N(c3ccc(-c4ccccc4)cc3)c3cc4ccccc4cc3-c3ccccc3)cc21 ; Cc1ccc2c(c1)C1(c3ccccc3-2)c2ccccc2-c2ccc(N(c3ccc(-c4ccccc4)cc3)c3ccc4ccccc4c3-c3ccccc3)cc21 ; Cc1ccc2c(c1)C1(c3ccccc3-2)c2ccccc2-c2ccc(N(c3ccc(-c4ccccc4)cc3)c3cc4ccccc4cc3-c3ccccc3-c3ccccc3)cc21]

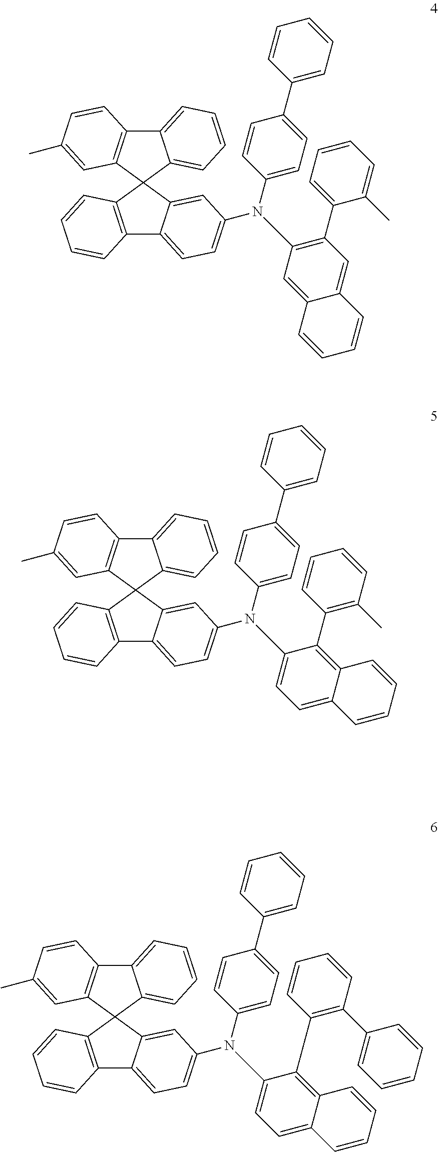

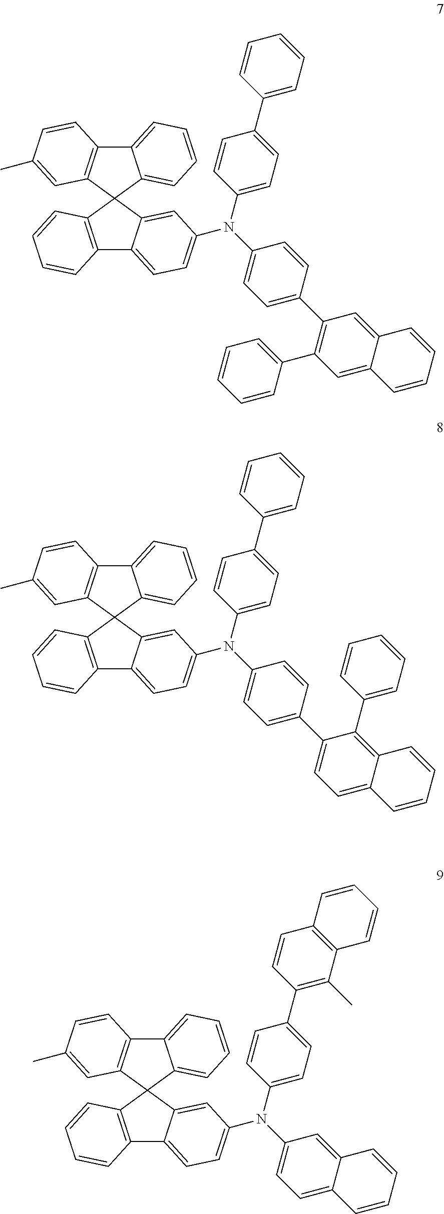 [SMILES: Cc1ccc2c(c1)C1(c3ccccc3-2)c2ccccc2-c2ccc(N(c3ccc(-c4ccccc4)cc3)c3ccc(-c4cc5ccccc5cc4-c4ccccc4)cc3)cc21 ; Cc1ccc2c(c1)C1(c3ccccc3-2)c2ccccc2-c2ccc(N(c3ccc(-c4ccccc4)cc3)c3ccc(-c4ccc5ccccc5c4-c4ccccc4)cc3)cc21 ; Cc1ccc2c(c1)C1(c3ccccc3-2)c2ccccc2-c2ccc(N(c3ccc(-c4ccc5ccccc5c4C)cc3)c3ccc4ccccc4c3)cc21]

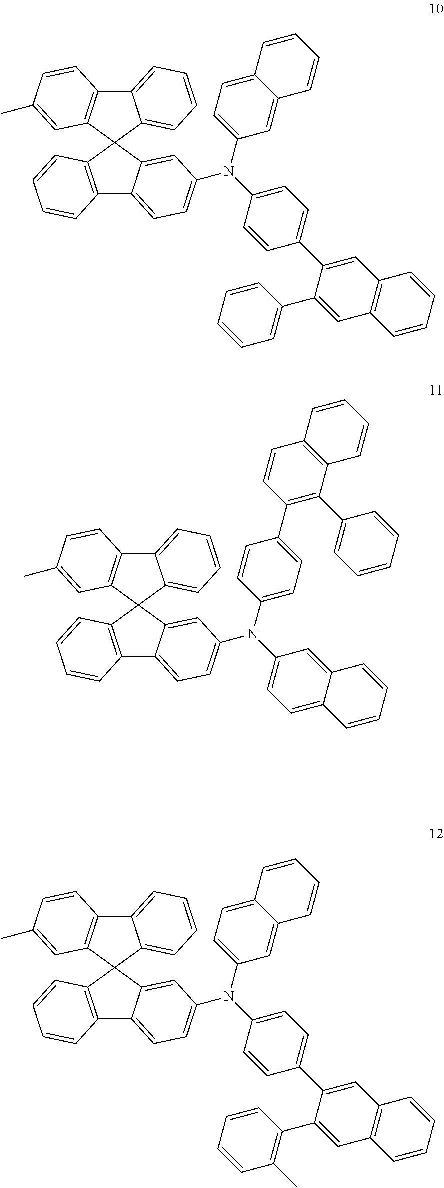 [SMILES: Cc1ccc2c(c1)C1(c3ccccc3-2)c2ccccc2-c2ccc(N(c3ccc(-c4cc5ccccc5cc4-c4ccccc4)cc3)c3ccc4ccccc4c3)cc21 ; Cc1ccc2c(c1)C1(c3ccccc3-2)c2ccccc2-c2ccc(N(c3ccc(-c4ccc5ccccc5c4-c4ccccc4)cc3)c3ccc4ccccc4c3)cc21 ; Cc1ccc2c(c1)C1(c3ccccc3-2)c2ccccc2-c2ccc(N(c3ccc(-c4cc5ccccc5cc4-c4ccccc4C)cc3)c3ccc4ccccc4c3)cc21]

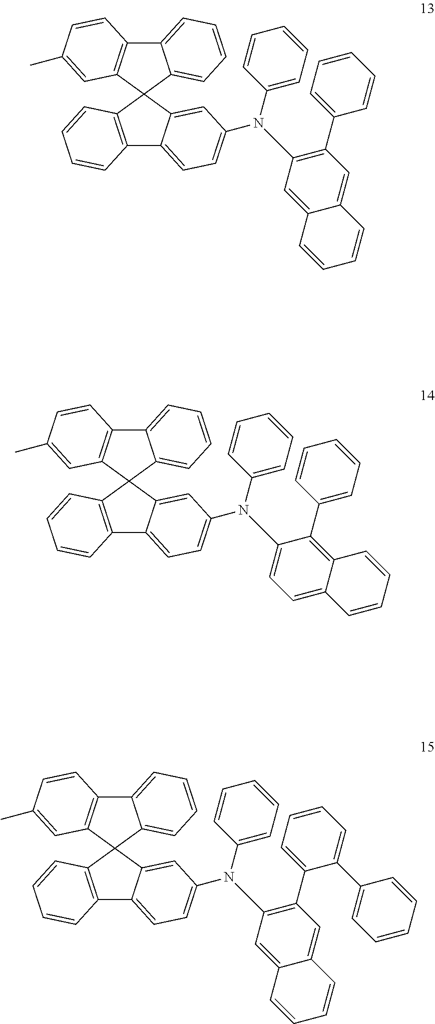 [SMILES: Cc1ccc2c(c1)C1(c3ccccc3-2)c2ccccc2-c2ccc(N(c3ccccc3)c3cc4ccccc4cc3-c3ccccc3)cc21 ; Cc1ccc2c(c1)C1(c3ccccc3-2)c2ccccc2-c2ccc(N(c3ccccc3)c3ccc4ccccc4c3-c3ccccc3)cc21 ; Cc1ccc2c(c1)C1(c3ccccc3-2)c2ccccc2-c2ccc(N(c3ccccc3)c3cc4ccccc4cc3-c3ccccc3-c3ccccc3)cc21]

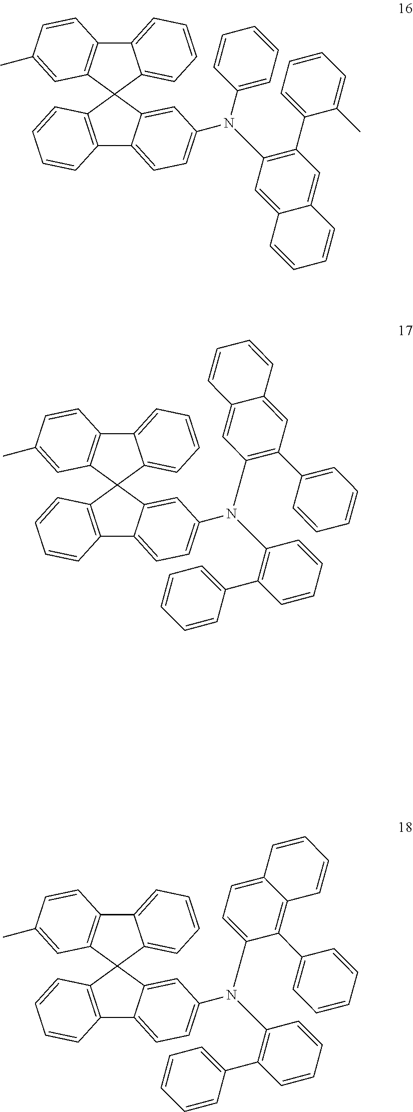

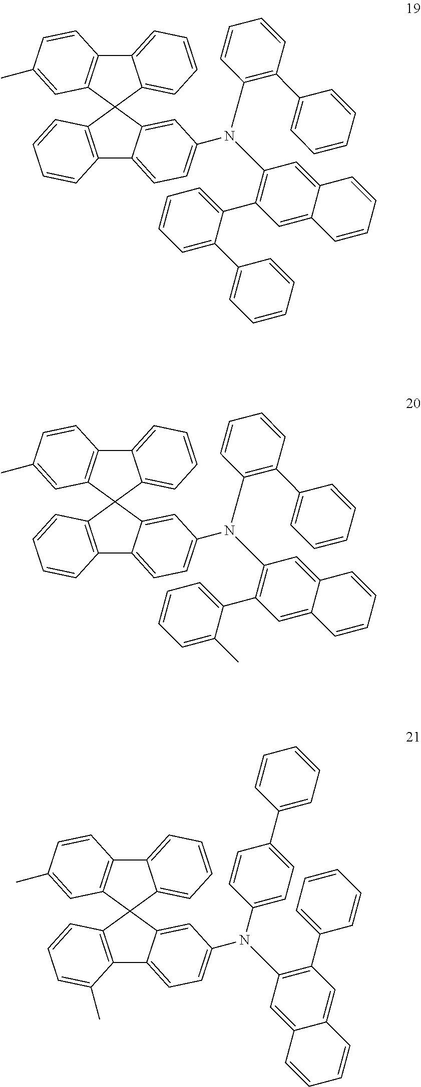 [SMILES: Cc1ccc2c(c1)C1(c3ccccc3-2)c2ccccc2-c2ccc(N(c3ccccc3-c3ccccc3)c3cc4ccccc4cc3-c3ccccc3-c3ccccc3)cc21 ; Cc1ccc2c(c1)C1(c3ccccc3-2)c2ccccc2-c2ccc(N(c3ccccc3-c3ccccc3)c3cc4ccccc4cc3-c3ccccc3C)cc21 ; Cc1ccc2c(c1)C1(c3ccccc3-2)c2cc(N(c3ccc(-c4ccccc4)cc3)c3cc4ccccc4cc3-c3ccccc3)ccc2-c2c(C)cccc21]

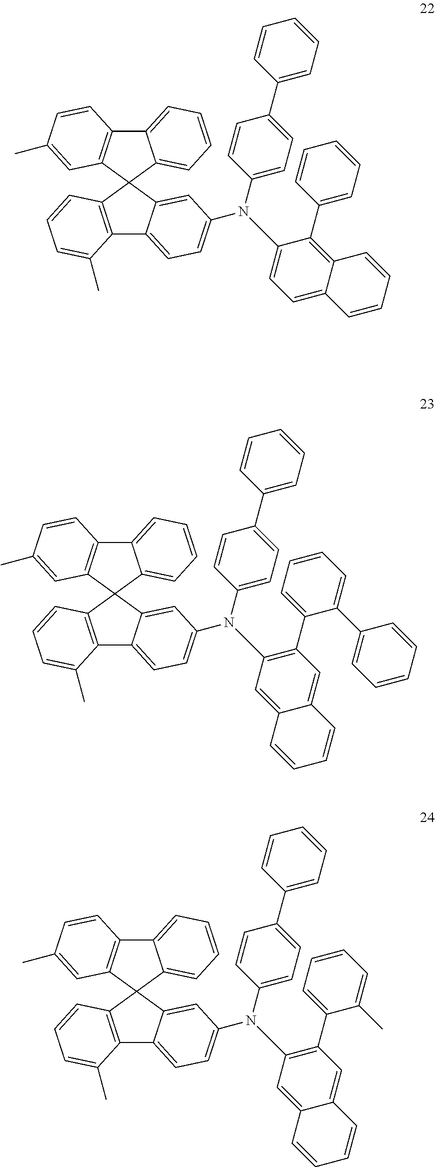

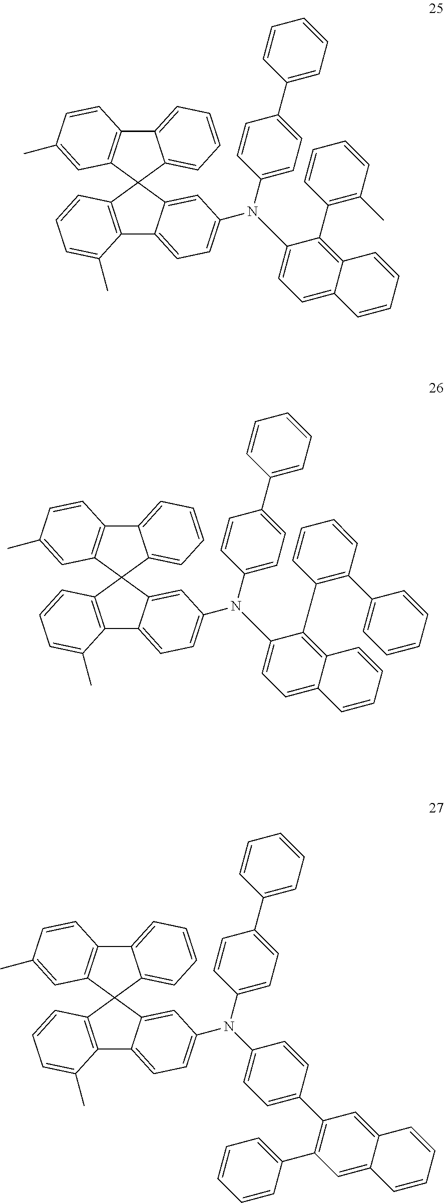 [SMILES: Cc1ccc2c(c1)C1(c3ccccc3-2)c2cc(N(c3ccc(-c4ccccc4)cc3)c3ccc4ccccc4c3-c3ccccc3C)ccc2-c2c(C)cccc21 ; Cc1ccc2c(c1)C1(c3ccccc3-2)c2cc(N(c3ccc(-c4ccccc4)cc3)c3ccc4ccccc4c3-c3ccccc3-c3ccccc3)ccc2-c2c(C)cccc21 ; Cc1ccc2c(c1)C1(c3ccccc3-2)c2cc(N(c3ccc(-c4ccccc4)cc3)c3ccc(-c4cc5ccccc5cc4-c4ccccc4)cc3)ccc2-c2c(C)cccc21]

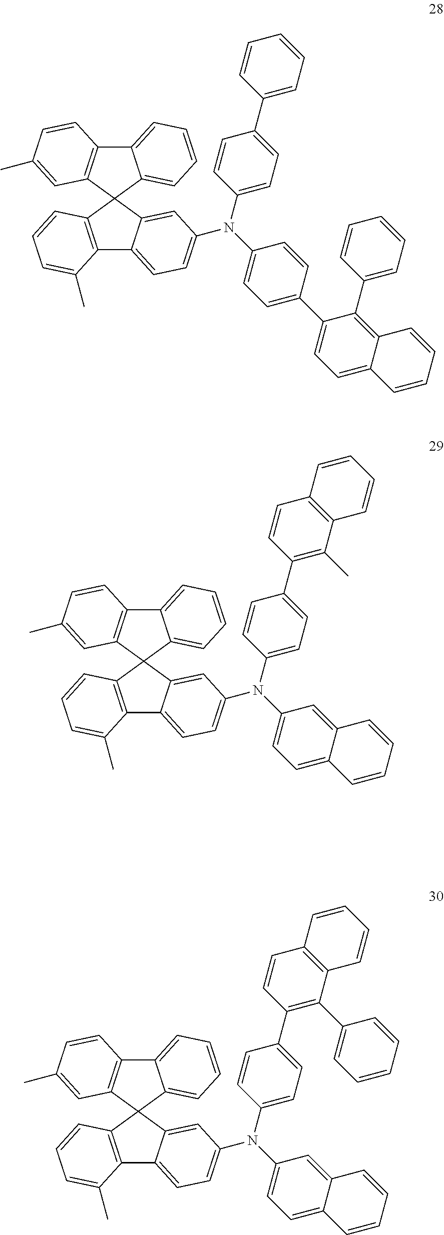 [SMILES: Cc1ccc2c(c1)C1(c3ccccc3-2)c2cc(N(c3ccc(-c4ccccc4)cc3)c3ccc(-c4ccc5ccccc5c4-c4ccccc4)cc3)ccc2-c2c(C)cccc21 ; Cc1ccc2c(c1)C1(c3ccccc3-2)c2cc(N(c3ccc(-c4ccc5ccccc5c4C)cc3)c3ccc4ccccc4c3)ccc2-c2c(C)cccc21 ; Cc1ccc2c(c1)C1(c3ccccc3-2)c2cc(N(c3ccc(-c4ccc5ccccc5c4-c4ccccc4)cc3)c3ccc4ccccc4c3)ccc2-c2c(C)cccc21]

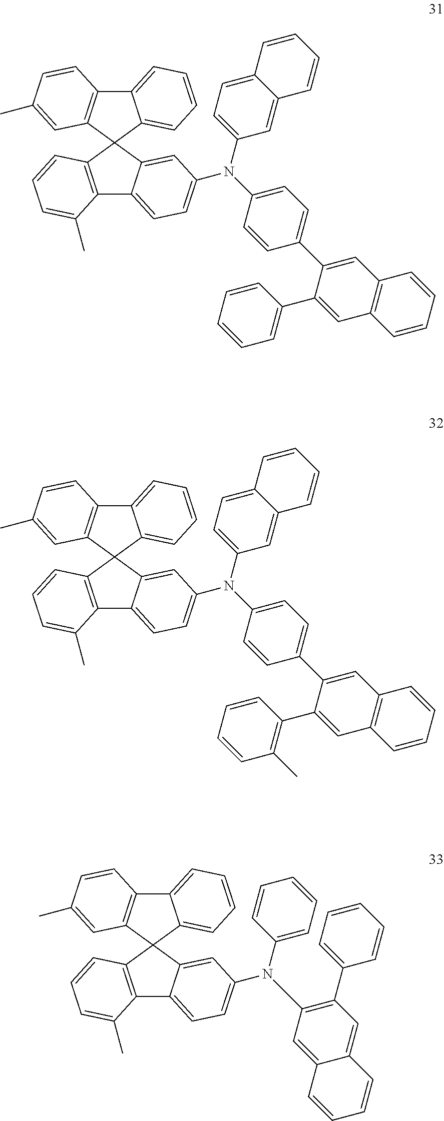 [SMILES: Cc1ccc2c(c1)C1(c3ccccc3-2)c2cc(N(c3ccc(-c4cc5ccccc5cc4-c4ccccc4)cc3)c3ccc4ccccc4c3)ccc2-c2c(C)cccc21 ; Cc1ccc2c(c1)C1(c3ccccc3-2)c2cc(N(c3ccc(-c4cc5ccccc5cc4-c4ccccc4C)cc3)c3ccc4ccccc4c3)ccc2-c2c(C)cccc21 ; Cc1ccc2c(c1)C1(c3ccccc3-2)c2cc(N(c3ccccc3)c3cc4ccccc4cc3-c3ccccc3)ccc2-c2c(C)cccc21]

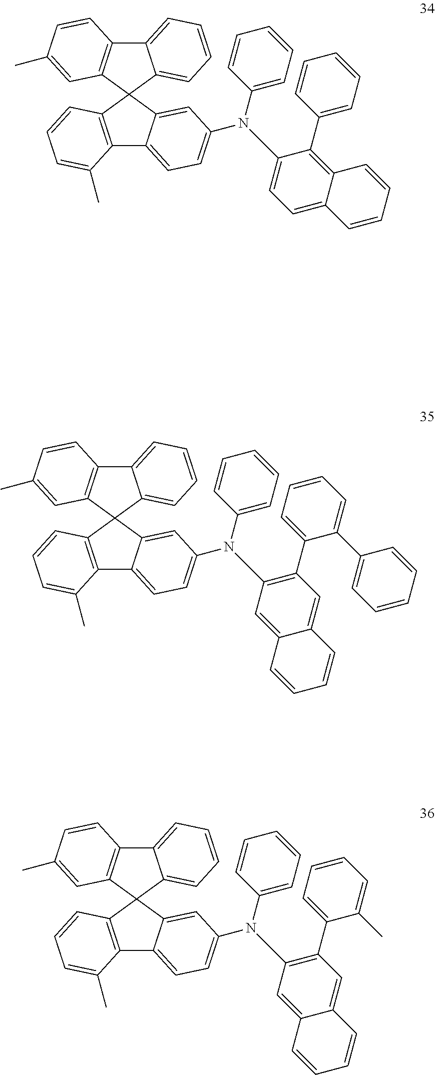

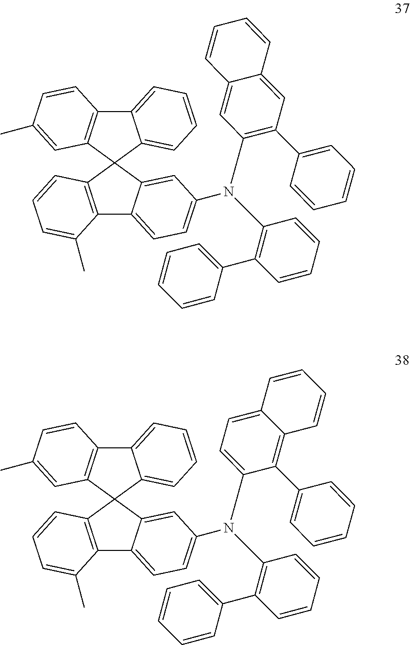 [SMILES: Cc1ccc2c(c1)C1(c3ccccc3-2)c2cc(N(c3ccccc3-c3ccccc3)c3cc4ccccc4cc3-c3ccccc3)ccc2-c2c(C)cccc21 ; Cc1ccc2c(c1)C1(c3ccccc3-2)c2cc(N(c3ccccc3-c3ccccc3)c3ccc4ccccc4c3-c3ccccc3)ccc2-c2c(C)cccc21]

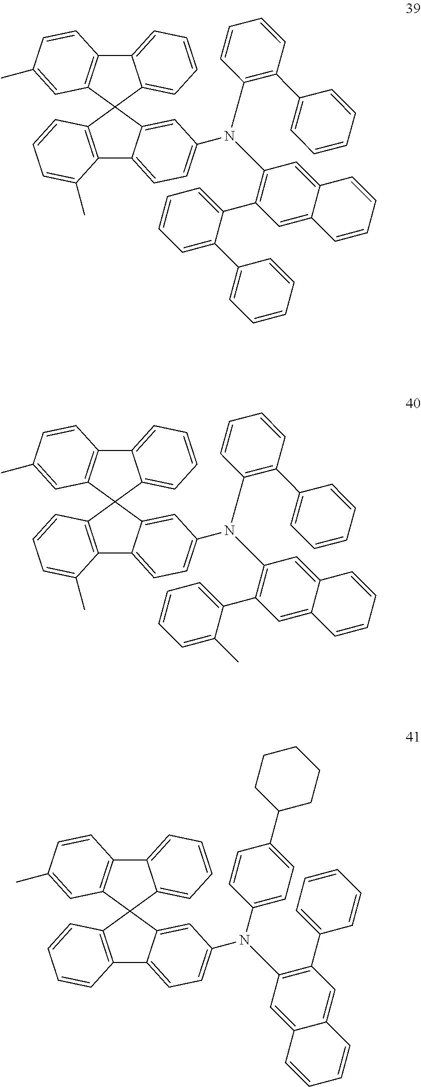 [SMILES: Cc1ccc2c(c1)C1(c3ccccc3-2)c2cc(N(c3ccccc3-c3ccccc3)c3cc4ccccc4cc3-c3ccccc3-c3ccccc3)ccc2-c2c(C)cccc21 ; Cc1ccc2c(c1)C1(c3ccccc3-2)c2cc(N(c3ccccc3-c3ccccc3)c3cc4ccccc4cc3-c3ccccc3C)ccc2-c2c(C)cccc21 ; Cc1ccc2c(c1)C1(c3ccccc3-2)c2ccccc2-c2ccc(N(c3ccc(C4CCCCC4)cc3)c3cc4ccccc4cc3-c3ccccc3)cc21]

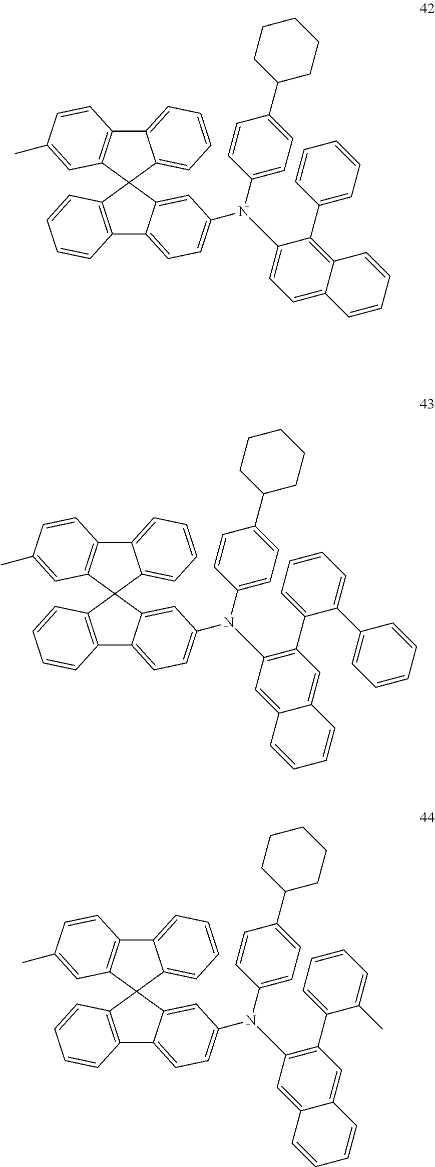

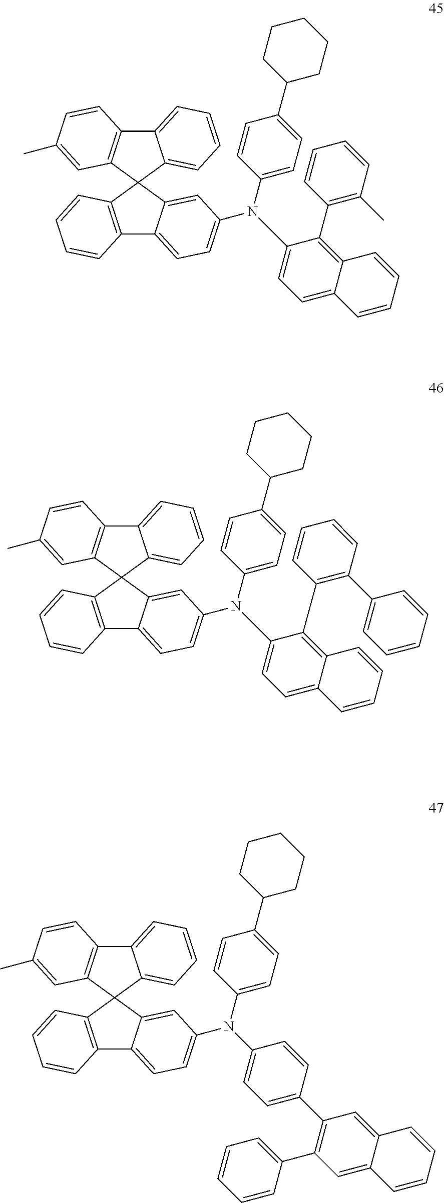 [SMILES: Cc1ccc2c(c1)C1(c3ccccc3-2)c2ccccc2-c2ccc(N(c3ccc(C4CCCCC4)cc3)c3ccc4ccccc4c3-c3ccccc3C)cc21 ; Cc1ccc2c(c1)C1(c3ccccc3-2)c2ccccc2-c2ccc(N(c3ccc(C4CCCCC4)cc3)c3ccc4ccccc4c3-c3ccccc3-c3ccccc3)cc21 ; Cc1ccc2c(c1)C1(c3ccccc3-2)c2ccccc2-c2ccc(N(c3ccc(-c4cc5ccccc5cc4-c4ccccc4)cc3)c3ccc(C4CCCCC4)cc3)cc21]

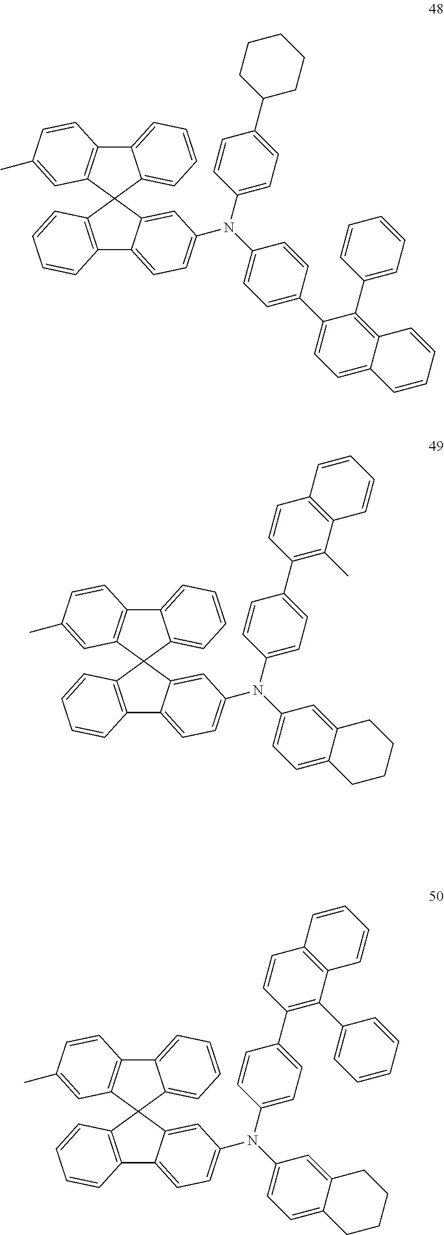 [SMILES: Cc1ccc2c(c1)C1(c3ccccc3-2)c2ccccc2-c2ccc(N(c3ccc(-c4ccc5ccccc5c4-c4ccccc4)cc3)c3ccc(C4CCCCC4)cc3)cc21 ; Cc1ccc2c(c1)C1(c3ccccc3-2)c2ccccc2-c2ccc(N(c3ccc(-c4ccc5ccccc5c4C)cc3)c3ccc4c(c3)CCCC4)cc21 ; Cc1ccc2c(c1)C1(c3ccccc3-2)c2ccccc2-c2ccc(N(c3ccc(-c4ccc5ccccc5c4-c4ccccc4)cc3)c3ccc4c(c3)CCCC4)cc21]

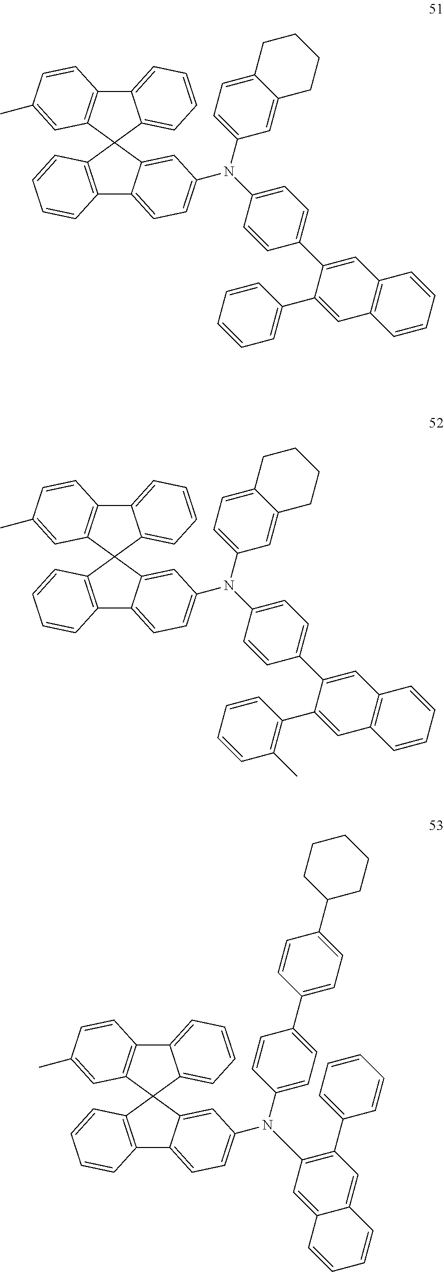 [SMILES: Cc1ccc2c(c1)C1(c3ccccc3-2)c2ccccc2-c2ccc(N(c3ccc(-c4cc5ccccc5cc4-c4ccccc4)cc3)c3ccc4c(c3)CCCC4)cc21 ; Cc1ccc2c(c1)C1(c3ccccc3-2)c2ccccc2-c2ccc(N(c3ccc(-c4cc5ccccc5cc4-c4ccccc4C)cc3)c3ccc4c(c3)CCCC4)cc21 ; Cc1ccc2c(c1)C1(c3ccccc3-2)c2ccccc2-c2ccc(N(c3ccc(-c4ccc(C5CCCCC5)cc4)cc3)c3cc4ccccc4cc3-c3ccccc3)cc21]

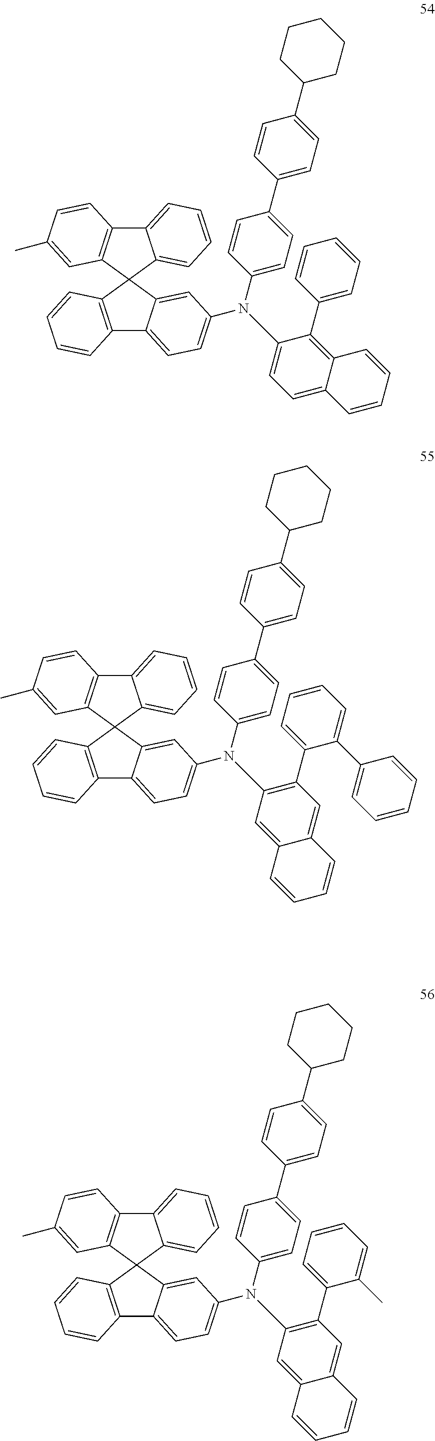

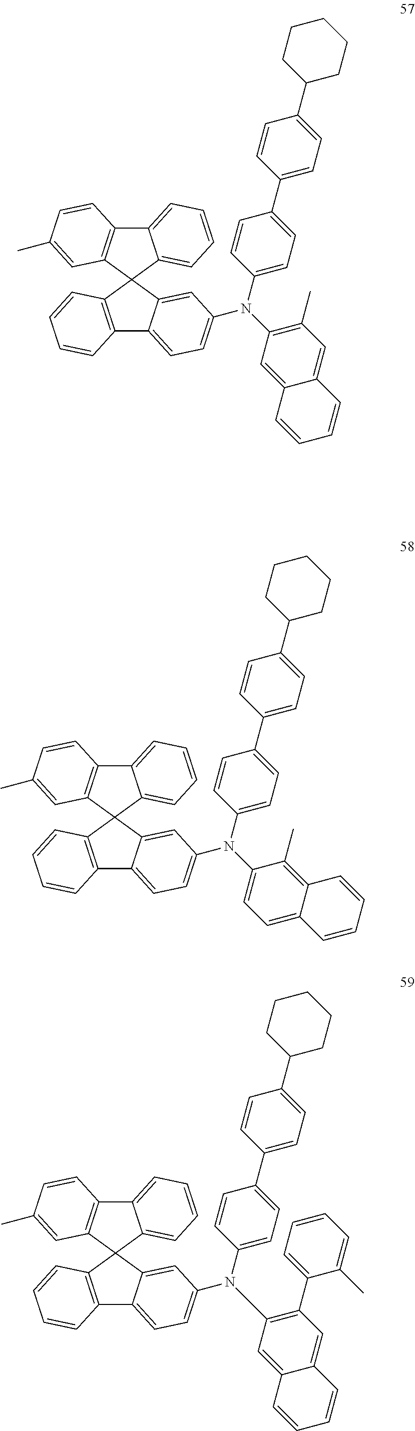

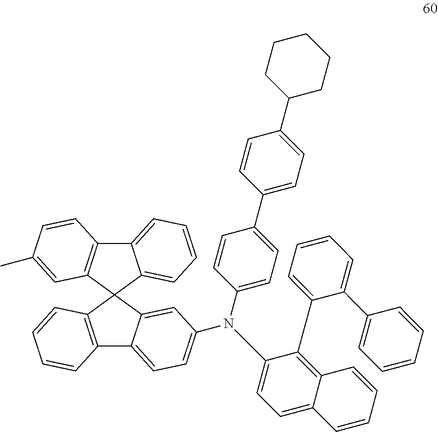 [SMILES: Cc1ccc2c(c1)C1(c3ccccc3-2)c2ccccc2-c2ccc(N(c3ccc(-c4ccc(C5CCCCC5)cc4)cc3)c3ccc4ccccc4c3-c3ccccc3-c3ccccc3)cc21]

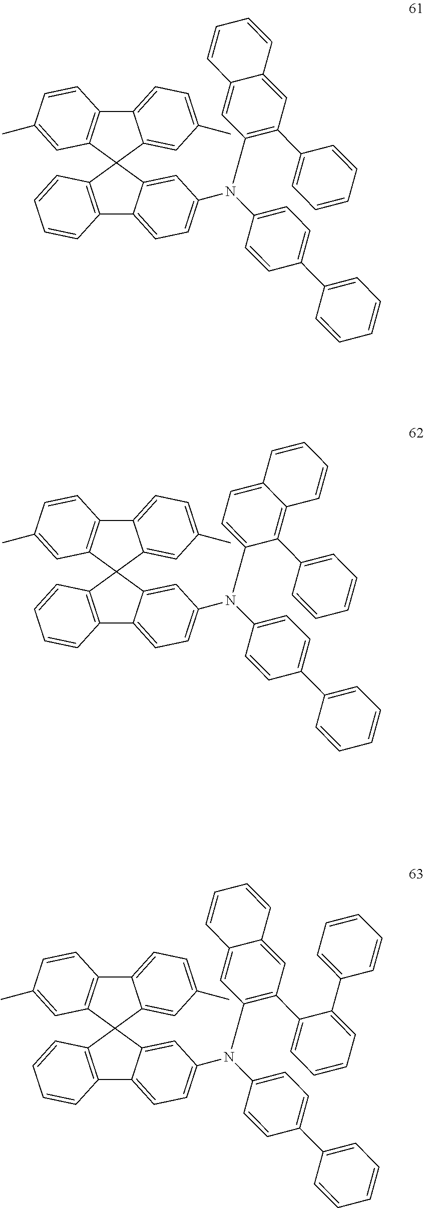 [SMILES: Cc1ccc2c(c1)C1(c3ccccc3-c3ccc(N(c4ccc(-c5ccccc5)cc4)c4cc5ccccc5cc4-c4ccccc4)cc31)c1cc(C)ccc1-2 ; Cc1ccc2c(c1)C1(c3ccccc3-c3ccc(N(c4ccc(-c5ccccc5)cc4)c4ccc5ccccc5c4-c4ccccc4)cc31)c1cc(C)ccc1-2 ; Cc1ccc2c(c1)C1(c3ccccc3-c3ccc(N(c4ccc(-c5ccccc5)cc4)c4cc5ccccc5cc4-c4ccccc4-c4ccccc4)cc31)c1cc(C)ccc1-2]

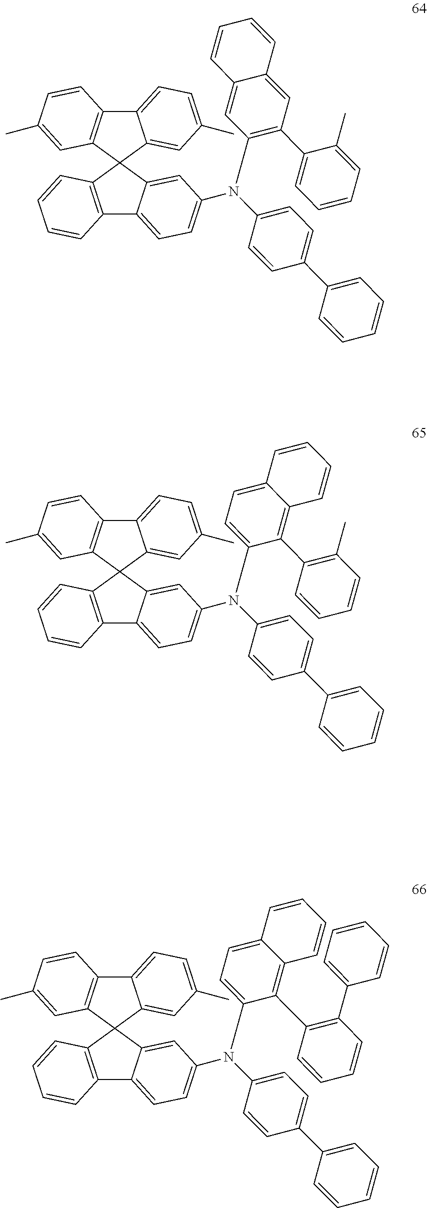 [SMILES: Cc1ccc2c(c1)C1(c3ccccc3-c3ccc(N(c4ccc(-c5ccccc5)cc4)c4cc5ccccc5cc4-c4ccccc4C)cc31)c1cc(C)ccc1-2 ; Cc1ccc2c(c1)C1(c3ccccc3-c3ccc(N(c4ccc(-c5ccccc5)cc4)c4ccc5ccccc5c4-c4ccccc4C)cc31)c1cc(C)ccc1-2 ; Cc1ccc2c(c1)C1(c3ccccc3-c3ccc(N(c4ccc(-c5ccccc5)cc4)c4ccc5ccccc5c4-c4ccccc4-c4ccccc4)cc31)c1cc(C)ccc1-2]

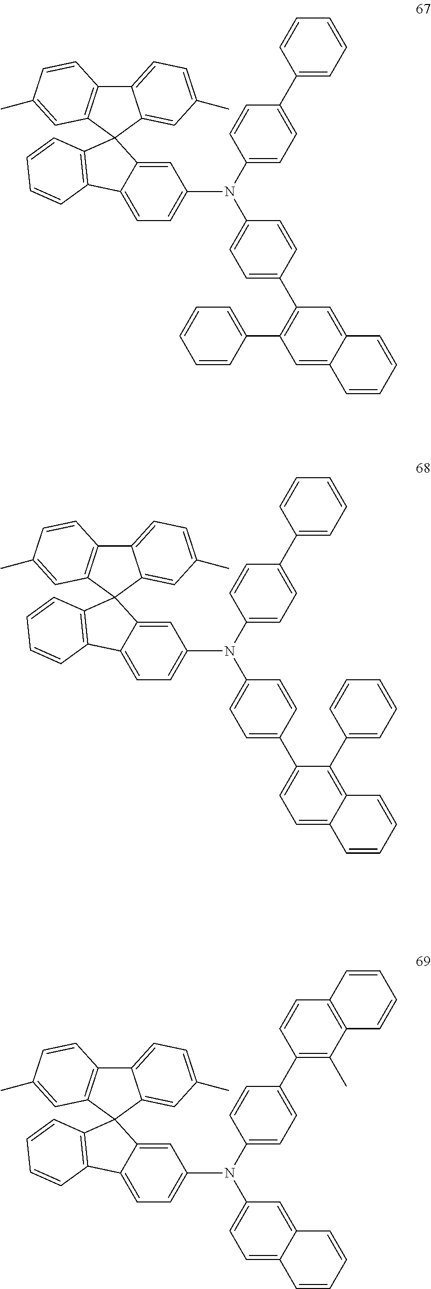 [SMILES: Cc1ccc2c(c1)C1(c3ccccc3-c3ccc(N(c4ccc(-c5ccccc5)cc4)c4ccc(-c5cc6ccccc6cc5-c5ccccc5)cc4)cc31)c1cc(C)ccc1-2 ; Cc1ccc2c(c1)C1(c3ccccc3-c3ccc(N(c4ccc(-c5ccccc5)cc4)c4ccc(-c5ccc6ccccc6c5-c5ccccc5)cc4)cc31)c1cc(C)ccc1-2 ; Cc1ccc2c(c1)C1(c3ccccc3-c3ccc(N(c4ccc(-c5ccc6ccccc6c5C)cc4)c4ccc5ccccc5c4)cc31)c1cc(C)ccc1-2]

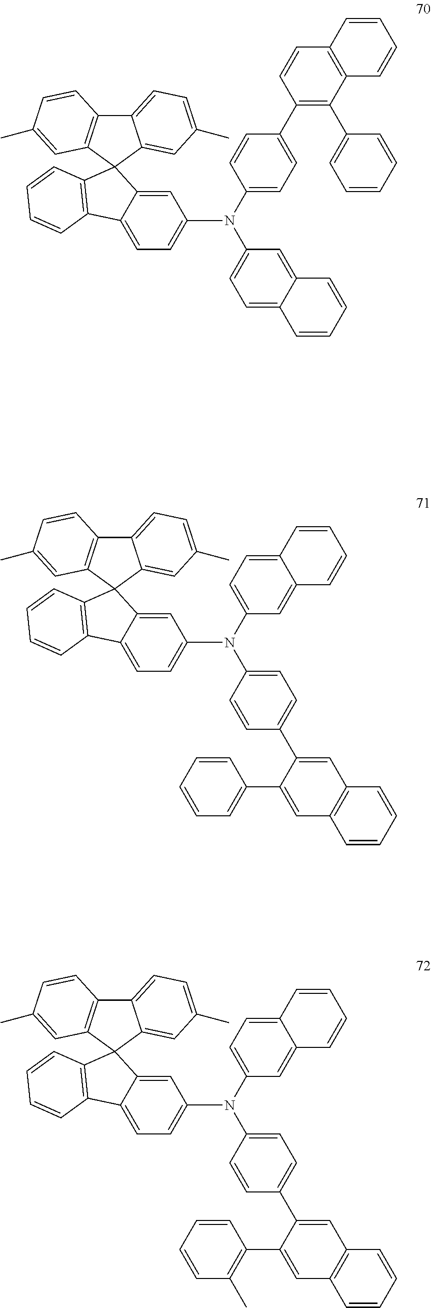 [SMILES: Cc1ccc2c(c1)C1(c3ccccc3-c3ccc(N(c4ccc(-c5ccc6ccccc6c5-c5ccccc5)cc4)c4ccc5ccccc5c4)cc31)c1cc(C)ccc1-2 ; Cc1ccc2c(c1)C1(c3ccccc3-c3ccc(N(c4ccc(-c5cc6ccccc6cc5-c5ccccc5)cc4)c4ccc5ccccc5c4)cc31)c1cc(C)ccc1-2 ; Cc1ccc2c(c1)C1(c3ccccc3-c3ccc(N(c4ccc(-c5cc6ccccc6cc5-c5ccccc5C)cc4)c4ccc5ccccc5c4)cc31)c1cc(C)ccc1-2]

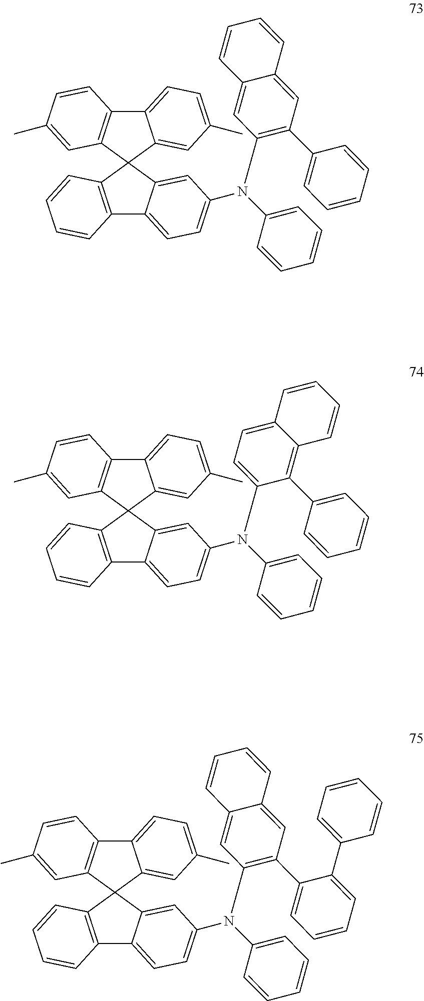 [SMILES: Cc1ccc2c(c1)C1(c3ccccc3-c3ccc(N(c4ccccc4)c4cc5ccccc5cc4-c4ccccc4)cc31)c1cc(C)ccc1-2 ; Cc1ccc2c(c1)C1(c3ccccc3-c3ccc(N(c4ccccc4)c4ccc5ccccc5c4-c4ccccc4)cc31)c1cc(C)ccc1-2 ; Cc1ccc2c(c1)C1(c3ccccc3-c3ccc(N(c4ccccc4)c4cc5ccccc5cc4-c4ccccc4-c4ccccc4)cc31)c1cc(C)ccc1-2]

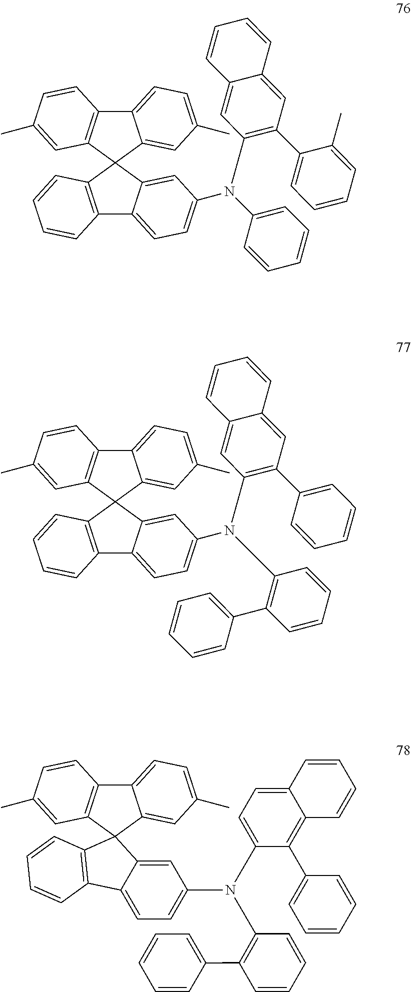 [SMILES: Cc1ccc2c(c1)C1(c3ccccc3-c3ccc(N(c4ccccc4)c4cc5ccccc5cc4-c4ccccc4C)cc31)c1cc(C)ccc1-2 ; Cc1ccc2c(c1)C1(c3ccccc3-c3ccc(N(c4ccccc4-c4ccccc4)c4cc5ccccc5cc4-c4ccccc4)cc31)c1cc(C)ccc1-2 ; Cc1ccc2c(c1)C1(c3ccccc3-c3ccc(N(c4ccccc4-c4ccccc4)c4ccc5ccccc5c4-c4ccccc4)cc31)c1cc(C)ccc1-2]

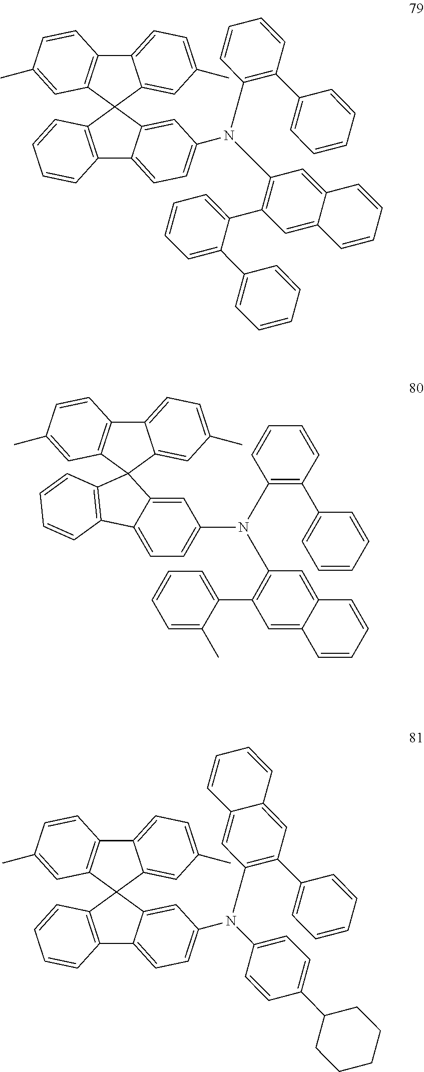 [SMILES: Cc1ccc2c(c1)C1(c3ccccc3-c3ccc(N(c4ccccc4-c4ccccc4)c4cc5ccccc5cc4-c4ccccc4-c4ccccc4)cc31)c1cc(C)ccc1-2 ; Cc1ccc2c(c1)C1(c3ccccc3-c3ccc(N(c4ccccc4-c4ccccc4)c4cc5ccccc5cc4-c4ccccc4C)cc31)c1cc(C)ccc1-2 ; Cc1ccc2c(c1)C1(c3ccccc3-c3ccc(N(c4ccc(C5CCCCC5)cc4)c4cc5ccccc5cc4-c4ccccc4)cc31)c1cc(C)ccc1-2]

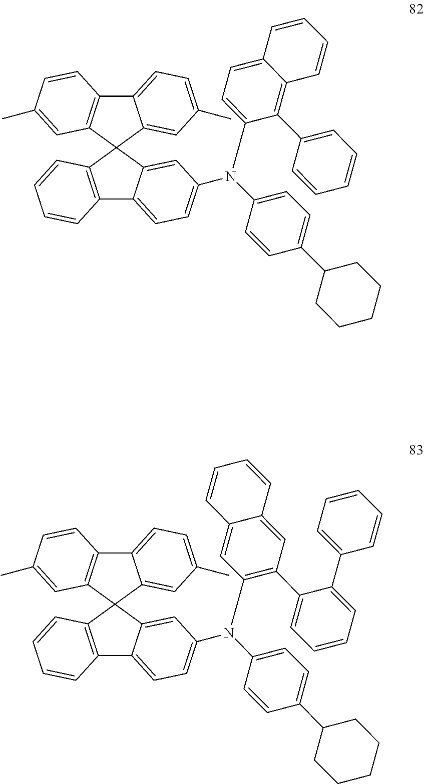 [SMILES: Cc1ccc2c(c1)C1(c3ccccc3-c3ccc(N(c4ccc(C5CCCCC5)cc4)c4ccc5ccccc5c4-c4ccccc4)cc31)c1cc(C)ccc1-2 ; Cc1ccc2c(c1)C1(c3ccccc3-c3ccc(N(c4ccc(C5CCCCC5)cc4)c4cc5ccccc5cc4-c4ccccc4-c4ccccc4)cc31)c1cc(C)ccc1-2]

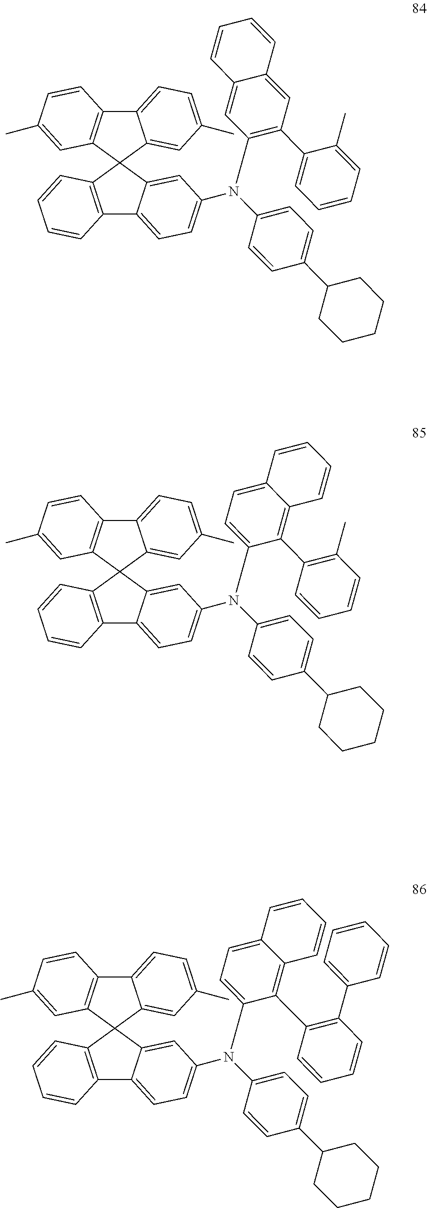 [SMILES: Cc1ccc2c(c1)C1(c3ccccc3-c3ccc(N(c4ccc(C5CCCCC5)cc4)c4cc5ccccc5cc4-c4ccccc4C)cc31)c1cc(C)ccc1-2 ; Cc1ccc2c(c1)C1(c3ccccc3-c3ccc(N(c4ccc(C5CCCCC5)cc4)c4ccc5ccccc5c4-c4ccccc4C)cc31)c1cc(C)ccc1-2 ; Cc1ccc2c(c1)C1(c3ccccc3-c3ccc(N(c4ccc(C5CCCCC5)cc4)c4ccc5ccccc5c4-c4ccccc4-c4ccccc4)cc31)c1cc(C)ccc1-2]

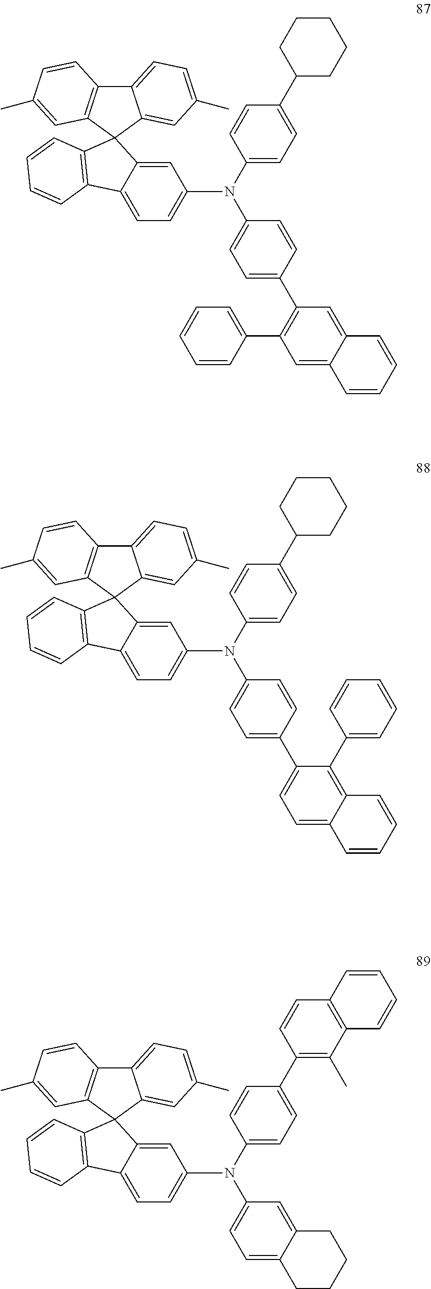 [SMILES: Cc1ccc2c(c1)C1(c3ccccc3-c3ccc(N(c4ccc(-c5cc6ccccc6cc5-c5ccccc5)cc4)c4ccc(C5CCCCC5)cc4)cc31)c1cc(C)ccc1-2 ; Cc1ccc2c(c1)C1(c3ccccc3-c3ccc(N(c4ccc(-c5ccc6ccccc6c5-c5ccccc5)cc4)c4ccc(C5CCCCC5)cc4)cc31)c1cc(C)ccc1-2 ; Cc1ccc2c(c1)C1(c3ccccc3-c3ccc(N(c4ccc(-c5ccc6ccccc6c5C)cc4)c4ccc5c(c4)CCCC5)cc31)c1cc(C)ccc1-2]

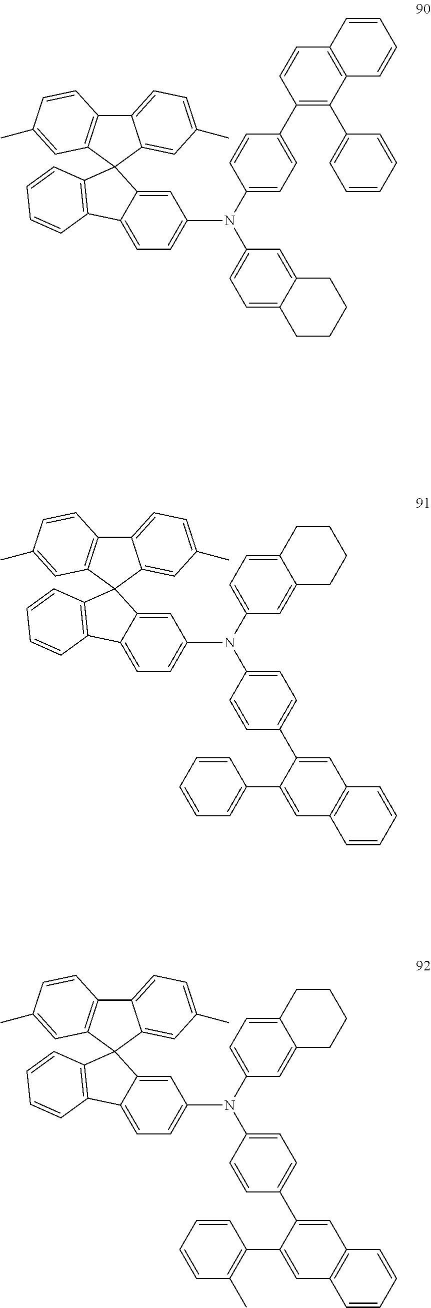 [SMILES: Cc1ccc2c(c1)C1(c3ccccc3-c3ccc(N(c4ccc(-c5ccc6ccccc6c5-c5ccccc5)cc4)c4ccc5c(c4)CCCC5)cc31)c1cc(C)ccc1-2 ; Cc1ccc2c(c1)C1(c3ccccc3-c3ccc(N(c4ccc(-c5cc6ccccc6cc5-c5ccccc5)cc4)c4ccc5c(c4)CCCC5)cc31)c1cc(C)ccc1-2 ; Cc1ccc2c(c1)C1(c3ccccc3-c3ccc(N(c4ccc(-c5cc6ccccc6cc5-c5ccccc5C)cc4)c4ccc5c(c4)CCCC5)cc31)c1cc(C)ccc1-2]

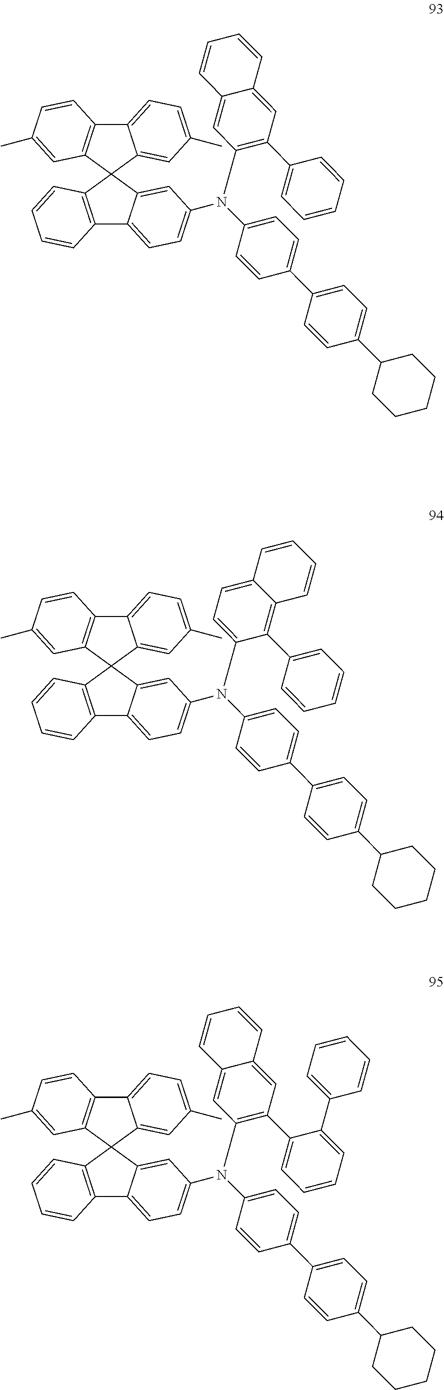 [SMILES: Cc1ccc2c(c1)C1(c3ccccc3-c3ccc(N(c4ccc(-c5ccc(C6CCCCC6)cc5)cc4)c4cc5ccccc5cc4-c4ccccc4)cc31)c1cc(C)ccc1-2 ; Cc1ccc2c(c1)C1(c3ccccc3-c3ccc(N(c4ccc(-c5ccc(C6CCCCC6)cc5)cc4)c4ccc5ccccc5c4-c4ccccc4)cc31)c1cc(C)ccc1-2 ; Cc1ccc2c(c1)C1(c3ccccc3-c3ccc(N(c4ccc(-c5ccc(C6CCCCC6)cc5)cc4)c4cc5ccccc5cc4-c4ccccc4-c4ccccc4)cc31)c1cc(C)ccc1-2]

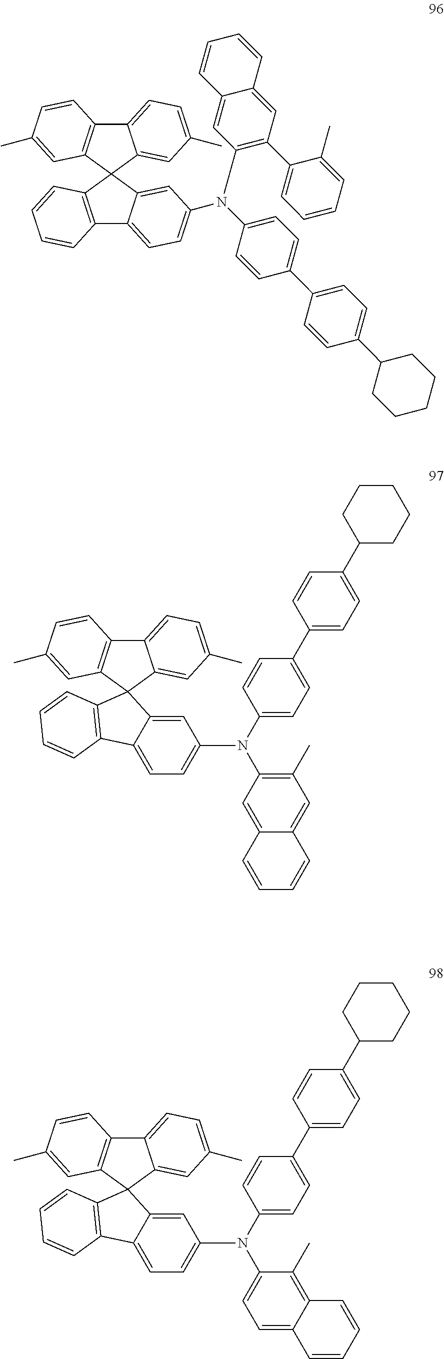 [SMILES: Cc1ccc2c(c1)C1(c3ccccc3-c3ccc(N(c4ccc(-c5ccc(C6CCCCC6)cc5)cc4)c4cc5ccccc5cc4-c4ccccc4C)cc31)c1cc(C)ccc1-2 ; Cc1ccc2c(c1)C1(c3ccccc3-c3ccc(N(c4ccc(-c5ccc(C6CCCCC6)cc5)cc4)c4cc5ccccc5cc4C)cc31)c1cc(C)ccc1-2 ; Cc1ccc2c(c1)C1(c3ccccc3-c3ccc(N(c4ccc(-c5ccc(C6CCCCC6)cc5)cc4)c4ccc5ccccc5c4C)cc31)c1cc(C)ccc1-2]

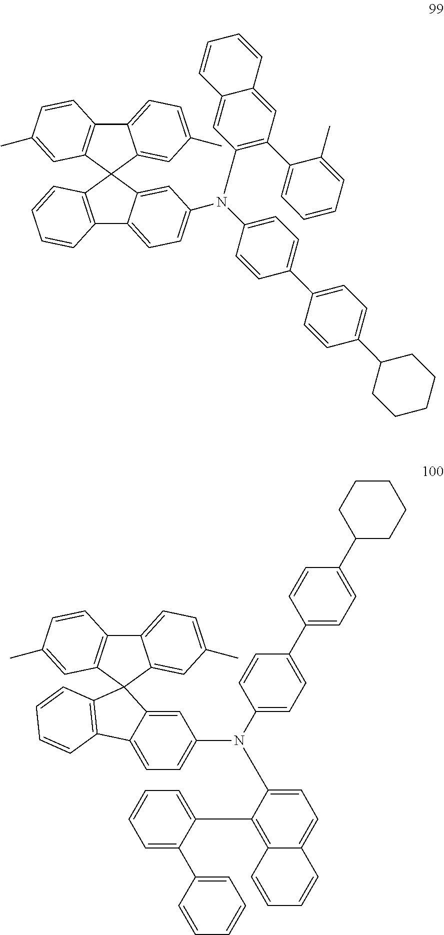 [SMILES: Cc1ccc2c(c1)C1(c3ccccc3-c3ccc(N(c4ccc(-c5ccc(C6CCCCC6)cc5)cc4)c4cc5ccccc5cc4-c4ccccc4C)cc31)c1cc(C)ccc1-2 ; Cc1ccc2c(c1)C1(c3ccccc3-c3ccc(N(c4ccc(-c5ccc(C6CCCCC6)cc5)cc4)c4ccc5ccccc5c4-c4ccccc4-c4ccccc4)cc31)c1cc(C)ccc1-2]

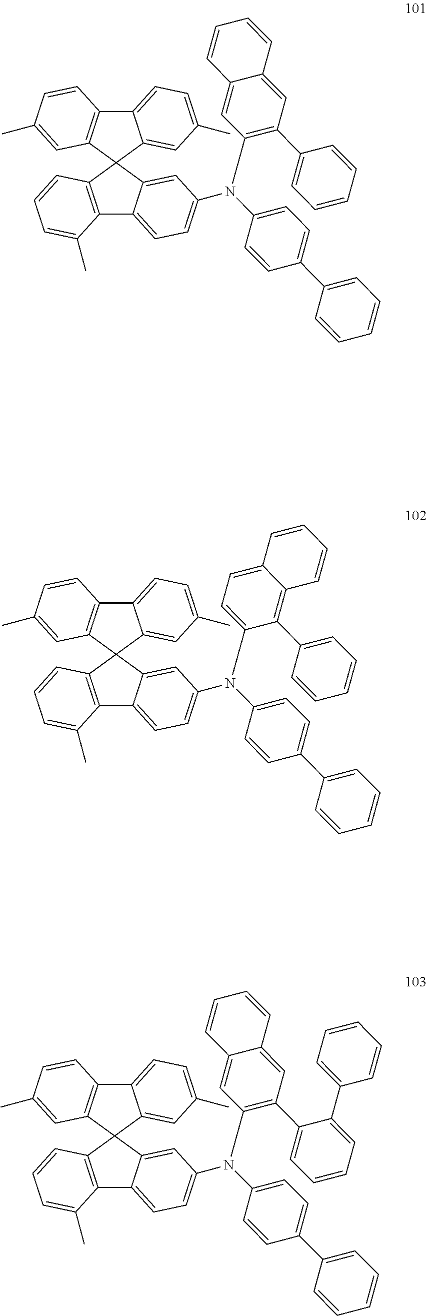 [SMILES: Cc1ccc2c(c1)C1(c3cc(C)ccc3-2)c2cc(N(c3ccc(-c4ccccc4)cc3)c3cc4ccccc4cc3-c3ccccc3)ccc2-c2c(C)cccc21 ; Cc1ccc2c(c1)C1(c3cc(C)ccc3-2)c2cc(N(c3ccc(-c4ccccc4)cc3)c3ccc4ccccc4c3-c3ccccc3)ccc2-c2c(C)cccc21 ; Cc1ccc2c(c1)C1(c3cc(C)ccc3-2)c2cc(N(c3ccc(-c4ccccc4)cc3)c3cc4ccccc4cc3-c3ccccc3-c3ccccc3)ccc2-c2c(C)cccc21]

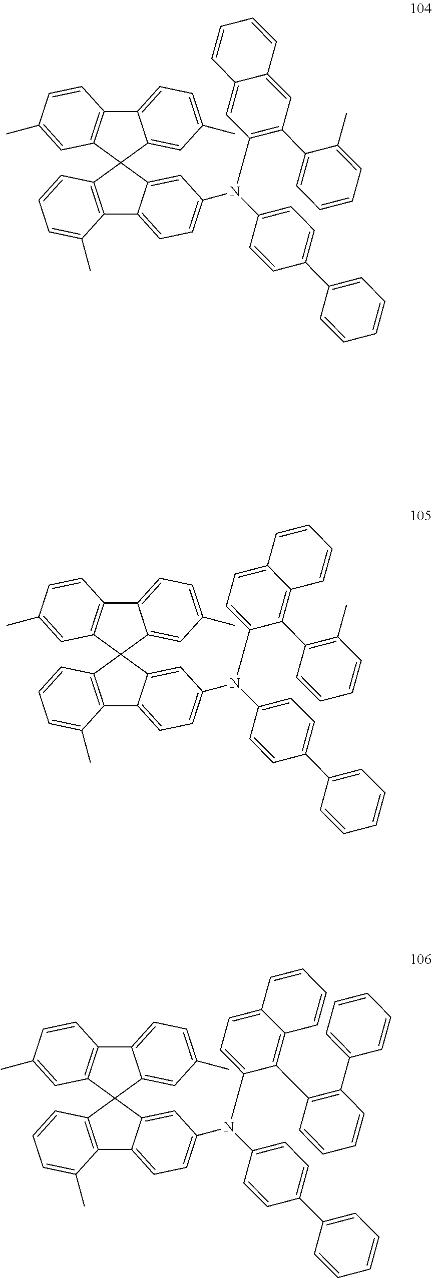 [SMILES: Cc1ccc2c(c1)C1(c3cc(C)ccc3-2)c2cc(N(c3ccc(-c4ccccc4)cc3)c3cc4ccccc4cc3-c3ccccc3C)ccc2-c2c(C)cccc21 ; Cc1ccc2c(c1)C1(c3cc(C)ccc3-2)c2cc(N(c3ccc(-c4ccccc4)cc3)c3ccc4ccccc4c3-c3ccccc3C)ccc2-c2c(C)cccc21 ; Cc1ccc2c(c1)C1(c3cc(C)ccc3-2)c2cc(N(c3ccc(-c4ccccc4)cc3)c3ccc4ccccc4c3-c3ccccc3-c3ccccc3)ccc2-c2c(C)cccc21]

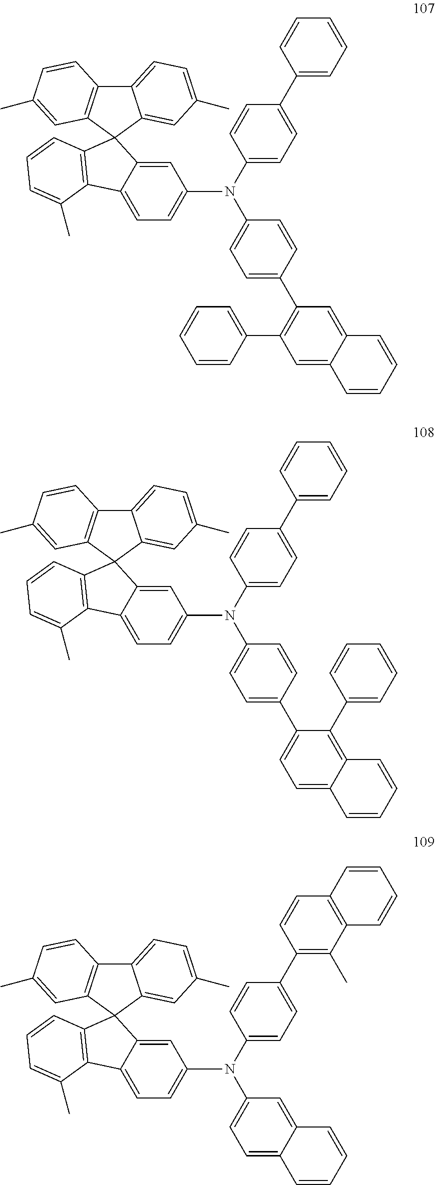 [SMILES: Cc1ccc2c(c1)C1(c3cc(C)ccc3-2)c2cc(N(c3ccc(-c4ccccc4)cc3)c3ccc(-c4cc5ccccc5cc4-c4ccccc4)cc3)ccc2-c2c(C)cccc21 ; Cc1ccc2c(c1)C1(c3cc(C)ccc3-2)c2cc(N(c3ccc(-c4ccccc4)cc3)c3ccc(-c4ccc5ccccc5c4-c4ccccc4)cc3)ccc2-c2c(C)cccc21 ; Cc1ccc2c(c1)C1(c3cc(C)ccc3-2)c2cc(N(c3ccc(-c4ccc5ccccc5c4C)cc3)c3ccc4ccccc4c3)ccc2-c2c(C)cccc21]

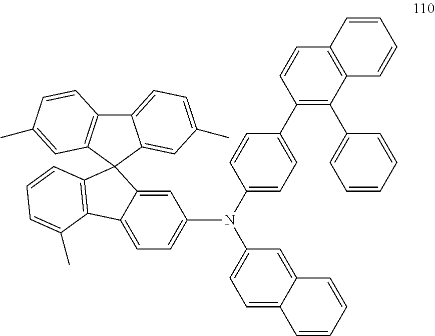 [SMILES: Cc1ccc2c(c1)C1(c3cc(C)ccc3-2)c2cc(N(c3ccc(-c4ccc5ccccc5c4-c4ccccc4)cc3)c3ccc4ccccc4c3)ccc2-c2c(C)cccc21]

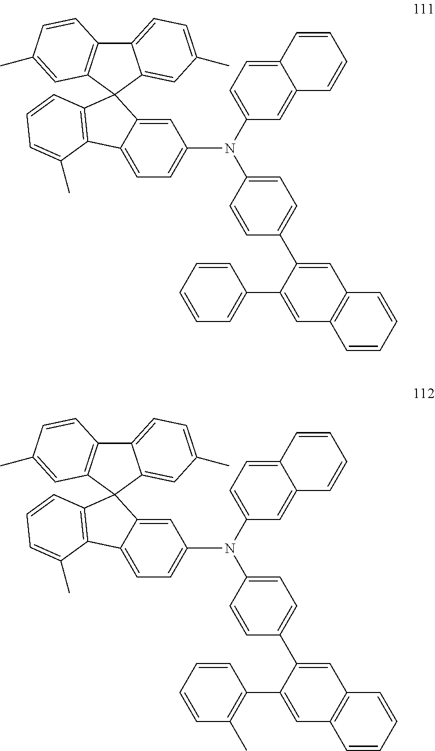 [SMILES: Cc1ccc2c(c1)C1(c3cc(C)ccc3-2)c2cc(N(c3ccc(-c4cc5ccccc5cc4-c4ccccc4)cc3)c3ccc4ccccc4c3)ccc2-c2c(C)cccc21 ; Cc1ccc2c(c1)C1(c3cc(C)ccc3-2)c2cc(N(c3ccc(-c4cc5ccccc5cc4-c4ccccc4C)cc3)c3ccc4ccccc4c3)ccc2-c2c(C)cccc21]

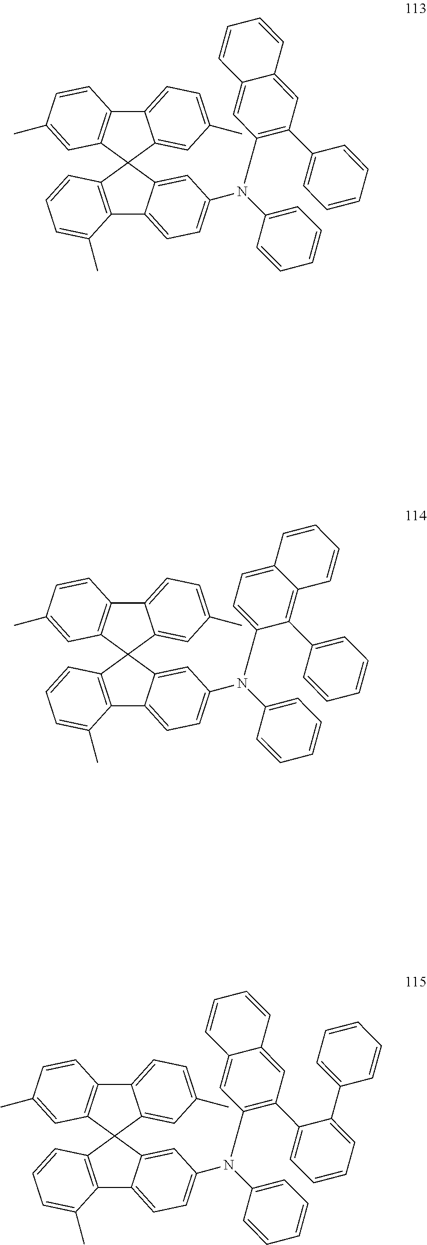 [SMILES: Cc1ccc2c(c1)C1(c3cc(C)ccc3-2)c2cc(N(c3ccccc3)c3cc4ccccc4cc3-c3ccccc3)ccc2-c2c(C)cccc21 ; Cc1ccc2c(c1)C1(c3cc(C)ccc3-2)c2cc(N(c3ccccc3)c3ccc4ccccc4c3-c3ccccc3)ccc2-c2c(C)cccc21 ; Cc1ccc2c(c1)C1(c3cc(C)ccc3-2)c2cc(N(c3ccccc3)c3cc4ccccc4cc3-c3ccccc3-c3ccccc3)ccc2-c2c(C)cccc21]

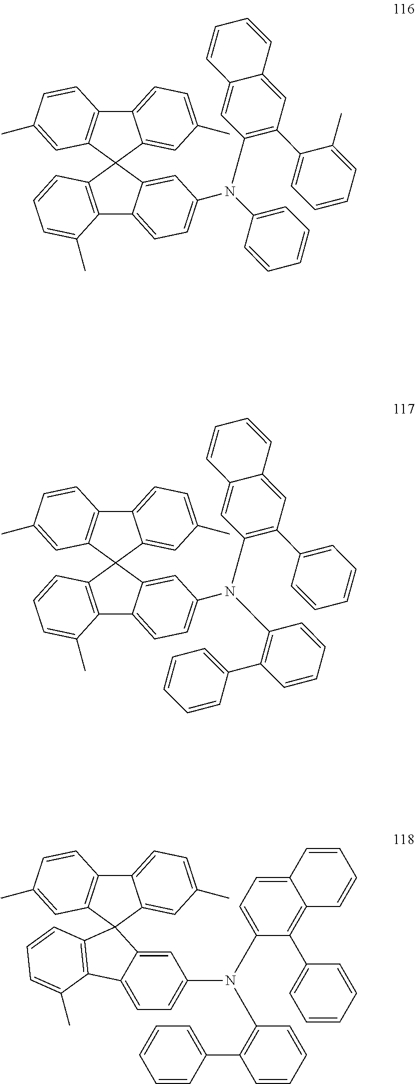 [SMILES: Cc1ccc2c(c1)C1(c3cc(C)ccc3-2)c2cc(N(c3ccccc3)c3cc4ccccc4cc3-c3ccccc3C)ccc2-c2c(C)cccc21 ; Cc1ccc2c(c1)C1(c3cc(C)ccc3-2)c2cc(N(c3ccccc3-c3ccccc3)c3cc4ccccc4cc3-c3ccccc3)ccc2-c2c(C)cccc21 ; Cc1ccc2c(c1)C1(c3cc(C)ccc3-2)c2cc(N(c3ccccc3-c3ccccc3)c3ccc4ccccc4c3-c3ccccc3)ccc2-c2c(C)cccc21]

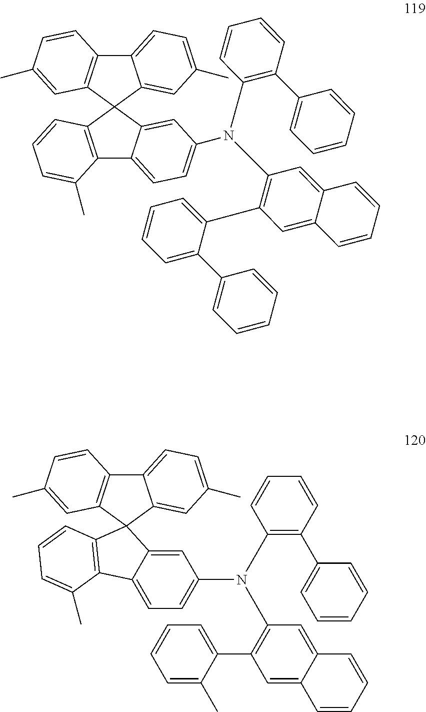 [SMILES: Cc1ccc2c(c1)C1(c3cc(C)ccc3-2)c2cc(N(c3ccccc3-c3ccccc3)c3cc4ccccc4cc3-c3ccccc3-c3ccccc3)ccc2-c2c(C)cccc21 ; Cc1ccc2c(c1)C1(c3cc(C)ccc3-2)c2cc(N(c3ccccc3-c3ccccc3)c3cc4ccccc4cc3-c3ccccc3C)ccc2-c2c(C)cccc21]

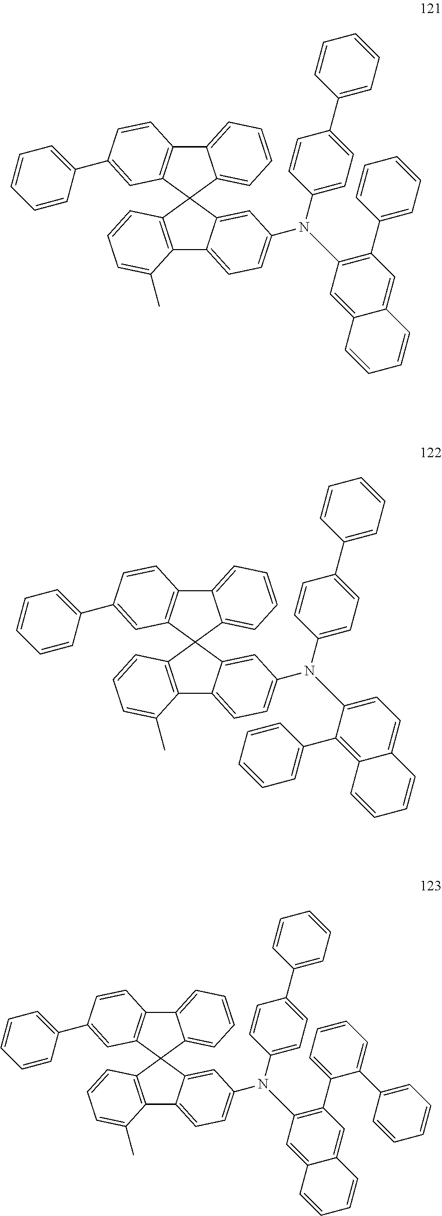 [SMILES: Cc1cccc2c1-c1ccc(N(c3ccc(-c4ccccc4)cc3)c3cc4ccccc4cc3-c3ccccc3)cc1C21c2ccccc2-c2ccc(-c3ccccc3)cc21 ; Cc1cccc2c1-c1ccc(N(c3ccc(-c4ccccc4)cc3)c3ccc4ccccc4c3-c3ccccc3)cc1C21c2ccccc2-c2ccc(-c3ccccc3)cc21 ; Cc1cccc2c1-c1ccc(N(c3ccc(-c4ccccc4)cc3)c3cc4ccccc4cc3-c3ccccc3-c3ccccc3)cc1C21c2ccccc2-c2ccc(-c3ccccc3)cc21]

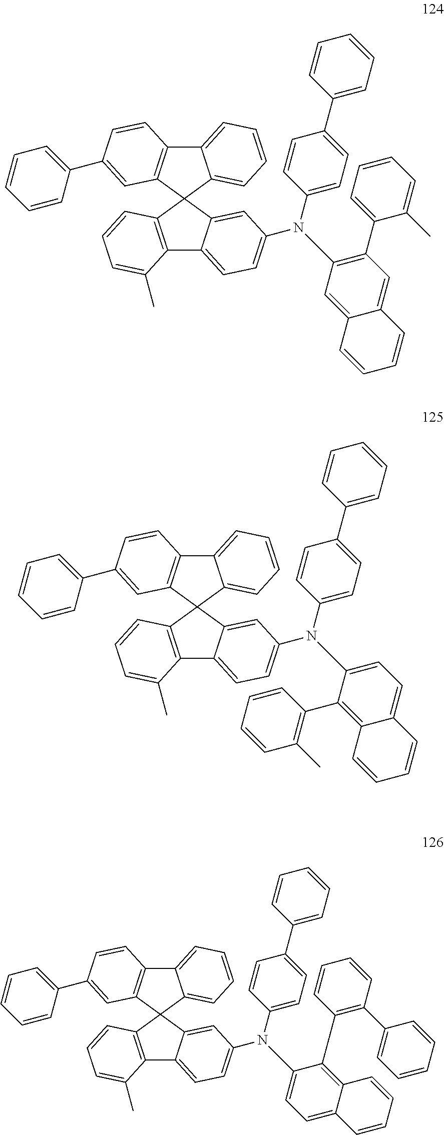 [SMILES: Cc1ccccc1-c1cc2ccccc2cc1N(c1ccc(-c2ccccc2)cc1)c1ccc2c(c1)C1(c3ccccc3-c3ccc(-c4ccccc4)cc31)c1cccc(C)c1-2 ; Cc1ccccc1-c1c(N(c2ccc(-c3ccccc3)cc2)c2ccc3c(c2)C2(c4ccccc4-c4ccc(-c5ccccc5)cc42)c2cccc(C)c2-3)ccc2ccccc12 ; Cc1cccc2c1-c1ccc(N(c3ccc(-c4ccccc4)cc3)c3ccc4ccccc4c3-c3ccccc3-c3ccccc3)cc1C21c2ccccc2-c2ccc(-c3ccccc3)cc21]

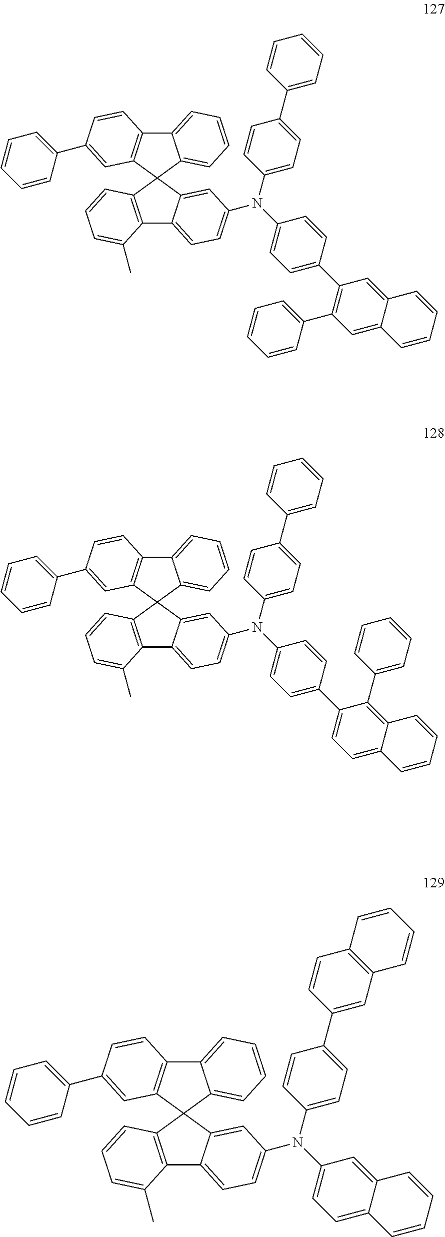 [SMILES: Cc1cccc2c1-c1ccc(N(c3ccc(-c4ccccc4)cc3)c3ccc(-c4cc5ccccc5cc4-c4ccccc4)cc3)cc1C21c2ccccc2-c2ccc(-c3ccccc3)cc21 ; Cc1cccc2c1-c1ccc(N(c3ccc(-c4ccccc4)cc3)c3ccc(-c4ccc5ccccc5c4-c4ccccc4)cc3)cc1C21c2ccccc2-c2ccc(-c3ccccc3)cc21 ; Cc1cccc2c1-c1ccc(N(c3ccc(-c4ccc5ccccc5c4)cc3)c3ccc4ccccc4c3)cc1C21c2ccccc2-c2ccc(-c3ccccc3)cc21]

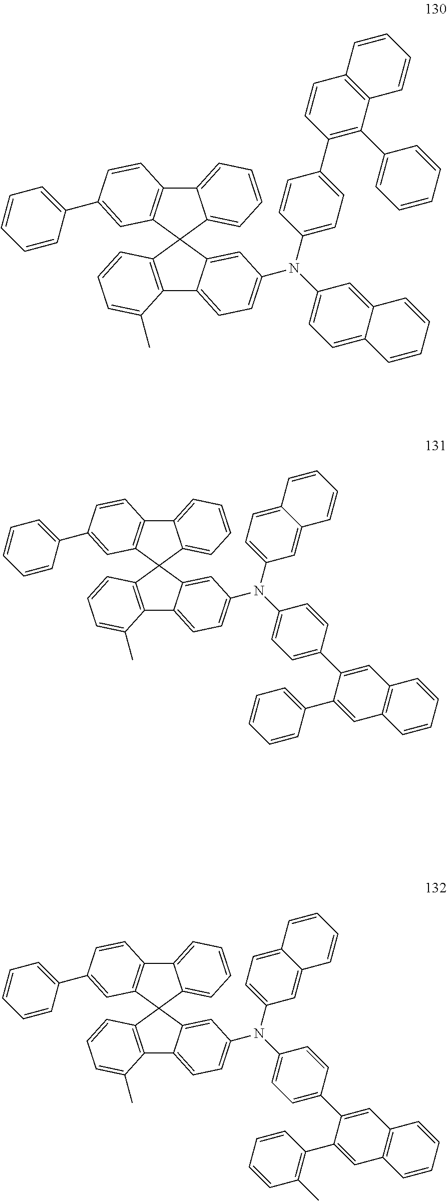 [SMILES: Cc1cccc2c1-c1ccc(N(c3ccc(-c4ccc5ccccc5c4-c4ccccc4)cc3)c3ccc4ccccc4c3)cc1C21c2ccccc2-c2ccc(-c3ccccc3)cc21 ; Cc1cccc2c1-c1ccc(N(c3ccc(-c4cc5ccccc5cc4-c4ccccc4)cc3)c3ccc4ccccc4c3)cc1C21c2ccccc2-c2ccc(-c3ccccc3)cc21 ; Cc1ccccc1-c1cc2ccccc2cc1-c1ccc(N(c2ccc3c(c2)C2(c4ccccc4-c4ccc(-c5ccccc5)cc42)c2cccc(C)c2-3)c2ccc3ccccc3c2)cc1]

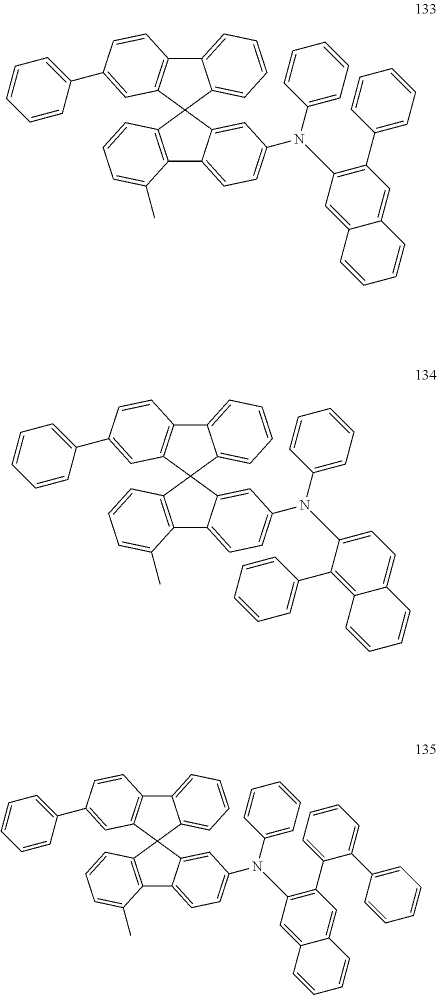 [SMILES: Cc1cccc2c1-c1ccc(N(c3ccccc3)c3cc4ccccc4cc3-c3ccccc3)cc1C21c2ccccc2-c2ccc(-c3ccccc3)cc21 ; Cc1cccc2c1-c1ccc(N(c3ccccc3)c3ccc4ccccc4c3-c3ccccc3)cc1C21c2ccccc2-c2ccc(-c3ccccc3)cc21 ; Cc1cccc2c1-c1ccc(N(c3ccccc3)c3cc4ccccc4cc3-c3ccccc3-c3ccccc3)cc1C21c2ccccc2-c2ccc(-c3ccccc3)cc21]

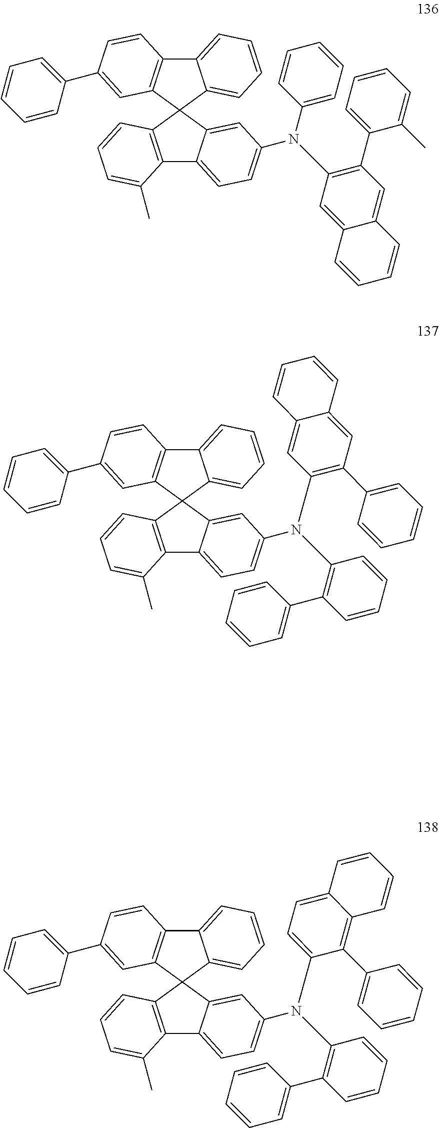 [SMILES: Cc1ccccc1-c1cc2ccccc2cc1N(c1ccccc1)c1ccc2c(c1)C1(c3ccccc3-c3ccc(-c4ccccc4)cc31)c1cccc(C)c1-2 ; Cc1cccc2c1-c1ccc(N(c3ccccc3-c3ccccc3)c3cc4ccccc4cc3-c3ccccc3)cc1C21c2ccccc2-c2ccc(-c3ccccc3)cc21 ; Cc1cccc2c1-c1ccc(N(c3ccccc3-c3ccccc3)c3ccc4ccccc4c3-c3ccccc3)cc1C21c2ccccc2-c2ccc(-c3ccccc3)cc21]

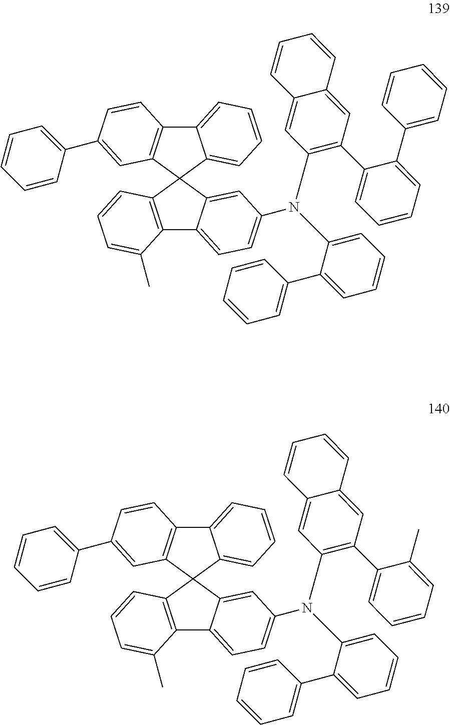 [SMILES: Cc1cccc2c1-c1ccc(N(c3ccccc3-c3ccccc3)c3cc4ccccc4cc3-c3ccccc3-c3ccccc3)cc1C21c2ccccc2-c2ccc(-c3ccccc3)cc21 ; Cc1ccccc1-c1cc2ccccc2cc1N(c1ccc2c(c1)C1(c3ccccc3-c3ccc(-c4ccccc4)cc31)c1cccc(C)c1-2)c1ccccc1-c1ccccc1]

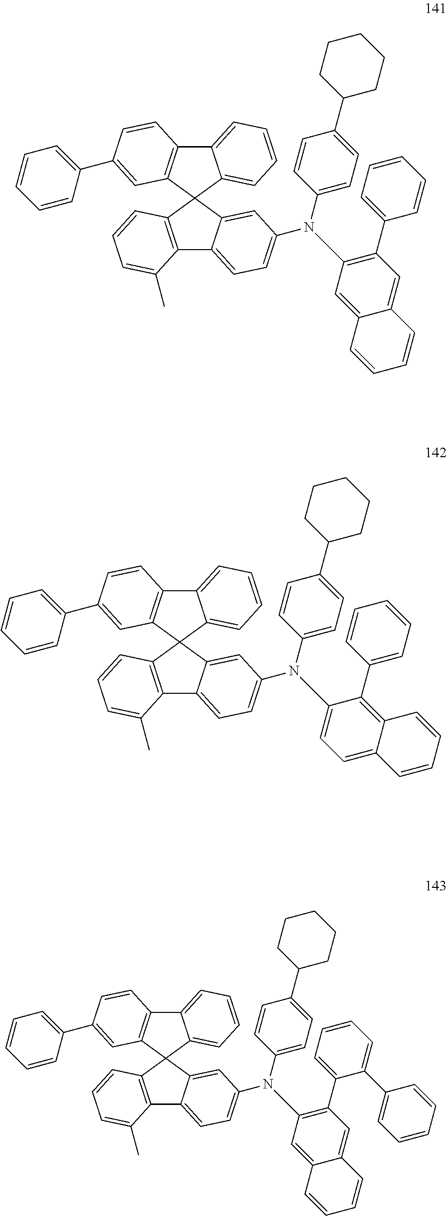 [SMILES: Cc1cccc2c1-c1ccc(N(c3ccc(C4CCCCC4)cc3)c3cc4ccccc4cc3-c3ccccc3)cc1C21c2ccccc2-c2ccc(-c3ccccc3)cc21 ; Cc1cccc2c1-c1ccc(N(c3ccc(C4CCCCC4)cc3)c3ccc4ccccc4c3-c3ccccc3)cc1C21c2ccccc2-c2ccc(-c3ccccc3)cc21 ; Cc1cccc2c1-c1ccc(N(c3ccc(C4CCCCC4)cc3)c3cc4ccccc4cc3-c3ccccc3-c3ccccc3)cc1C21c2ccccc2-c2ccc(-c3ccccc3)cc21]

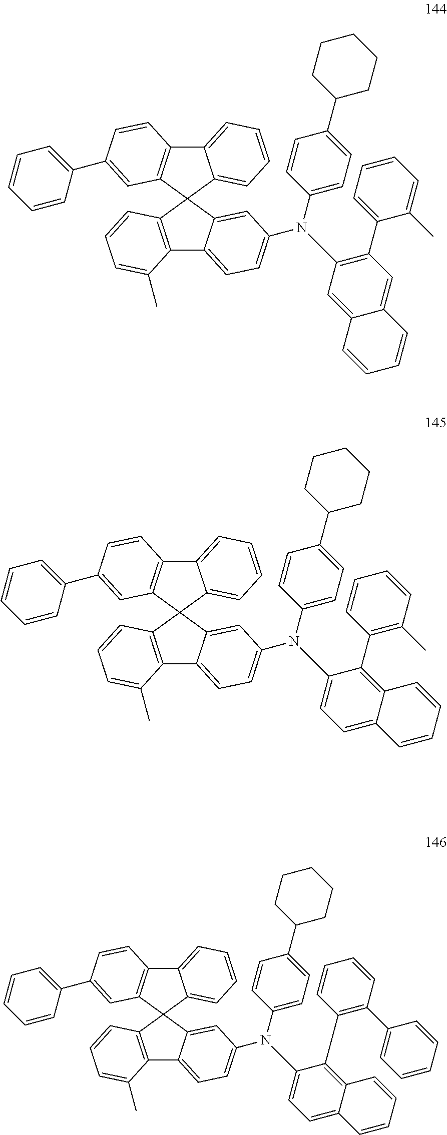 [SMILES: Cc1ccccc1-c1cc2ccccc2cc1N(c1ccc(C2CCCCC2)cc1)c1ccc2c(c1)C1(c3ccccc3-c3ccc(-c4ccccc4)cc31)c1cccc(C)c1-2 ; Cc1ccccc1-c1c(N(c2ccc(C3CCCCC3)cc2)c2ccc3c(c2)C2(c4ccccc4-c4ccc(-c5ccccc5)cc42)c2cccc(C)c2-3)ccc2ccccc12 ; Cc1cccc2c1-c1ccc(N(c3ccc(C4CCCCC4)cc3)c3ccc4ccccc4c3-c3ccccc3-c3ccccc3)cc1C21c2ccccc2-c2ccc(-c3ccccc3)cc21]

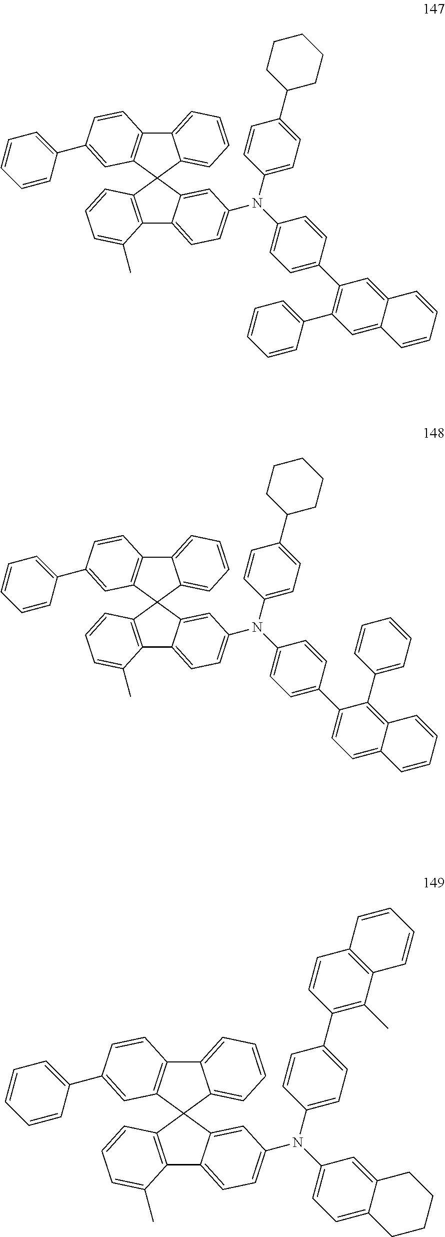 [SMILES: Cc1cccc2c1-c1ccc(N(c3ccc(-c4cc5ccccc5cc4-c4ccccc4)cc3)c3ccc(C4CCCCC4)cc3)cc1C21c2ccccc2-c2ccc(-c3ccccc3)cc21 ; Cc1cccc2c1-c1ccc(N(c3ccc(-c4ccc5ccccc5c4-c4ccccc4)cc3)c3ccc(C4CCCCC4)cc3)cc1C21c2ccccc2-c2ccc(-c3ccccc3)cc21 ; Cc1cccc2c1-c1ccc(N(c3ccc(-c4ccc5ccccc5c4C)cc3)c3ccc4c(c3)CCCC4)cc1C21c2ccccc2-c2ccc(-c3ccccc3)cc21]

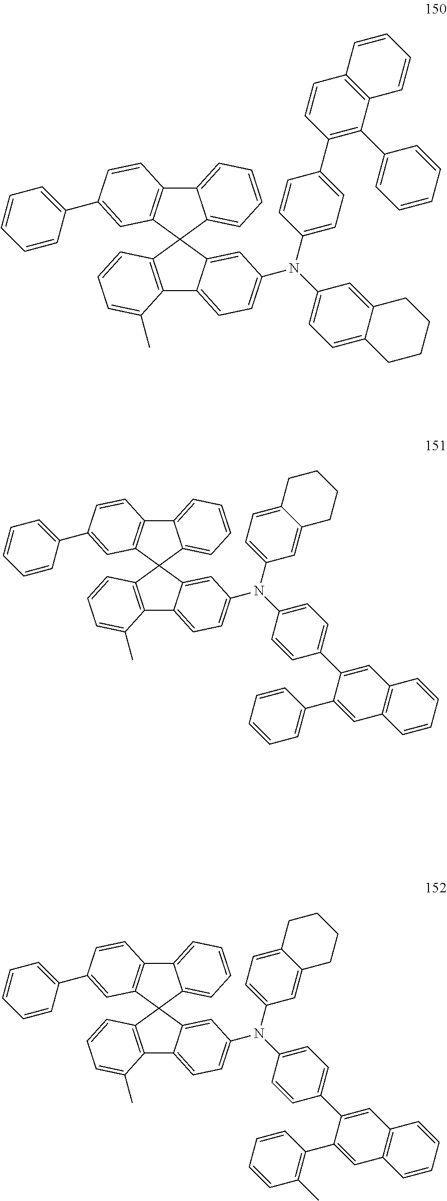 [SMILES: Cc1cccc2c1-c1ccc(N(c3ccc(-c4ccc5ccccc5c4-c4ccccc4)cc3)c3ccc4c(c3)CCCC4)cc1C21c2ccccc2-c2ccc(-c3ccccc3)cc21 ; Cc1cccc2c1-c1ccc(N(c3ccc(-c4cc5ccccc5cc4-c4ccccc4)cc3)c3ccc4c(c3)CCCC4)cc1C21c2ccccc2-c2ccc(-c3ccccc3)cc21 ; Cc1ccccc1-c1cc2ccccc2cc1-c1ccc(N(c2ccc3c(c2)CCCC3)c2ccc3c(c2)C2(c4ccccc4-c4ccc(-c5ccccc5)cc42)c2cccc(C)c2-3)cc1]

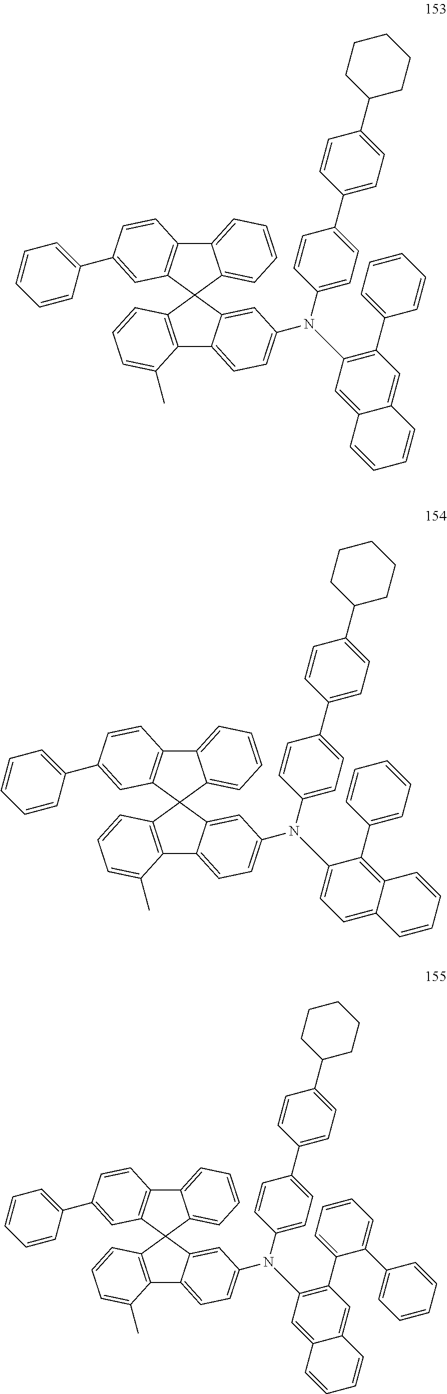 [SMILES: Cc1cccc2c1-c1ccc(N(c3ccc(-c4ccc(C5CCCCC5)cc4)cc3)c3cc4ccccc4cc3-c3ccccc3)cc1C21c2ccccc2-c2ccc(-c3ccccc3)cc21 ; Cc1cccc2c1-c1ccc(N(c3ccc(-c4ccc(C5CCCCC5)cc4)cc3)c3ccc4ccccc4c3-c3ccccc3)cc1C21c2ccccc2-c2ccc(-c3ccccc3)cc21 ; Cc1cccc2c1-c1ccc(N(c3ccc(-c4ccc(C5CCCCC5)cc4)cc3)c3cc4ccccc4cc3-c3ccccc3-c3ccccc3)cc1C21c2ccccc2-c2ccc(-c3ccccc3)cc21]

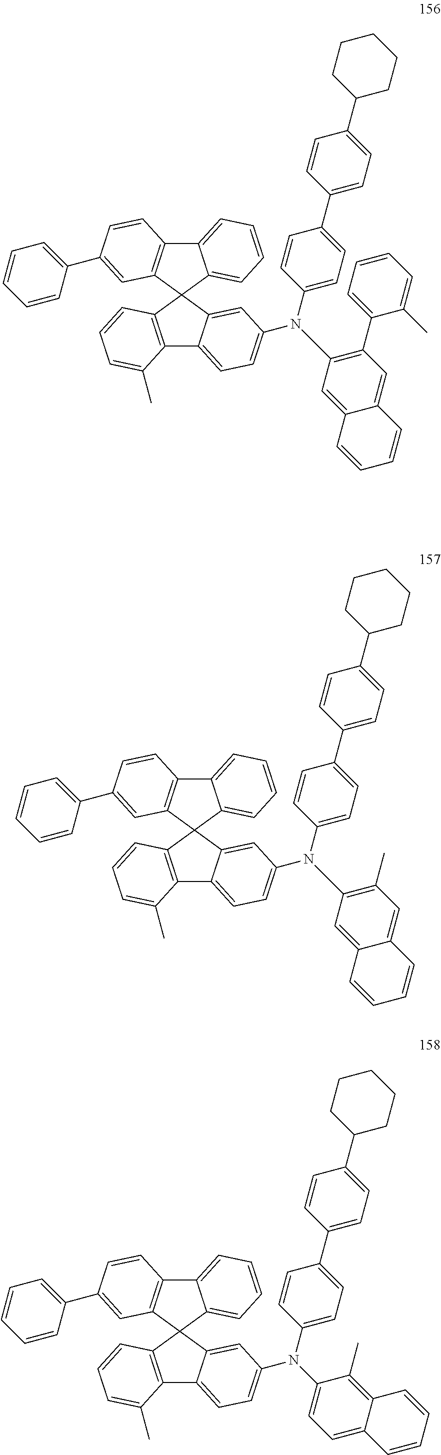 [SMILES: Cc1ccccc1-c1cc2ccccc2cc1N(c1ccc(-c2ccc(C3CCCCC3)cc2)cc1)c1ccc2c(c1)C1(c3ccccc3-c3ccc(-c4ccccc4)cc31)c1cccc(C)c1-2 ; Cc1cc2ccccc2cc1N(c1ccc(-c2ccc(C3CCCCC3)cc2)cc1)c1ccc2c(c1)C1(c3ccccc3-c3ccc(-c4ccccc4)cc31)c1cccc(C)c1-2 ; Cc1cccc2c1-c1ccc(N(c3ccc(-c4ccc(C5CCCCC5)cc4)cc3)c3ccc4ccccc4c3C)cc1C21c2ccccc2-c2ccc(-c3ccccc3)cc21]

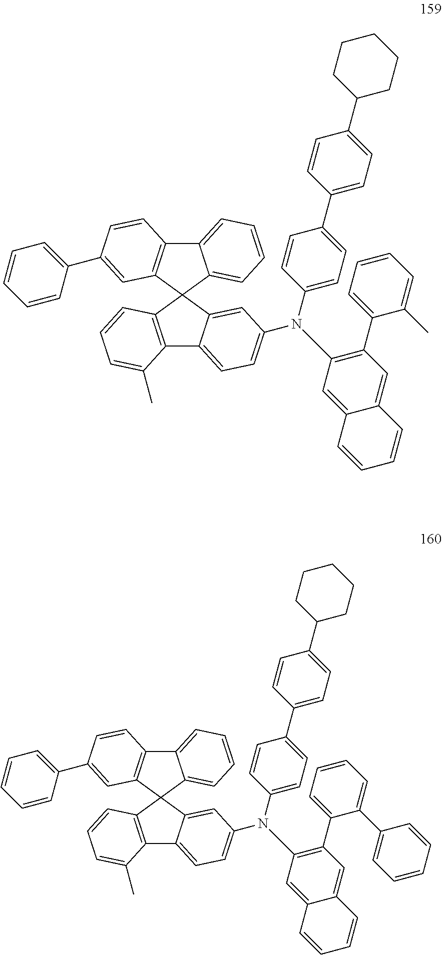 [SMILES: Cc1ccccc1-c1cc2ccccc2cc1N(c1ccc(-c2ccc(C3CCCCC3)cc2)cc1)c1ccc2c(c1)C1(c3ccccc3-c3ccc(-c4ccccc4)cc31)c1cccc(C)c1-2 ; Cc1cccc2c1-c1ccc(N(c3ccc(-c4ccc(C5CCCCC5)cc4)cc3)c3cc4ccccc4cc3-c3ccccc3-c3ccccc3)cc1C21c2ccccc2-c2ccc(-c3ccccc3)cc21]

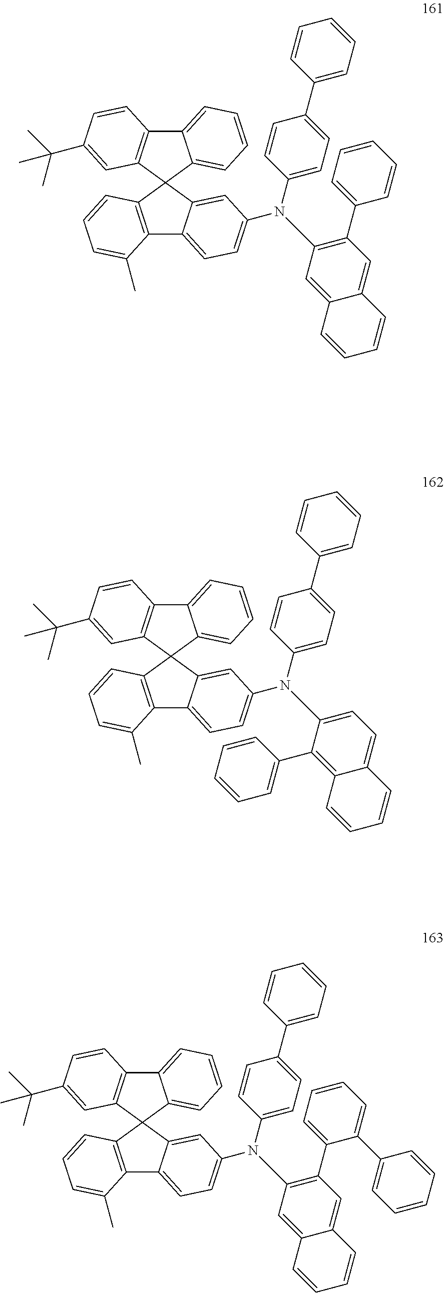 [SMILES: Cc1cccc2c1-c1ccc(N(c3ccc(-c4ccccc4)cc3)c3cc4ccccc4cc3-c3ccccc3)cc1C21c2ccccc2-c2ccc(C(C)(C)C)cc21 ; Cc1cccc2c1-c1ccc(N(c3ccc(-c4ccccc4)cc3)c3ccc4ccccc4c3-c3ccccc3)cc1C21c2ccccc2-c2ccc(C(C)(C)C)cc21 ; Cc1cccc2c1-c1ccc(N(c3ccc(-c4ccccc4)cc3)c3cc4ccccc4cc3-c3ccccc3-c3ccccc3)cc1C21c2ccccc2-c2ccc(C(C)(C)C)cc21]

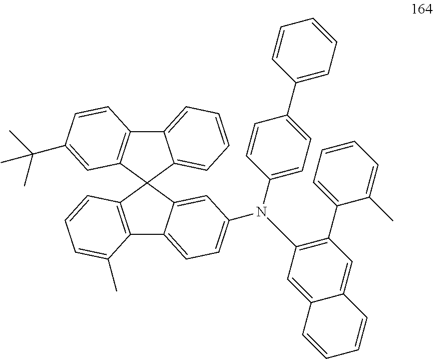 [SMILES: Cc1ccccc1-c1cc2ccccc2cc1N(c1ccc(-c2ccccc2)cc1)c1ccc2c(c1)C1(c3ccccc3-c3ccc(C(C)(C)C)cc31)c1cccc(C)c1-2]

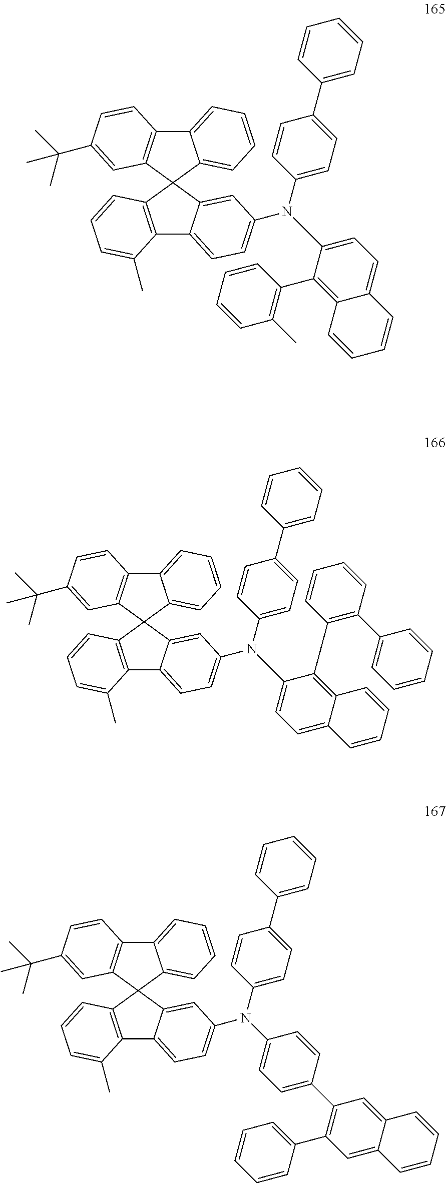 [SMILES: Cc1ccccc1-c1c(N(c2ccc(-c3ccccc3)cc2)c2ccc3c(c2)C2(c4ccccc4-c4ccc(C(C)(C)C)cc42)c2cccc(C)c2-3)ccc2ccccc12 ; Cc1cccc2c1-c1ccc(N(c3ccc(-c4ccccc4)cc3)c3ccc4ccccc4c3-c3ccccc3-c3ccccc3)cc1C21c2ccccc2-c2ccc(C(C)(C)C)cc21 ; Cc1cccc2c1-c1ccc(N(c3ccc(-c4ccccc4)cc3)c3ccc(-c4cc5ccccc5cc4-c4ccccc4)cc3)cc1C21c2ccccc2-c2ccc(C(C)(C)C)cc21]

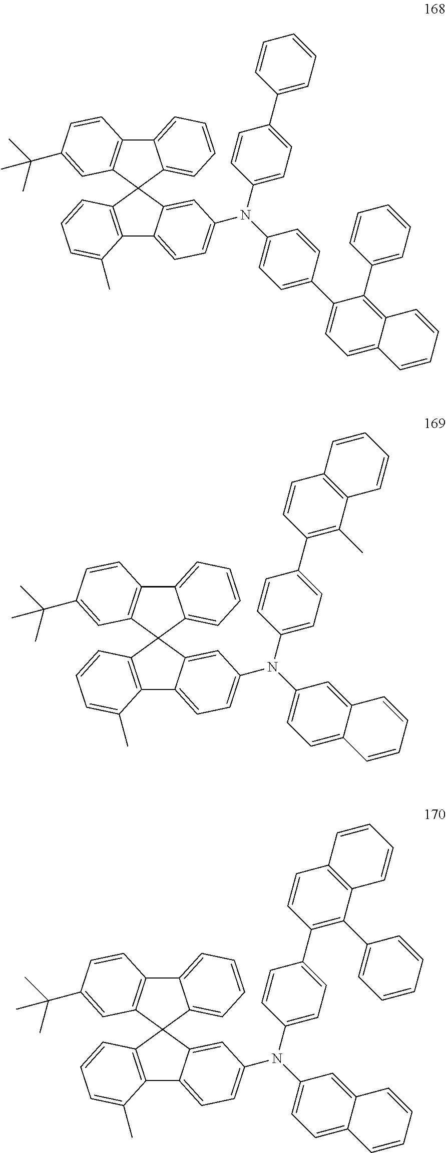 [SMILES: Cc1cccc2c1-c1ccc(N(c3ccc(-c4ccccc4)cc3)c3ccc(-c4ccc5ccccc5c4-c4ccccc4)cc3)cc1C21c2ccccc2-c2ccc(C(C)(C)C)cc21 ; Cc1cccc2c1-c1ccc(N(c3ccc(-c4ccc5ccccc5c4C)cc3)c3ccc4ccccc4c3)cc1C21c2ccccc2-c2ccc(C(C)(C)C)cc21 ; Cc1cccc2c1-c1ccc(N(c3ccc(-c4ccc5ccccc5c4-c4ccccc4)cc3)c3ccc4ccccc4c3)cc1C21c2ccccc2-c2ccc(C(C)(C)C)cc21]

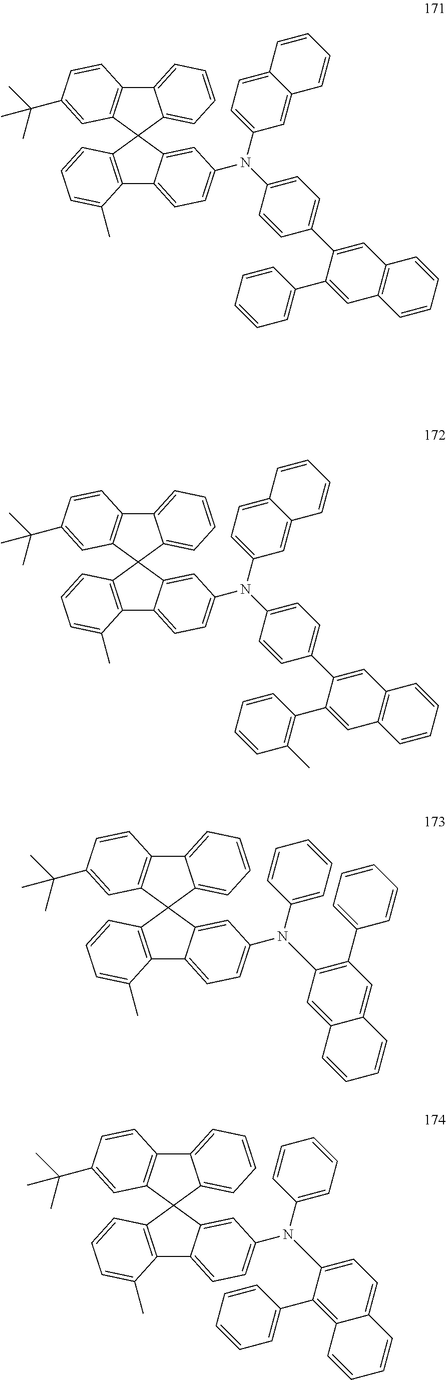 [SMILES: Cc1cccc2c1-c1ccc(N(c3ccc(-c4cc5ccccc5cc4-c4ccccc4)cc3)c3ccc4ccccc4c3)cc1C21c2ccccc2-c2ccc(C(C)(C)C)cc21 ; Cc1ccccc1-c1cc2ccccc2cc1-c1ccc(N(c2ccc3c(c2)C2(c4ccccc4-c4ccc(C(C)(C)C)cc42)c2cccc(C)c2-3)c2ccc3ccccc3c2)cc1 ; Cc1cccc2c1-c1ccc(N(c3ccccc3)c3cc4ccccc4cc3-c3ccccc3)cc1C21c2ccccc2-c2ccc(C(C)(C)C)cc21 ; Cc1cccc2c1-c1ccc(N(c3ccccc3)c3ccc4ccccc4c3-c3ccccc3)cc1C21c2ccccc2-c2ccc(C(C)(C)C)cc21]

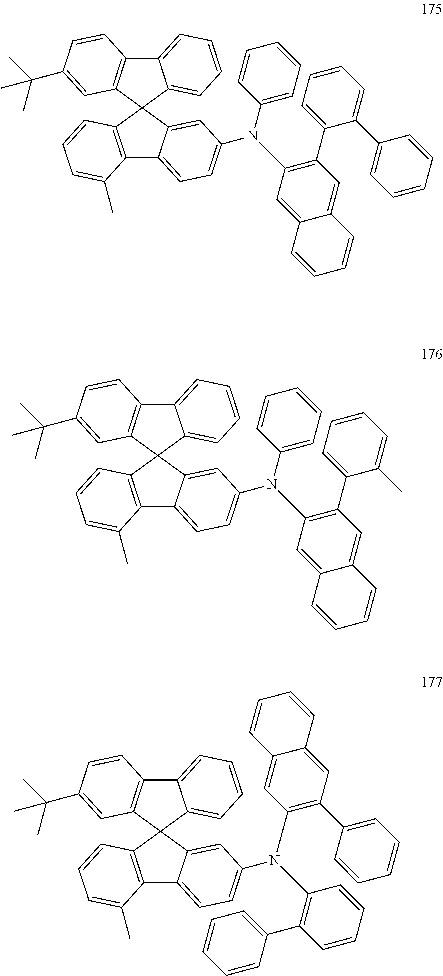

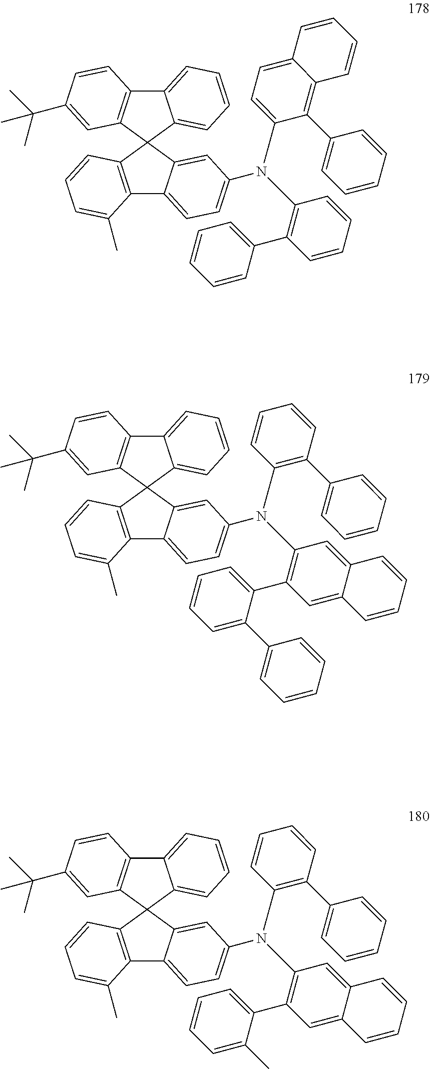 [SMILES: Cc1cccc2c1-c1ccc(N(c3ccccc3-c3ccccc3)c3ccc4ccccc4c3-c3ccccc3)cc1C21c2ccccc2-c2ccc(C(C)(C)C)cc21 ; Cc1cccc2c1-c1ccc(N(c3ccccc3-c3ccccc3)c3cc4ccccc4cc3-c3ccccc3-c3ccccc3)cc1C21c2ccccc2-c2ccc(C(C)(C)C)cc21 ; Cc1ccccc1-c1cc2ccccc2cc1N(c1ccc2c(c1)C1(c3ccccc3-c3ccc(C(C)(C)C)cc31)c1cccc(C)c1-2)c1ccccc1-c1ccccc1]

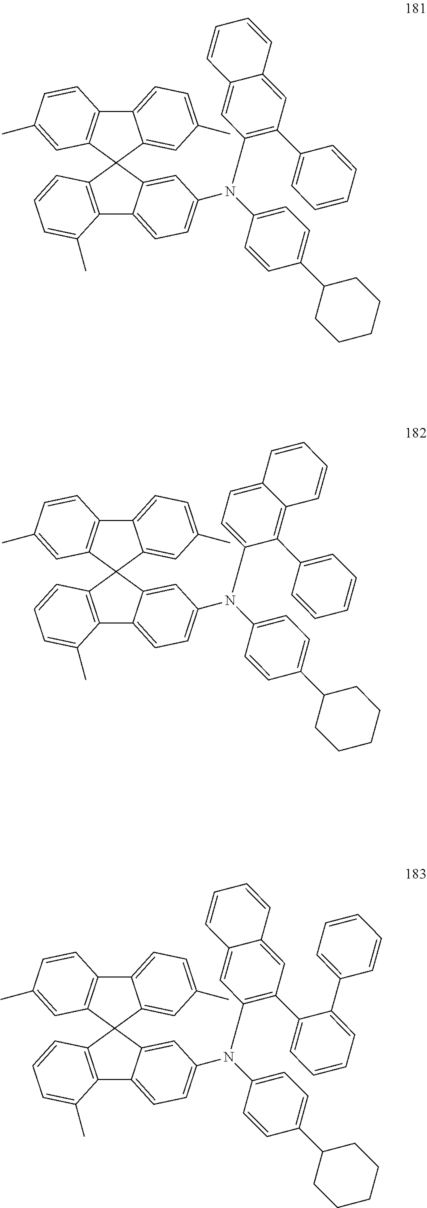 [SMILES: Cc1ccc2c(c1)C1(c3cc(C)ccc3-2)c2cc(N(c3ccc(C4CCCCC4)cc3)c3cc4ccccc4cc3-c3ccccc3)ccc2-c2c(C)cccc21 ; Cc1ccc2c(c1)C1(c3cc(C)ccc3-2)c2cc(N(c3ccc(C4CCCCC4)cc3)c3ccc4ccccc4c3-c3ccccc3)ccc2-c2c(C)cccc21 ; Cc1ccc2c(c1)C1(c3cc(C)ccc3-2)c2cc(N(c3ccc(C4CCCCC4)cc3)c3cc4ccccc4cc3-c3ccccc3-c3ccccc3)ccc2-c2c(C)cccc21]

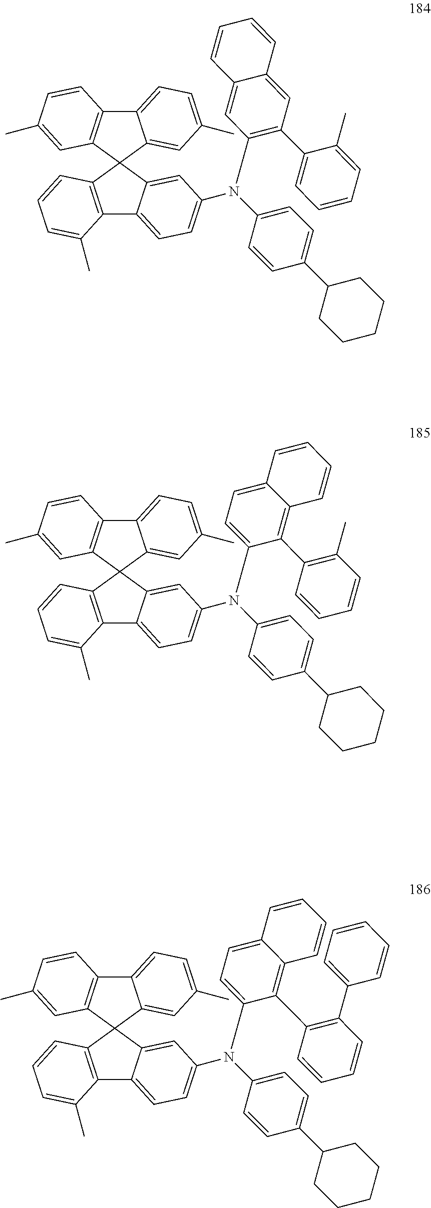 [SMILES: Cc1ccc2c(c1)C1(c3cc(C)ccc3-2)c2cc(N(c3ccc(C4CCCCC4)cc3)c3cc4ccccc4cc3-c3ccccc3C)ccc2-c2c(C)cccc21 ; Cc1ccc2c(c1)C1(c3cc(C)ccc3-2)c2cc(N(c3ccc(C4CCCCC4)cc3)c3ccc4ccccc4c3-c3ccccc3C)ccc2-c2c(C)cccc21 ; Cc1ccc2c(c1)C1(c3cc(C)ccc3-2)c2cc(N(c3ccc(C4CCCCC4)cc3)c3ccc4ccccc4c3-c3ccccc3-c3ccccc3)ccc2-c2c(C)cccc21]

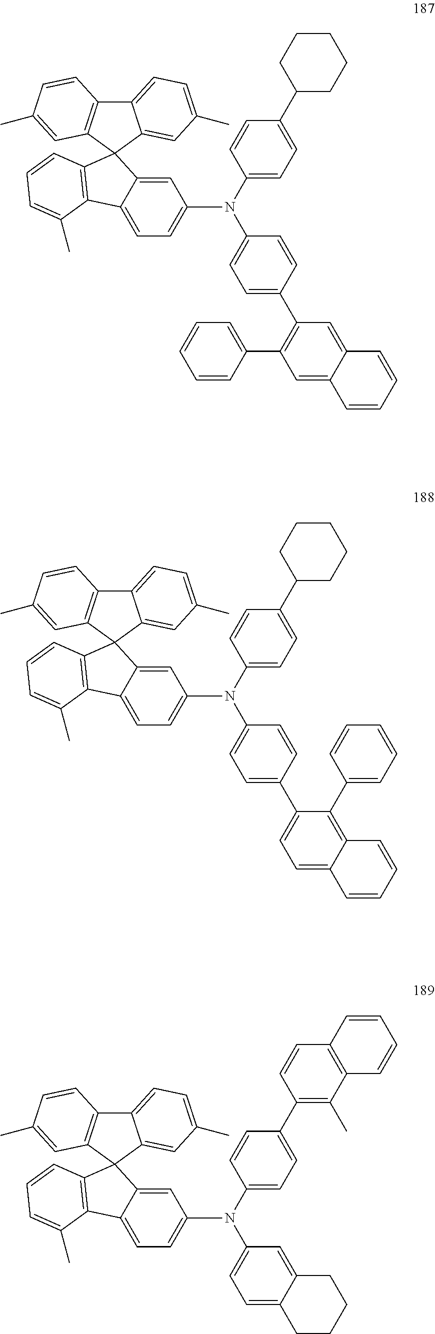 [SMILES: Cc1ccc2c(c1)C1(c3cc(C)ccc3-2)c2cc(N(c3ccc(-c4cc5ccccc5cc4-c4ccccc4)cc3)c3ccc(C4CCCCC4)cc3)ccc2-c2c(C)cccc21 ; Cc1ccc2c(c1)C1(c3cc(C)ccc3-2)c2cc(N(c3ccc(-c4ccc5ccccc5c4-c4ccccc4)cc3)c3ccc(C4CCCCC4)cc3)ccc2-c2c(C)cccc21 ; Cc1ccc2c(c1)C1(c3cc(C)ccc3-2)c2cc(N(c3ccc(-c4ccc5ccccc5c4C)cc3)c3ccc4c(c3)CCCC4)ccc2-c2c(C)cccc21]

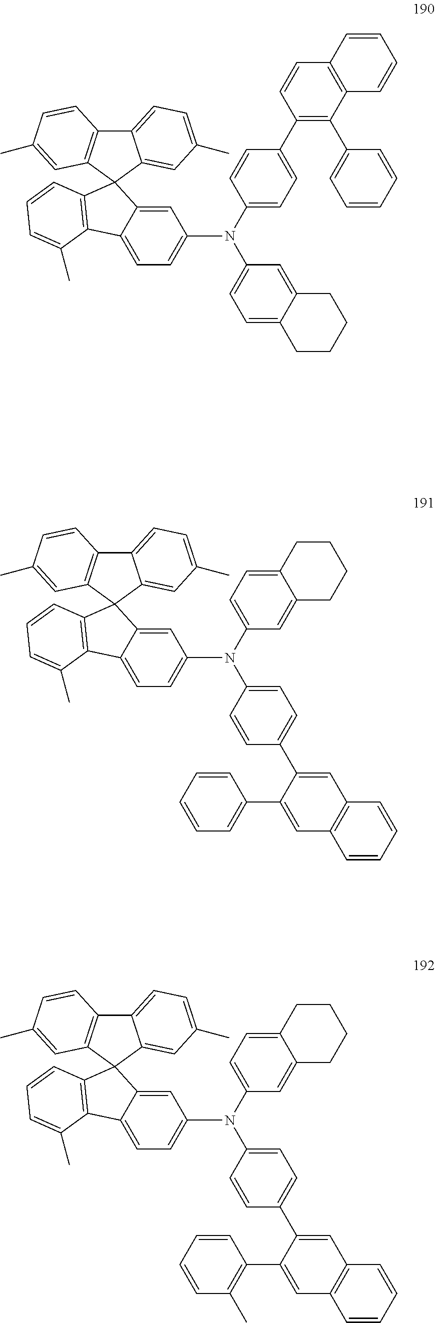 [SMILES: Cc1ccc2c(c1)C1(c3cc(C)ccc3-2)c2cc(N(c3ccc(-c4ccc5ccccc5c4-c4ccccc4)cc3)c3ccc4c(c3)CCCC4)ccc2-c2c(C)cccc21 ; Cc1ccc2c(c1)C1(c3cc(C)ccc3-2)c2cc(N(c3ccc(-c4cc5ccccc5cc4-c4ccccc4)cc3)c3ccc4c(c3)CCCC4)ccc2-c2c(C)cccc21 ; Cc1ccc2c(c1)C1(c3cc(C)ccc3-2)c2cc(N(c3ccc(-c4cc5ccccc5cc4-c4ccccc4C)cc3)c3ccc4c(c3)CCCC4)ccc2-c2c(C)cccc21]

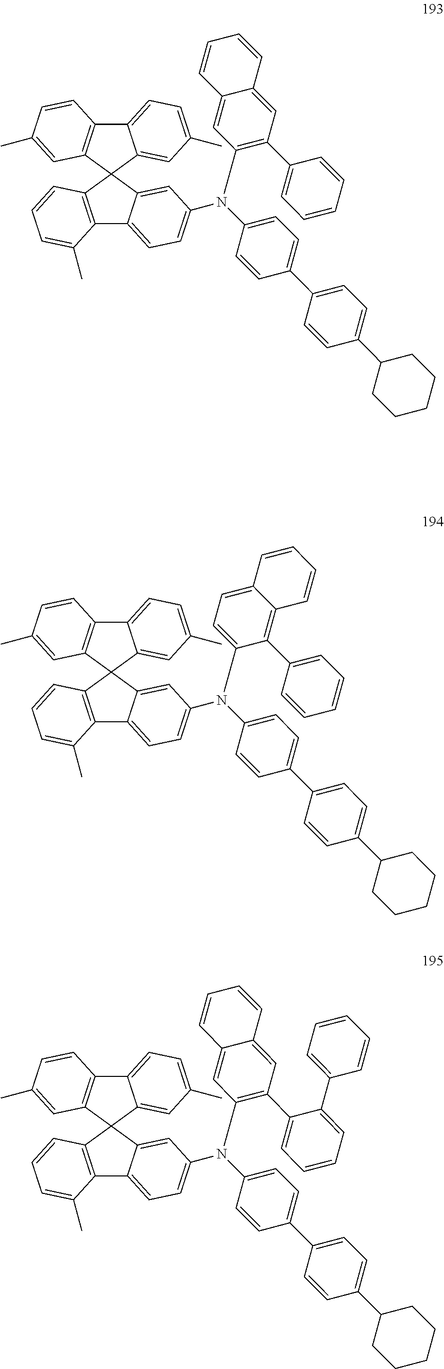 [SMILES: Cc1ccc2c(c1)C1(c3cc(C)ccc3-2)c2cc(N(c3ccc(-c4ccc(C5CCCCC5)cc4)cc3)c3cc4ccccc4cc3-c3ccccc3)ccc2-c2c(C)cccc21 ; Cc1ccc2c(c1)C1(c3cc(C)ccc3-2)c2cc(N(c3ccc(-c4ccc(C5CCCCC5)cc4)cc3)c3ccc4ccccc4c3-c3ccccc3)ccc2-c2c(C)cccc21 ; Cc1ccc2c(c1)C1(c3cc(C)ccc3-2)c2cc(N(c3ccc(-c4ccc(C5CCCCC5)cc4)cc3)c3cc4ccccc4cc3-c3ccccc3-c3ccccc3)ccc2-c2c(C)cccc21]

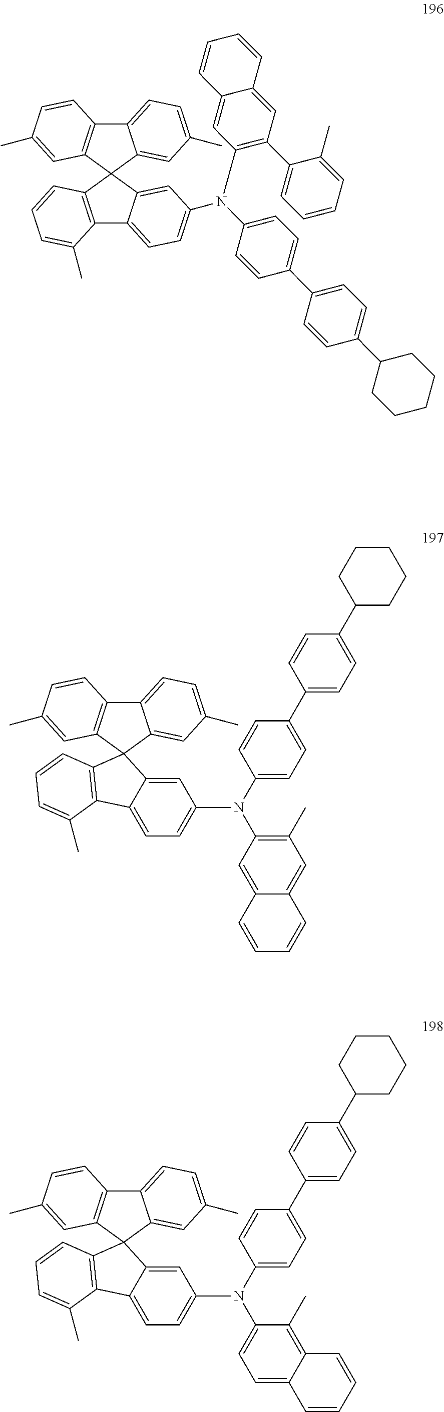 [SMILES: Cc1ccc2c(c1)C1(c3cc(C)ccc3-2)c2cc(N(c3ccc(-c4ccc(C5CCCCC5)cc4)cc3)c3cc4ccccc4cc3-c3ccccc3C)ccc2-c2c(C)cccc21 ; Cc1ccc2c(c1)C1(c3cc(C)ccc3-2)c2cc(N(c3ccc(-c4ccc(C5CCCCC5)cc4)cc3)c3cc4ccccc4cc3C)ccc2-c2c(C)cccc21 ; Cc1ccc2c(c1)C1(c3cc(C)ccc3-2)c2cc(N(c3ccc(-c4ccc(C5CCCCC5)cc4)cc3)c3ccc4ccccc4c3C)ccc2-c2c(C)cccc21]

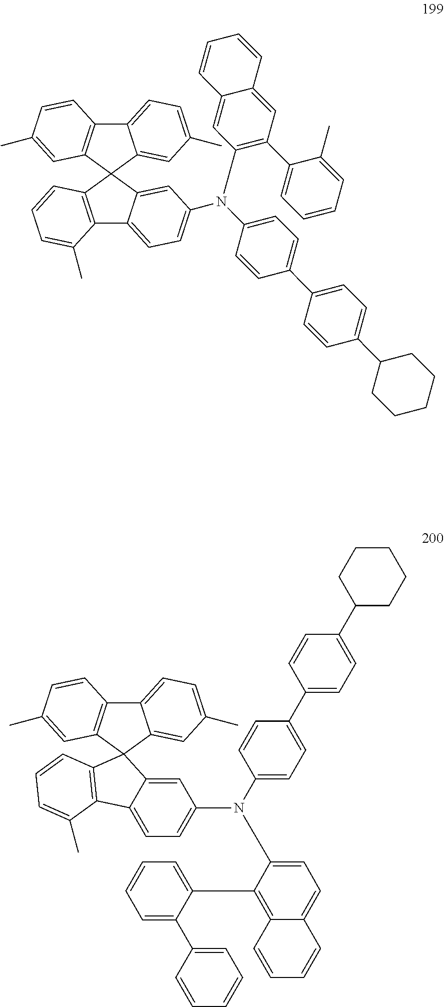 [SMILES: Cc1ccc2c(c1)C1(c3cc(C)ccc3-2)c2cc(N(c3ccc(-c4ccc(C5CCCCC5)cc4)cc3)c3cc4ccccc4cc3-c3ccccc3C)ccc2-c2c(C)cccc21 ; Cc1ccc2c(c1)C1(c3cc(C)ccc3-2)c2cc(N(c3ccc(-c4ccc(C5CCCCC5)cc4)cc3)c3ccc4ccccc4c3-c3ccccc3-c3ccccc3)ccc2-c2c(C)cccc21]

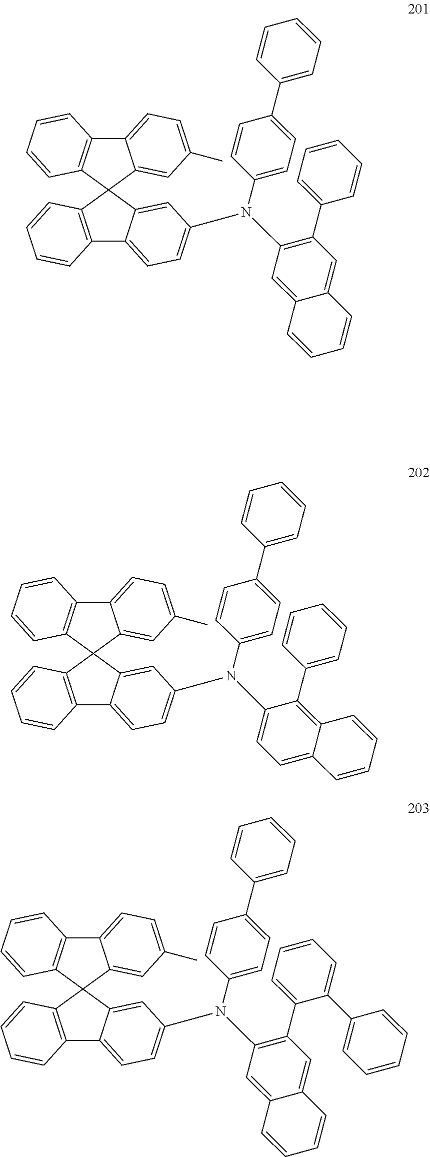 [SMILES: Cc1ccc2c(c1)C1(c3ccccc3-2)c2ccccc2-c2ccc(N(c3ccc(-c4ccccc4)cc3)c3cc4ccccc4cc3-c3ccccc3)cc21 ; Cc1ccc2c(c1)C1(c3ccccc3-2)c2ccccc2-c2ccc(N(c3ccc(-c4ccccc4)cc3)c3ccc4ccccc4c3-c3ccccc3)cc21 ; Cc1ccc2c(c1)C1(c3ccccc3-2)c2ccccc2-c2ccc(N(c3ccc(-c4ccccc4)cc3)c3cc4ccccc4cc3-c3ccccc3-c3ccccc3)cc21]

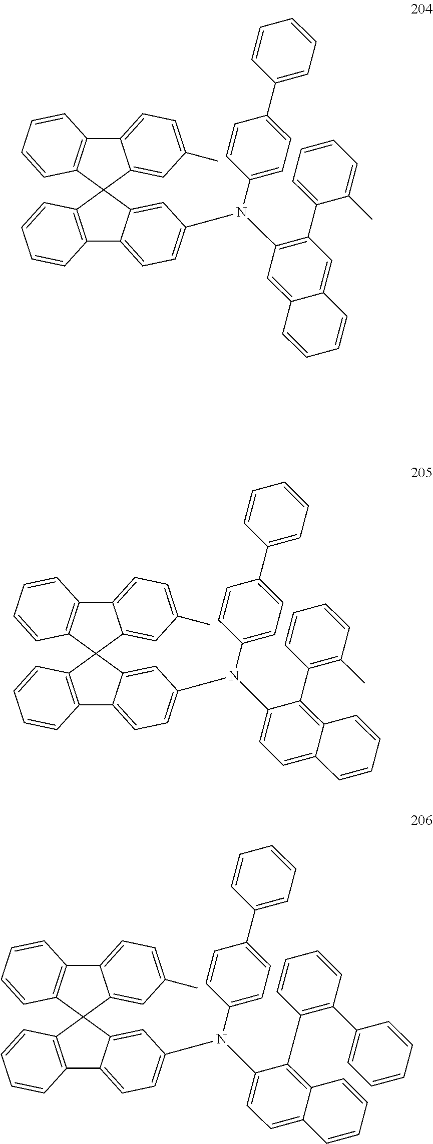 [SMILES: Cc1ccc2c(c1)C1(c3ccccc3-2)c2ccccc2-c2ccc(N(c3ccc(-c4ccccc4)cc3)c3cc4ccccc4cc3-c3ccccc3C)cc21 ; Cc1ccc2c(c1)C1(c3ccccc3-2)c2ccccc2-c2ccc(N(c3ccc(-c4ccccc4)cc3)c3ccc4ccccc4c3-c3ccccc3C)cc21 ; Cc1ccc2c(c1)C1(c3ccccc3-2)c2ccccc2-c2ccc(N(c3ccc(-c4ccccc4)cc3)c3ccc4ccccc4c3-c3ccccc3-c3ccccc3)cc21]

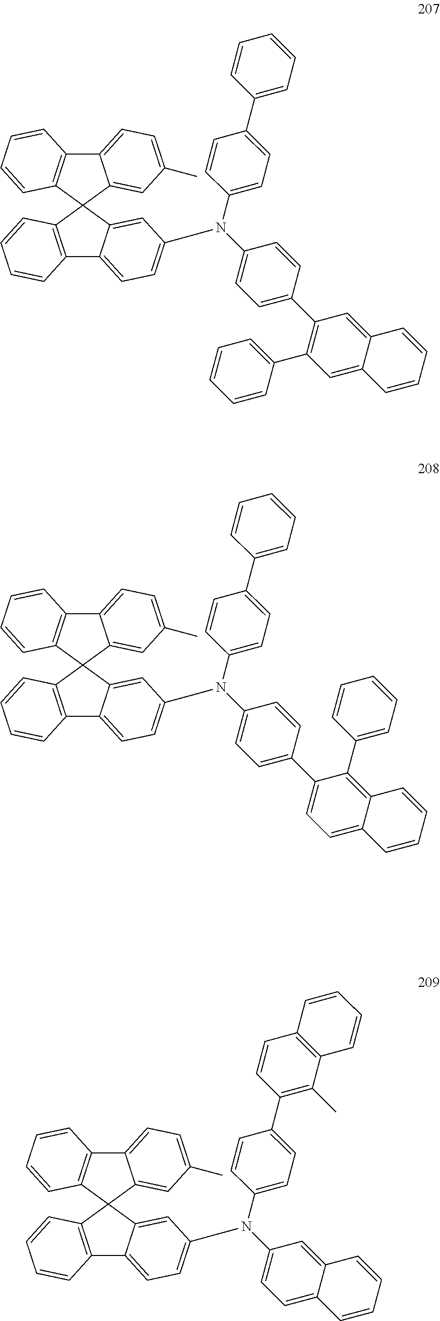 [SMILES: Cc1ccc2c(c1)C1(c3ccccc3-2)c2ccccc2-c2ccc(N(c3ccc(-c4ccccc4)cc3)c3ccc(-c4cc5ccccc5cc4-c4ccccc4)cc3)cc21 ; Cc1ccc2c(c1)C1(c3ccccc3-2)c2ccccc2-c2ccc(N(c3ccc(-c4ccccc4)cc3)c3ccc(-c4ccc5ccccc5c4-c4ccccc4)cc3)cc21 ; Cc1ccc2c(c1)C1(c3ccccc3-2)c2ccccc2-c2ccc(N(c3ccc(-c4ccc5ccccc5c4C)cc3)c3ccc4ccccc4c3)cc21]

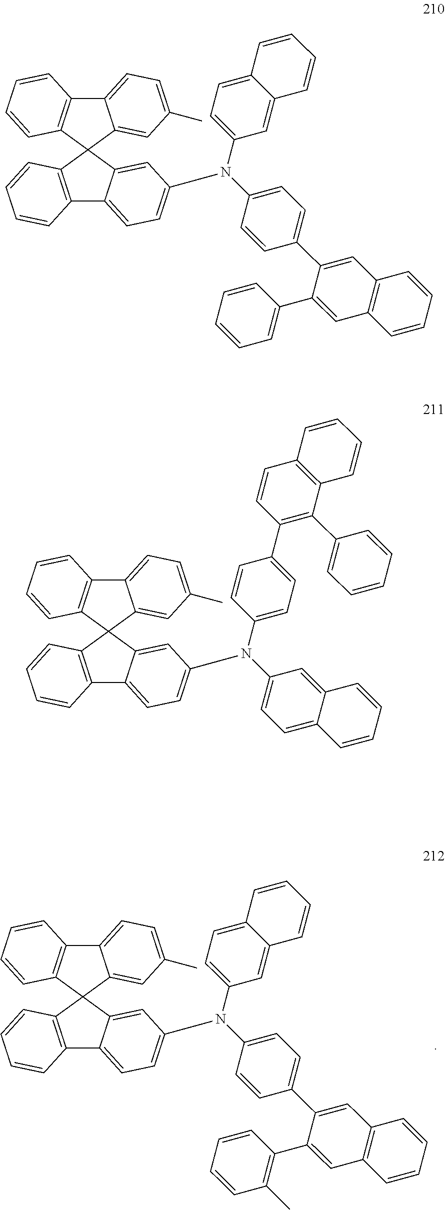 [SMILES: Cc1ccc2c(c1)C1(c3ccccc3-2)c2ccccc2-c2ccc(N(c3ccc(-c4cc5ccccc5cc4-c4ccccc4)cc3)c3ccc4ccccc4c3)cc21 ; Cc1ccc2c(c1)C1(c3ccccc3-2)c2ccccc2-c2ccc(N(c3ccc(-c4ccc5ccccc5c4-c4ccccc4)cc3)c3ccc4ccccc4c3)cc21 ; Cc1ccc2c(c1)C1(c3ccccc3-2)c2ccccc2-c2ccc(N(c3ccc(-c4cc5ccccc5cc4-c4ccccc4C)cc3)c3ccc4ccccc4c3)cc21]

14. An amine compound represented by Formula 1: 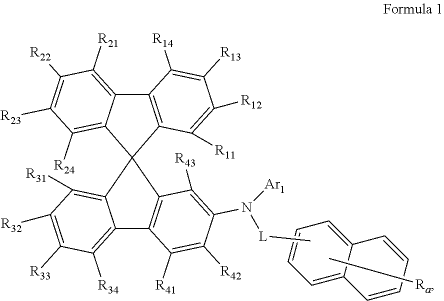
wherein, in Formula 1,
at least one selected from among R₁₁ to R₁₄, R₂₁ to R₂₄, R₃₁ to R₃₄, and R₄₁ to R₄₃ is a substituted or unsubstituted methyl group, and the remainder are each independently hydrogen, deuterium, a halogen, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, and/or combined with an adjacent group to form a ring,
Ar₁ is a substituted or unsubstituted aryl group of 6 to 30 ring-forming carbon atoms, or a substituted or unsubstituted heteroaryl group of 2 to 30 ring-forming carbon atoms,
L is a direct linkage, a substituted or unsubstituted arylene group of 6 to 30 ring-forming carbon atoms, or a substituted or unsubstituted heteroarylene group of 2 to 30 ring-forming carbon atoms, and
Rₐ is hydrogen, deuterium, a halogen, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group from each other to form a ring.

15. The amine compound of claim 14, wherein, in Formula 1, at least one selected from among R₁₂, R₂₃, and R₃₄ is an unsubstituted methyl group.

16. The amine compound of claim 15, wherein Formula 1 is represented by any one selected from among Formula 1-1 to Formula 1-4: 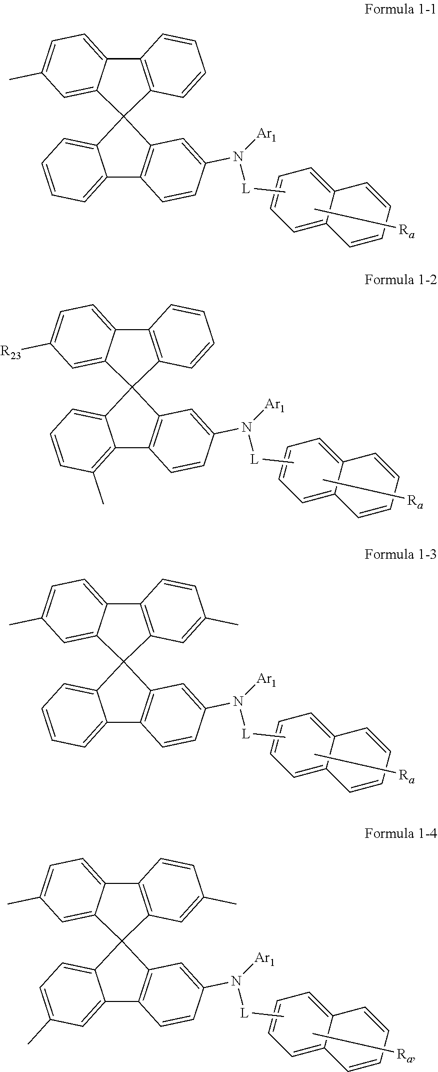
and
wherein, in Formula 1-1 to Formula 1-4, R₂₃, Ar₁, L, and Rₐ are the same as defined in Formula 1.

17. The amine compound of claim 16, wherein, in Formula 1-2, R₂₃ is any one selected from among hydrogen, an unsubstituted methyl group, an unsubstituted phenyl group, and an unsubstituted t-butyl group.

18. The amine compound of claim 14, wherein Formula 1 is represented by Formula 2: 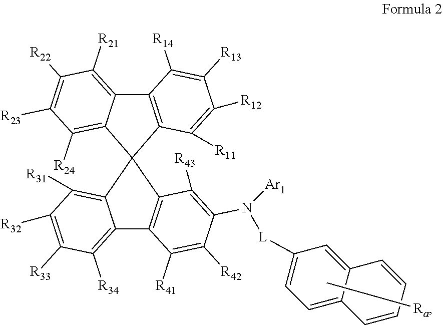
and
wherein, in Formula 2, R₁₁ to R₁₄, R₂₁ to R₂₄, R₃₁ to R₃₄, R₄₁ to R₄₃, Ar₁, L, and Rₐ are the same as defined in Formula 1.

19. The amine compound of claim 18, wherein Formula 2 is represented by Formula 3-1 or Formula 3-2: 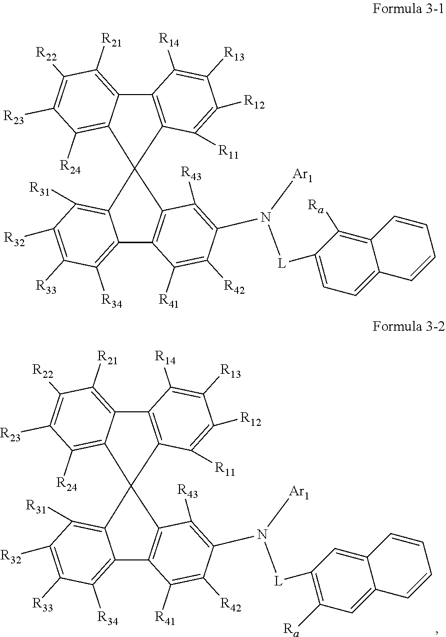
and
wherein, in Formula 3-1 and Formula 3-2, R₁₁ to R₁₄, R₂₁ to R₂₄, R₃₁ to R₃₄, R₄₁ to R₄₃, Ar₁, L, and Rₐ are the same as defined in Formula 1.

20. The amine compound of claim 14, wherein, in Formula 1, Rₐ is a substituted or unsubstituted phenyl group, or a substituted or unsubstituted methyl group.

21. The amine compound of claim 14, wherein Formula 1 is represented by Formula 4-1 or Formula 4-2: 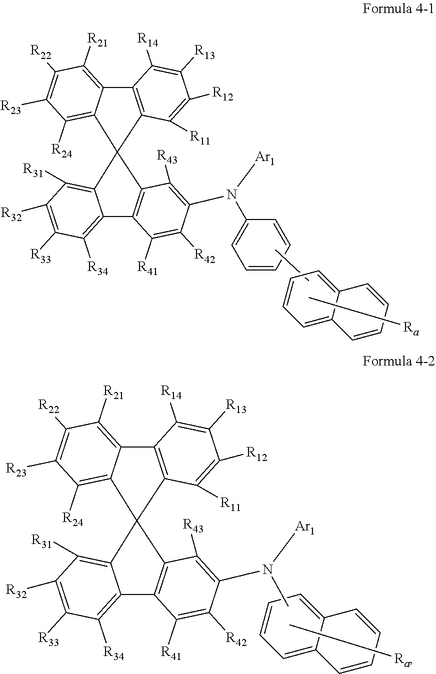
and
wherein, in Formula 4-1 and Formula 4-2, R₁₁ to R₁₄, R₂₁ to R₂₄, R₃₁ to R₃₄, R₄₁ to R₄₃, Ar₁, and Rₐ are the same as defined in Formula 1.

22. The amine compound of claim 21, wherein Formula 4-1 is represented by Formula 5: 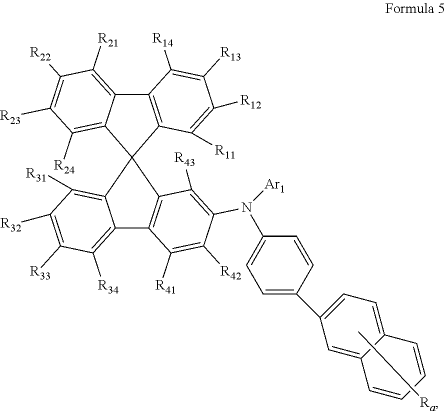
and
wherein, in Formula 5, R₁₁ to R₁₄, R₂₁ to R₂₄, R₃₁ to R₃₄, R₄₁ to R₄₃, Ar₁, and Rₐ are the same as defined in Formula 1.

23. The amine compound of claim 14, wherein Formula 1 is represented by any one selected from among compounds in Compound Group 1: 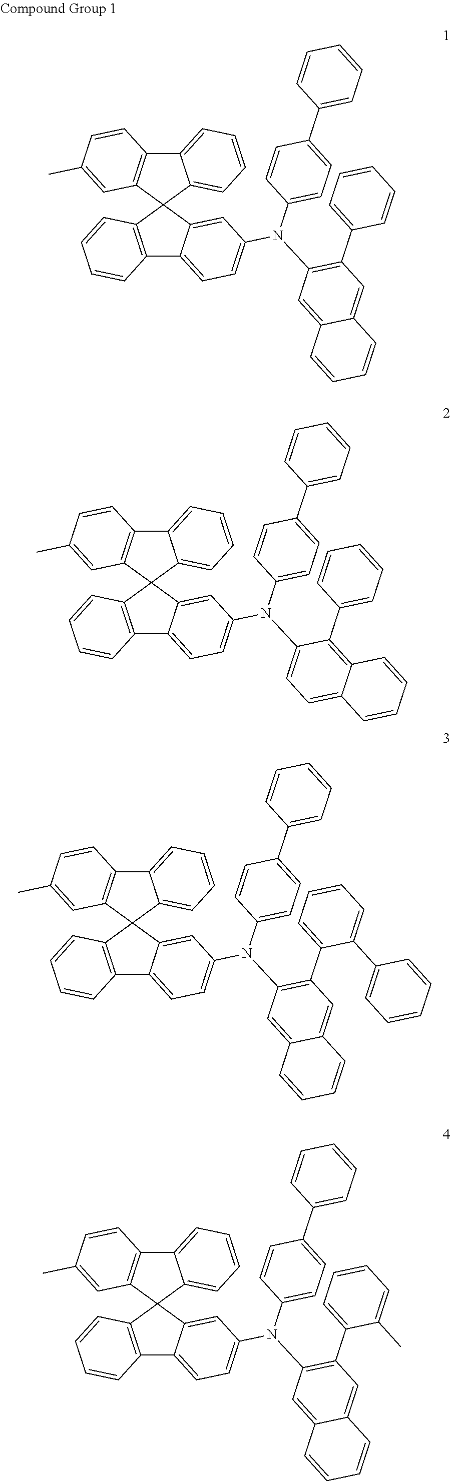 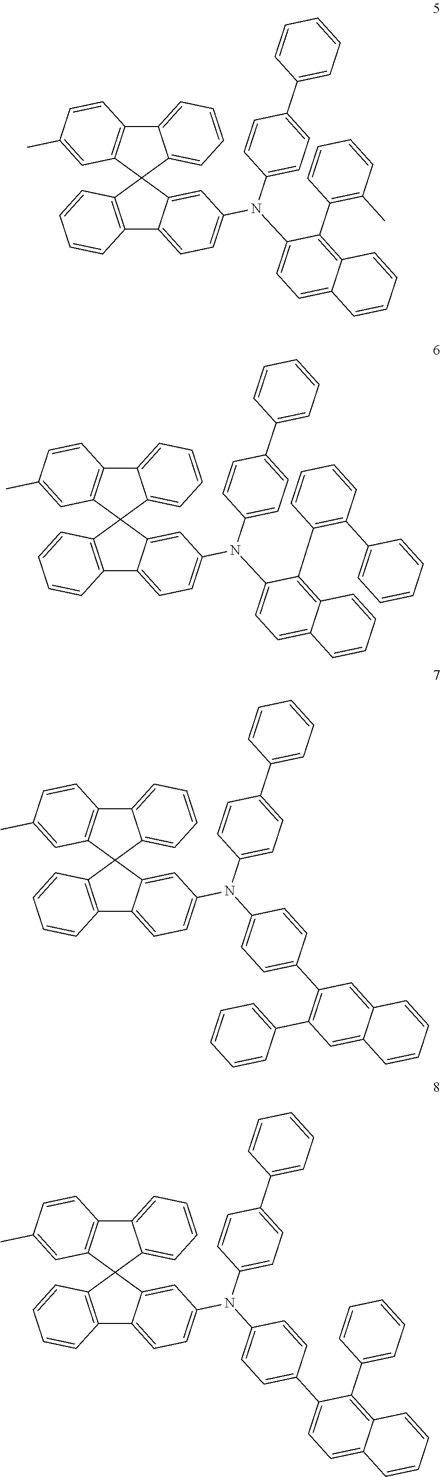 [SMILES: Cc1ccc2c(c1)C1(c3ccccc3-2)c2ccccc2-c2ccc(N(c3ccc(-c4ccccc4)cc3)c3ccc4ccccc4c3-c3ccccc3C)cc21 ; Cc1ccc2c(c1)C1(c3ccccc3-2)c2ccccc2-c2ccc(N(c3ccc(-c4ccccc4)cc3)c3ccc4ccccc4c3-c3ccccc3-c3ccccc3)cc21 ; Cc1ccc2c(c1)C1(c3ccccc3-2)c2ccccc2-c2ccc(N(c3ccc(-c4ccccc4)cc3)c3ccc(-c4cc5ccccc5cc4-c4ccccc4)cc3)cc21 ; Cc1ccc2c(c1)C1(c3ccccc3-2)c2ccccc2-c2ccc(N(c3ccc(-c4ccccc4)cc3)c3ccc(-c4ccc5ccccc5c4-c4ccccc4)cc3)cc21] 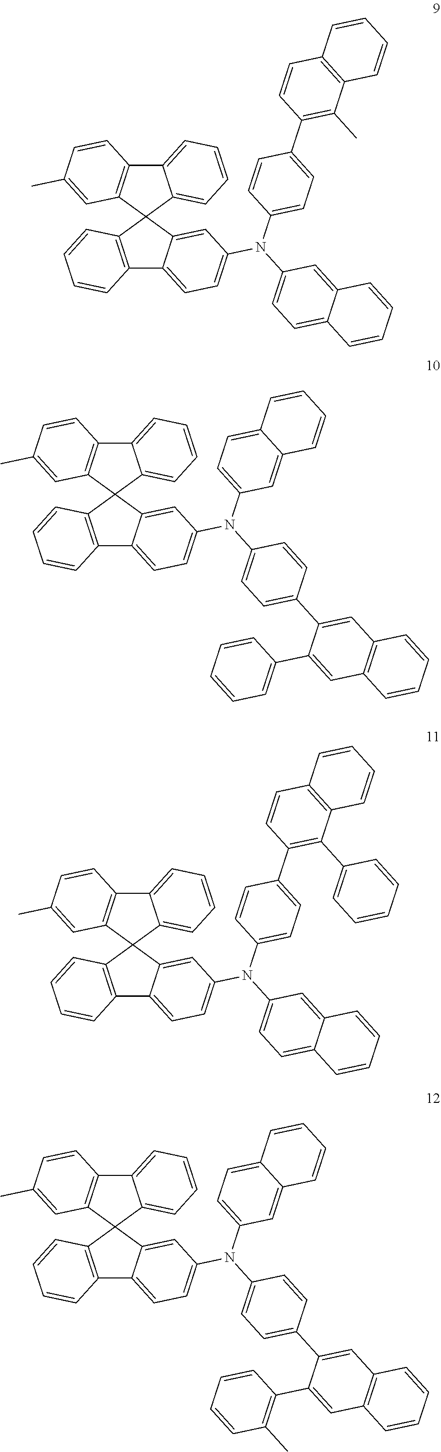 [SMILES: Cc1ccc2c(c1)C1(c3ccccc3-2)c2ccccc2-c2ccc(N(c3ccc(-c4ccc5ccccc5c4C)cc3)c3ccc4ccccc4c3)cc21 ; Cc1ccc2c(c1)C1(c3ccccc3-2)c2ccccc2-c2ccc(N(c3ccc(-c4cc5ccccc5cc4-c4ccccc4)cc3)c3ccc4ccccc4c3)cc21 ; Cc1ccc2c(c1)C1(c3ccccc3-2)c2ccccc2-c2ccc(N(c3ccc(-c4ccc5ccccc5c4-c4ccccc4)cc3)c3ccc4ccccc4c3)cc21 ; Cc1ccc2c(c1)C1(c3ccccc3-2)c2ccccc2-c2ccc(N(c3ccc(-c4cc5ccccc5cc4-c4ccccc4C)cc3)c3ccc4ccccc4c3)cc21] 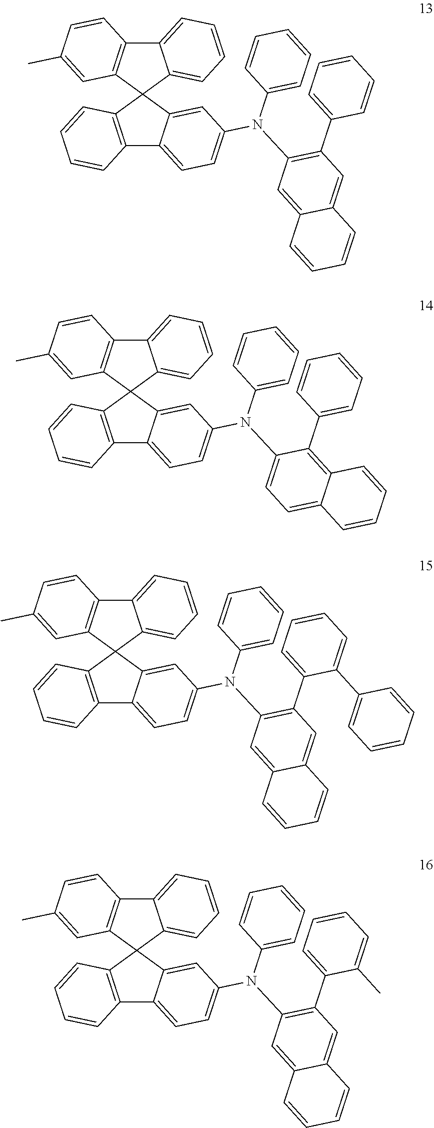 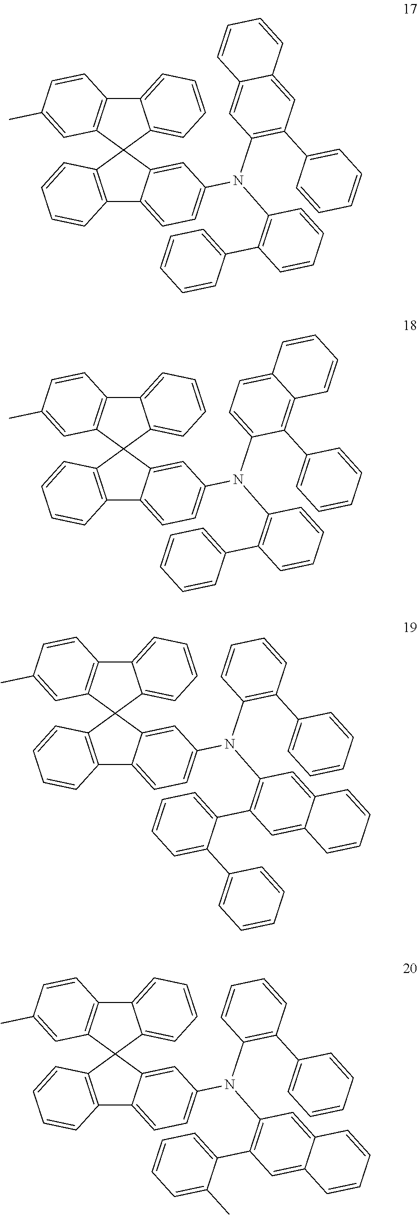 [SMILES: Cc1ccc2c(c1)C1(c3ccccc3-2)c2ccccc2-c2ccc(N(c3ccccc3-c3ccccc3)c3cc4ccccc4cc3-c3ccccc3)cc21 ; Cc1ccc2c(c1)C1(c3ccccc3-2)c2ccccc2-c2ccc(N(c3ccccc3-c3ccccc3)c3ccc4ccccc4c3-c3ccccc3)cc21 ; Cc1ccc2c(c1)C1(c3ccccc3-2)c2ccccc2-c2ccc(N(c3ccccc3-c3ccccc3)c3cc4ccccc4cc3-c3ccccc3-c3ccccc3)cc21 ; Cc1ccc2c(c1)C1(c3ccccc3-2)c2ccccc2-c2ccc(N(c3ccccc3-c3ccccc3)c3cc4ccccc4cc3-c3ccccc3C)cc21] 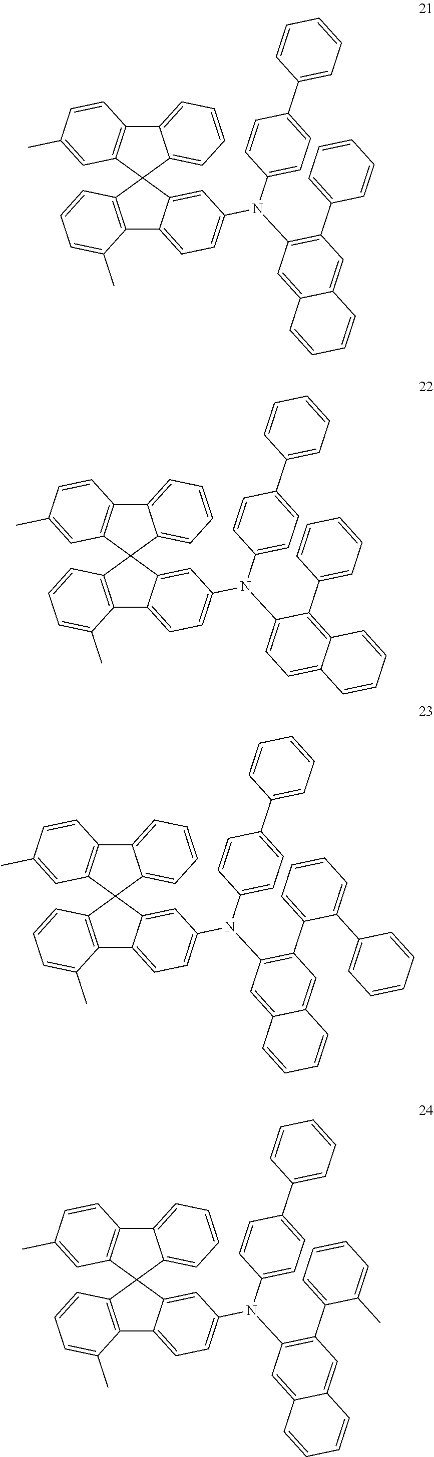 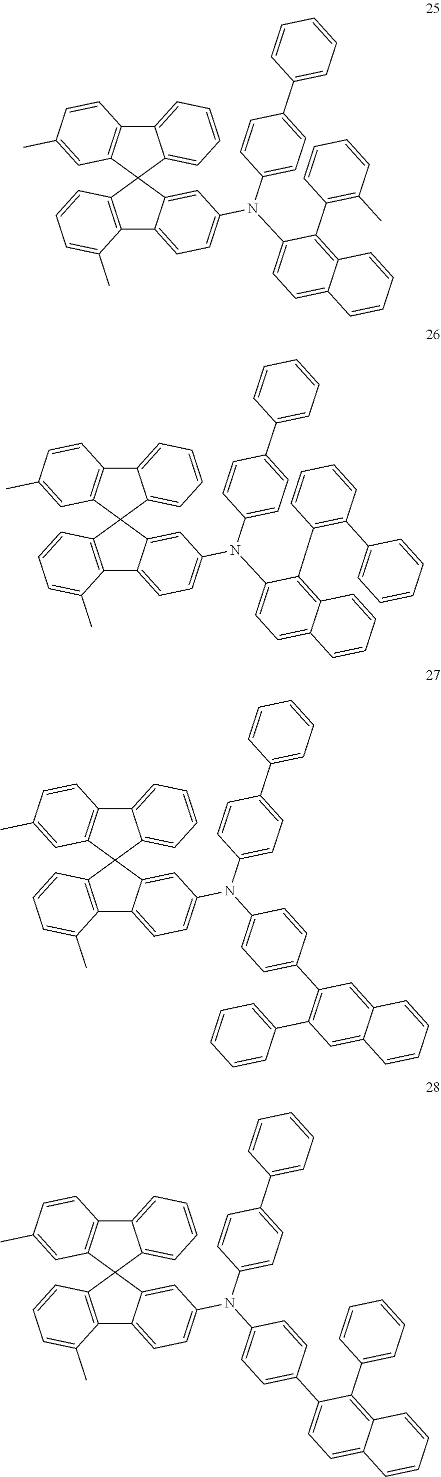 [SMILES: Cc1ccc2c(c1)C1(c3ccccc3-2)c2cc(N(c3ccc(-c4ccccc4)cc3)c3ccc4ccccc4c3-c3ccccc3C)ccc2-c2c(C)cccc21 ; Cc1ccc2c(c1)C1(c3ccccc3-2)c2cc(N(c3ccc(-c4ccccc4)cc3)c3ccc4ccccc4c3-c3ccccc3-c3ccccc3)ccc2-c2c(C)cccc21 ; Cc1ccc2c(c1)C1(c3ccccc3-2)c2cc(N(c3ccc(-c4ccccc4)cc3)c3ccc(-c4cc5ccccc5cc4-c4ccccc4)cc3)ccc2-c2c(C)cccc21 ; Cc1ccc2c(c1)C1(c3ccccc3-2)c2cc(N(c3ccc(-c4ccccc4)cc3)c3ccc(-c4ccc5ccccc5c4-c4ccccc4)cc3)ccc2-c2c(C)cccc21] 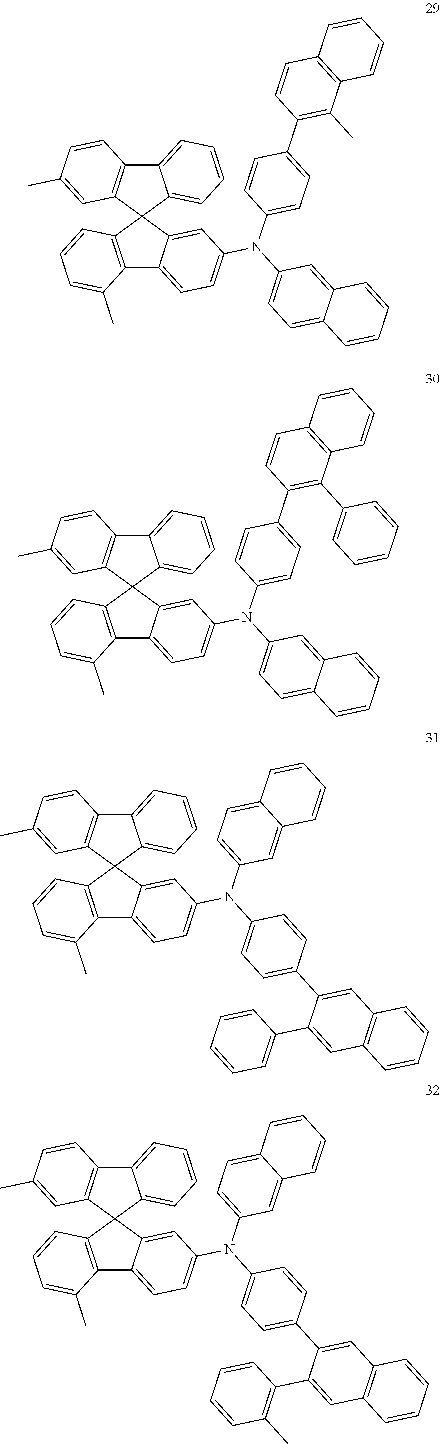 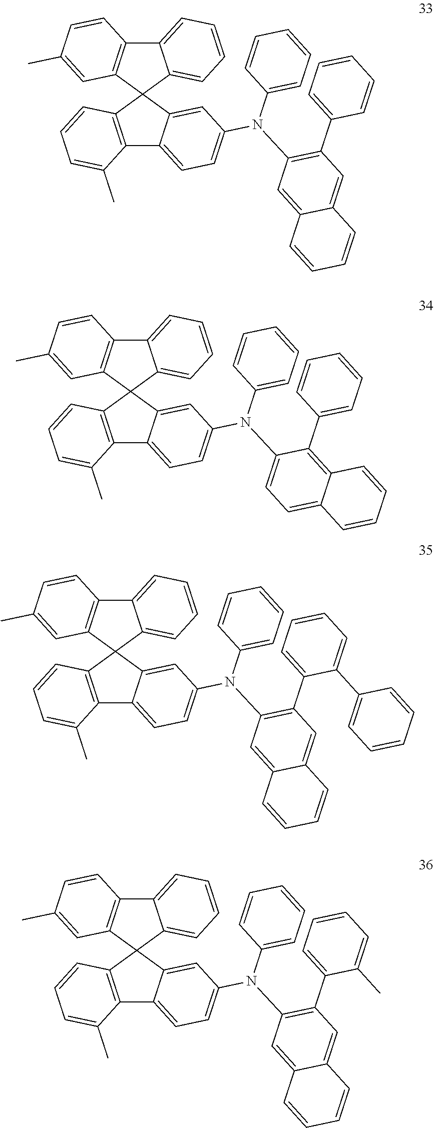 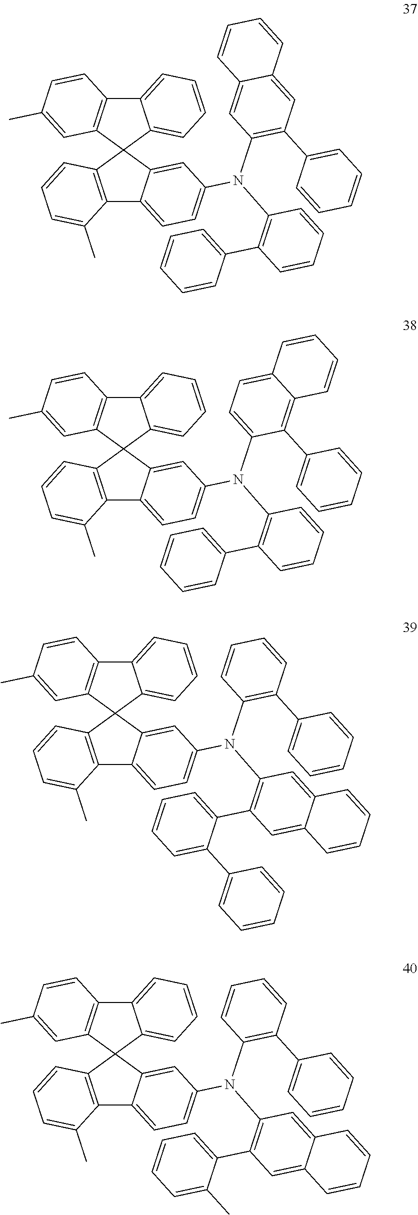 [SMILES: Cc1ccc2c(c1)C1(c3ccccc3-2)c2cc(N(c3ccccc3-c3ccccc3)c3cc4ccccc4cc3-c3ccccc3)ccc2-c2c(C)cccc21 ; Cc1ccc2c(c1)C1(c3ccccc3-2)c2cc(N(c3ccccc3-c3ccccc3)c3ccc4ccccc4c3-c3ccccc3)ccc2-c2c(C)cccc21 ; Cc1ccc2c(c1)C1(c3ccccc3-2)c2cc(N(c3ccccc3-c3ccccc3)c3cc4ccccc4cc3-c3ccccc3-c3ccccc3)ccc2-c2c(C)cccc21 ; Cc1ccc2c(c1)C1(c3ccccc3-2)c2cc(N(c3ccccc3-c3ccccc3)c3cc4ccccc4cc3-c3ccccc3C)ccc2-c2c(C)cccc21] 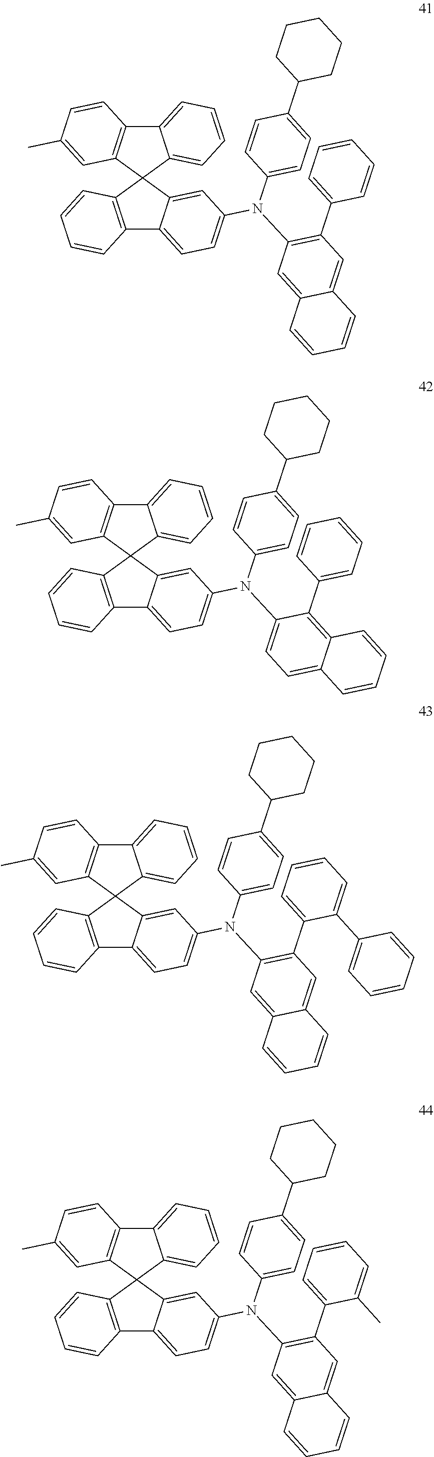 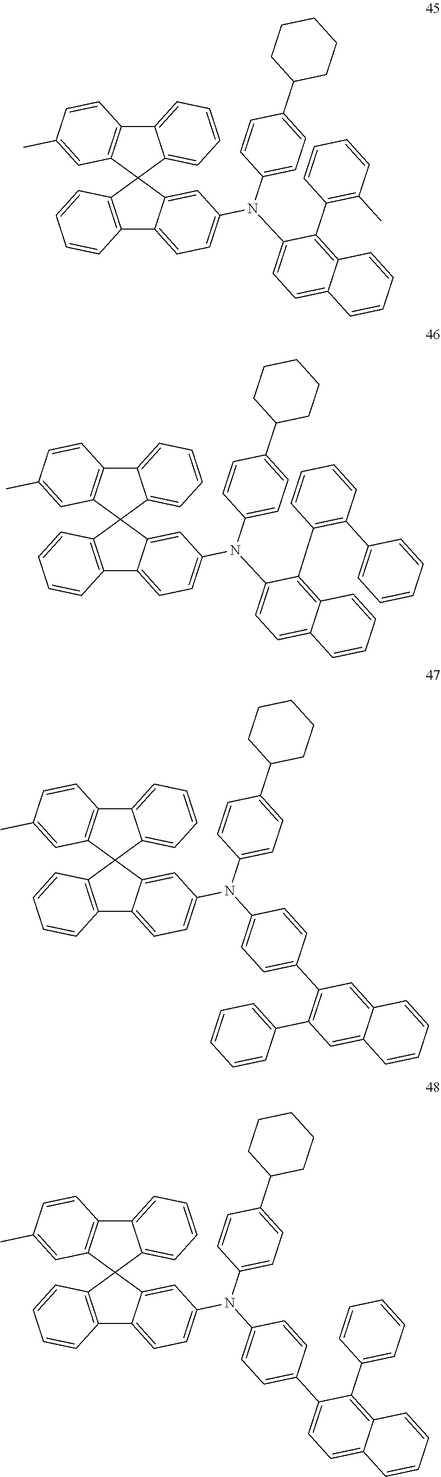 [SMILES: Cc1ccc2c(c1)C1(c3ccccc3-2)c2ccccc2-c2ccc(N(c3ccc(C4CCCCC4)cc3)c3ccc4ccccc4c3-c3ccccc3C)cc21 ; Cc1ccc2c(c1)C1(c3ccccc3-2)c2ccccc2-c2ccc(N(c3ccc(C4CCCCC4)cc3)c3ccc4ccccc4c3-c3ccccc3-c3ccccc3)cc21 ; Cc1ccc2c(c1)C1(c3ccccc3-2)c2ccccc2-c2ccc(N(c3ccc(-c4cc5ccccc5cc4-c4ccccc4)cc3)c3ccc(C4CCCCC4)cc3)cc21 ; Cc1ccc2c(c1)C1(c3ccccc3-2)c2ccccc2-c2ccc(N(c3ccc(-c4ccc5ccccc5c4-c4ccccc4)cc3)c3ccc(C4CCCCC4)cc3)cc21] 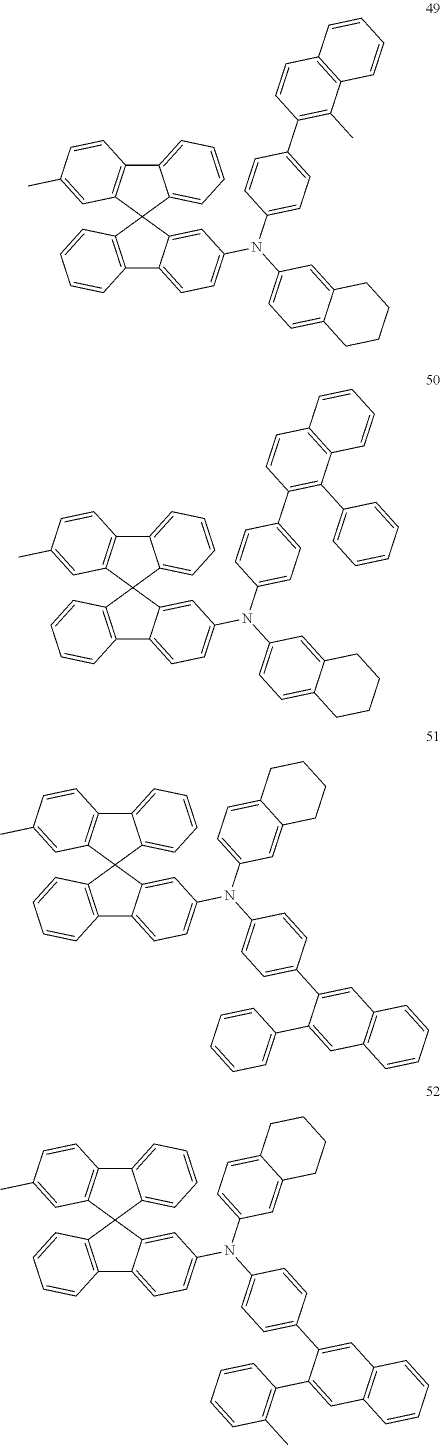 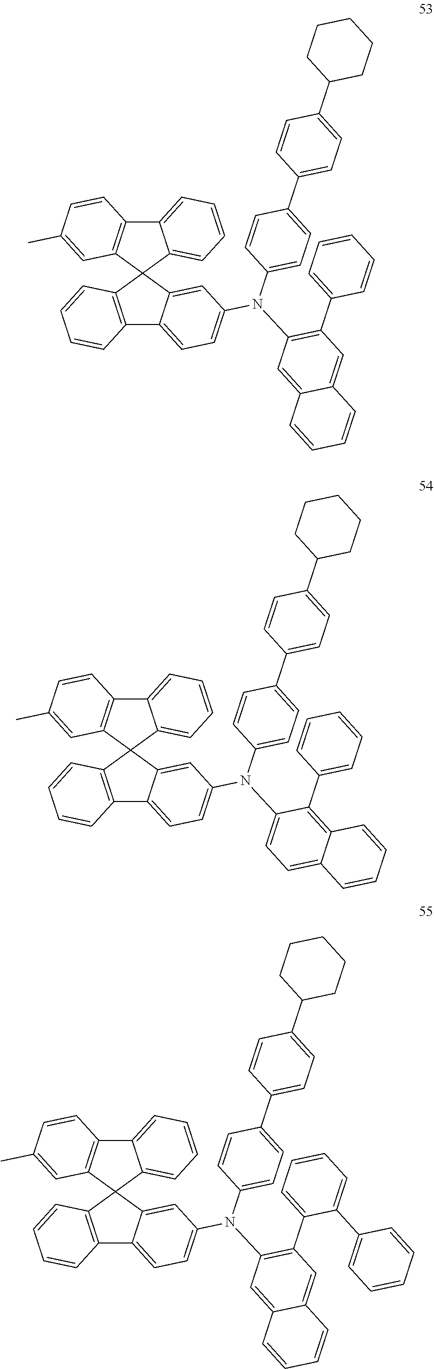 [SMILES: Cc1ccc2c(c1)C1(c3ccccc3-2)c2ccccc2-c2ccc(N(c3ccc(-c4ccc(C5CCCCC5)cc4)cc3)c3cc4ccccc4cc3-c3ccccc3)cc21 ; Cc1ccc2c(c1)C1(c3ccccc3-2)c2ccccc2-c2ccc(N(c3ccc(-c4ccc(C5CCCCC5)cc4)cc3)c3ccc4ccccc4c3-c3ccccc3)cc21 ; Cc1ccc2c(c1)C1(c3ccccc3-2)c2ccccc2-c2ccc(N(c3ccc(-c4ccc(C5CCCCC5)cc4)cc3)c3cc4ccccc4cc3-c3ccccc3-c3ccccc3)cc21] 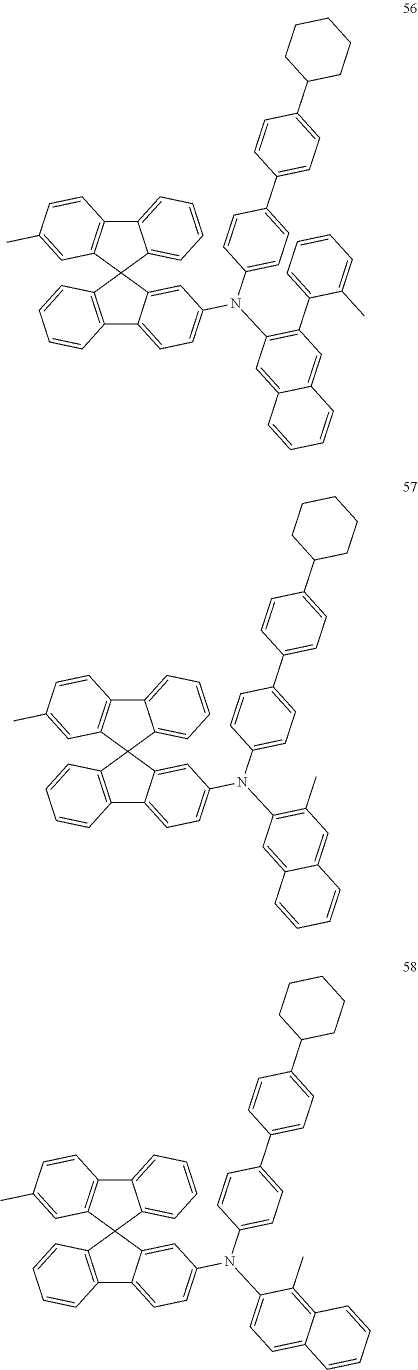 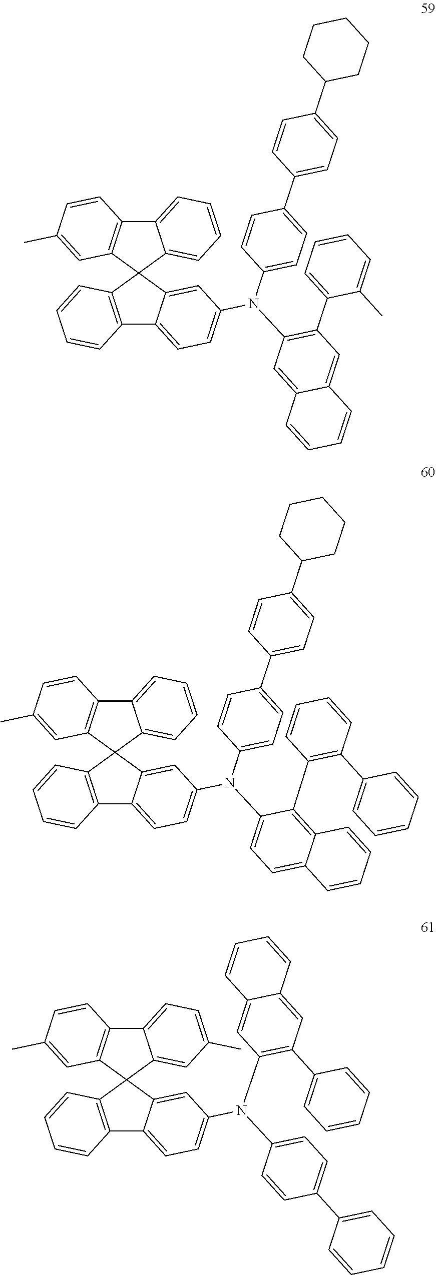 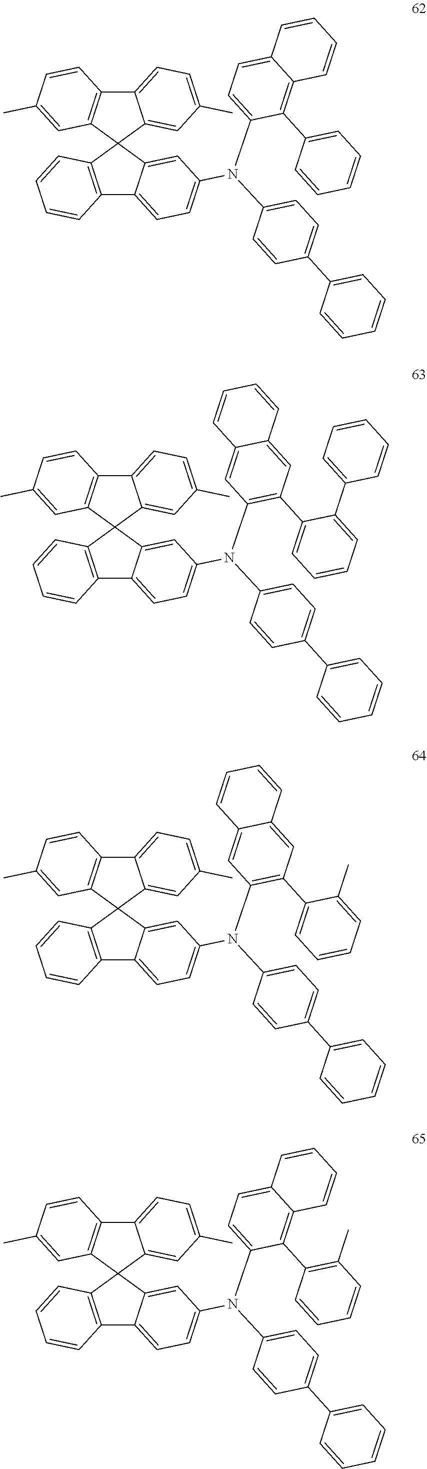 [SMILES: Cc1ccc2c(c1)C1(c3ccccc3-c3ccc(N(c4ccc(-c5ccccc5)cc4)c4ccc5ccccc5c4-c4ccccc4)cc31)c1cc(C)ccc1-2 ; Cc1ccc2c(c1)C1(c3ccccc3-c3ccc(N(c4ccc(-c5ccccc5)cc4)c4cc5ccccc5cc4-c4ccccc4-c4ccccc4)cc31)c1cc(C)ccc1-2 ; Cc1ccc2c(c1)C1(c3ccccc3-c3ccc(N(c4ccc(-c5ccccc5)cc4)c4cc5ccccc5cc4-c4ccccc4C)cc31)c1cc(C)ccc1-2 ; Cc1ccc2c(c1)C1(c3ccccc3-c3ccc(N(c4ccc(-c5ccccc5)cc4)c4ccc5ccccc5c4-c4ccccc4C)cc31)c1cc(C)ccc1-2] 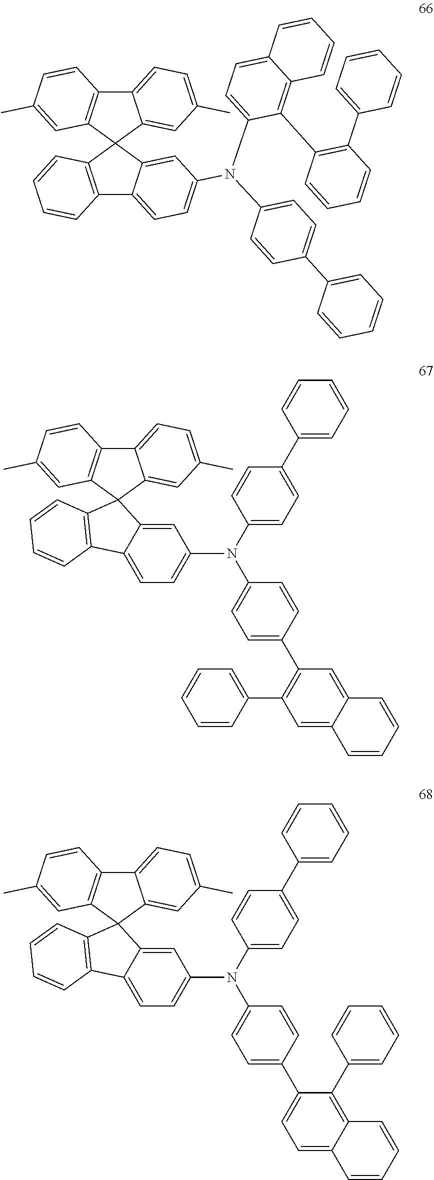 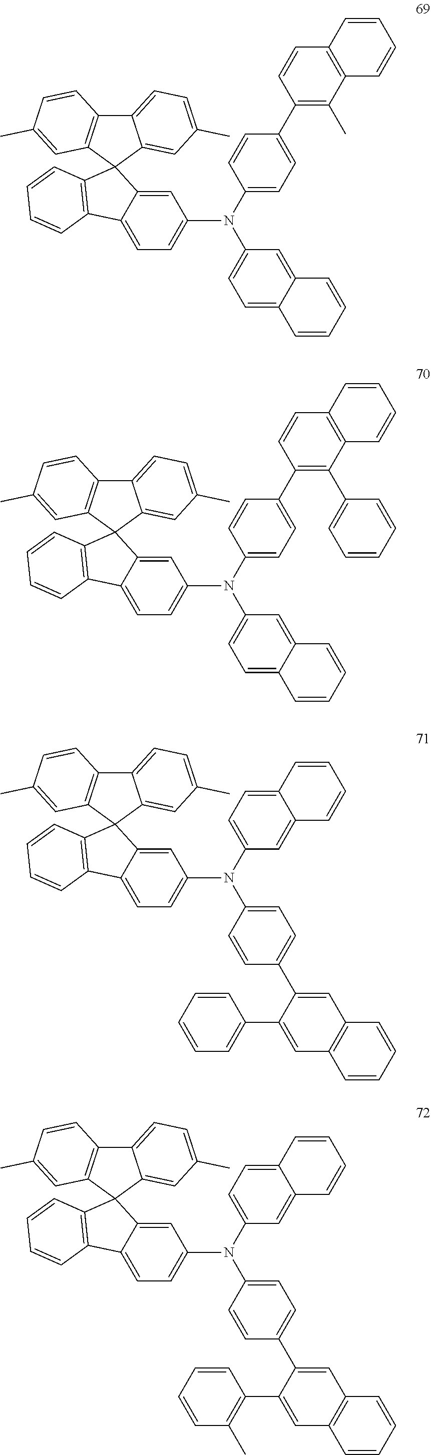 [SMILES: Cc1ccc2c(c1)C1(c3ccccc3-c3ccc(N(c4ccc(-c5ccc6ccccc6c5C)cc4)c4ccc5ccccc5c4)cc31)c1cc(C)ccc1-2 ; Cc1ccc2c(c1)C1(c3ccccc3-c3ccc(N(c4ccc(-c5ccc6ccccc6c5-c5ccccc5)cc4)c4ccc5ccccc5c4)cc31)c1cc(C)ccc1-2 ; Cc1ccc2c(c1)C1(c3ccccc3-c3ccc(N(c4ccc(-c5cc6ccccc6cc5-c5ccccc5)cc4)c4ccc5ccccc5c4)cc31)c1cc(C)ccc1-2 ; Cc1ccc2c(c1)C1(c3ccccc3-c3ccc(N(c4ccc(-c5cc6ccccc6cc5-c5ccccc5C)cc4)c4ccc5ccccc5c4)cc31)c1cc(C)ccc1-2] 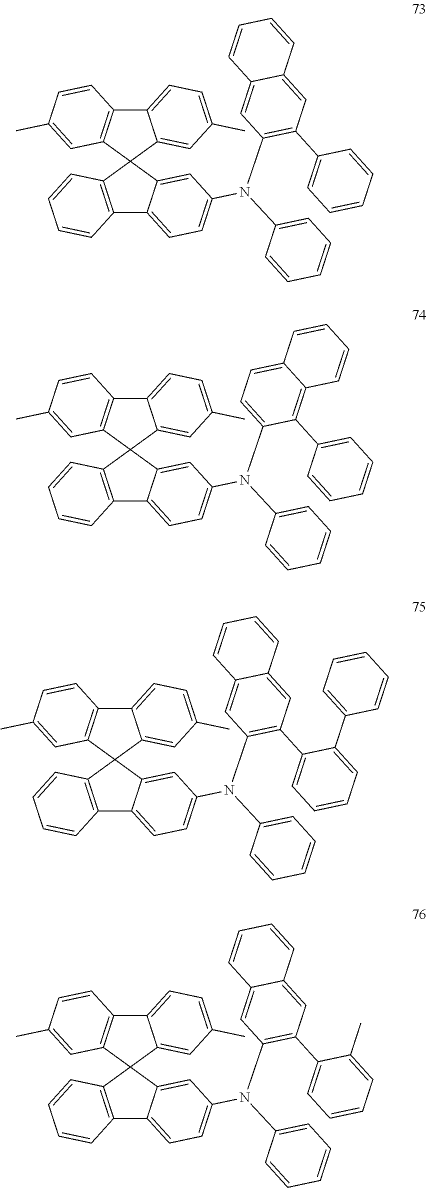 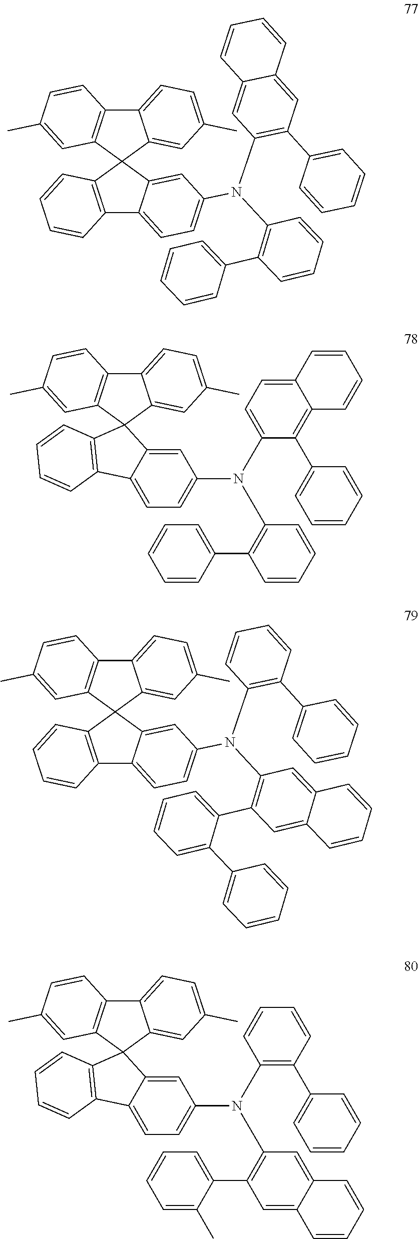 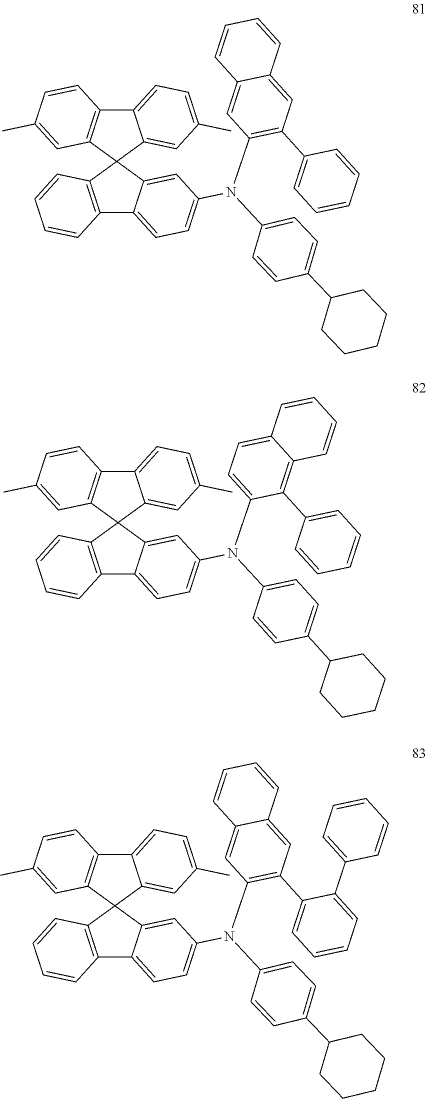 [SMILES: Cc1ccc2c(c1)C1(c3ccccc3-c3ccc(N(c4ccc(C5CCCCC5)cc4)c4cc5ccccc5cc4-c4ccccc4)cc31)c1cc(C)ccc1-2 ; Cc1ccc2c(c1)C1(c3ccccc3-c3ccc(N(c4ccc(C5CCCCC5)cc4)c4ccc5ccccc5c4-c4ccccc4)cc31)c1cc(C)ccc1-2 ; Cc1ccc2c(c1)C1(c3ccccc3-c3ccc(N(c4ccc(C5CCCCC5)cc4)c4cc5ccccc5cc4-c4ccccc4-c4ccccc4)cc31)c1cc(C)ccc1-2] 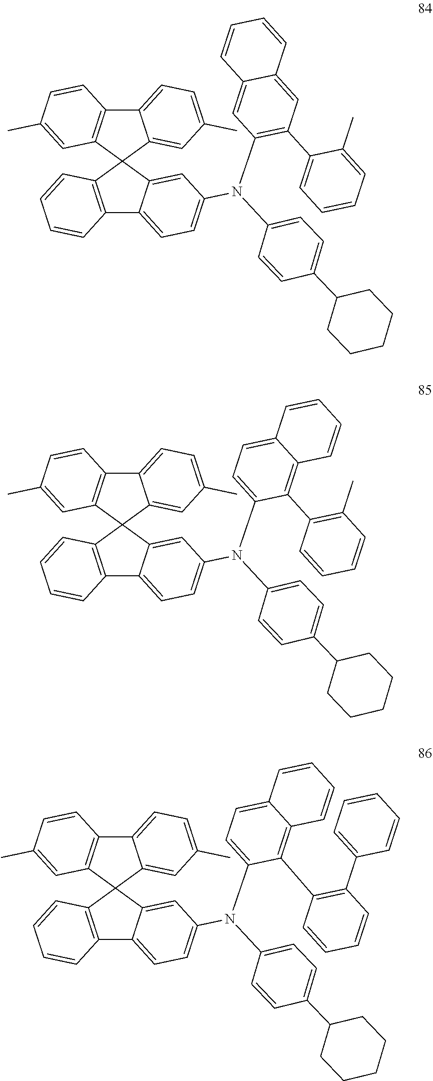 [SMILES: Cc1ccc2c(c1)C1(c3ccccc3-c3ccc(N(c4ccc(C5CCCCC5)cc4)c4cc5ccccc5cc4-c4ccccc4C)cc31)c1cc(C)ccc1-2 ; Cc1ccc2c(c1)C1(c3ccccc3-c3ccc(N(c4ccc(C5CCCCC5)cc4)c4ccc5ccccc5c4-c4ccccc4C)cc31)c1cc(C)ccc1-2 ; Cc1ccc2c(c1)C1(c3ccccc3-c3ccc(N(c4ccc(C5CCCCC5)cc4)c4ccc5ccccc5c4-c4ccccc4-c4ccccc4)cc31)c1cc(C)ccc1-2] 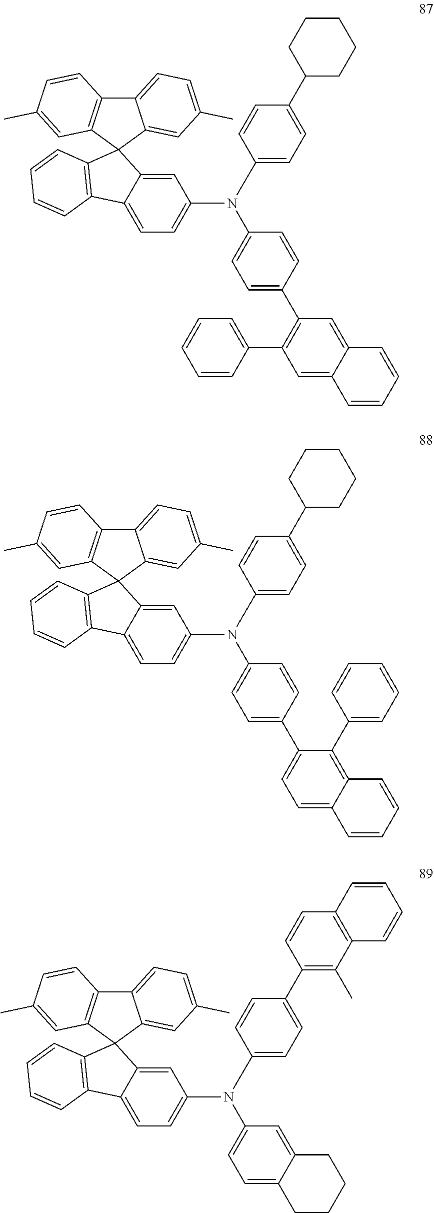 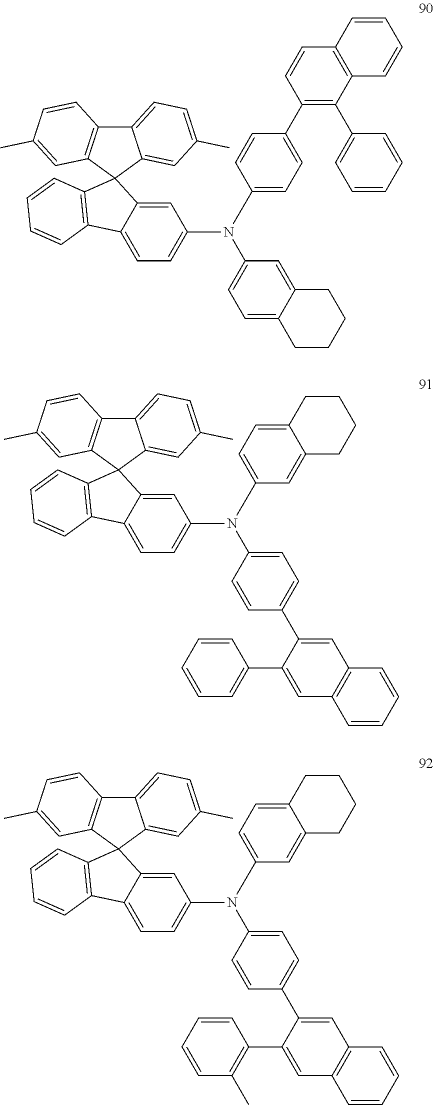 [SMILES: Cc1ccc2c(c1)C1(c3ccccc3-c3ccc(N(c4ccc(-c5ccc6ccccc6c5-c5ccccc5)cc4)c4ccc5c(c4)CCCC5)cc31)c1cc(C)ccc1-2 ; Cc1ccc2c(c1)C1(c3ccccc3-c3ccc(N(c4ccc(-c5cc6ccccc6cc5-c5ccccc5)cc4)c4ccc5c(c4)CCCC5)cc31)c1cc(C)ccc1-2 ; Cc1ccc2c(c1)C1(c3ccccc3-c3ccc(N(c4ccc(-c5cc6ccccc6cc5-c5ccccc5C)cc4)c4ccc5c(c4)CCCC5)cc31)c1cc(C)ccc1-2] 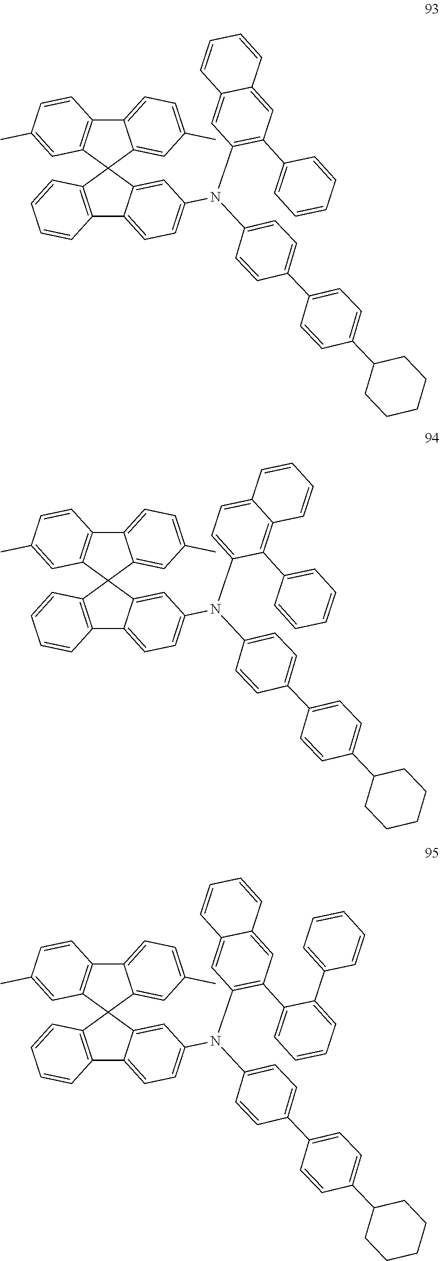 [SMILES: Cc1ccc2c(c1)C1(c3ccccc3-c3ccc(N(c4ccc(-c5ccc(C6CCCCC6)cc5)cc4)c4cc5ccccc5cc4-c4ccccc4)cc31)c1cc(C)ccc1-2 ; Cc1ccc2c(c1)C1(c3ccccc3-c3ccc(N(c4ccc(-c5ccc(C6CCCCC6)cc5)cc4)c4ccc5ccccc5c4-c4ccccc4)cc31)c1cc(C)ccc1-2 ; Cc1ccc2c(c1)C1(c3ccccc3-c3ccc(N(c4ccc(-c5ccc(C6CCCCC6)cc5)cc4)c4cc5ccccc5cc4-c4ccccc4-c4ccccc4)cc31)c1cc(C)ccc1-2] 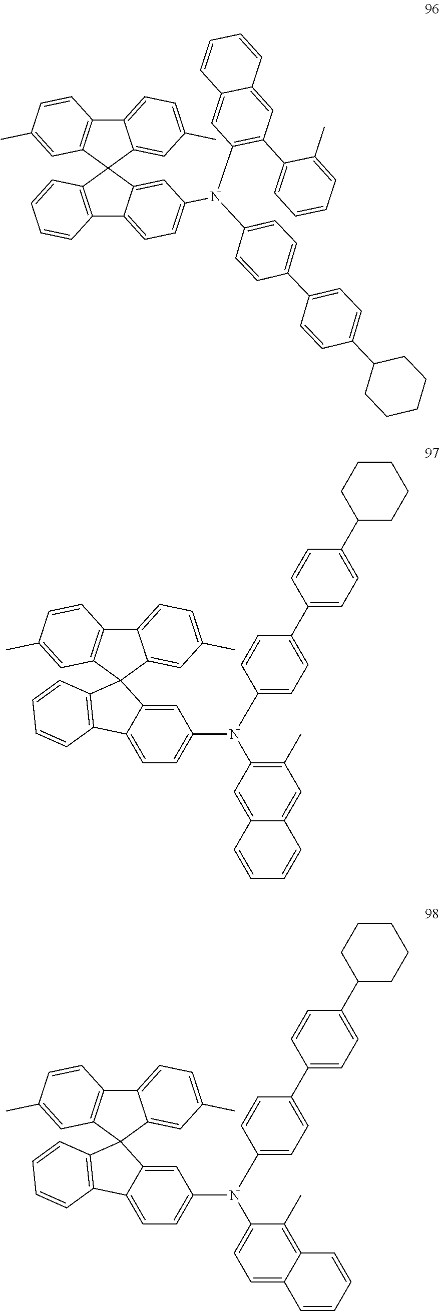 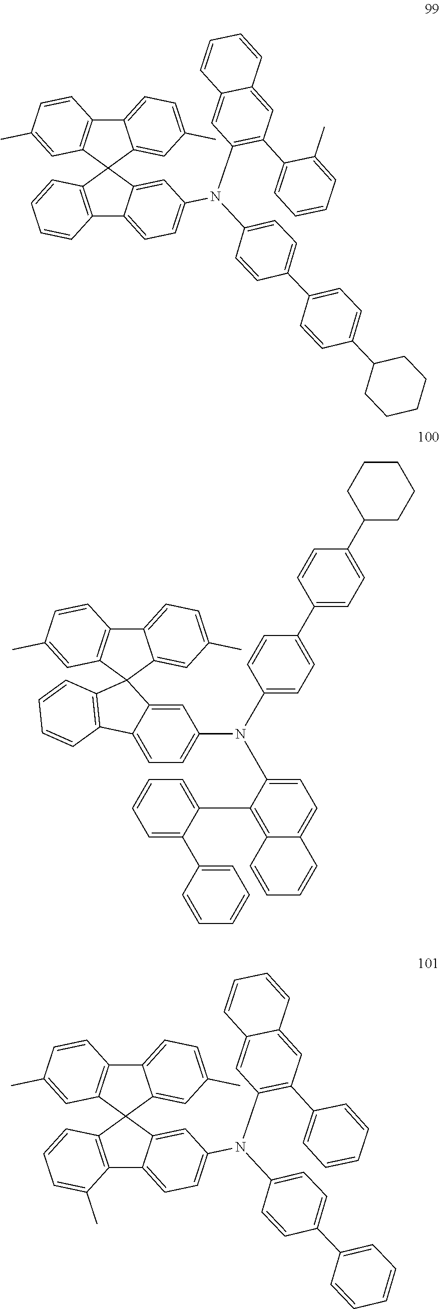 [SMILES: Cc1ccc2c(c1)C1(c3ccccc3-c3ccc(N(c4ccc(-c5ccc(C6CCCCC6)cc5)cc4)c4cc5ccccc5cc4-c4ccccc4C)cc31)c1cc(C)ccc1-2 ; Cc1ccc2c(c1)C1(c3ccccc3-c3ccc(N(c4ccc(-c5ccc(C6CCCCC6)cc5)cc4)c4ccc5ccccc5c4-c4ccccc4-c4ccccc4)cc31)c1cc(C)ccc1-2 ; Cc1ccc2c(c1)C1(c3cc(C)ccc3-2)c2cc(N(c3ccc(-c4ccccc4)cc3)c3cc4ccccc4cc3-c3ccccc3)ccc2-c2c(C)cccc21] 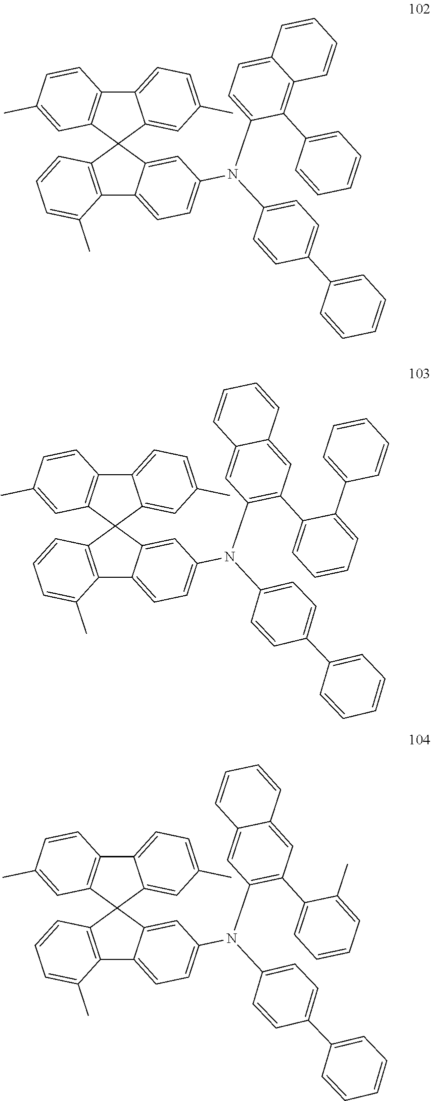 [SMILES: Cc1ccc2c(c1)C1(c3cc(C)ccc3-2)c2cc(N(c3ccc(-c4ccccc4)cc3)c3ccc4ccccc4c3-c3ccccc3)ccc2-c2c(C)cccc21 ; Cc1ccc2c(c1)C1(c3cc(C)ccc3-2)c2cc(N(c3ccc(-c4ccccc4)cc3)c3cc4ccccc4cc3-c3ccccc3-c3ccccc3)ccc2-c2c(C)cccc21 ; Cc1ccc2c(c1)C1(c3cc(C)ccc3-2)c2cc(N(c3ccc(-c4ccccc4)cc3)c3cc4ccccc4cc3-c3ccccc3C)ccc2-c2c(C)cccc21] 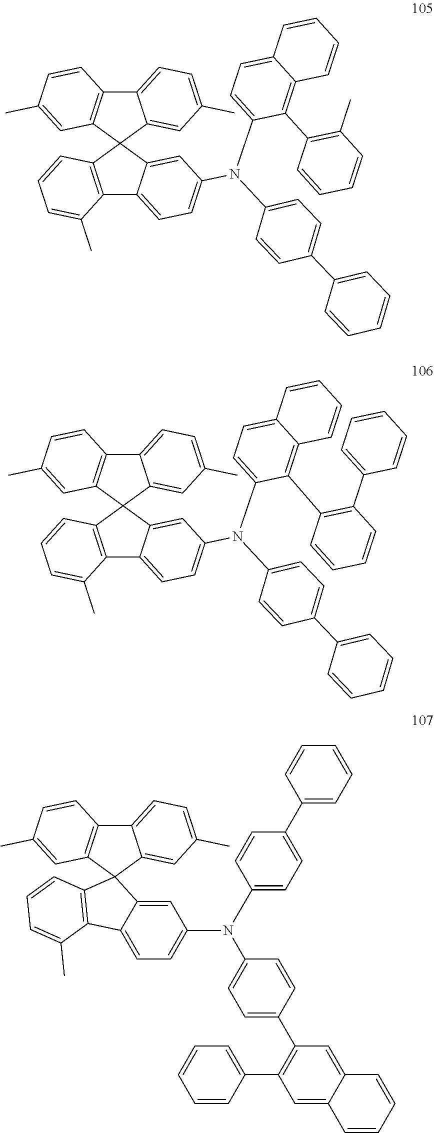 [SMILES: Cc1ccc2c(c1)C1(c3cc(C)ccc3-2)c2cc(N(c3ccc(-c4ccccc4)cc3)c3ccc4ccccc4c3-c3ccccc3C)ccc2-c2c(C)cccc21 ; Cc1ccc2c(c1)C1(c3cc(C)ccc3-2)c2cc(N(c3ccc(-c4ccccc4)cc3)c3ccc4ccccc4c3-c3ccccc3-c3ccccc3)ccc2-c2c(C)cccc21 ; Cc1ccc2c(c1)C1(c3cc(C)ccc3-2)c2cc(N(c3ccc(-c4ccccc4)cc3)c3ccc(-c4cc5ccccc5cc4-c4ccccc4)cc3)ccc2-c2c(C)cccc21] 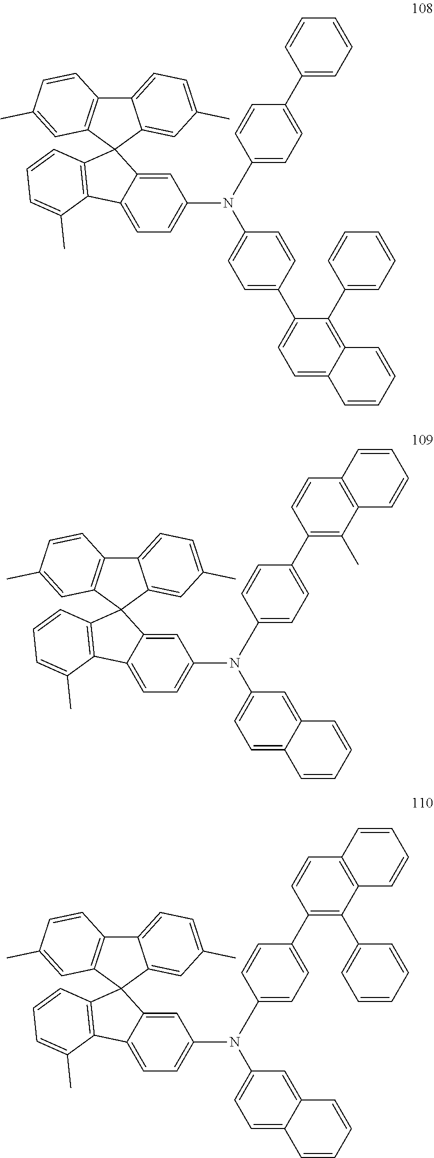 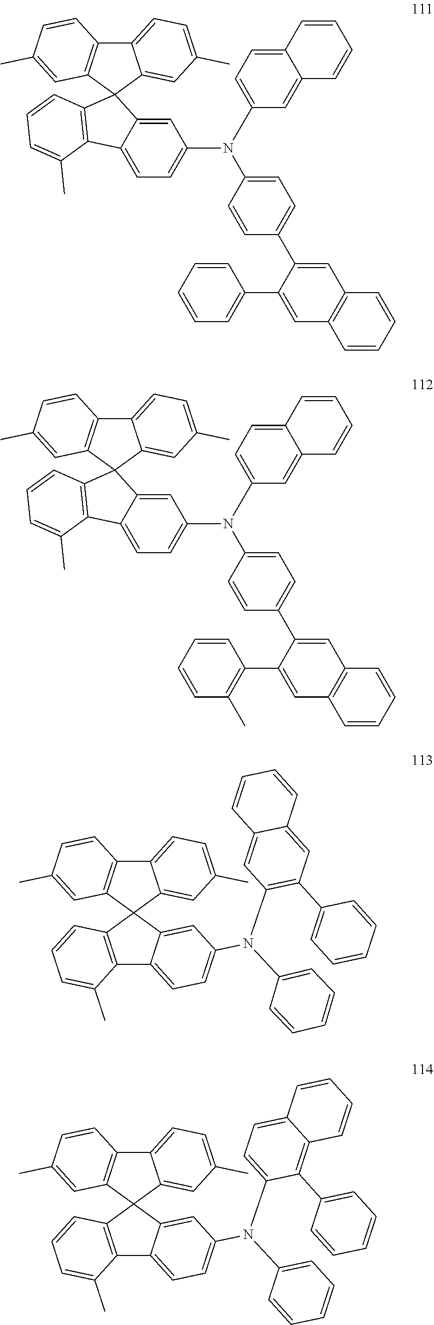 [SMILES: Cc1ccc2c(c1)C1(c3cc(C)ccc3-2)c2cc(N(c3ccc(-c4cc5ccccc5cc4-c4ccccc4)cc3)c3ccc4ccccc4c3)ccc2-c2c(C)cccc21 ; Cc1ccc2c(c1)C1(c3cc(C)ccc3-2)c2cc(N(c3ccc(-c4cc5ccccc5cc4-c4ccccc4C)cc3)c3ccc4ccccc4c3)ccc2-c2c(C)cccc21 ; Cc1ccc2c(c1)C1(c3cc(C)ccc3-2)c2cc(N(c3ccccc3)c3cc4ccccc4cc3-c3ccccc3)ccc2-c2c(C)cccc21 ; Cc1ccc2c(c1)C1(c3cc(C)ccc3-2)c2cc(N(c3ccccc3)c3ccc4ccccc4c3-c3ccccc3)ccc2-c2c(C)cccc21] 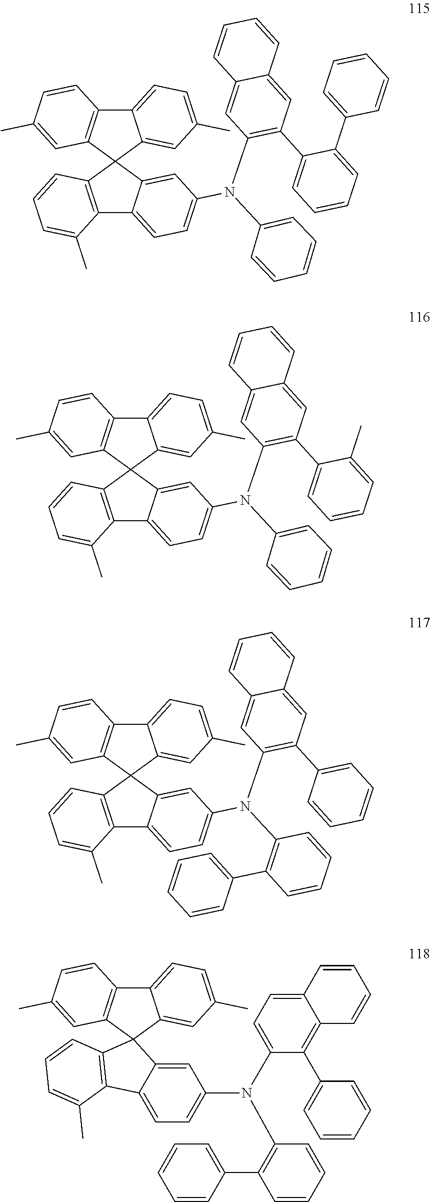 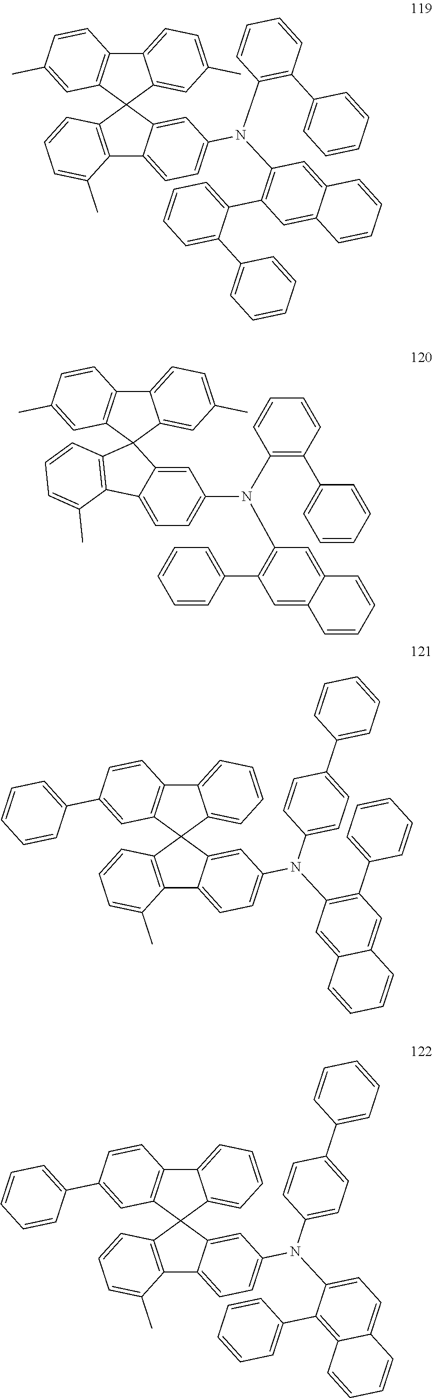 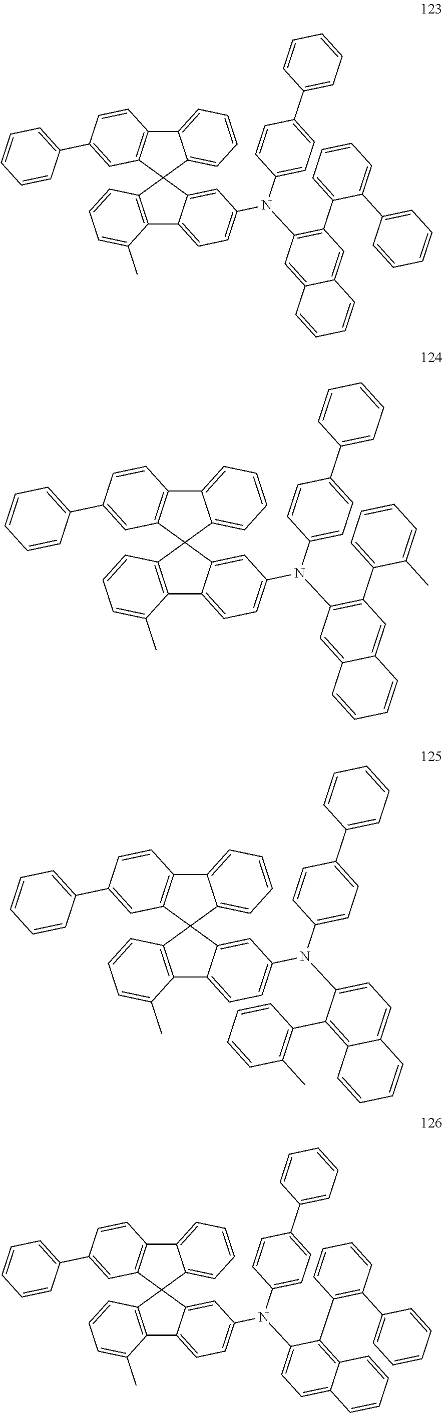 [SMILES: Cc1cccc2c1-c1ccc(N(c3ccc(-c4ccccc4)cc3)c3cc4ccccc4cc3-c3ccccc3-c3ccccc3)cc1C21c2ccccc2-c2ccc(-c3ccccc3)cc21 ; Cc1ccccc1-c1cc2ccccc2cc1N(c1ccc(-c2ccccc2)cc1)c1ccc2c(c1)C1(c3ccccc3-c3ccc(-c4ccccc4)cc31)c1cccc(C)c1-2 ; Cc1ccccc1-c1c(N(c2ccc(-c3ccccc3)cc2)c2ccc3c(c2)C2(c4ccccc4-c4ccc(-c5ccccc5)cc42)c2cccc(C)c2-3)ccc2ccccc12 ; Cc1cccc2c1-c1ccc(N(c3ccc(-c4ccccc4)cc3)c3ccc4ccccc4c3-c3ccccc3-c3ccccc3)cc1C21c2ccccc2-c2ccc(-c3ccccc3)cc21] 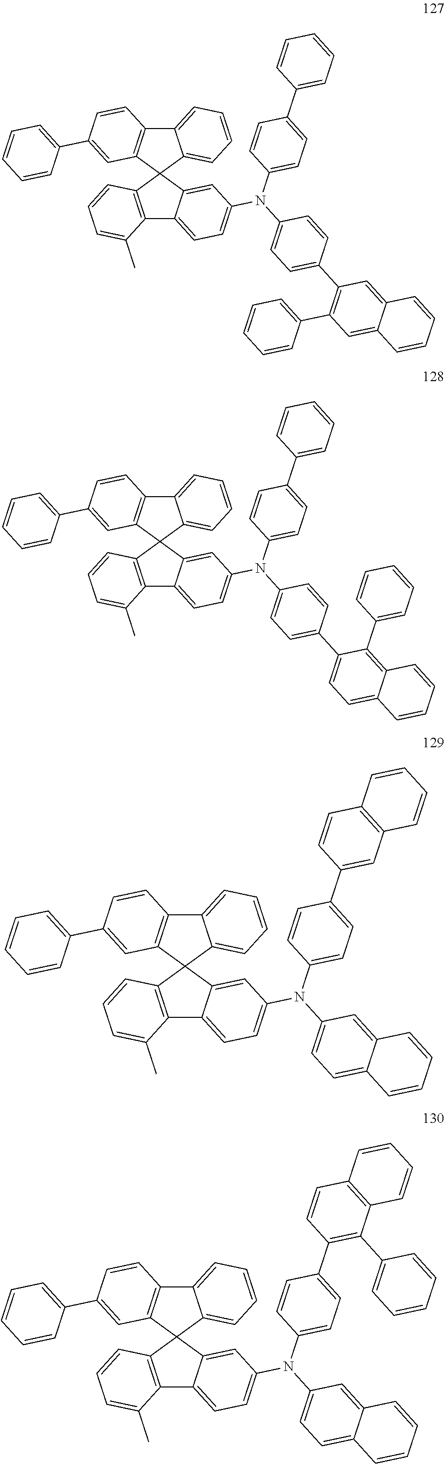 [SMILES: Cc1cccc2c1-c1ccc(N(c3ccc(-c4ccccc4)cc3)c3ccc(-c4cc5ccccc5cc4-c4ccccc4)cc3)cc1C21c2ccccc2-c2ccc(-c3ccccc3)cc21 ; Cc1cccc2c1-c1ccc(N(c3ccc(-c4ccccc4)cc3)c3ccc(-c4ccc5ccccc5c4-c4ccccc4)cc3)cc1C21c2ccccc2-c2ccc(-c3ccccc3)cc21 ; Cc1cccc2c1-c1ccc(N(c3ccc(-c4ccc5ccccc5c4)cc3)c3ccc4ccccc4c3)cc1C21c2ccccc2-c2ccc(-c3ccccc3)cc21 ; Cc1cccc2c1-c1ccc(N(c3ccc(-c4ccc5ccccc5c4-c4ccccc4)cc3)c3ccc4ccccc4c3)cc1C21c2ccccc2-c2ccc(-c3ccccc3)cc21] 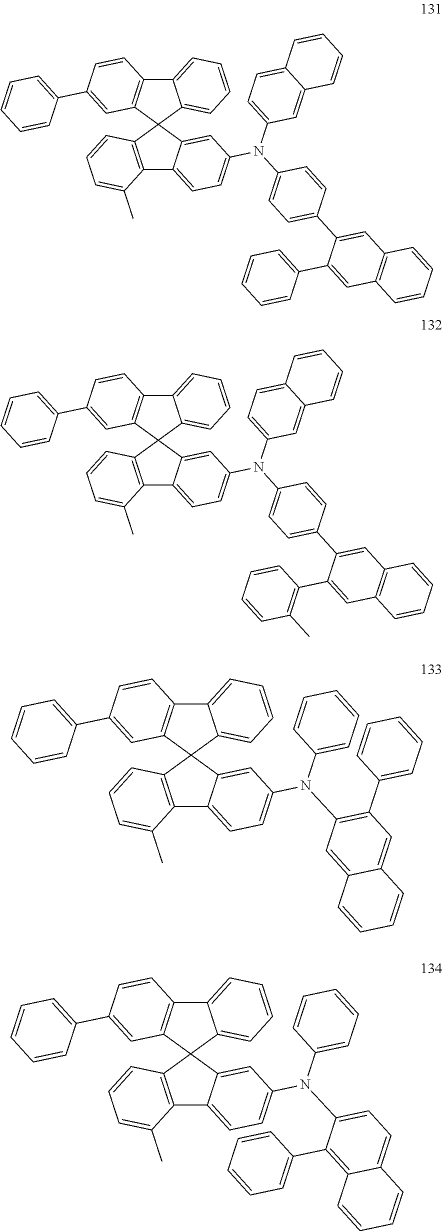 [SMILES: Cc1cccc2c1-c1ccc(N(c3ccc(-c4cc5ccccc5cc4-c4ccccc4)cc3)c3ccc4ccccc4c3)cc1C21c2ccccc2-c2ccc(-c3ccccc3)cc21 ; Cc1ccccc1-c1cc2ccccc2cc1-c1ccc(N(c2ccc3c(c2)C2(c4ccccc4-c4ccc(-c5ccccc5)cc42)c2cccc(C)c2-3)c2ccc3ccccc3c2)cc1 ; Cc1cccc2c1-c1ccc(N(c3ccccc3)c3cc4ccccc4cc3-c3ccccc3)cc1C21c2ccccc2-c2ccc(-c3ccccc3)cc21 ; Cc1cccc2c1-c1ccc(N(c3ccccc3)c3ccc4ccccc4c3-c3ccccc3)cc1C21c2ccccc2-c2ccc(-c3ccccc3)cc21] 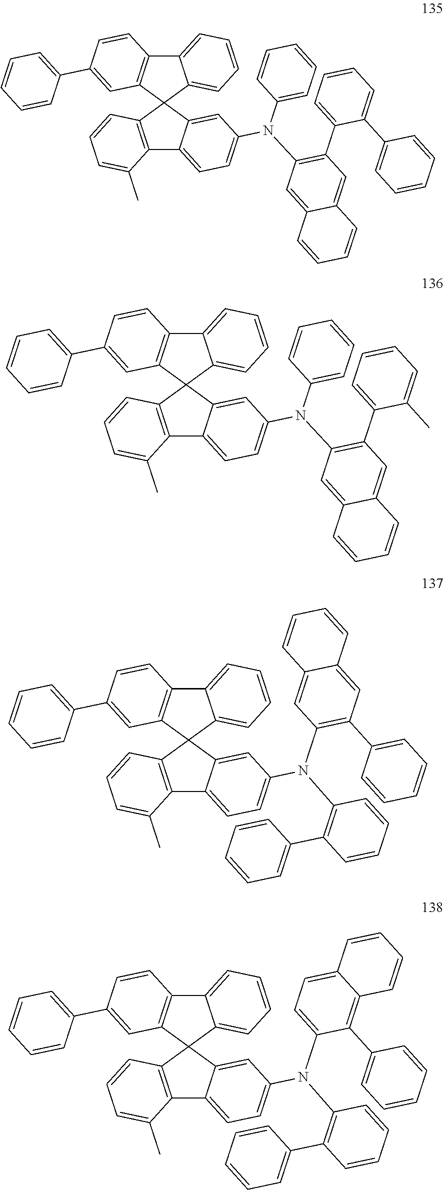 [SMILES: Cc1cccc2c1-c1ccc(N(c3ccccc3)c3cc4ccccc4cc3-c3ccccc3-c3ccccc3)cc1C21c2ccccc2-c2ccc(-c3ccccc3)cc21 ; Cc1ccccc1-c1cc2ccccc2cc1N(c1ccccc1)c1ccc2c(c1)C1(c3ccccc3-c3ccc(-c4ccccc4)cc31)c1cccc(C)c1-2 ; Cc1cccc2c1-c1ccc(N(c3ccccc3-c3ccccc3)c3cc4ccccc4cc3-c3ccccc3)cc1C21c2ccccc2-c2ccc(-c3ccccc3)cc21 ; Cc1cccc2c1-c1ccc(N(c3ccccc3-c3ccccc3)c3ccc4ccccc4c3-c3ccccc3)cc1C21c2ccccc2-c2ccc(-c3ccccc3)cc21] 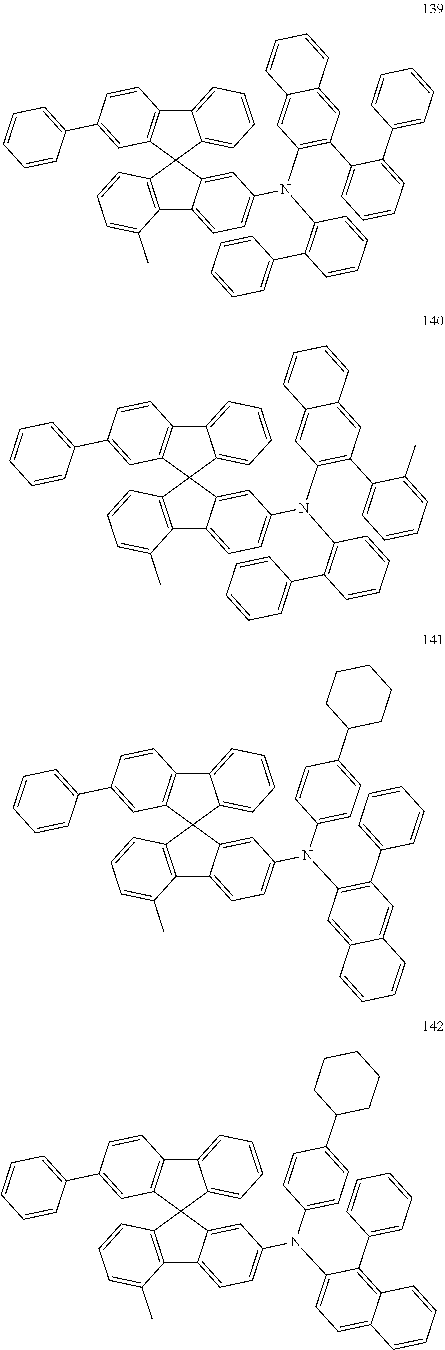 [SMILES: Cc1cccc2c1-c1ccc(N(c3ccccc3-c3ccccc3)c3cc4ccccc4cc3-c3ccccc3-c3ccccc3)cc1C21c2ccccc2-c2ccc(-c3ccccc3)cc21 ; Cc1ccccc1-c1cc2ccccc2cc1N(c1ccc2c(c1)C1(c3ccccc3-c3ccc(-c4ccccc4)cc31)c1cccc(C)c1-2)c1ccccc1-c1ccccc1 ; Cc1cccc2c1-c1ccc(N(c3ccc(C4CCCCC4)cc3)c3cc4ccccc4cc3-c3ccccc3)cc1C21c2ccccc2-c2ccc(-c3ccccc3)cc21 ; Cc1cccc2c1-c1ccc(N(c3ccc(C4CCCCC4)cc3)c3ccc4ccccc4c3-c3ccccc3)cc1C21c2ccccc2-c2ccc(-c3ccccc3)cc21] 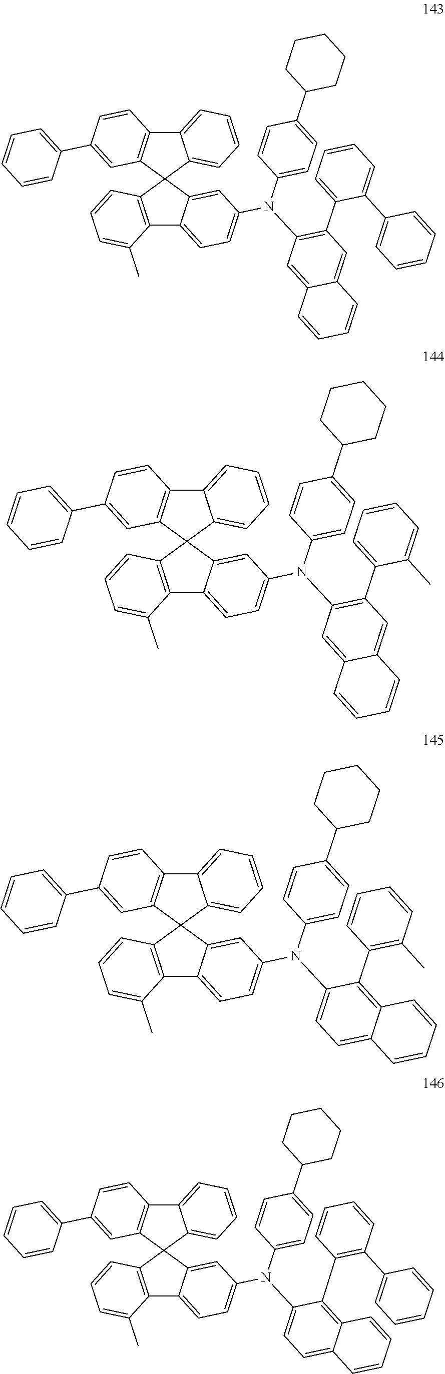 [SMILES: Cc1cccc2c1-c1ccc(N(c3ccc(C4CCCCC4)cc3)c3cc4ccccc4cc3-c3ccccc3-c3ccccc3)cc1C21c2ccccc2-c2ccc(-c3ccccc3)cc21 ; Cc1ccccc1-c1cc2ccccc2cc1N(c1ccc(C2CCCCC2)cc1)c1ccc2c(c1)C1(c3ccccc3-c3ccc(-c4ccccc4)cc31)c1cccc(C)c1-2 ; Cc1ccccc1-c1c(N(c2ccc(C3CCCCC3)cc2)c2ccc3c(c2)C2(c4ccccc4-c4ccc(-c5ccccc5)cc42)c2cccc(C)c2-3)ccc2ccccc12 ; Cc1cccc2c1-c1ccc(N(c3ccc(C4CCCCC4)cc3)c3ccc4ccccc4c3-c3ccccc3-c3ccccc3)cc1C21c2ccccc2-c2ccc(-c3ccccc3)cc21] 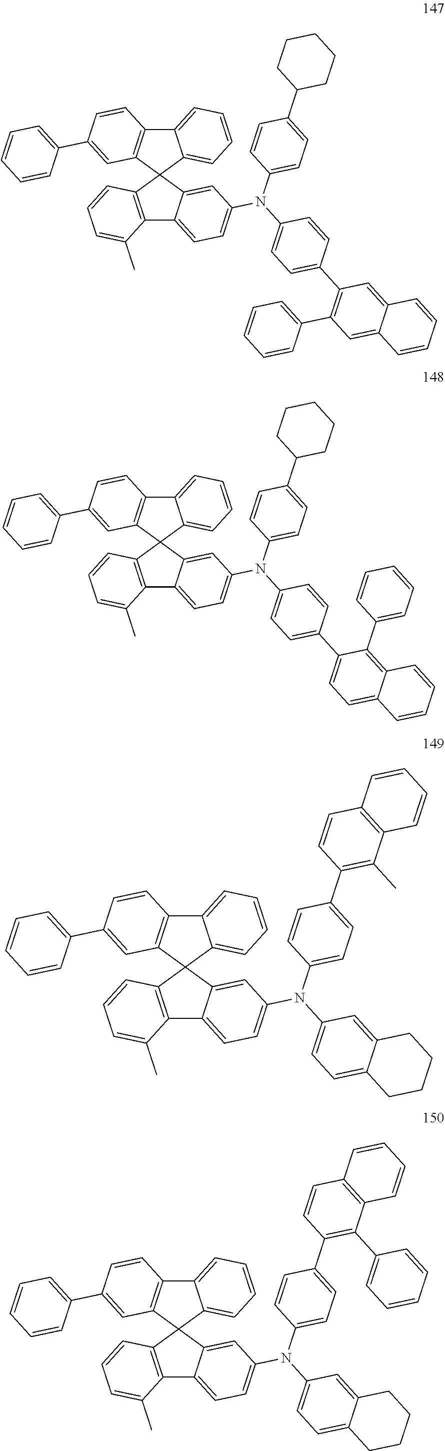 [SMILES: Cc1cccc2c1-c1ccc(N(c3ccc(-c4cc5ccccc5cc4-c4ccccc4)cc3)c3ccc(C4CCCCC4)cc3)cc1C21c2ccccc2-c2ccc(-c3ccccc3)cc21 ; Cc1cccc2c1-c1ccc(N(c3ccc(-c4ccc5ccccc5c4-c4ccccc4)cc3)c3ccc(C4CCCCC4)cc3)cc1C21c2ccccc2-c2ccc(-c3ccccc3)cc21 ; Cc1cccc2c1-c1ccc(N(c3ccc(-c4ccc5ccccc5c4C)cc3)c3ccc4c(c3)CCCC4)cc1C21c2ccccc2-c2ccc(-c3ccccc3)cc21 ; Cc1cccc2c1-c1ccc(N(c3ccc(-c4ccc5ccccc5c4-c4ccccc4)cc3)c3ccc4c(c3)CCCC4)cc1C21c2ccccc2-c2ccc(-c3ccccc3)cc21] 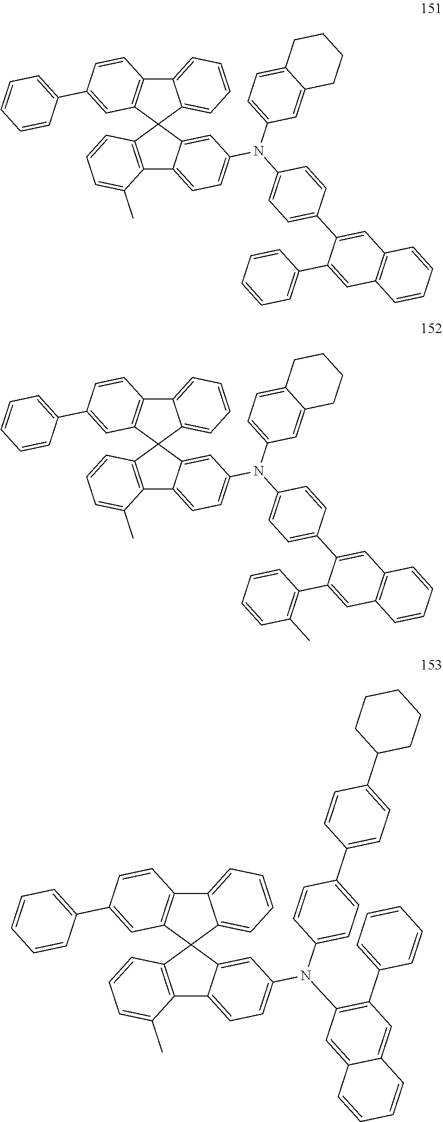 [SMILES: Cc1cccc2c1-c1ccc(N(c3ccc(-c4cc5ccccc5cc4-c4ccccc4)cc3)c3ccc4c(c3)CCCC4)cc1C21c2ccccc2-c2ccc(-c3ccccc3)cc21 ; Cc1ccccc1-c1cc2ccccc2cc1-c1ccc(N(c2ccc3c(c2)CCCC3)c2ccc3c(c2)C2(c4ccccc4-c4ccc(-c5ccccc5)cc42)c2cccc(C)c2-3)cc1 ; Cc1cccc2c1-c1ccc(N(c3ccc(-c4ccc(C5CCCCC5)cc4)cc3)c3cc4ccccc4cc3-c3ccccc3)cc1C21c2ccccc2-c2ccc(-c3ccccc3)cc21] 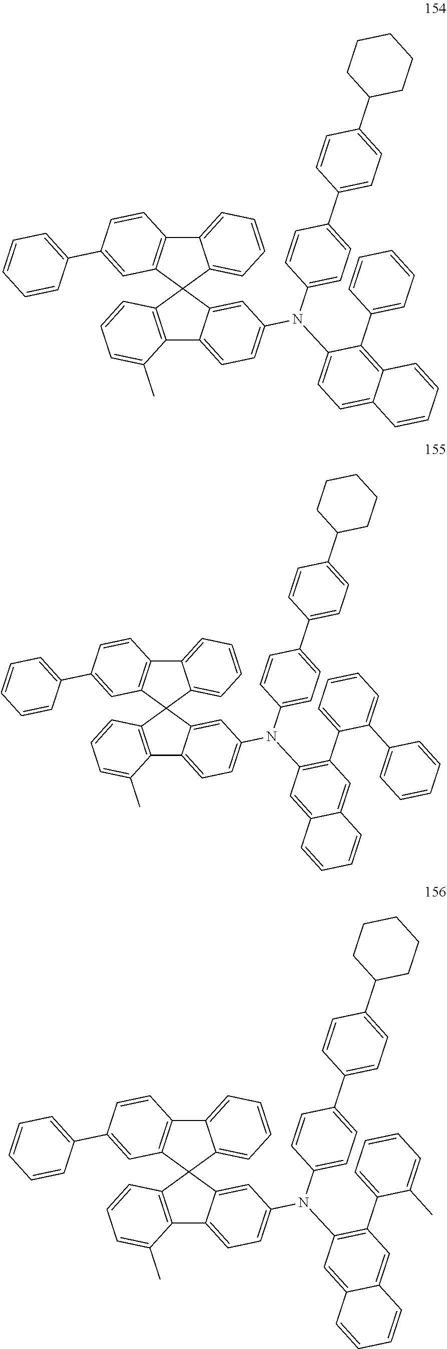 [SMILES: Cc1cccc2c1-c1ccc(N(c3ccc(-c4ccc(C5CCCCC5)cc4)cc3)c3ccc4ccccc4c3-c3ccccc3)cc1C21c2ccccc2-c2ccc(-c3ccccc3)cc21 ; Cc1cccc2c1-c1ccc(N(c3ccc(-c4ccc(C5CCCCC5)cc4)cc3)c3cc4ccccc4cc3-c3ccccc3-c3ccccc3)cc1C21c2ccccc2-c2ccc(-c3ccccc3)cc21 ; Cc1ccccc1-c1cc2ccccc2cc1N(c1ccc(-c2ccc(C3CCCCC3)cc2)cc1)c1ccc2c(c1)C1(c3ccccc3-c3ccc(-c4ccccc4)cc31)c1cccc(C)c1-2] 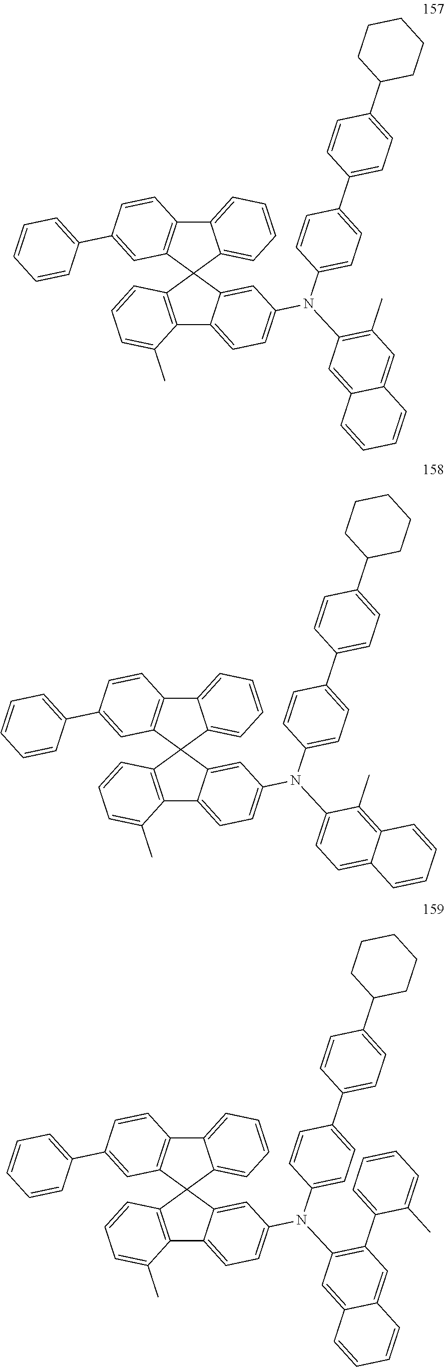 [SMILES: Cc1cc2ccccc2cc1N(c1ccc(-c2ccc(C3CCCCC3)cc2)cc1)c1ccc2c(c1)C1(c3ccccc3-c3ccc(-c4ccccc4)cc31)c1cccc(C)c1-2 ; Cc1cccc2c1-c1ccc(N(c3ccc(-c4ccc(C5CCCCC5)cc4)cc3)c3ccc4ccccc4c3C)cc1C21c2ccccc2-c2ccc(-c3ccccc3)cc21 ; Cc1ccccc1-c1cc2ccccc2cc1N(c1ccc(-c2ccc(C3CCCCC3)cc2)cc1)c1ccc2c(c1)C1(c3ccccc3-c3ccc(-c4ccccc4)cc31)c1cccc(C)c1-2] 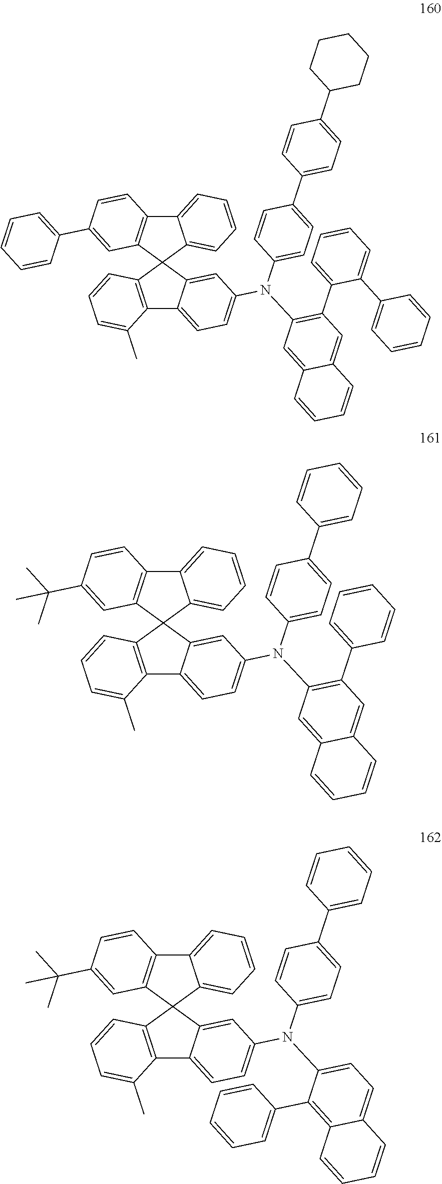 [SMILES: Cc1cccc2c1-c1ccc(N(c3ccc(-c4ccc(C5CCCCC5)cc4)cc3)c3cc4ccccc4cc3-c3ccccc3-c3ccccc3)cc1C21c2ccccc2-c2ccc(-c3ccccc3)cc21 ; Cc1cccc2c1-c1ccc(N(c3ccc(-c4ccccc4)cc3)c3cc4ccccc4cc3-c3ccccc3)cc1C21c2ccccc2-c2ccc(C(C)(C)C)cc21 ; Cc1cccc2c1-c1ccc(N(c3ccc(-c4ccccc4)cc3)c3ccc4ccccc4c3-c3ccccc3)cc1C21c2ccccc2-c2ccc(C(C)(C)C)cc21] 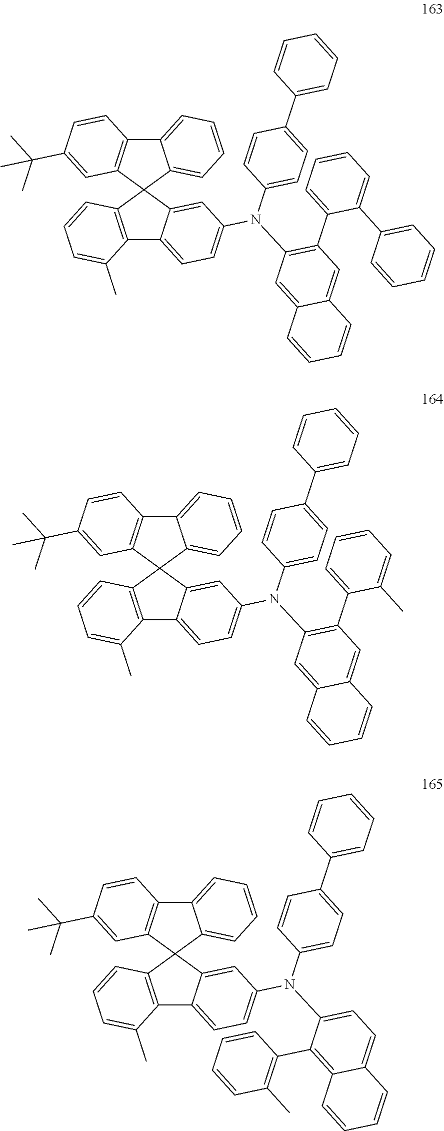 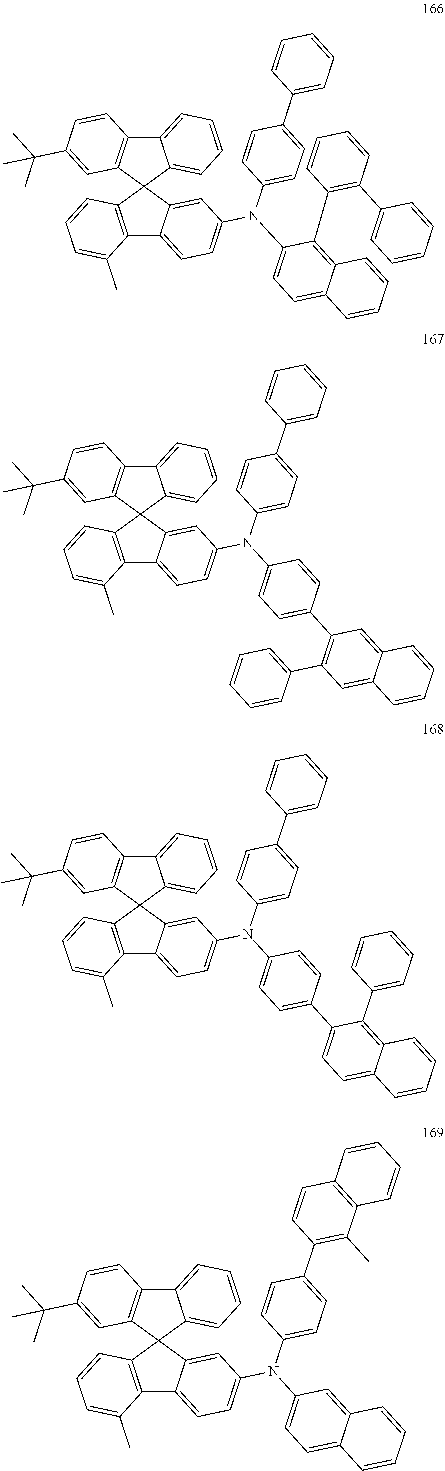 [SMILES: Cc1cccc2c1-c1ccc(N(c3ccc(-c4ccccc4)cc3)c3ccc4ccccc4c3-c3ccccc3-c3ccccc3)cc1C21c2ccccc2-c2ccc(C(C)(C)C)cc21 ; Cc1cccc2c1-c1ccc(N(c3ccc(-c4ccccc4)cc3)c3ccc(-c4cc5ccccc5cc4-c4ccccc4)cc3)cc1C21c2ccccc2-c2ccc(C(C)(C)C)cc21 ; Cc1cccc2c1-c1ccc(N(c3ccc(-c4ccccc4)cc3)c3ccc(-c4ccc5ccccc5c4-c4ccccc4)cc3)cc1C21c2ccccc2-c2ccc(C(C)(C)C)cc21 ; Cc1cccc2c1-c1ccc(N(c3ccc(-c4ccc5ccccc5c4C)cc3)c3ccc4ccccc4c3)cc1C21c2ccccc2-c2ccc(C(C)(C)C)cc21] 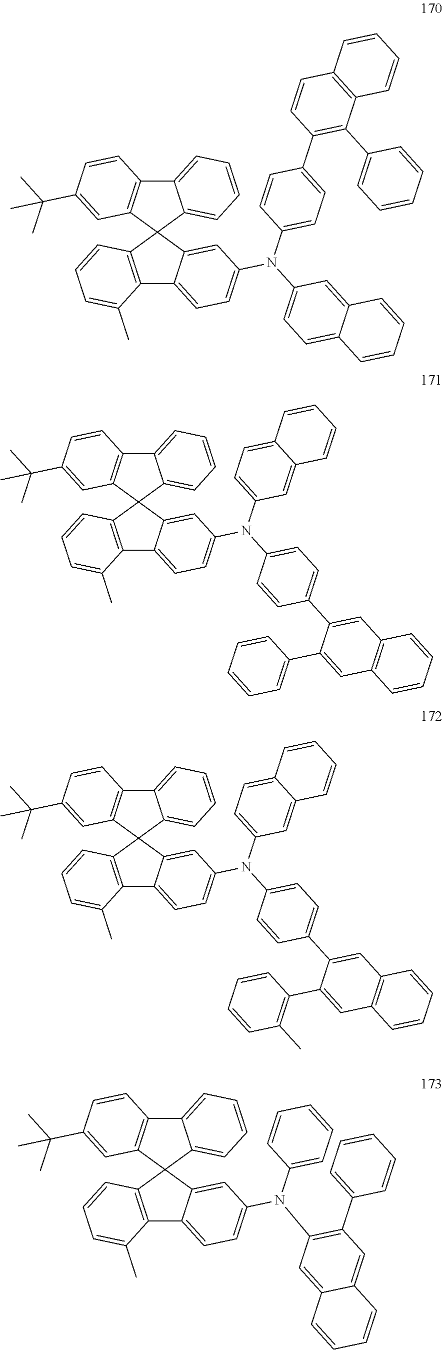 [SMILES: Cc1cccc2c1-c1ccc(N(c3ccc(-c4ccc5ccccc5c4-c4ccccc4)cc3)c3ccc4ccccc4c3)cc1C21c2ccccc2-c2ccc(C(C)(C)C)cc21 ; Cc1cccc2c1-c1ccc(N(c3ccc(-c4cc5ccccc5cc4-c4ccccc4)cc3)c3ccc4ccccc4c3)cc1C21c2ccccc2-c2ccc(C(C)(C)C)cc21 ; Cc1ccccc1-c1cc2ccccc2cc1-c1ccc(N(c2ccc3c(c2)C2(c4ccccc4-c4ccc(C(C)(C)C)cc42)c2cccc(C)c2-3)c2ccc3ccccc3c2)cc1 ; Cc1cccc2c1-c1ccc(N(c3ccccc3)c3cc4ccccc4cc3-c3ccccc3)cc1C21c2ccccc2-c2ccc(C(C)(C)C)cc21] 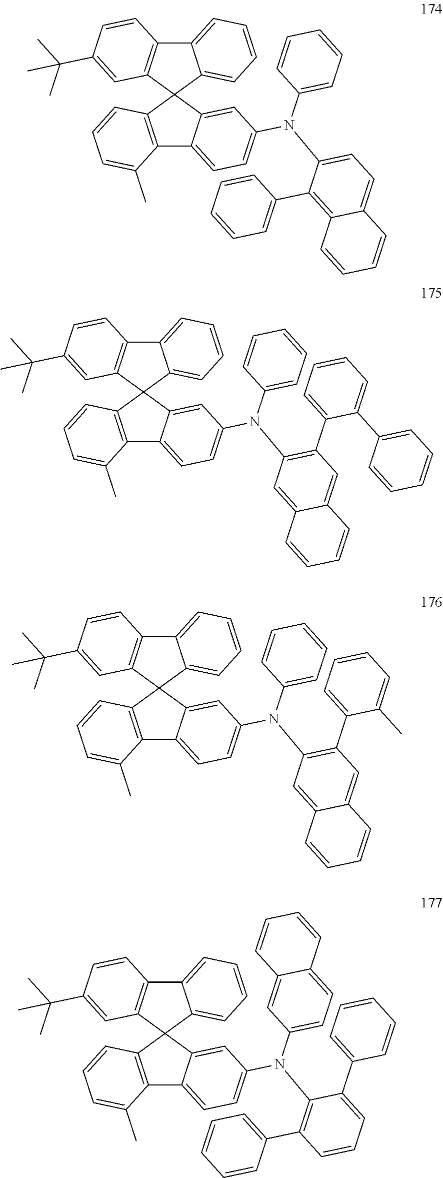 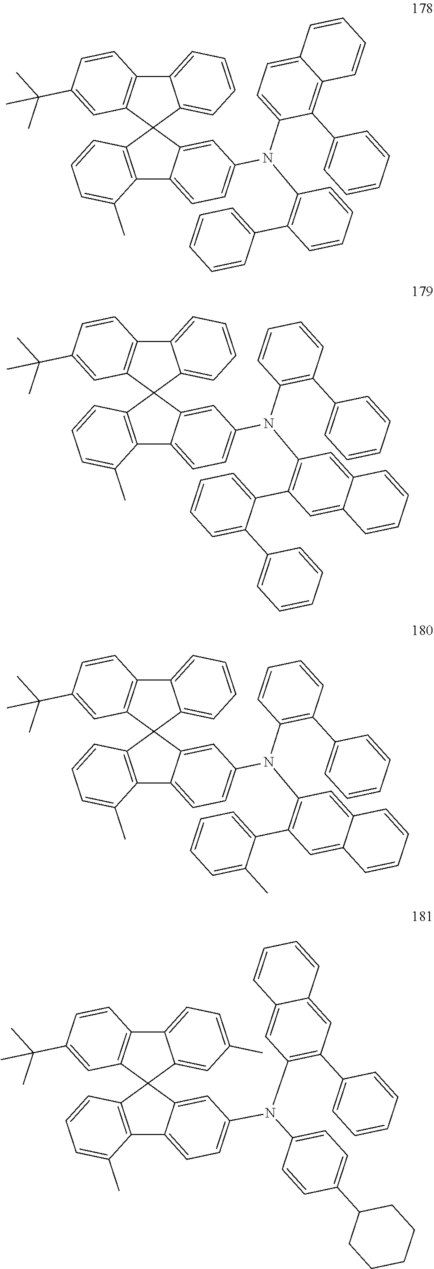 [SMILES: Cc1cccc2c1-c1ccc(N(c3ccccc3-c3ccccc3)c3ccc4ccccc4c3-c3ccccc3)cc1C21c2ccccc2-c2ccc(C(C)(C)C)cc21 ; Cc1cccc2c1-c1ccc(N(c3ccccc3-c3ccccc3)c3cc4ccccc4cc3-c3ccccc3-c3ccccc3)cc1C21c2ccccc2-c2ccc(C(C)(C)C)cc21 ; Cc1ccccc1-c1cc2ccccc2cc1N(c1ccc2c(c1)C1(c3ccccc3-c3ccc(C(C)(C)C)cc31)c1cccc(C)c1-2)c1ccccc1-c1ccccc1 ; Cc1ccc2c(c1)C1(c3cc(C(C)(C)C)ccc3-2)c2cc(N(c3ccc(C4CCCCC4)cc3)c3cc4ccccc4cc3-c3ccccc3)ccc2-c2c(C)cccc21] 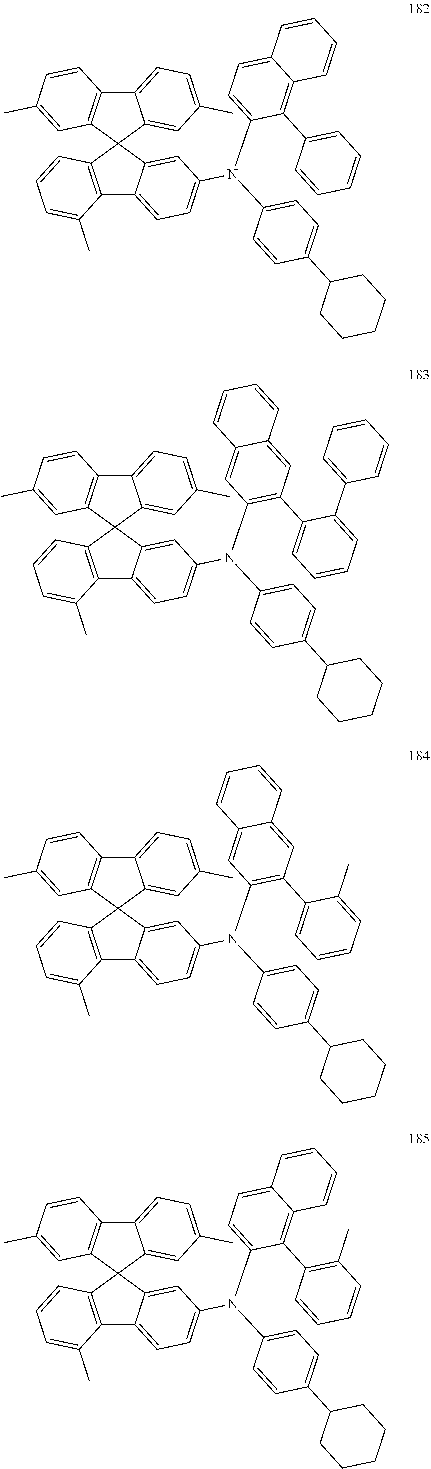 [SMILES: Cc1ccc2c(c1)C1(c3cc(C)ccc3-2)c2cc(N(c3ccc(C4CCCCC4)cc3)c3ccc4ccccc4c3-c3ccccc3)ccc2-c2c(C)cccc21 ; Cc1ccc2c(c1)C1(c3cc(C)ccc3-2)c2cc(N(c3ccc(C4CCCCC4)cc3)c3cc4ccccc4cc3-c3ccccc3-c3ccccc3)ccc2-c2c(C)cccc21 ; Cc1ccc2c(c1)C1(c3cc(C)ccc3-2)c2cc(N(c3ccc(C4CCCCC4)cc3)c3cc4ccccc4cc3-c3ccccc3C)ccc2-c2c(C)cccc21 ; Cc1ccc2c(c1)C1(c3cc(C)ccc3-2)c2cc(N(c3ccc(C4CCCCC4)cc3)c3ccc4ccccc4c3-c3ccccc3C)ccc2-c2c(C)cccc21] 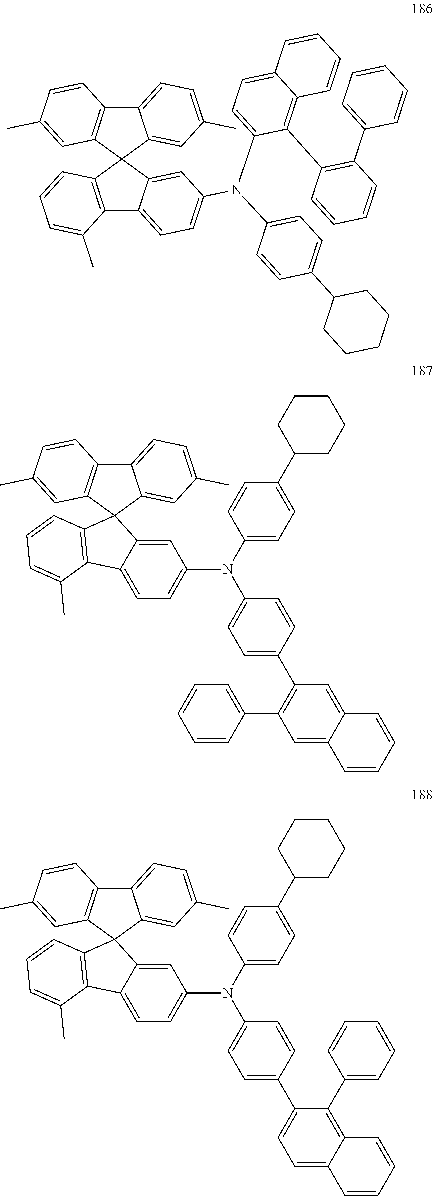 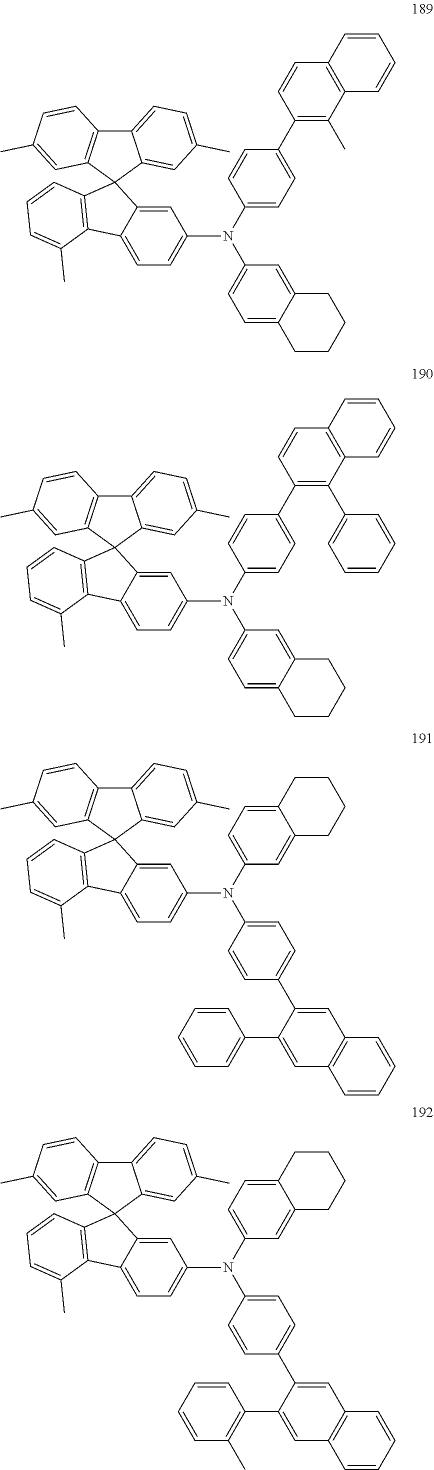 [SMILES: Cc1ccc2c(c1)C1(c3cc(C)ccc3-2)c2cc(N(c3ccc(-c4ccc5ccccc5c4C)cc3)c3ccc4c(c3)CCCC4)ccc2-c2c(C)cccc21 ; Cc1ccc2c(c1)C1(c3cc(C)ccc3-2)c2cc(N(c3ccc(-c4ccc5ccccc5c4-c4ccccc4)cc3)c3ccc4c(c3)CCCC4)ccc2-c2c(C)cccc21 ; Cc1ccc2c(c1)C1(c3cc(C)ccc3-2)c2cc(N(c3ccc(-c4cc5ccccc5cc4-c4ccccc4)cc3)c3ccc4c(c3)CCCC4)ccc2-c2c(C)cccc21 ; Cc1ccc2c(c1)C1(c3cc(C)ccc3-2)c2cc(N(c3ccc(-c4cc5ccccc5cc4-c4ccccc4C)cc3)c3ccc4c(c3)CCCC4)ccc2-c2c(C)cccc21] 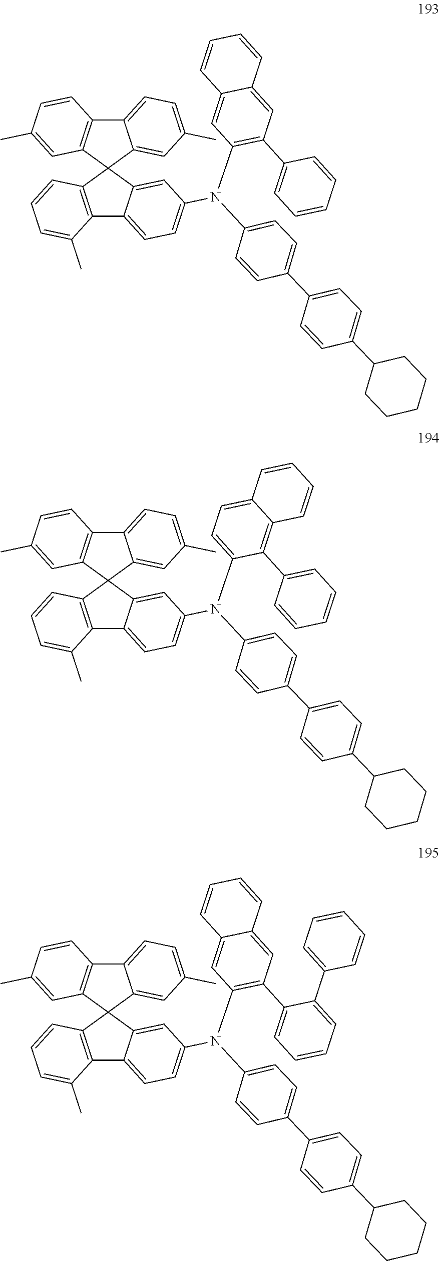 [SMILES: Cc1ccc2c(c1)C1(c3cc(C)ccc3-2)c2cc(N(c3ccc(-c4ccc(C5CCCCC5)cc4)cc3)c3cc4ccccc4cc3-c3ccccc3)ccc2-c2c(C)cccc21 ; Cc1ccc2c(c1)C1(c3cc(C)ccc3-2)c2cc(N(c3ccc(-c4ccc(C5CCCCC5)cc4)cc3)c3ccc4ccccc4c3-c3ccccc3)ccc2-c2c(C)cccc21 ; Cc1ccc2c(c1)C1(c3cc(C)ccc3-2)c2cc(N(c3ccc(-c4ccc(C5CCCCC5)cc4)cc3)c3cc4ccccc4cc3-c3ccccc3-c3ccccc3)ccc2-c2c(C)cccc21] 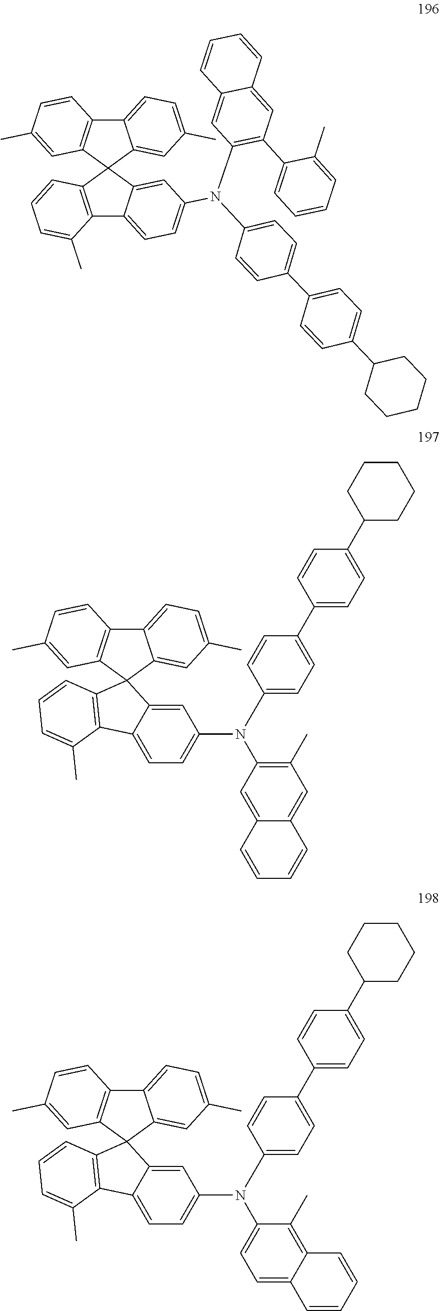 [SMILES: Cc1ccc2c(c1)C1(c3cc(C)ccc3-2)c2cc(N(c3ccc(-c4ccc(C5CCCCC5)cc4)cc3)c3cc4ccccc4cc3-c3ccccc3C)ccc2-c2c(C)cccc21 ; Cc1ccc2c(c1)C1(c3cc(C)ccc3-2)c2cc(N(c3ccc(-c4ccc(C5CCCCC5)cc4)cc3)c3cc4ccccc4cc3C)ccc2-c2c(C)cccc21 ; Cc1ccc2c(c1)C1(c3cc(C)ccc3-2)c2cc(N(c3ccc(-c4ccc(C5CCCCC5)cc4)cc3)c3ccc4ccccc4c3C)ccc2-c2c(C)cccc21] 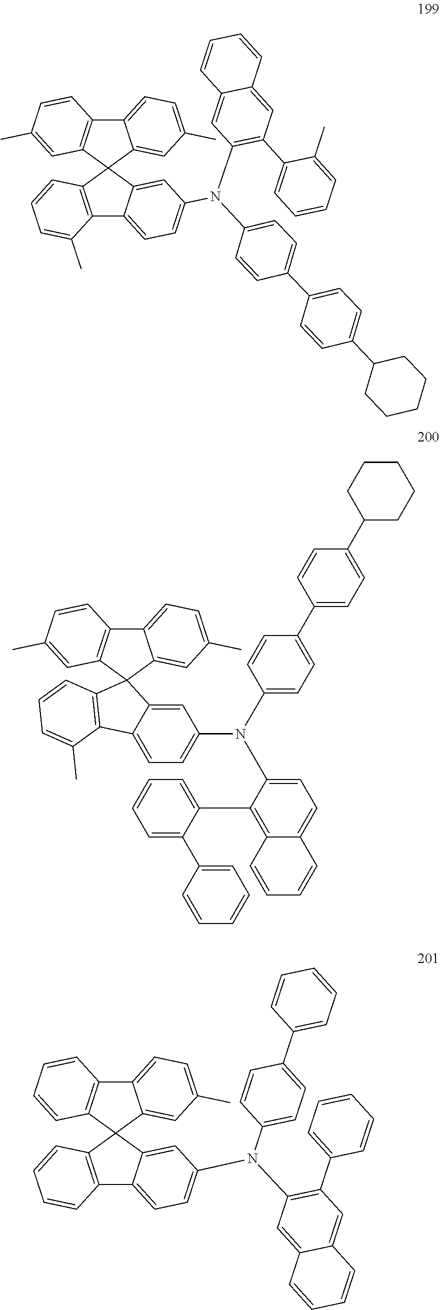 [SMILES: Cc1ccc2c(c1)C1(c3cc(C)ccc3-2)c2cc(N(c3ccc(-c4ccc(C5CCCCC5)cc4)cc3)c3cc4ccccc4cc3-c3ccccc3C)ccc2-c2c(C)cccc21 ; Cc1ccc2c(c1)C1(c3cc(C)ccc3-2)c2cc(N(c3ccc(-c4ccc(C5CCCCC5)cc4)cc3)c3ccc4ccccc4c3-c3ccccc3-c3ccccc3)ccc2-c2c(C)cccc21 ; Cc1ccc2c(c1)C1(c3ccccc3-2)c2ccccc2-c2ccc(N(c3ccc(-c4ccccc4)cc3)c3cc4ccccc4cc3-c3ccccc3)cc21] 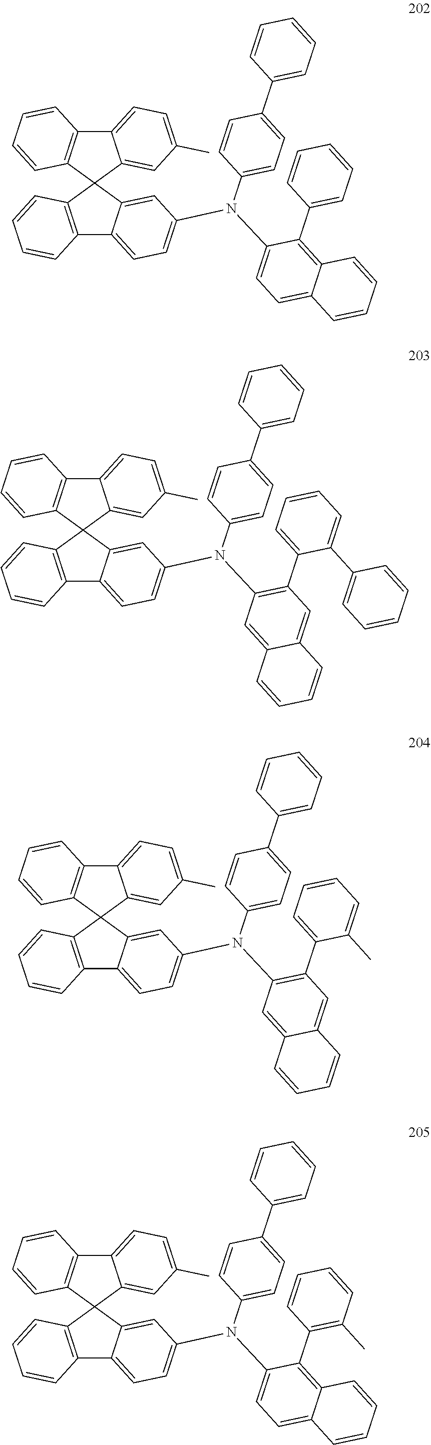 [SMILES: Cc1ccc2c(c1)C1(c3ccccc3-2)c2ccccc2-c2ccc(N(c3ccc(-c4ccccc4)cc3)c3ccc4ccccc4c3-c3ccccc3)cc21 ; Cc1ccc2c(c1)C1(c3ccccc3-2)c2ccccc2-c2ccc(N(c3ccc(-c4ccccc4)cc3)c3cc4ccccc4cc3-c3ccccc3-c3ccccc3)cc21 ; Cc1ccc2c(c1)C1(c3ccccc3-2)c2ccccc2-c2ccc(N(c3ccc(-c4ccccc4)cc3)c3cc4ccccc4cc3-c3ccccc3C)cc21 ; Cc1ccc2c(c1)C1(c3ccccc3-2)c2ccccc2-c2ccc(N(c3ccc(-c4ccccc4)cc3)c3ccc4ccccc4c3-c3ccccc3C)cc21] 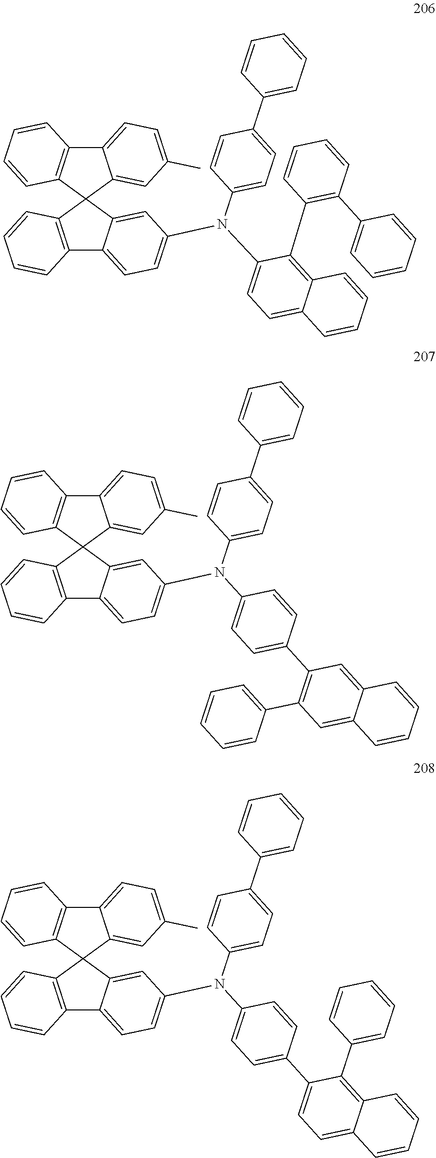 [SMILES: Cc1ccc2c(c1)C1(c3ccccc3-2)c2ccccc2-c2ccc(N(c3ccc(-c4ccccc4)cc3)c3ccc4ccccc4c3-c3ccccc3-c3ccccc3)cc21 ; Cc1ccc2c(c1)C1(c3ccccc3-2)c2ccccc2-c2ccc(N(c3ccc(-c4ccccc4)cc3)c3ccc(-c4cc5ccccc5cc4-c4ccccc4)cc3)cc21 ; Cc1ccc2c(c1)C1(c3ccccc3-2)c2ccccc2-c2ccc(N(c3ccc(-c4ccccc4)cc3)c3ccc(-c4ccc5ccccc5c4-c4ccccc4)cc3)cc21] 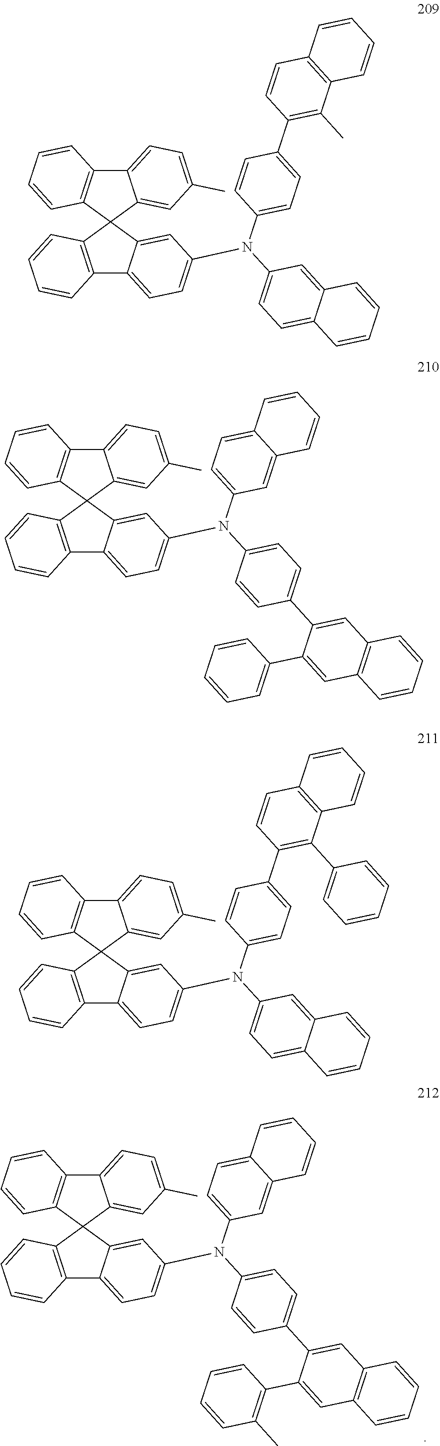 [SMILES: Cc1ccc2c(c1)C1(c3ccccc3-2)c2ccccc2-c2ccc(N(c3ccc(-c4ccc5ccccc5c4C)cc3)c3ccc4ccccc4c3)cc21 ; Cc1ccc2c(c1)C1(c3ccccc3-2)c2ccccc2-c2ccc(N(c3ccc(-c4cc5ccccc5cc4-c4ccccc4)cc3)c3ccc4ccccc4c3)cc21 ; Cc1ccc2c(c1)C1(c3ccccc3-2)c2ccccc2-c2ccc(N(c3ccc(-c4ccc5ccccc5c4-c4ccccc4)cc3)c3ccc4ccccc4c3)cc21 ; Cc1ccc2c(c1)C1(c3ccccc3-2)c2ccccc2-c2ccc(N(c3ccc(-c4cc5ccccc5cc4-c4ccccc4C)cc3)c3ccc4ccccc4c3)cc21]
